(19) United States
(12) Patent Application Publication
(10) Pub. No.: US 2008/0255155 A1
(43) Pub. Date: Oct. 16, 2008

(54) KINASE INHIBITORS AND USES THEREOF

(76) Inventors: Stephane Raeppel, St. Lazare (CA); Oscar Saavedra, Outremont (CA); Stephen Claridge, Montreal (CA); Arkadii Vaisburg, Kirkland (CA); Frederic Gaudette, Laval (CA); Ljubomir Isakovic, Montreal (CA); Robert Deziel, Mount-Royal (CA)

Correspondence Address: KEOWN & ZUCCHERO, LLP, 500 WEST CUMMINGS PARK, SUITE 1200, WOBURN, MA, 01801, US

(21) Appl. No.: 11/874,261

(22) Filed: Oct. 18, 2007

Related U.S. Application Data
(60) Provisional application No. 60/852,455, filed on Oct. 18, 2006.

(51) Int. Cl. A61K 31/506; C07D 239/42; C07D 413/02; A61K 31/4439; C12N 5/00; A61P 35/00; C12N 9/99; C07D 401/02; C07D 211/72

(52) U.S. Cl. 514/256; 544/319; 546/274.4; 546/301; 546/279.1; 546/272.1; 514/343; 514/340; 435/184

(57) ABSTRACT

This invention relates to compounds that inhibit protein tyrosine kinase activity. In particular the invention relates to compounds, compositions and methods for the inhibition of kinase activity. The invention also provides compounds, compositions and methods for treating cell proliferative diseases and conditions.

### RELATED APPLICATIONS

This application claims the benefit of U.S. Provisional Application Ser. No. 60/852,455, filed on Oct. 18, 2006, which is incorporated herein, in its entirety, by reference.

### BACKGROUND OF THE INVENTION

1. Field of the Invention

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling and HGF receptor signaling.

2. Summary of the Related Art

Angiogenesis is an important component of certain normal physiological processes such as embryogenesis and wound healing, but aberrant angiogenesis contributes to some pathological disorders and in particular to tumor growth.^{1,2} VEGF-A (vascular endothelial growth factor A) is a key factor promoting neovascularization (angiogenesis) of tumors.³⁻⁷ VEGF induces endothelial cell proliferation and migration by signaling through two high affinity receptors, the fms-like tyrosine kinase receptor, Flt-1, and the kinase insert domain-containing receptor, KDR.^{8,9,10}. These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain¹¹. The kinase activity autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases 12

Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies^{13,14,15}, receptor antagonists¹⁶, soluble receptors¹⁷, antisense constructs¹⁸ and dominant-negative strategies¹⁹.

Despite the attractiveness of anti-angiogenic therapy by VEGF inhibition alone, several issues may limit this approach. VEGF expression levels can themselves be elevated by numerous diverse stimuli and perhaps most importantly, the hypoxic state of tumors resulting from VEGFr inhibition, can lead to the induction of factors that themselves promote tumor invasion and metastasis thus, potentially undermining the impact of VEGF inhibitors as cancer therapeutics²⁰.

The HGF (hepatocyte growth factor) and the HGF receptor, c-met, are implicated in the ability of tumor cells to undermine the activity of VEGF inhibition²⁰. HGF derived from either stromal fibroblasts surrounding tumor cells or expressed from the tumor itself has been suggested to play a critical role in tumor angiogenesis, invasion and metastasis^{21,22}. For example, invasive growth of certain cancer cells is drastically enhanced by tumor-stromal interactions involving the HGF/c-Met (HGF receptor) pathway^{23,24,25}. HGF, which was originally identified as a potent mitogen for hepatocytes^{26,27} is primarily secreted from stromal cells, and the secreted HGF can promote motility and invasion of various cancer cells that express c-Met in a paracrine manner^{28,29,30}. Binding of HGF to c-Met leads to receptor phosphorylation and activation of Ras/mitogen-activated protein kinase (MAPK) signaling pathway, thereby enhancing malignant behaviors of cancer cells^{30,31}. Moreover, stimulation of the HGF/c-met pathway itself can lead to the induction of VEGF expression, itself contributing directly to angiogenic activity³².

Thus, anti-tumor anti-angiogenic strategies or approaches that target both VEGF/VEGFr signaling and HGF/c-met signaling may circumvent the ability of tumor cells to overcome VEGF inhibition alone and may represent improved cancer therapeutics.

Here we describe small molecules that are potent inhibitors of protein tyrosine kinase activity, such as that of, for example, both the VEGF receptor KDR and the HGF receptor c-met.

### BRIEF SUMMARY OF THE INVENTION

The present invention provides new compounds and methods for treating cell proliferative diseases. The compounds of the invention are inhibitors of protein tyrosine kinase activity. Preferably, the compounds of the invention are dual function inhibitors, capable of inhibiting both VEGF and HGF receptor signaling. Accordingly, the invention provides new inhibitors of protein tyrosine kinase receptor signaling, such as for example, VEGF receptor signaling and HGF receptor signaling, including the VEGF receptor KDR and the HGF receptor c-met.

In a first aspect, the invention provides compounds of formula A, and N-oxides, hydrates, solvates, pharmaceutically acceptable salts, prodrugs and complexes thereof, and racemic and scalemic mixtures, diastereomers and enantiomers thereof, that are useful as kinase inhibitors Because compounds of Formula (A) are useful as kinase inhibitors they are, therefore, useful research tools for the study of the role of kinases in both normal and disease states. Preferrably, the invention provides compounds of Formula (I) that are useful as inhibitors of VEGF receptor signaling and HGF receptor signaling and, therefore, are useful research tools for the study of the role of VEGF and HGF in both normal and disease states.

In a second aspect, the invention provides compositions comprising a compound that is an inhibitor of protein tyrosine kinase, or an N-oxide, hydrate, solvate, pharmaceutically acceptable salt, prodrug or complex thereof, or a racemic or scalemic mixture, diastereomers or enantiomer thereof, and a pharmaceutically acceptable carrier, excipient or diluent. Preferably, the invention provides compositions comprising a compound that is an inhibitor of VEGF receptor signaling and HGF receptor signaling, or a pharmaceutically acceptable salt thereof, and a pharmaceutically acceptable carrier, excipient, or diluent.

In a third aspect, the invention provides a method of inhibiting kinase activity, preferably protein tyrosine kinase, the method comprising contacting the kinase with a compound according to the present invention, or with a composition according to the present invention. Preferably the invention provides a method of inhibiting receptor type tyrosine kinase signaling, preferably inhibiting VEGF receptor signaling and HGF receptor signaling, the method comprising contacting the receptor with a compound according to the present invention, or with a composition according to the present invention. Inhibition can be in a cell or a multicellular organism. If in a cell, the method according to this aspect of the invention comprises contacting the cell with a compound according to the present invention, or with a composition according to the present invention. If in a multicellular organism, the method according to this aspect of the invention comprises administering to the organism a compound according to the present invention, or a composition according to the present invention. Preferably the organism is a mammal, more preferably a human.

The foregoing merely summarizes certain aspects of the invention and is not intended to be limiting in nature. These aspects and other aspects and embodiments are described more fully below.

### DETAILED DESCRIPTION OF THE PREFERRED EMBODIMENTS

The invention provides compounds and methods for inhibiting kinase activity, preferably protein tyrosine kinase activity, preferably receptor protein kinase activity, preferably the VEGF receptor KDR and the HGF receptor c-met. The invention also provides compositions and methods for treating cell proliferative diseases and conditions. The patent and scientific literature referred to herein reflects knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

For purposes of the present invention, the following definitions will be used (unless expressly stated otherwise):

Reference to “a compound of the formula (I), formula (II), etc.,” (or equivalently, “a compound according to the first aspect”, or “a compound of the present invention”, and the like), herein is understood to include reference to N-oxides, hydrates, solvates, pharmaceutically acceptable salts, prodrugs and complexes thereof, and racemic and scalemic mixtures, diastereomers, enantiomers and tautomers thereof, unless otherwise indicated.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—. Also, a number of moieties disclosed herein exist in multiple tautomeric forms, all of which are intended to be encompassed by any given tautomeric structure.

The term “hydrocarbyl” as employed herein refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” as employed herein refers to straight and branched chain aliphatic groups having from 1 to 12 carbon atoms, preferably 1-8 carbon atoms, and more preferably 1-6 carbon atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond (like “C₀” hydrocarbyl).

The term “alkenyl” as employed herein means an unsaturated straight or branched chain aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” as employed herein means an unsaturated straight or branched chain aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “carbocycle” as employed herein is intended to mean a cycloalkyl or aryl moiety. The term “carbocycle” also includes a cycloalkenyl moiety having at least one carbon-carbon double bond.

The term “cycloalkyl” as employed herein includes saturated and partially unsaturated cyclic hydrocarbon groups having 3 to 12 carbons, preferably 3 to 8 carbons, more preferably 3 to 6 carbons and more preferably still 5 or 6 carbons. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” as employed herein refers to a hydrocarbyl group, as defined hereinabove, wherein one or more carbon atoms in the group are independently replaced by a heteroatom selected from the group consisting of O, S and N. In some preferred embodiments the one or more carbon atoms are independently replaced by an atom or moiety selected from the group consisting of O, S, NH, N-alkyl, SO, SO₂, SO₂NH, or NHSO₂.

An “aryl” group is a C₅-C₁₄ aromatic moiety comprising one to three aromatic rings. Preferably, the aryl group is a C₆-C₁₄ aryl group, more preferably a C₆-C₁₀ aryl group, and more preferably a C₆ aryl group. In certain preferred embodiments, the aryl group is preferably a C₅ aryl group. Preferred aryl groups include, without limitation, phenyl, naphthyl, anthracenyl, and fluorenyl. An “aralkyl” or “arylalkyl” group comprises an aryl group covalently linked to an alkyl group. Preferably, the aralkyl group is (C₁-C₆)alk(C₆-C₁₀)aryl, including, without limitation, benzyl, phenethyl, and naphthylmethyl. In such types of groups, if the group is stated as being optionally substituted, either or both the aryl and the corresponding alkyl radical portion of an aralkyl group may be substituted. A “lower arylalkyl” refers to an arylalkyl where the “alkyl” portion of the group has one to six carbons. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic, in which case the group is also known as a heterocycloalkyl. In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 π-electrons shared in a cyclic array; and having, in addition to carbon atoms, from one to three heteroatoms per ring independently selected from the group consisting of N, O, and S. The term “heteroaryl” is also meant to encompass monocyclic, bicyclic and polycyclic groups. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl. A “heteroaralkyl” or “heteroarylalkyl” group comprises a heteroaryl group covalently linked to an alkyl group. In such types of groups, if the group is stated as being optionally substituted, either or both the heteroaryl and the corresponding alkyl radical portion of a heteroarylalkyl group may be substituted. Preferred heteroaralkyl groups comprise a C₁-C₆ alkyl group and a heteroaryl group having 5, 6, 9, or 10 ring atoms. Examples of preferred heteroaralkyl groups include pyridylmethyl, pyridylethyl, pyrrolylmethyl, pyrrolylethyl, imidazolylmethyl, imidazolylethyl, thiazolylmethyl, and thiazolylethyl. Specifically excluded from the scope of this term are compounds having adjacent annular O and/or S atoms.

For simplicity, reference to a “Cₙ-Cₘ” heterocyclyl or heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetaryl includes, for example, pyridyl and pyrimidyl.

An “arylene,” “heteroarylene,” or “heterocyclylene” group is an aryl, heteroaryl, or heterocyclyl group, as defined hereinabove, that is positioned between and serves to connect two other chemical groups.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Preferred azolyl groups include, but are not limited to, imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

A heteroalicyclic group refers specifically to a non-aromatic heterocyclyl radical. A heteroalicyclic may contain unsaturation, but is not aromatic.

A heterocyclylalkyl group refers to a residue in which a heterocyclyl is attached to a parent structure via one of an alkylene, alkylidene, or alkylidyne radical. Examples include (4-methylpiperazin-1-yl)methyl, (morpholin-4-yl)methyl, (pyridine-4-yl)methyl, 2-(oxazolin-2-yl)ethyl, 4-(4-methylpiperazin-1-yl)-2-butenyl, and the like. In such types of groups, if the group is stated as being optionally substituted, either or both the heterocyclyl and the corresponding alkylene, alkylidene, or alkylidyne radical portion of a heterocyclylalkyl group may be substituted. A “lower heterocyclylalkyl” refers to a heterocyclylalkyl where the “alkyl” portion of the group has one to six carbons.

A heteroalicyclylalkyl group refers specifically to a heterocyclylalkyl where the heterocyclyl portion of the group is non-aromatic.

Preferred heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as furo[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholinyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

A “monocycle” or “monocyclic moiety” is a single ring structure, which may be a saturated or unsaturated cycloalkyl or heterocycloalkyl group, or an aryl, or heteroaryl group, as further described herein.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, independently selected non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

Preferred substituents, which are themselves not further substituted (unless expressly stated otherwise) are:
a) halo, cyano, oxo, carboxy, formyl, nitro, amino, amidino, guanidino,
b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₁-C₈alkenyl, C₁-C₈alkoxy, C₁-C₈alkylamino, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
c) —(CR³²R³³)—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, C₁-C₈alkyl, C₁-C₈heteroalkyl, C₁-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰-Y³¹-), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —CO—C₃alkyl-C₂-C₈alkenyl-CO—C₃alkyl, C₀-C₃alkyl-C₂-C₈alkynyl-C₀-C₃alkyl, CO—C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—CO—C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (preferably one to four, preferably from one to three and more preferably one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH2—) substituted with oxygen to form carbonyl —CO—

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, a hydrocarbyl, heteroalkyl, heterocyclic and/or aryl group is unsubstituted.

In other preferred embodiments, a hydrocarbyl, heteroalkyl, heterocyclic and/or aryl group is substituted with from 1 to 3 independently selected substituents.

Preferred substituents on alkyl groups include, but are not limited to, hydroxyl, halogen (e.g., a single halogen substituent or multiple halo substituents; in the latter case, groups such as CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(O)₂R^{e}, S(═O)₂OR^{e}, P(═O)₂OR^{e}, —NR^{b}R^{c}, —NR^{b}S(═O)₂R^{e}, —NR^{b}P(═O)₂R^{e}, —S(═O)NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited above as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cylcoalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cyclic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In certain preferred embodiments, a heterocyclic group is substituted on carbon, nitrogen and/or sulfur at one or more positions. Preferred substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on ring groups, such as aryl, heteroaryl, cycloalkyl and heterocyclyl, include halogen, alkoxy and alkyl.

Especially preferred substituents on alkyl groups include halogen and hydroxy.

A “halohydrocarbyl” as employed herein is a hydrocarbyl moiety, in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

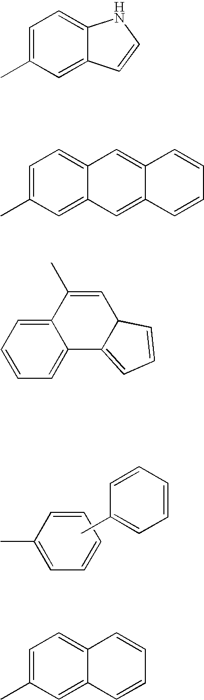

An “unsubstituted” moiety (e.g., unsubstituted cycloalkyl, unsubstituted heteroaryl, etc.) means a moiety as defined above that does not have any optional substituents.

A saturated or unsaturated three- to eight-membered carbocyclic ring is preferably a four- to seven-membered, more preferably five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated three- to eight-membered heterocyclic ring contains at least one heteroatom selected from oxygen, nitrogen, and sulfur atoms. The saturated or unsaturated three- to eight-membered heterocyclic ring preferably contains one or two heteroatoms with the remaining ring-constituting atoms being carbon atoms. The saturated or unsaturated three- to eight-membered heterocyclic ring is preferably a saturated or unsaturated four- to seven-membered heterocyclic ring, more preferably a saturated or unsaturated five- or six-membered heterocyclic ring. Examples of saturated or unsaturated three- to eight-membered heterocyclic groups include thienyl, pyridyl, 1,2,3-triazolyl, imidazolyl, isoxazolyl, pyrazolyl, piperazinyl, piperazino, piperidyl, piperidino, morpholinyl, morpholino, homopiperazinyl, homopiperazino, thiomorpholinyl, thiomorpholino, tetrahydropyrrolyl, and azepanyl.

A saturated or unsaturated carboxylic and heterocyclic group may condense with another saturated or heterocyclic group to form a bicyclic group, preferably a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁₋₆ alkyl groups, the two alkyl groups may combine together to form an alkylene chain, preferably a C₁₋₃ alkylene chain. Carbocyclic or heterocyclic groups having this crosslinked structure include bicyclo[2.2.2]octanyl and norbornanyl.

The terms “kinase inhibitor” and “inhibitor of kinase activity”, and the like, are used to identify a compound which is capable of interacting with a kinase and inhibiting its enzymatic activity.

The term “inhibiting kinase enzymatic activity” is used to mean reducing the ability of a kinase to transfer a phosphate group from a donor molecule, such as ATP, to a specific target molecule (substrate). For example, the inhibition of kinase activity may be at least about 10%. In some preferred embodiments of the invention, such reduction of kinase activity is at least about 50%, more preferably at least about 75%, and still more preferably at least about 90%. In other preferred embodiments, kinase activity is reduced by at least 95% and even more preferably by at least 99%. The IC₅₀ value is the concentration of kinase inhibitor which reduces the activity of a kinase to 50% of the uninhibited enzyme.

The terms “inhibitor of VEGF receptor signaling” and “inhibitor of HGF receptor signaling” are used to identify a compound having a structure as defined herein, which is capable, respectively, of interacting with a VEGF receptor and a HGF receptor and inhibiting the activity of the VEGF receptor and the HGF receptor. In some preferred embodiments, such reduction of activity is at least about 50%, more preferably at least about 75%, and still more preferably at least about 90%. In other preferred embodiments, activity is reduced by at least 95% and even more preferably by at least 99%.

The term “inhibiting effective amount” is meant to denote a dosage sufficient to cause inhibition of kinase activity. The kinase may be in a cell, which in turn may be in a multicellular organism. The multicellular organism may be, for example, a plant, a fungus or an animal, preferably a mammal and more preferably a human. The fungus may be infecting a plant or a mammal, preferably a human, and could therefore be located in and/or on the plant or mammal. If the kinase is in a multicellular organism, the method according to this aspect of the invention comprises the step of administering to the organism a compound or composition according to the present invention. Administration may be by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other preferred embodiments, administration may preferably be by the oral route.

Preferably, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. Preferably, the concentration of the inhibitor required for kinase inhibitory activity is at least 2-fold lower, more preferably at least 5-fold lower, even more preferably at least 10-fold lower, and most preferably at least 20-fold lower than the concentration required to produce an unrelated biological effect.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. Further, the therapeutic effect can be inhibition of kinase activity. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

The term “patient” as employed herein for the purposes of the present invention includes humans and other animals, particularly mammals, and other organisms. Thus the compounds, compositions and methods of the present invention are applicable to both human therapy and veterinary applications. In a preferred embodiment the patient is a mammal, and in a most preferred embodiment the patient is human.

The terms “treating”, “treatment”, or the like, as used herein covers the treatment of a disease-state in an animal and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment of the present invention the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The compounds of the present invention form salts which are also within the scope of this invention. Reference to a compound of the invention, for example a compound of Formula (I), herein is understood to include reference to salts thereof, unless otherwise indicated.

The term “salt(s)”, as employed herein, denotes acidic and/or basic salts formed with inorganic and/or organic acids and bases. In addition, when a compound of Formula (I) contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic (exhibiting minimal or no undesired toxicological effects), physiologically acceptable) salts are preferred, although other salts are also useful, e.g., in isolation or purification steps which may be employed during preparation. Salts of the compounds of the invention may be formed, for example, by reacting a compound of the present invention with an amount of acid or base, such as an equivalent amount, in a medium such as one in which the salts precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfanotes (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl and butyl chlorides, bromides and iodides), dialkyl sulfates (e.g. dimethyl, diethyl, dibuty and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable salts” is intended to mean salts that retain the desired biological activity of the above-identified compounds and exhibit minimal or no undesired toxicological effects.

Another aspect of the invention provides compositions including a compound, N-oxide, hydrate, solvate, pharmaceutically acceptable salt, complex or prodrug of a compound according to the present invention as described herein, or a racemic mixture, diastereomer, enantiomer or tautomer thereof. For example, in one embodiment of the invention, a composition comprises a compound, N-oxide, hydrate, solvate, pharmaceutically acceptable salt, complex or prodrug of a compound according to the present invention as described herein present in at least about 30% enantiomeric or diastereomeric excess. In certain desirable embodiments of the invention, the compound, N-oxide, hydrates, solvate, pharmaceutically acceptable salt, complex or prodrug is present in at least about 50%, at least about 80%, or even at least about 90% enantiomeric or diastereomeric excess. In certain other desirable embodiments of the invention, the compound, N-oxide, hydrate, solvate, pharmaceutically acceptable salt, complex or prodrug is present in at least about 95%, more preferably at least about 98% and even more preferably at least about 99% enantiomeric or diastereomeric excess. In other embodiments of the invention, a compound, N-oxide, hydrate, solvate, pharmaceutically acceptable salt, complex or prodrug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of Formula (I) in at least about 30% diastereomeric or enantiomeric excess. In certain embodiments of the invention, the compound is present in at least about 50% enantiomeric or diastereomeric excess, in at least about 80% enantiomeric or diastereomeric excess, or even in at least about 90% enantiomeric or diastereomeric excess. In certain more preferred embodiments of the invention, the compound is present in at least about 95%, even more preferably in at least about 98% enantiomeric or diastereomeric excess, and most preferably in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The present invention also includes prodrugs of compounds of the invention. The term “prodrug” is intended to represent covalently bonded carriers, which are capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like.

The compounds of the invention may be administered as is or as a prodrug, for example in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁-C₆alkoxymethyl esters (e.g., methoxymethyl), C₁-C₆alkanoyloxymethyl esters (e.g., for example pivaloyloxymethyl), phthalidyl esters, C₃-C₈cycloalkoxycarbonyloxy-C₁-C₆alkyl esters (e.g., 1-cyclohexylcarbonyloxyethyl); 1,3-dioxolen-2-onylmethyl esters (e.g., 5-methyl-1,3-dioxolen-2-onylmethyl; and C₁-C₆alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention, or a salt and/or solvate thereof. Solvates of the compounds of the present invention include, for example, hydrates.

Throughout the specification, preferred embodiments of one or more chemical substituents are identified. Also preferred are combinations of preferred embodiments. For example, preferred embodiments of R⁷ in the compounds of the present invention and preferred embodiments of G in the compounds of the present invention are disclosed. Thus, also contemplated as within the scope of the invention are compounds having both preferred R⁷ and preferred G as are described in the specification.

### Compounds

In the first aspect, the invention comprises compounds of formula (A):

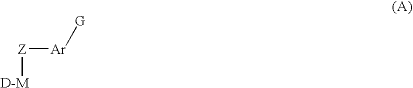

and N-oxides, hydrates, solvates, pharmaceutically acceptable salts, prodrugs and complexes thereof, and racemic and scalemic mixtures, diastereomers and enantiomers thereof, wherein,
M is an optionally substituted monocyclic moiety;
D is selected from the group consisting of R⁷, R¹ and R²¹, wherein
R⁷ is selected from the group consisting of —H, halogen, nitro, azido, C₁-C₆ alkyl, C₃-C₁₀ cycloalkyl, —C(O)NR⁴²R⁴³, —Y—NR⁴²R⁴³, NR⁴²C(═O)R⁴³, —NR⁴²C(═O)NR⁴³, NR⁴²C(═O)NR⁴³—R¹⁰¹, —SO₂R⁴², —SO₂NR⁴²R⁴³, —NR³⁷SO₂R⁴², —NR³⁷SO₂NR⁴²R⁴³, —C(═N—OR⁴²)R⁴³, —C(═NR⁴²)R⁴³, —NR³⁷C(═NR⁴²)R⁴³, C(═NR⁴²)NR³⁷R⁴³, NR³⁷C(═NR⁴²)NR³⁷R⁴³, —C(O)R⁴², —CO₂R⁴², —C(O)(heterocyclyl), —C(O)(C₆-C₁₀ aryl), —C(O)(heteroaryl), —Y—(C₆-C₁₀ aryl), —Y-(heteroaryl), —Y-(5-10 membered heterocyclyl), —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, —S(O)R^{6a}, —SO₂R^{6a}, —SO₃R^{6a}, —SO₂N^{6a}R^{6b}, —SO₂NR⁴²R⁴³, COR^{6a}, —CO₂R^{6a}, —CONR^{6a}R^{6b}, —(C₁-C₄)fluoroalkyl, —(C₁-C₄)fluoroalkoxy, —(CZ³Z⁴)ₙCN, wherein n is an integer ranging from 0 to 6, and the aforementioned R⁷ groups other than —H and halogen are optionally substituted by 1 to 5 R³⁸, or R⁷ is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶))ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O)NHZ⁷, —OC(O)NZ⁷Z⁸, —NHC(O)Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(OH)₃, —OP(OH)₃, —P(O)(OH)₂, OP(OZ⁷)₃, —S(O)₃H, —S(O)Z⁷, —S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, (CZ⁹Z¹⁰)ᵣNZ⁷Z⁸, —X⁶(CZ⁹Z¹⁰)ᵣ-(C₃-C₈)cycloalkyl, —X⁶(CZ⁹Z¹⁰)ᵣ—(C₅-C₈)cycloalkenyl, —X⁶(CZ⁹Z¹⁰)ᵣ-aryl and —X⁶(CZ⁹Z¹⁰)ᵣ-heterocycle, wherein
r is 1, 2, 3 or 4; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, halogen (preferably F), a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl];
R¹ is —C≡CH or —C≡C—(CR⁴⁵R⁴)ₙ—R⁴⁶;
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙC(O)R⁴⁹—C(O)₂R⁴⁹, —(CH₂)ₙSR⁴⁹, —(CH₂)ₙS(O)R⁴⁹, —(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring;
n is an integer ranging from 0 to 6; and
R²¹ is the group defined by -(Z¹¹)-(Z¹²)ₘ-(Z¹³)ₘ₁, wherein
Z¹¹ is heterocyclyl, when m and m1 are 0, or heterocyclylene, when either m or m1 are 1,
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³ (C₁-C₃)alkyl, —(CH₂)_{q}OR⁵³, —C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³;
R⁵⁶ is selected from the group consisting of NH₂, (C₁-C₃)alkyl and OR⁵²;
Ar is a 5 to 7 membered cycloalkyl, aryl, heterocylic or heteroaryl ring system, any of which is optionally substituted with 0 to 4 R² groups;
R² at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³—N(R³)SO₂R³, —N(R³)C(O)R³), —N(R³)CO₂R³, —C(O)R³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
G is a group B-L-T, wherein
   B is selected from the group consisting of absent, —CH₂—NH—, —NH—CH₂—, —N(R¹³)—, —N(SO₂R¹³)—, —O—, —S(O)₀₋₂ and —C(═X)—;
   L is selected from the group consisting of absent, —C(═X)N(R¹³)—, —SO₂N(R¹³)—, —SO₂—, —N(R¹³)—, —C(═X)C₁₋₂alkyl-N(R¹³)—, —N(R¹³)C₁₋₂alkyl-C(═X)—, —C(═X)C₀₋₁alkyl-C(═X)N(R¹³)—, —C₀₋₄alkylene, —C(═X)C₀₋₁alkyl-C(═X)O—, —C(═X)—C₀₋₁alkyl-C(═X)—, —C(═X)—, —C(═X)C₀₋₁alkyl-C(═X)—, —C(═X)—O—C(═X)— and an optionally substituted four to nine-membered heterocyclyl preferably containing between one and three annular heteroatoms and preferably including at least one nitrogen, and wherein an alkyl group of the aforementioned L group is optionally substituted; and
   T is selected from the group consisting of —H, —R¹³, —C₀₋₅alkyl, —C₀₋₅alkyl-Q, —O—C₀₋₅alkyl-Q, —C₀₋₅alkyl-O-Q, —N(R¹³)—C₀₋₅alkyl-Q, —C₀₋₅alkyl-SO₂—C₀₋₅alkyl-Q, —C(═X)—C₀₋₅alkyl-Q, —C(═X)—C₀₋₅-alkyl-Q, —C(X)—C₀₋₅-alkyl-Q, —C₀₋₅alkyl-N(R¹³)-Q, —C(X)—N(R¹³)—C₀₋₅alkyl-Q, —C(X)—N(R¹³)—C₀₋₅alkyl-Q, —C(X)—N(R¹³)—C₀₋₅alkyl-Q, —(C₀₋₅alkyl-C(O))₀₋₁—C₀₋₅alkyl-Q wherein each C₀₋₅alkyl is optionally substituted;
   wherein X is selected from the group consisting of O, S, NH, N-alkyl, N—OH, N—O-alkyl, and NCN;
   or G is selected from the group consisting of

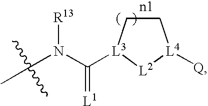

wherein
L¹ is selected from the group consisting of O, S and N(R¹⁴);
L² is selected from the group consisting of —C(O)—, —C(S)—, —C(NH)—, >C═N(C₁-C₆ alkyl) and —CH₂—;
L³ is selected from the group consisting of —CH—, —C(C₁-C₆ alkyl)- and N;
L⁴ is selected from the group consisting of —CH— and N; and
n1 is an integer from 0 to 5;

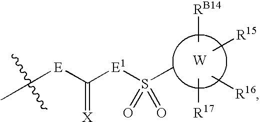

wherein
E is selected from the group consisting of —N(H)—, —N(C₁-C₆alkyl)-, —CH₂N(H)— and —N(H)CH₂—,
X is selected from the group consisting of O, S, NH, N-alkyl, N—OH, N—O-alkyl, and NCN,
E¹ is selected from the group consisting of —N(H)—, —N(C₁-C₆alkyl)-, —CH₂N(H)— and —N(H)CH₂—,
W is a five- to ten-membered cycloalkyl, aryl, heterocylic or heteroaryl ring system, which is optionally substituted, and
R^{B14}, R¹⁵, R¹⁶ and R¹⁷ are independently selected from the group consisting of R²⁰;

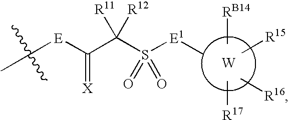

wherein
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl, or
R¹¹ and R¹² taken together with the atom to which they are attached form a C₃-C₇ ring system, wherein said ring system is optionally substituted;

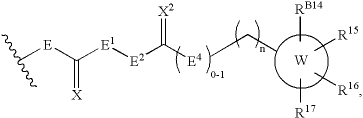

wherein
n is 0, 1, 2, 3 or 4;
X² is selected from the group consisting of O, S, NH, NOH, NOMe, NOEt and NCN,
E² is selected from the group consisting of —N(H)—, —N(C₁-C₆alkyl)-, —CH₂N(H)— and —N(H)CH₂—, and
E⁴ is —N(H)— or —N(C₁-C₆alkyl)-; and

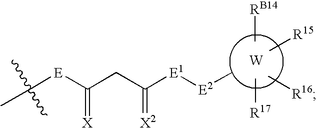

R¹³ is selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³—N(R³)C(O)R³, —N(R³)CO₂R³, —C(O)R³, —C(O)SR³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), —(CH₂)₀₋₅(cycloalkyl), C₁-C₆ alkyl, C₂-C₆ alkenyl, C₂-C₆ alkynyl, —CH₂(CH₂)₀₋₄-T², an optionally substituted C₁₋₄ alkylcarbonyl, and a saturated or unsaturated three- to seven-membered carboxyclic or heterocyclic group, wherein the aryl, heteroaryl, C₁-C₆ alkyl, C₂-C₆ alkenyl, and C₂-C₆ alkynyl are optionally substituted; wherein
two R¹³, together with the atom or atoms to which they are attached, can combine to form a heteroalicyclic optionally substituted with between one and four of R⁶⁰, wherein the heteroalicyclic can have up to four annular heteroatoms, and the heteroalicyclic can have an aryl or heteroaryl fused thereto, in which case the aryl or heteroaryl is optionally substituted with an additional one to four of R⁶⁰;
R¹⁴ is selected from the group —H, —NO₂, —NH₂, —N(R³)R⁴, —CN, —OR³, an optionally substituted (C₁-C₆)alkyl, an optionally substituted heteroalicyclylalkyl, an optionally substituted aryl, an optionally substituted arylalkyl and an optionally substituted heteroalicyclic,
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
R⁶⁰ is selected from the group consisting of —H, halogen, trihalomethyl, —CN, —NO₂, —NH₂, —OR³, —NR³R⁴, —S(O)₀₋₂R³, —SO₂NR³R³, —CO₂R³, —C(O)NR³R³, —N(R³)SO₂R³, —N(R³)C(O)R³, —N(R³)CO₂R³, —C(O)R³, an optionally substituted (C₁-C₆)alkyl, an optionally substituted aryl, an optionally substituted heteroarylalkyl and an optionally substituted arylalkyl; or
two R⁶⁰, when attached to a non-aromatic carbon, can be oxo;
Q is a five- to ten-membered ring system, optionally substituted with between zero and four of R²⁰;
R²⁰ is selected from the group consisting of —H, halogen, trihalomethyl, —O-trihalomethyl, oxo, —CN, —NO₂, —NH₂, —P(═O)(C₁-C₆alkyl)₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)O R³, —C(O)R³, —C(O)SR³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carboxyclic or heterocyclic group and wherein the aryl, heteroaryl, C₁-C₆ alkyl, C₂-C₆ alkenyl, and C₂-C₆ alkynyl are optionally substituted;
each R³⁸ is independently selected from halo, cyano, nitro, trifluoromethoxy, trifluoromethyl, azido, optionally substituted C₁-C₆ alkyl, —C(O)O—(CH₂)ₙNR³⁶R³⁹, —C(O)(CH₂)ⱼNR³⁹(CH₂)ₙR³⁶, —(CH₂)ₙP(═O)(C₁-C₆alkyl)₂, —(CH₂)ⱼNR³⁹CH₂(CH₂)ₙP(═O)(C₁-C₆alkyl)₂, —NR¹³C(X³)NR¹³—C₁-C₆alkyl-P(═O)(C₁-C₆alkyl)₂, —NR¹³C(X³)NR¹³-arylP(═O)(C₁-C₆alkyl)₂ and —NR¹³C(X³)NR¹³-heteroarylP(═O)(C₁-C₆alkyl)₂, —(CH₂)ⱼNR³⁹(CH₂)ᵢ[O(CH₂)ᵢ]ₓ(CH₂)ⱼR⁹⁹, —(CH₂)ⱼNR³⁹(CH₂)ᵢSO₍₀₋₂₎(CH₂)ᵢ[O(CH₂)ᵢ]ⱼ(CH₂)ⱼR⁹⁹, —(CH₂)ⱼNR³⁹(CH₂)ⱼR¹⁰⁰, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, C₁-C₆ alkyl, —(CH₂)ⱼ—O—(CH₂)ᵢNR³⁶R³⁹, —(CH₂)ₙ—O—(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl); —C(O)(CH₂)ₙ(C₆-C₁₀ aryl), —(CH₂)ₙ—O—(CH₂)ⱼ(C₆-C₁₀ aryl), —(CH₂)ₙ—O—(CH₂)ᵢ(5-10 membered heterocyclyl), —C(O)(CH₂)ₙ(5-10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹(CH₂)ₙR³⁶, —SO₂(CH₂)ₙ(C₆-C₁₀ aryl), —SO₂(CH₂)ₙ(5-10 membered heterocyclyl), —(CH₂)ₙNR³⁶R³⁹, —NR³⁷SO₂NR³⁶R³⁹, SO₂R³⁶, C₂-C₆ alkenyl, C₃-C₁₀ cycloalkyl and C₁-C₆ alkylamino, wherein j is an integer ranging from 0 to 4 and preferably 0-2, n is an integer ranging from 0 to 6, x is an integer ranging from 1-6 and preferably 2-3, and i is an integer ranging from 2 to 6, preferably 2-3, the —(CH₂)ᵢ— and —(CH₂)ₙ— moieties of the foregoing R³⁸ groups optionally include a carbon-carbon double or triple bond where n is an integer between 2 and 6, and the alkyl, aryl and heterocyclyl moieties of the foregoing R³⁸ groups are optionally substituted by one or more substituents independently selected from halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, and —(CH₂)ₙOR³⁷;
X³ is selected from the group consisting of O, S, CH₂, N—CN, N—O-alkyl, NH and N(C₁-C₆alkyl);
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, —(CH₂)ₙCN(CH₂)ₙOR³⁷, —(CH₂)ₙCN(CH₂)ₙR³⁷, and —(CH₂)ₙA⁴R³⁷, wherein n is an integer ranging from 0 to 6 and i is an integer ranging from 2 to 6, A⁴ is selected from the group consisting of O, S, SO, SO₂, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙO(CH₂)ᵢOR³⁷, and —(CH₂)ₙOR³⁷, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6;
each R³⁷ and R⁴¹ is independently selected from H, OR³⁶, C₁-C₆ alkyl and C₃-C₁₀ cycloalkyl;
each R⁴² and R⁴³ is independently selected from the group consisting of H, C₁-C₆ alkyl, —Y—(C₃-C₁₀ cycloalkyl), —Y—(C₆-C₁₀ aryl), —Y—(C₆-C₁₀ heteroaryl), —Y-(5-10 membered heterocyclyl), —Y—O—Y¹—OR³⁷, —Y¹—CO₂—R³⁷, and —Y—OR³⁷;
Y is a bond or is —(C(R³⁷)(H))ₙ, wherein n is an integer ranging from 1 to 6;
Y¹ is —(C(R³⁷)(H))ₙ;
and the alkyl, cycloalkyl, aryl, heteroaryl and heterocyclyl moieties of the foregoing R⁴² and R⁴³ groups are optionally substituted by 1 or more substituents independently selected from R⁴⁴; or
R⁴² and R⁴³ taken together with the nitrogen to which they are attached form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted by 1 to 5 R⁴⁴ substituents, with the proviso that R⁴² and R⁴³ are not both bonded to the nitrogen directly through an oxygen;
each R⁴⁴ is independently selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, —SO₂R³⁶, —NR³⁶SO₂R³⁹, —NR³⁶SO₂NR³⁷R⁴¹, C₁-C₆ alkyl, C₂-C₆ alkenyl, C₂-C₆ alkynyl, C₃-C₁₀ cycloalkyl, —C₁-C₆ alkylamino, —(CH₂)ⱼ—O—(CH₂)ᵢNR³⁶R³⁹, —(CH₂)ₙ—O—(CH₂)ᵢR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl), —C(O)(CH₂)ⱼ(C₆-C₁₀ aryl), —(CH₂)ₙ—O—(CH₂)ⱼ(C₆-C₁₀ aryl), —(CH₂)ₙ—O—(CH₂)ᵢ(5 to 10 membered heterocyclyl), —C(O)(CH₂)ₙ(5 to 10 membered heterocyclyl), —(CH₂)ⱼNR³⁹(CH₂)ₙNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)NR³⁹(CH₂)ₙR³⁶, —SO₂(CH₂)ₙ(C₆-C₁₀ aryl), and —SO₂(CH₂)ₙ(5 to 10 membered heterocyclyl) wherein, j is an integer from 0 to 2, n is an integer from 0 to 6 and i is an integer ranging from 2 to 6, the —(CH₂)ᵢ— and —(CH₂)ₙ— moieties of the foregoing R⁴⁴ groups optionally include a carbon-carbon double or triple bond wherein n is then an integer from 2 to 6, and the alkyl, aryl and heterocyclyl moieties of the foregoing R⁴⁴ groups are optionally substituted by 1 or more substituents independently selected from the group consisting of halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, —SO₂R³⁶, —SO₂NR³⁶R³⁹, C₁-C₆ alkyl, C₃-C₁₀ cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷;
Z is selected from the group consisting of covalent bond, —O—, —O—CH₂—, —CH₂—O—, —S—, —CH₂—, —N(R⁵)—, —N(R⁵)—CH₂—, —CH₂—N(R⁵)—, —N(R⁵)—C(O)—N(R⁵)—, C₂alkynylene, —N(R⁵)—C(O)—, —C(O)—N(R⁵)—, —N(R⁵)—SO₂— and —SO₂—N(R⁵)—, wherein R⁵ is selected from the group consisting of H, an optionally substituted (C₁-C₅)acyl and C₁-C₆ alkyl-O—C(O), wherein C₁-C₆ alkyl is optionally substituted;
R⁹⁹ at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, P(═O)(OH)₂, —P(═O)(C₁-C₆alkyl)₂, —SO₃H—C(O)R³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), -, C₁-C₆ alkyl, C₂-C₆ alkenyl, C₂-C₆ alkynyl, —CH₂(CH₂)₀₋₄-T², wherein the aryl, heteroaryl, C₁-C₆ alkyl, C₂-C₆ alkenyl, and C₂-C₆ alkynyl are optionally substituted;
R¹⁰⁰ is a 12 to 24-membered optionally substituted heteroalicyclic macrocycle containing 4 to 8 oxygen atoms, preferentially 15-crown-5,18-crown-6, or 21-crown-7; and
R¹⁰¹ is selected from the group consisting of H, C₁-C₆alkyl, C₂-C₆alkenyl, —C₁-C₆alkyl-heterocycle and C₁-C₆alkyl-P(O)(C₁-C₆alkyl)₂,
with the proviso that when B is —N(R¹³)—; L is —C(═O)N(R¹³)— or —C(S)—N(R¹³); T is C(═O)-Q; R¹³ is H or C₁₋₆alkyl; R²⁰ is other than trihalomethyl, —O-trihalomethyl, —N(R³)C(O)OR³, C(O)SR³, —O—(CH₂)₀₋₆aryl and —O—(CH₂)₀₋₆heteroaryl; and D-NHC(O)R^{p1}, then R^{p1} is not C₁₋₆alkyl, C₂₋₆alkenyl, C₂₋₆alkynyl, C₃₋₁₀cycloalkyl, C₆₋₁₀aryl, C₁₋₆alkoxy, 5-to 10-membered heteoraryl, 3- to 10-membered non-aromatic heterocyclic group or a group represented by the formula —NR^{p2}R^{p3}, wherein R^{p2} and R^{p3} may be the same or different and each represents H, C₁₋₆alkyl, C₃₋₆alkenyl, C₃₋₆alkynyl, C₃₋₁₀cycloalkyl, C₆₋₁₀aryl, C₁₋₆alkoxy, 5- to 10-membered heteroaryl or a 4- to 1-membered non-aromatic heterocyclic group, and wherein R^{p1}, R^{p2} and R^{p3} are optionally substituted;
and with the proviso that Formula (A) excludes those compounds wherein Z is O or —CH₂—O—; and Ar is

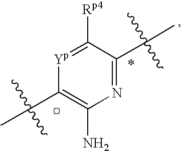

wherein [symbol] represents the point of attachment to Z, and * represents the point of attachment to G; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; when Y^{p} is —N(R³)CO₂R³; or when L² is —C(O)—, —C(S)—, —C(NH)— or >C═N(₁₋₆alkyl); and
with the proviso that Formula (A) excludes those compounds having the following structures

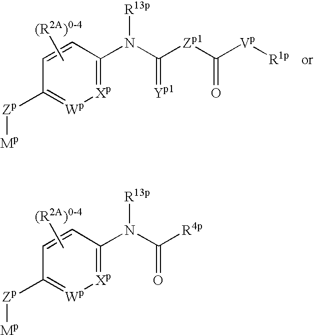

wherein Mp is selected from the group consisting of

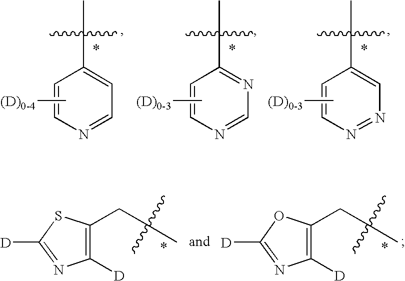

D is selected from the group consisting of H, halogen, NR^{p5}R^{p6}, OR^{p7}, CO2R^{p9}R^{p10}, CONR^{p9}R^{p10}, SO2R^{p11}, alkyl, cycloalkyl, alkenyl, alkynyl, CN, aryl, heteroaryl and heterocycloalkyl, wherein the alkyl, cycloalkyl, alkenyl, alkynyl, aryl, heteroaryl and heterocycloalkyl are optionally substituted; wherein R^{p5} to R^{p11} are independently selected from the group consisting of H, alkyl, alkenyl, alkynyl, cycloalkyl, alkoxycarbonyl, aryl, heteroaryl, heterocyclo and heterocycloalkyl, wherein the alkyl, alkenyl, alkynyl, cycloalkyl, aryl, heteroaryl, heterocyclo and heterocycloalkyl are optionally substituted; Z^{p} is selected from the group consisting of O, S and NH; WP and XP are each independently C or N; each R^{2A} is independently H, halogen, cyano, NO₂, OR^{p5}, NR^{p6}R^{p7}, alkyl, cycloalkyl, aryl, heteroaryl, heterocyclo, aryalkyl and heterocycloalkyl, wherein each of the alkyl, cycloalkyl, aryl, heteroaryl, heterocyclo, aryalkyl and heterocycloalkyl are optionally substituted; Y^{p1} is O, S and NP¹⁴ when Z comprises an N; or Y^{p1} is O when Z is alkyl or substituted alkyl; V^{p} is NR^{11p} or —(CR^{37p}R^{38p})₁₋₄, wherein if V^{p} is NR^{11p} then R^{1p} is alkyl or cycloalkyl; R^{11p} and R^{13p} are independently selected from the group consisting of H, alkyl, alkenyl, alkynyl, cycloalkyl, aryl, heteroaryl and heterocyclo, each of which is optionally substituted; R^{1p} is H, alkyl, cycloalkyl, arylalkyl, aryl, alkenyl, alkynyl, heteroaryl, heterocyclo, heteroarylalkyl, heterocycloalkyl, each of which is optionally substituted; R^{37p} and R^{38p} are independently selected from H, halogen and alkyl; and R^{4p} is selected from the group consisting of aryl, heteroaryl, heterocycloalkyl, each of which is optionally substituted; and
with the proviso that Formula (A) excludes those compounds wherein M is an optionally substituted pyrrole or an optionally substituted imidazole, Z is a covalent bond, and Ar is an optionally substituted pyrazole;
with the proviso that Formula (A) excludes

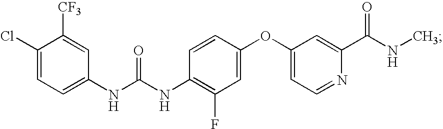

and
with the proviso that Formula (A) excludes those compounds wherein M is six-membered aryl or heteroaryl, wherein the heteroatom is N, and wherein M is optionally substituted with alkyl, alkenyl, alkylthio, mercapto, free, etherified or esterified hydroxyl, unsubstituted, mono or disubstituted amino, or halogen; Z is —O—, —S— or —NH—; Ar is an optionally substituted pyridine; and G is —N(R³³¹)—(CH₂)₀₋₂—Y³³¹ or —N(R³³¹)—(C(alkyl)(alkyl))₀₋₂—Y³³¹; wherein R³³¹ is H or alkyl and Y³³¹ is H, aryl, heterocyclic or optionally substituted cycloalkyl; and
with the proviso that Formula (A) excludes those compounds wherein (1) M is pyridine substituted with morpholinyl, NHC(O)C₁₋₆alkyl or O-phenyl, wherein said phenyl is optionally substituted with C₁₋₆alkyl, C₁-6alkoxy, halo or CF₃; Z is NH; Ar is pyrimidine substituted with halo; and G is —N(H)—(CH₂)₀₋₂-phenyl, wherein said phenyl is substituted with 1 or 2 substituents independently selected from SO₂NH₂ and halo; and (2) M is phenyl substituted with a substituent selected from —C(O)OH, —NHC(O)-phenyl, a five membered heterocycle and imadazol[1,2-a]pyridinyl; Z is —NH—; Ar is pyrimidine substituted with halo; and G is —N(H)-pyridine-O-phenyl, wherein said phenyl is substituted with one of H, C₁₋₆alkoxy, CF₃ or halo; and
with the proviso that Formula (A) excludes those compounds wherein D is —C(O)—NR⁴²R⁴³ or —C(O)NR^{6a}R^{6b}; M is phenyl optionally substituted with halogen or alkyl; Z is —NH—; and G is pyrimidine-pyridine; and
with the proviso that Formula (A) excludes those compounds wherein Z is selected from the group consisting of —O—, —O—CH₂—, —CH₂—O—, —S—, —CH₂—, —N(H)—, —N(H)—CH₂— and —CH₂—N(H)—; and G is selected from the group consisting of —N(R¹³)—C(O)—C(O)—N(R¹³)-Q, —N(R¹³)—C(═NR¹⁴)—C(O)—N(R¹³)-Q, —N(R¹³)—C(O)—C(S)—N(R¹³)-Q and —N(R¹³)—C(O)—C(═NR⁴)—N(R¹³)Q.

In a preferred embodiment of the present invention, the invention provides compounds of Formula (A-1):

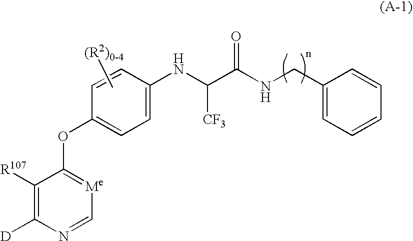

and N-oxides, hydrates, solvates, pharmaceutically acceptable salts, prodrugs and complexes thereof, and racemic and scalemic mixtures, diastereomers and enantiomers thereof, wherein
D, R² and n, are as defined previously,
M^{e} is N or CR¹⁰⁷, and
R¹⁰⁷ is selected from the group consisting of hydrogen, halogen, CN, nitro, azido, C₁-C₁₂alkyl, —C₁-C₁₂alkyl-cycloalkyl, —C₁-C₁₂alkyl-aryl, —C₁-C₁₂alkyl-heterocyclyl, —C₁-C₁₂alkyl-heteroaryl, —C₁-C₁₂heteroalkyl-cycloalkyl, —C₁-C₁₂heteroalkyl-aryl, —C₁-C₁₂heteroalkyl-heterocyclyl, —C₁-C₁₂heteroalkyl-heteroaryl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)₀₋₂R¹⁰⁸, —SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄OR¹⁰⁸, —CN, —C(O)R¹⁰⁸, —OC(O)R¹⁰⁸, —O(CR110R¹¹¹)₀₋₄R¹⁰⁸, —NR¹⁰⁸C(O)R¹⁰⁹, —(CR¹¹⁰R¹¹¹)₀₋₄C(O)OR¹⁰⁸, —(CR¹¹⁰R¹¹¹)₀₋₄NR¹⁰⁸R¹⁰⁹, —C(═NR¹¹⁰)NR¹⁰⁸R¹⁰⁹, —NR¹⁰⁸C(O)NR¹⁰⁹R¹¹⁰, —NR¹⁰⁸S(O)₁₋₂R¹⁰⁹, —C(O)NR¹⁰⁸R¹⁰⁹, —CH═CH—C₆-C₁₂aryl, —CH═CH-(3-12 membered heteroalicyclic), —CH═CH-(5-12 membered heteroaryl), —CH═CH—S(O)₀₋₂R¹⁰⁸, —CH═CH—SO₂NR¹⁰⁸R¹⁰⁹, —CH═CH—S(O)₂OR¹⁰⁸, —CH═CH—NO₂, —CH═CH—NR¹⁰⁸R¹⁰⁹, —CH═CH—(CR¹¹⁰R¹¹¹)₀₋₄OR¹⁰⁸, —CH═CH—CN, —CH═CH—C(O)R¹⁰⁸, —CH═CH—OC(O)R¹⁰⁸, —CH═CH—O(CR110R¹¹¹)₀₋₄R¹⁰⁸, —CH═CH—NR¹⁰⁸C(O)R¹⁰⁹, —CH═CH—(CR¹¹⁰R¹¹¹)₀₋₄C(O)OR¹⁰⁸, —CH═CH—(CR¹¹⁰R¹¹¹)₀₋₄NR¹⁰⁸R¹⁰⁹, —CH═CH—C(═NR¹¹⁰)NR¹⁰⁸R¹⁰⁹, —CH═CH—NR¹⁰⁸C(O)NR¹⁰⁹R¹¹⁰, —CH═CH—NR¹⁰⁸S(O)₁₋₂R¹⁰⁹, —CH═CH—C(O)NR¹⁰⁸R¹⁰⁹, —C≡C—C₆-C₁₂aryl, —C≡C-(3-12 membered heteroalicyclic), —C≡C-(5-12 membered heteroaryl), —C≡C—S(O)₀₋₂R¹⁰⁸, —C≡C—SO₂NR¹⁰⁸R¹⁰⁹, —C≡C—S(O)₂OR¹⁰⁸, —C≡C—NO₂, —C≡C—NR¹⁰⁸R¹⁰⁹—C≡C—(CR¹¹⁰R¹¹¹)₀₋₄OR¹⁰⁸, —C≡C—CN, —C≡C—C(O)R¹⁰⁸, —C≡C—OC(O)R¹⁰⁸, —C≡C—O(CR110R¹¹¹)₀₋₄R¹⁰⁸, —C≡C—NR¹⁰⁸C(O)R¹⁰⁹, —C≡C—(CR¹¹⁰R¹¹¹)₀₋₄C(O)OR¹⁰⁸, —C≡C—(CR¹¹⁰R¹¹¹)₀₋₄NR¹⁰⁸R¹⁰⁹, —C≡C—C(═NR¹¹⁰)NR¹⁰⁸R¹⁰⁹, —C≡C—NR¹⁰⁸C(O)NR¹⁰⁹R¹¹⁰, —C≡C—NR¹⁰⁸S(O)₁₋₂R¹⁰⁹ and —C≡C—C(O)NR¹⁰⁸R¹⁰⁹,
wherein each hydrogen of which is optionally substituted by an R¹¹⁷ group;
each R¹⁰⁸, R¹⁰⁹, R¹¹⁰ and R¹¹¹, which may be the same or different, is independently selected from hydrogen, halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic and 5-12 membered heteroaryl, or any two of R¹⁰⁸, R¹⁰⁹, R¹¹⁰ and R¹¹¹ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O and S, or any two of R¹⁰⁸, R¹⁰⁹, R¹¹⁰ and R¹¹¹ bound to the same carbon atom may be combined to form a C₃-C₁₂ cycloalkyl, C₆-C₁₂ aryl, 3-12 membered heteroalicyclic or 5-12 membered heteroaryl group, and each hydrogen of R¹⁰⁸, R¹⁰⁹, R¹¹⁰ and R¹¹¹ is optionally substituted by from 1 to 6 R¹¹⁷ groups;
each R¹¹⁷, which may be the same or different, is independently selected from halogen, C₁-C₁₂ alkyl, C₂-C₁₂ alkenyl, C₂-C₁₂ alkynyl, C₃-C₁₂ cycloalkyl, C₆-C₁₂ aryl, 3-12 membered heteroalicyclic, 5-12 membered heteroaryl, —CN, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic) and —O—(CH₂)₀₋₄(5 to 12 membered heteroaryl), —C(O)R¹¹⁹, —C(O)OR¹¹⁹ and —C(O)NR¹¹⁹R¹²⁰, and each hydrogen in R¹¹⁷ is optionally substituted by an R¹¹⁸ group;
each R¹¹⁸, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic, 5-12 membered heteroaryl, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic), —O—(CH₂)₀₋₄(5-12 membered heteroaryl) and —CN, and each hydrogen in R¹¹⁸ is optionally substituted by a group selected from halogen, —OH, —CN, —C₁-C₁₂alkyl which may be partially or fully halogenated, —O—C₁-C₁₂ alkyl which may be partially or fully halogenated, —CO, —SO, —SO₂ and —SO₃H; and
   each R¹¹⁹ and R¹²⁰, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic and 5-12 membered heteroaryl, and each R¹¹⁹ and R¹²⁰ is optionally substituted by a group selected from halogen, —OH, —CN, —C₁-C₁₂ alkyl which may be partially or fully halogenated, —O—C₁-C₁₂ alkyl which may be partially or fully halogenated and SO₃H, or R¹¹⁹ and R¹²⁰, taken together with the nitrogen atom to which they are attached, may form a 3-12 membered heteroalicyclic ring optionally substituted by from 1 to 6 R¹¹⁸ groups.

In another preferred embodiment of the present invention, the invention provides compounds of Formula (B):

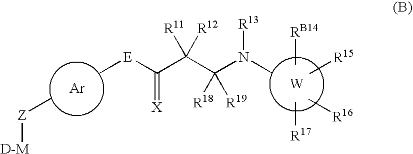

and N-oxides, hydrates, solvates, pharmaceutically acceptable salts, prodrugs and complexes thereof, and racemic and scalemic mixtures, diastereomers and enantiomers thereof, wherein,
D, M, Z, Ar, E, X, R¹³, W, R^{B14}, R¹⁵, R¹⁶ and R¹⁷ are as defined previously;
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; or
R¹² and R¹³ taken together with the atoms to which they are attached optionally form a 4 to 8 membered cycloalkyl or heterocyclic ring system, which ring system is optionally substituted; or
R¹³ and R^{B14} taken together with the atoms to which they are attached optionally form a 4 to 8 membered cycloalkyl or heterocyclic ring system, which ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or

R¹⁸ and R¹⁹ together with the atom to which they are attached form a 3 to 6 membered cycloalkyl or heterocycle, each of which is optionally substituted with 1 to 4 halo, preferably F.

In a preferred embodiment of the compounds of Formula (B), R¹³ is selected from the group consisting of H, C₁-C₆alkyl, substituted C₁-C₆alkyl, cycloalkyl, substituted cycloalkyl, OH, unsubstituted —O—(C₁-C₆alkyl), substituted —O—(C₁-C₆alkyl).

In another preferred embodiment of the present invention, the invention provides compounds of formula (B-1):

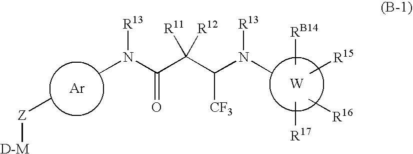

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹¹, R¹², R¹³, W, R^{B14}, R¹⁵, R¹⁶ and R¹⁷ are as defined previously.

In a preferred embodiment of the compounds according to Formula (B-1), W is phenyl.

In another preferred embodiment of the present invention, the invention provides compounds of Formula (C):

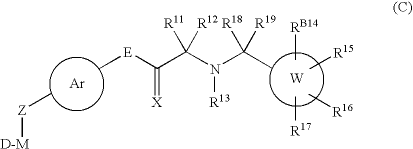

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, R¹¹, R¹², R¹³, R^{B14}, R¹⁵, R¹⁶, R¹⁷, R¹⁸, R¹⁹ and W are as defined previously.

In a preferred embodiment of the Formula (C), the invention provides compounds of formula (C-1):

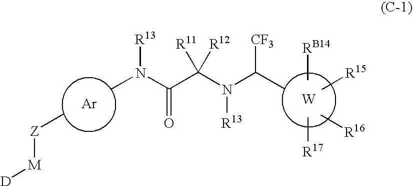

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹¹, R¹², R¹³, R^{B14}R¹⁵, R¹⁶, R¹⁷ and W are as defined previously.

In a preferred embodiment of the compounds according to Formula (C-1), W is phenyl.

In another preferred embodiment of the present invention, the invention provides compounds of Formula (D):

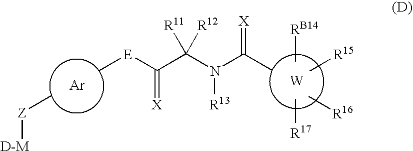

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, R¹¹, R¹², R¹³, R^{B14}, R¹⁵, R¹⁶, R¹⁷ and W are as defined previously.

In another preferred embodiment of the present invention, the invention provides compounds of Formula (E):

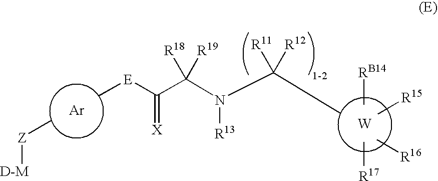

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, R¹¹, R¹², R¹³, R^{B14}, R¹⁵, R¹⁶, R¹⁷, R¹⁸, R¹⁹ and W are as defined previously.

In a preferred embodiment of Formula (E), the invention provides compounds of formula (E-1):

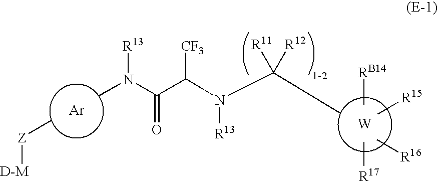

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹¹, R¹², R¹³, R^{B14}, R¹⁵, R¹⁶, R¹⁷ and W are as defined previously.

In a preferred embodiment of the compounds according to Formula (E-1), W is phenyl.

In another preferred embodiment of the Formula (E), the invention provides compounds of Formula (E-2):

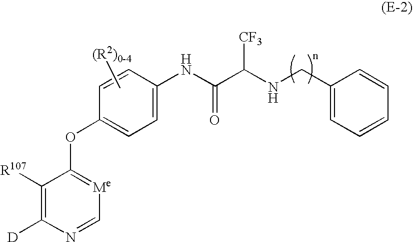

and N-oxides, hydrates, solvates, pharmaceutically acceptable salts, prodrugs and complexes thereof, and racemic and scalemic mixtures, diastereomers and enantiomers thereof, wherein R¹⁰⁷, D, M^{e}, R² and n are as defined previously.

In another preferred embodiment of the present invention, the invention provides compounds of Formula (F):

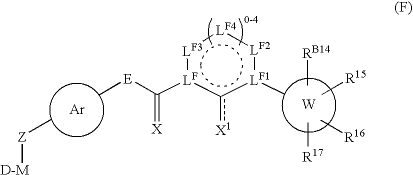

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, R^{B14}, R¹⁵, R¹⁶, R¹⁷ and W are as defined previously; [symbol] is a single or double bond;
X¹ is selected from the group consisting of O, S, CH₂, N—CN, N—O-alkyl, NH and N(C₁-C₆alkyl) when [symbol] is a double bond, or
X¹ is selected from the group consisting of H, halogen, alkyl, alkenyl, alkynyl, CN, alkoxy, NH(alkyl) and alkyl-thio, when [symbol] is a single bond;
L^{F} and L^{F1} are independently selected from the group consisting of —CH—, —N—, —C(halogen)- and —C(C₁-C₆alkyl)-;
L^{F2} and L^{F3} are independently selected from the group consisting of CH, CH₂, N, O and S;
L^{F4} is selected from the group consisting of absent, CH, CH₂, N, O and S; and the group

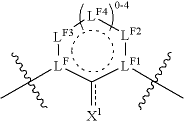

is aromatic or non-aromatic, provided that two O are not adjacent to each other;
and with the proviso that Formula (F) excludes those compounds wherein Z is O or —CH₂O—; Ar is

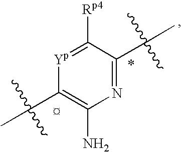

wherein [symbol] represents the point of attachment to Z, and * represents the point of attachment to E; E is —N(H)— or —N(alkyl)-; X is O; [symbol] is a single bond; and X¹ is H, halogen, alkyl, alkenyl, alkynyl, CN, alkoxy; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³;
with the proviso that Formula (F) excludes those compounds having the following structure or

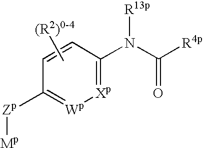

wherein Mp is selected from the group consisting of

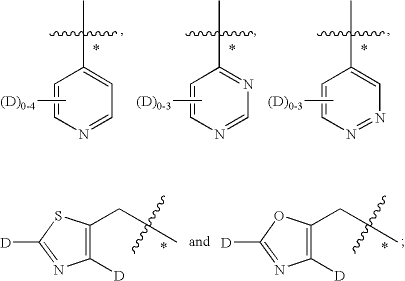

D is selected from the group consisting of H, halogen, NR^{p5}R^{p6}, OR^{p7}, CO2R^{p8}, CONR^{p9}R^{p10}, SO2R^{p11}, alkyl, cycloalkyl, alkenyl, alkynyl, CN, aryl, heteroaryl and heterocycloalkyl, wherein the alkyl, cycloalkyl, alkenyl, alkynyl, aryl, heteroaryl and heterocycloalkyl are optionally substituted; wherein R^{p5} to R^{p11} are independently selected from the group consisting of H, alkyl, alkenyl, alkynyl, cycloalkyl, alkoxycarbonyl, aryl, heteroaryl, heterocyclo and heterocycloalkyl, wherein the alkyl, alkenyl, alkynyl, cycloalkyl, aryl, heteroaryl, heterocyclo and heterocycloalkyl are optionally substituted; Z^{p} is selected from the group consisting of O, S and NH; W^{p} and X^{p} are each independently C or N; each R² is independently H, halogen, cyano, NO₂, OR^{p5}, NR^{p6}R^{p7}, alkyl, cycloalkyl, aryl, heteroaryl, heterocyclo, aryalkyl and heterocycloalkyl, wherein each of the alkyl, cycloalkyl, aryl, heteroaryl, heterocyclo, aryalkyl and heterocycloalkyl are optionally substituted; R^{13p} is selected from the group consisting of H, alkyl, alkenyl, alkynyl, cycloalkyl, aryl, heteroaryl and heterocyclo, each of which is optionally substituted; and R^{4p} is selected from the group consisting of aryl, heteroaryl, heterocycloalkyl, wherein the aryl is optionally substituted with halogen, alkyl, alkoxy, amino, cycloalkyl, aryl, heteroaryl, cyano, alkyl S(O)₀₋₂ or thiol, the heteroaryl is optionally substituted with halogen, alkyl, alkenyl, alkynyl, aryl, cyano, alkoxy, thioalkyl, ═O, phenyl, benzyl, phenylethyl, phenyloxy, phenylthio, cycloalkyl, heterocyclo, heteroaryl and NH(alkyl), and the heterocycloalkyl is optionally substituted with alkyl, alkoxy, nitro, monoalkylamino, dialkylamino, cyano, halo, haloalkyl, alkanoyl, aminocarbonyl, monoalkylaminocarbonyl, dialkylaminocarbonyl, alkyl amido, alkoxyalkyl, alkoxycarbonyl, alkylcarbonyloxy and aryl, said aryl further optionally substituted with halo, C₁₋₆alkyl or C₁₋₆alkoxy; and
with the proviso that Formula (F) excludes those compounds wherein M is an optionally substituted pyrrole or an optionally substituted imidazole, Z is a covalent bond, and Ar is an optionally substituted pyrazole.

In a preferred embodiment of Formula F, [symbol] is a double bond, X¹ is O, and L^{F2}, L^{F3} and L^{F4}, if present, are saturated.

In a preferred embodiment of Formula F, the invention provides compounds of Formula (F-1):

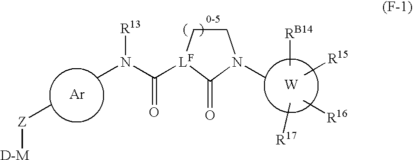

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹³, R^{B14}, R¹⁵, R¹⁶, R¹⁷ and W are as defined previously, and L^{F} is either —CH— or N.

In a preferred embodiment of the compounds according to Formula (F-1), W is phenyl.

In another preferred embodiment of the present invention, the invention provides compounds of Formula (G):

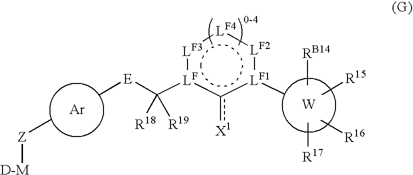

and N-oxides, hydrates, solvates, pharmaceutically acceptable salts, prodrugs and complexes thereof, and racemic and scalemic mixtures, diastereomers and enantiomers thereof, wherein D, M, Z, Ar, E, R^{B14}, R¹⁵, R¹⁶, R¹⁷, R¹⁸, R¹⁹ and W are as defined previously; [symbol]
is a single or double bond;
X¹ is selected from the group consisting of O, S, CH₂, N—CN, N—O-alkyl, NH and N(C₁-C₆alkyl) when [symbol] is a double bond or
X¹ is selected from the group consisting of H, halogen, alkyl, alkenyl, alkynyl, CN, alkoxy, NH(alkyl) and alkyl-thio, when [symbol] is a single bond;
L^{F} and L^{F1} are independently selected from the group consisting of —CH—, —N—, —C(halogen)- and —C(C₁-C₆alkyl)-;
L^{F2} and L^{F3} are independently selected from the group consisting of CH, CH₂, N, O and S;
L^{F4} is selected from the group consisting of absent, CH, CH₂, N, O and S; and the group

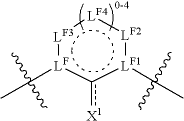

is aromatic or non-aromatic, provided that two 0 are not adjacent to each other;
and with the proviso that Formula (G) excludes those compounds wherein Z is O or —CH₂—O—; Ar is

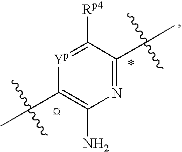

wherein [symbol] represents the point of attachment to Z, and * represents the point of attachment to E; E is —N(H)— or —N(alkyl)-; [symbol] is a single bond; and X¹ is H, halogen, alkyl, alkenyl, alkynyl, CN, alkoxy; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

In a preferred embodiment of Formula (G), the invention provides compounds of Formula (G-1):

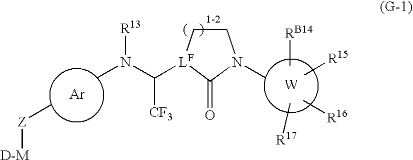

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹³, R^{B14}, R¹⁵, R¹⁶, R¹⁷ and W are as defined previously, and L^{F} is either —CH— or N.

In a preferred embodiment of the compounds according to Formula (G-1), W is phenyl.

In another preferred embodiment of the present invention, the invention provides compounds of Formula (H):

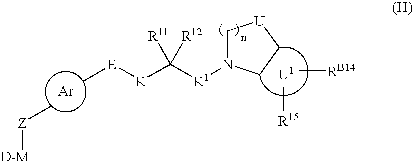

and N-oxides, hydrates, solvates, pharmaceutically acceptable salts, prodrugs and complexes thereof, and racemic and scalemic mixtures, diastereomers and enantiomers thereof, wherein D, M, Z, Ar, E, R¹¹, R¹², R^{B14} and R¹⁵ are as defined previously;
K and K¹ are independently selected from the group consisting of —C(O)—, —C(S)—, —C(NH)—, —C(NCN)— and —C(R¹⁸R¹⁹)—;
wherein R¹⁸ and R¹⁹ are as defined previously;

U is selected from the group consisting of O, S, SO₂, NH, and N(C₁-C₆alkyl), wherein the C₁-C₆alkyl is optionally substituted with a substituent selected from the group consisting of —OH, -alkoxy, amino, NH(C₁-C₆alkyl), N(C₁-C₆alkyl)₂,

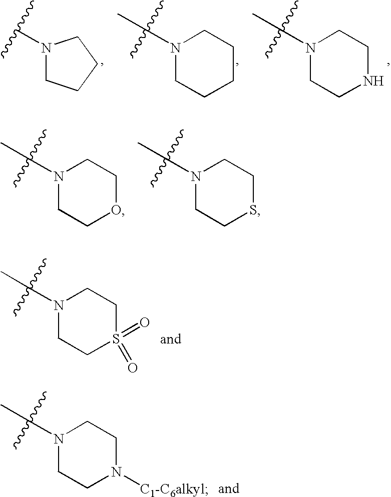

U¹ is a ring system selected from the group consisting of cycloalkyl, substituted cycloalkyl, heterocyclyl, substituted heterocyclyl, aryl, substituted aryl, heteroaryl and substituted heteroaryl;
and with the proviso that Formula (H) excludes those compounds wherein Z is O or —CH₂—O—; Ar is

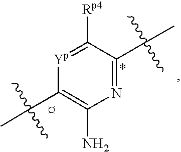

wherein [symbol] represents the point of attachment to Z, and * represents the point of attachment to E; E is —N(H)— or —N(alkyl)-; K is C(O) and K¹ is —C(R¹⁸R¹⁹)—, or K and K¹ are both —C(R¹⁸R¹⁹)—; and R¹⁸ and R¹⁹ are independently selected from the group consisting of H, halogen, —O-alkyl, alkyl, fluorinated alkyl and CN; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

In a preferred embodiment of Formula (H), the invention provides compounds of Formula (H-1):

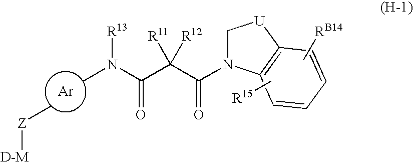

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹³, R¹¹, R¹², R^{B14}, R¹⁵ and U are as defined previously.

In another preferred embodiment of Formula (H), the invention provides compounds of Formula (H-2):

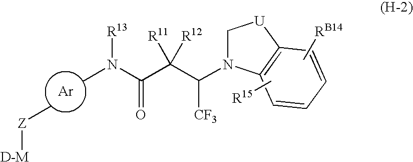

and N-oxides, hydrates, solvates, pharmaceutically acceptable salts, prodrugs and complexes thereof, and racemic and scalemic mixtures, diastereomers and enantiomers thereof, wherein D, M, Z, Ar, U, R¹¹, R¹², R13, R^{B14} and R¹⁵ are as defined previously.

In another preferred embodiment of Formula (H), the invention provides compounds of Formula (H-3):

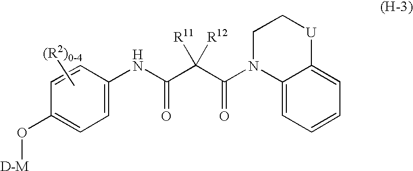

and N-oxides, hydrates, solvates, pharmaceutically acceptable salts, prodrugs and complexes thereof, and racemic and scalemic mixtures, diastereomers and enantiomers thereof, wherein D, M, R², R¹¹, R¹² and U are as defined previously.

In a preferred embodiment of the compounds according to Formula (H-3), U is NH or N(C₁₋₆alkyl), wherein the C₁₋₆alkyl is optionally substituted as defined in Formula (H).

In a preferred embodiment of the compounds according to Formula (H-3), M is an optionally substituted heteroaryl, preferably pyridine.

In another preferred embodiment of the present invention, the invention provides compounds of Formula (I):

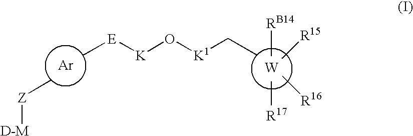

and N-oxides, hydrates, solvates, pharmaceutically acceptable salts, prodrugs and complexes thereof, and racemic and scalemic mixtures, diastereomers and enantiomers thereof, wherein D, M, Z, Ar, E, K, K¹, R^{B14}, R¹⁵, R¹⁶, R¹⁷ and W are as defined previously,
and with the proviso that Formula (I) excludes those compounds wherein Z is O or —CH₂—O—; Ar is

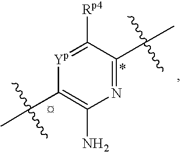

wherein [symbol] represents the point of attachment to Z, and * represents the point of attachment to E; E is —N(H)— or —N(alkyl)-; K and K¹ are both —C(R¹⁸R¹⁹)—; and R¹⁸ and R¹⁹ are independently selected from the group consisting of H, halogen, —O-alkyl, alkyl, fluorinated alkyl and CN; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

In a preferred embodiment of the Formula (I), the invention provides compounds of Formula (I-1):

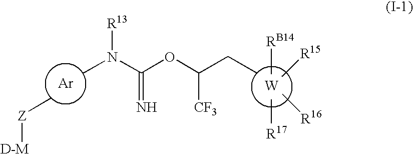

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹³, R^{B14}, R¹⁵, R¹⁶, R¹⁷ and W are as defined previously.

In a preferred embodiment of the compounds according to Formula (I-1), W is phenyl.

In a preferred embodiment of the present invention, the invention provides compounds of Formula (J):

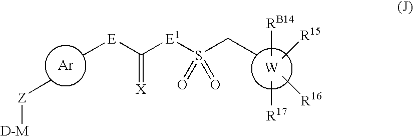

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, W R^{B14}, R¹⁵, R¹⁶ and R¹⁷ are as defined previously; and
E¹ is selected from the group consisting of —N(H)—, —N(C₁-C₆alkyl)-, —CH₂N(H)— and —N(H)CH₂—.

In a preferred embodiment of Formula (J), the invention provides compounds of Formula (J-1):

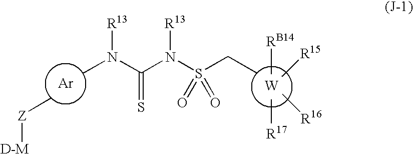

wherein D, M, Z, Ar, R¹³, R^{B14}, R¹⁵, R¹⁶, R¹⁷ and W are as defined previously.

In a preferred embodiment of the compounds according to Formula (J-1), W is phenyl.

In another preferred embodiment of the present invention, the invention provides compounds of Formula (K):

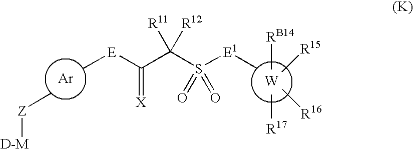

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, R¹¹, R¹², E¹, R^{B14}, R¹⁵, R¹⁶, R¹⁷ and W are as defined previously.

In a preferred embodiment of the Formula (K), the invention provides compounds of Formula (K-1):

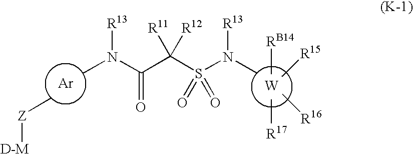

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹¹, R¹², R¹³, R^{B14}, R¹⁵, R¹⁶, R¹⁷ and W are as defined previously.

In a preferred embodiment of the compounds according to Formula (K-1), W is phenyl.

In another embodiment of the present invention, the invention provides compounds of Formula (L):

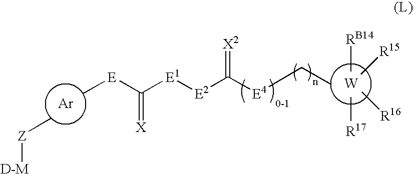

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, W, R^{B14}, R¹⁵, R¹⁶ and R¹⁷ are as defined previously;
n is 0, 1, 2, 3 or 4;
X² is selected from the group consisting of O, S, NH, NOH, NOMe, NOEt and NCN;
E¹ and E² are independently selected from the group consisting of —N(H)—, —N(C₁-C₆alkyl)-, —CH₂N(H)— and —N(H)CH₂—; and
E⁴ is —N(H)— or —N(C₁-C₆alkyl)-.

In another preferred embodiment of the present invention, the invention provides compounds of Formula (M):

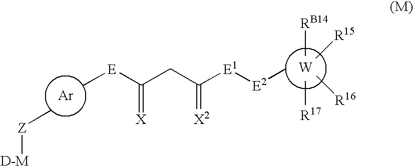

and N-oxides, hydrates, solvates, pharmaceutically acceptable salts, prodrugs and complexes thereof, and racemic and scalemic mixtures, diastereomers and enantiomers thereof, wherein D, M, Z, Ar, E, X, X², E¹, E², R^{B14}, R¹⁵, R¹⁶, R¹⁷ and W are as defined previously.

In another preferred embodiment of the present invention, the invention provides compounds of Formula (N):

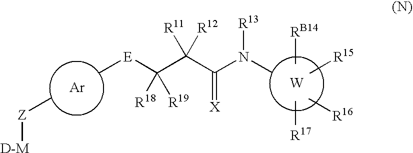

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹³, E, X, W, R¹¹, R¹², R^{B14}, R¹⁵, R¹⁶, R¹⁷, R¹⁸ and R¹⁹ are as defined previously.

In a preferred embodiment of Formula (N), the invention provides compounds of Formula (N-1):

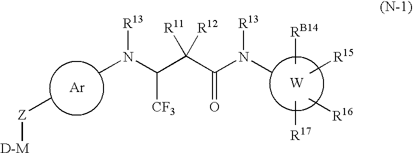

and N-oxides, hydrates, solvates, pharmaceutically acceptable salts, prodrugs and complexes thereof, and racemic and scalemic mixtures, diastereomers and enantiomers thereof, wherein D, M, Z, Ar, R¹¹, R¹², R¹³, R^{B14}, R¹⁵, R¹⁶, R¹⁷ and W are as defined previously.

In a preferred embodiment of the compounds according to Formula (N-1), W is phenyl.

In another preferred embodiment of Formula (N), the invention provides compounds of Formula (N-2):

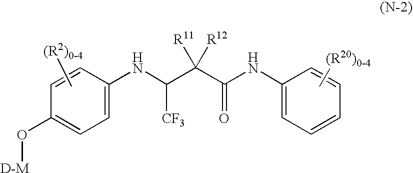

and N-oxides, hydrates, solvates, pharmaceutically acceptable salts, prodrugs and complexes thereof, and racemic and scalemic mixtures, diastereomers and enantiomers thereof, wherein D, M, R², R¹¹, R¹² and R²⁰ are as defined previously.

In a preferred embodiment of the compounds according to Formula (N-2), M is an optionally substituted heteroaryl, preferably pyridine.

In another preferred embodiment of the present invention, the invention provides compounds of Formula (O):

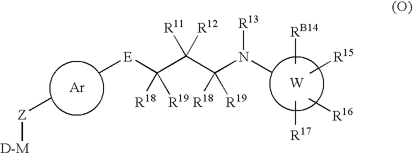

and N-oxides, hydrates, solvates, pharmaceutically acceptable salts, prodrugs and complexes thereof, and racemic and scalemic mixtures, diastereomers and enantiomers thereof, wherein D, M, Z, Ar, E, R¹¹, R¹², R¹³, R^{B14}, R¹⁵, R¹⁶, R¹⁷, R¹⁸, R¹⁹ and W are as defined previously; and wherein
each R¹⁸ and R¹⁹ is independent of each other R¹⁸ and R¹⁹.

In a preferred embodiment of the compounds according to the present invention, D is defined by the group R⁷, wherein R⁷ is selected from the group consisting of —H, halogen, C₁-C₆ alkyl, C₃-C₁₀ cycloalkyl, —C(O)NR⁴²R⁴³, —C(O)(C₆-C₁₀ aryl), —C(O)(heterocyclyl), —C(O)(heteroaryl), —Y—(C₆-C₁₀ aryl), —Y-(5-10 membered heterocyclyl), —Y-(heteroaryl), —S-aryl, —S—C₁-C₆ alkyl, —SO—C₁-C₆ alkyl, —SO₂—C₁-C₆ alkyl, —Y—NR⁴²R⁴³, —SO₂NR⁴²R⁴³ and —C(O)OR^{6a}, wherein the aforementioned R⁷ groups other than —H and halogen are optionally substituted by 1 to 5 R³⁸.

In a preferred embodiment of the compounds according to the present invention, D is defined by the group R⁷, wherein R⁷ is selected from the group consisting of —H, —C(O)NR⁴²R⁴³, —Y-(5 to 10 membered heterocyclyl), —Y—(C₆-C₁₀ aryl), —Y-(heteroaryl), —Y—NR⁴²R⁴³, SO₂NR⁴²R⁴³ and C(O)OR⁴², wherein the aforementioned R⁷ groups other than —H are optionally substituted by 1 to 5 R³⁸.

In a preferred embodiment of the compounds according to the present invention, R⁷ is selected from the group consisting of —(CH₂)ₙ(5 to 10 membered heterocyclyl), —C(O)NR⁴²R⁴³, —SO₂NR⁴²R⁴³ and —CO₂R⁴², wherein said R⁷ group —(CH₂)ₙ(5 to 10 membered heterocyclyl) is unsubstituted or substituted by one or more R³⁸ groups.

In a preferred embodiment of the compounds according to the present invention, R⁷ is selected from the group consisting of —(CH₂)ₙ(5 to 10 membered heterocyclyl), and —C(O)NR⁴²R⁴¹.

In a preferred embodiment of the compounds according to the present invention, R⁷ is —C(O)NR⁴²R⁴³, wherein R⁴² and R⁴³ are independently selected from H, (C₁-C₆)alkyl, (C₃-C₁₀)cycloalkyl, —(CH₂)ₙ(C₃-C₁₀ cycloalkyl), —(CH₂)ₙ(C₆-C₁₀ aryl), —(CH₂)ₙ(5 to 10 membered heterocyclyl), —(CH₂)ₙ—O—(CH₂)ᵢOR³⁷, —(CH₂)ₙOR³⁷, wherein n is an integer from 0 to 6, i is an integer from 2 to 6, and the alkyl, aryl and heterocyclyl moieties of said R⁴² and R⁴³ groups are unsubstituted or substituted with one or more substituents independently selected from R³⁸, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, where R⁴² and R⁴³ are not both bonded to the nitrogen directly through an oxygen.

In a preferred embodiment of the compounds according to the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₈ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, thiomorpholinyl, isoquinolinyl, or dihydroisoquinolinyl ring, wherein said pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidinyl, piperidinyl, piperazinyl, morpholinyl, or thiomorpholinyl ring, wherein said pyrrolidinyl, piperidinyl, piperazinyl, morpholinyl, or thiomorpholinyl rings are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidinyl or piperidinyl ring, wherein said pyrrolidinyl or piperidinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidinyl ring, wherein said pyrrolidinyl is unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidin-1-yl ring, wherein said pyrrolidin-1-yl is unsubstituted or substituted by 1 to 5 R³⁸ substituents.

In a preferred embodiment of the compounds according to the present invention, R⁷ is —(CH₂)ₙ(5 to 10 membered heterocyclyl) group, wherein said —(CH₂)ₙ(5 to 10 membered heterocyclyl) group is unsubstituted or substituted by 1 to 5 R³⁸ groups.

In a preferred embodiment of the compounds according to the present invention, R⁷ is —(CH₂)ₙ(5-8 membered heterocyclyl) group, said —(CH₂)ₙ(5-8 membered heterocyclyl) group is unsubstituted or substituted by 1 to 5 R³⁸ groups.

In a preferred embodiment of the compounds according to the present invention, R⁷ is —(CH₂)ₙ(5 or 6 membered heterocyclyl) group, said —(CH₂)ₙ(5 or 6 membered heterocyclyl) group is unsubstituted or substituted by 1 to 5 R³⁸ groups.

In a preferred embodiment of the compounds according to the present invention, R⁷ is —(CH₂)ₙ(5 membered heterocyclyl) group, said —(CH₂)ₙ(5 membered heterocyclyl) group is unsubstituted or substituted by 1 to 5 R³⁸ groups.

In a preferred embodiment of the compounds according to the present invention, R⁷ is —(CH₂)ₙthiazolyl, wherein n is an integer from 0 to 6, said —(CH₂)ₙthiazolyl is unsubstituted or substituted by 1 to 5 R³⁸ groups.

In a preferred embodiment of the compounds according to the present invention, R⁷ is a thiazolyl, said thiazolyl is unsubstituted or substituted by 1 to 5 R³⁸ groups.

In a preferred embodiment of the compounds according to the present invention, R⁷ is an imidazolyl, said imidazolyl is unsubstituted or substituted by 1 to 5 R³⁸ groups.

In a preferred embodiment of the compounds according to the present invention, R⁷ is selected from the group consisting of imidazolyl, oxazolyl, oxadiazolyl, isoxazolyl, thiazolyl and thiadiazolyl, wherein the imidazolyl, oxazolyl, oxadiazolyl, isoxazolyl, thiazolyl and thiadiazolyl, each of which is optionally substituted by 1 to 5 R³⁸ groups.

In a preferred embodiment of the compounds according to the present invention, R⁷ is selected from the group consisting of halo, —CO₂H, —CONH₂ and —CSNH₂.

In a preferred embodiment of the compounds according to the present invention, R⁷ is a heteroaryl group optionally substituted by one or more moiety selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁷, —OR³⁷, —SO₂NR³⁶R³⁹, (C₁-C₆)alkyl, (C₃-C₁₀)cycloalkyl, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5 to 10 membered heterocyclyl), —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH2)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, (CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹, —(CH₂)ₙR³⁶, —SO₂(CH₂)ₙ(C₆-C₁₀ aryl), and —SO₂(CH₂)ₙ(5 to 10 membered heterocyclyl), wherein j is an integer from 0 to 2, n is an integer from 0 to 6, i is an integer from 2 to 6, the —(CH₂)ᵢ— and —(CH₂)ₙ— moieties of the said substituent groups optionally include a carbon-carbon double or triple bond where n is an integer between 2 and 6, and the alkyl, aryl and heterocyclyl moieties of the substituent groups are unsubstituted or substituted with one or more substituents independently selected from halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙ—O—(CH₂)ᵢOR³⁷ and —(CH₂)ₙOR³⁷ wherein n is an integer from 0 to 6 and i is an integer from 2 to 6, and wherein R³⁶ and R³⁹ are independently selected from the group consisting of H, —OH, (C₁-C₆)alkyl, (C₃-C₁₀)cycloalkyl, —(CH₂)ₙ(C₆-C₁₀ aryl), —(CH₂)ₙ(5 to 10 membered heterocyclyl), —(CH₂)ₙ—O—(CH₂)ⁱOR³⁷ and —(CH₂)ₙOR³⁷, wherein n is an integer from 0 to 6 and i is an integer from 2 to 6, and the alkyl, aryl and heterocyclyl moieties of the R³⁶ and R³⁹ groups are unsubstituted or substituted with one or more substituents independently selected from hydroxy, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴⁰, C(O)NR³⁷R⁴¹, —NR³⁷R⁴¹, (C₁-C₆)alkyl, —(CH₂)ₙ(C₆-C₁₀ aryl), —(CH₂)ₙ(5 to 10 membered heterocyclyl), —(CH₂)ₙ—O—(CH₂)ᵢOR³⁷ and —(CH₂)ₙOR³⁷ wherein n is an integer from 0 to 6 and i is an integer from 2 to 6, where when R³⁶ and R³⁹ are both attached to the same nitrogen, then R³⁶ and R³⁹ are not both bonded to the nitrogen directly through an oxygen.

In a preferred embodiment of the compounds according to the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted by 1 to 5 R³⁸ groups. Preferably R⁷ is —(CH₂)ₙ(C₆-C₁₀ aryl) and —(CH₂)ₙ(5 to 10 membered heterocyclyl), optionally substituted by 1 to 5 R³⁸ groups, more preferably phenyl or pyridyl, optionally substituted by 1 to 5 R³⁸ groups.

In a preferred embodiment of the compounds according to the present invention, R⁷ is selected from the group consisting of H, —(C₁-C₆)alkyl, —C(O)NR³⁶R³⁷, —C(O)(C₆-C₁₀ aryl), —(CH₂)ₙ(C₆-C₁₀ aryl) and —(CH₂)ₙ(5 to 10 membered heterocyclyl), wherein the R⁷ groups other than H are optionally substituted by tert-butyl-dimethylsilanyl and 1 to 3 R³⁸ groups.

In a preferred embodiment of the compounds according to the present invention, R⁷ is selected from the group consisting of —C(O)NR⁴²R⁴³, —(CH₂)ₙNR⁴²R⁴³, —NR⁴²C(═O)R⁴³, —SO₂R⁴², —SO₂NR⁴²R⁴³, —NR³⁷SO₂R⁴², —NR³⁷SO₂NR⁴²R⁴³, —C(═N—OR⁴²)R⁴³, C(═NR⁴²)R⁴³, —NR³⁷C(═NR⁴²)R⁴³, —C(═NR⁴²)NR³⁷R⁴³, —NR³⁷C(═NR⁴²)NR³⁷R⁴³, —C(O)R⁴², —CO₂R⁴², wherein each R⁴² and R⁴³ is independently selected from the group consisting of H, (C₁-C₆)alkyl, —(CH₂)ₙ(C₃-C₁₀)cycloalkyl), —(CH₂)ₙ(C₆-C₁₀ aryl), —(CH₂)ₙ(5 to 10 membered heterocyclyl), —(CH₂)ₙ—O—(CH₂)ᵢOR³⁷, —(CH₂)ₙOR³⁷, wherein n is an integer from 0 to 6 and i is an integer from 2 to 6, and the alkyl, aryl and heterocyclyl moieties of the foregoing R⁴² and R⁴³ groups are optionally substituted by 1 to 3 substituents independently from R³⁸, or R⁴² and R⁴³ are taken together with the nitrogen to which they are attached to form a C₅-C₈ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

In a preferred embodiment of the compounds according to the present invention, R⁷ is selected from the group consisting of —C(O)NR⁴²R⁴³, —SO₂R⁴², —SO₂NR⁴²R⁴³, —C(═N—OR⁴²)R⁴³ and —C(═NR⁴²)R⁴³.

In a preferred embodiment of the compounds according to the present invention, R⁷ is —C(O)NR⁴²R⁴³, wherein each R⁴² and R⁴³ is independently selected from the group consisting of H, (C₁-C₆)alkyl, —(CH₂)ₙOR³⁷, wherein n is an integer from 0 to 6 and the alkyl moiety of the foregoing R⁴² and R⁴³ groups are optionally substituted by 1 to 3 substituents independently from halo, cyano, trifluoromethyl, —C(O)R⁴⁰, —NR³⁷C(O)R⁴¹, —C(O)NR³⁷R⁴¹, —NR³⁷R⁴¹, (C₁-C₆)alkyl, —(CH₂)ₙ(C₆-C₁₀ aryl), —(CH₂)ₙ(5 to 10 membered heterocyclyl), —(CH₂)ₙ—O—(CH₂)ᵢOR³⁷ and —(CH₂)ₙOR₃₇, wherein n is an integer from 0 to 6 and i is an integer from 2 to 6, or R⁴² and R⁴³ are taken together with the nitrogen to which they are attached to form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are unsubstituted or substituted with 1 to 5 R³⁸ substituents, with the proviso that R⁴² and R⁴³ are not both bonded to the nitrogen directly through an oxygen.

In a preferred embodiment of the compounds according to the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl, pyrrolidinyl, piperidinyl, piperazinyl or morpholinyl ring, wherein said C₅-C₉ azabicyclic, aziridinyl, azetidinyl, pyrrolidinyl, piperidinyl, piperazinyl or morpholinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a C₅-C₉ azabicyclic, aziridinyl, azetidinyl or pyrrolidinyl ring, wherein said C₅-C₈ azabicyclic, aziridinyl, azetidinyl or pyrrolidinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a C₅-C₉ azabicyclic, azetidinyl or pyrrolidinyl ring, wherein said C₅-C₉ azabicyclic, azetidinyl or pyrrolidinyl ring are unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a C₅-C₉ azabicyclic ring, wherein said C₅-C₉ azabicyclic ring is unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a azetidinyl ring, wherein said azetidinyl ring is unsubstituted or substituted with 1 to 5 R³⁸ substituents.

In a preferred embodiment of the compounds according to the present invention, R⁷ is —C(O)NR⁴²R⁴³, wherein R⁴² and R⁴³ are taken together with the nitrogen to which they are attached to form a pyrrolidinyl ring, wherein said pyrrolidinyl ring is unsubstituted or substituted with 1 to 5 R³⁸ substituents.

In a preferred embodiment of the compounds according to the present invention, R⁷ is selected from the group consisting of —H, halogen, nitro, azido, —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, S(O)R^{6a}, —SO₂R^{6a}, —SO₃R^{6a}, —SO₂NR^{6a}R^{6b}, —COR^{6a}, —CO₂R^{6a}, —CONR^{6a}R^{6b}, —(C₁-C₄)fluoroalkyl, —(C₁-C₄)fluoroalkoxy, —(CZ³Z⁴)ₐCN, and a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C¹-C⁶)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶))ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ⁵Z⁶ units may be the same or different, or
R^{6a} and R^{6b} taken together with adjacent atoms form atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O)NHZ⁷, —OC(O)NZ⁷Z⁸, —NHC(O)Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(OH)₃, —OP(OH)₃, —P(O)(OH)₂, OP(OZ⁷)₃, —S(O)₃H, —S(O)Z⁷, S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ₙNHZ³, —(CZ⁹Z¹⁰)ᵣNZ⁷Z⁸, —X⁶(CZ⁹Z¹⁰)ᵣ-(C₃-C₈)cycloalkyl, —X⁶(CZ⁹Z¹⁰)ᵣ—(C₅-C₈)cycloalkenyl, —X⁶(CZ⁹Z¹⁰)ᵣ-aryl and —X⁶(CZ⁹Z¹⁰)ᵣ-heterocycle, wherein
r is 1, 2, 3 or 4;
X⁶ is selected from the group consisting of O, S, NH, —C(O)—, —C(O)NH—, —C(O)O—, —S(O)—, —S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, F, a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y² or Y³ groups attached to the same or adjacent carbon atoms may be selected together to form a carbocycle or heterocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl].

In a preferred embodiment of the compounds according to the present invention R⁷ is selected from the group consisting of —H, —Y-(aryl), —Y-(heteroaryl) and C(O)-heterocyclyl, each of which, except for —H, is optionally substituted with 1 to 5 R³⁸.

In a preferred embodiment of the compounds according to the present invention, D is selected from the group consisting of

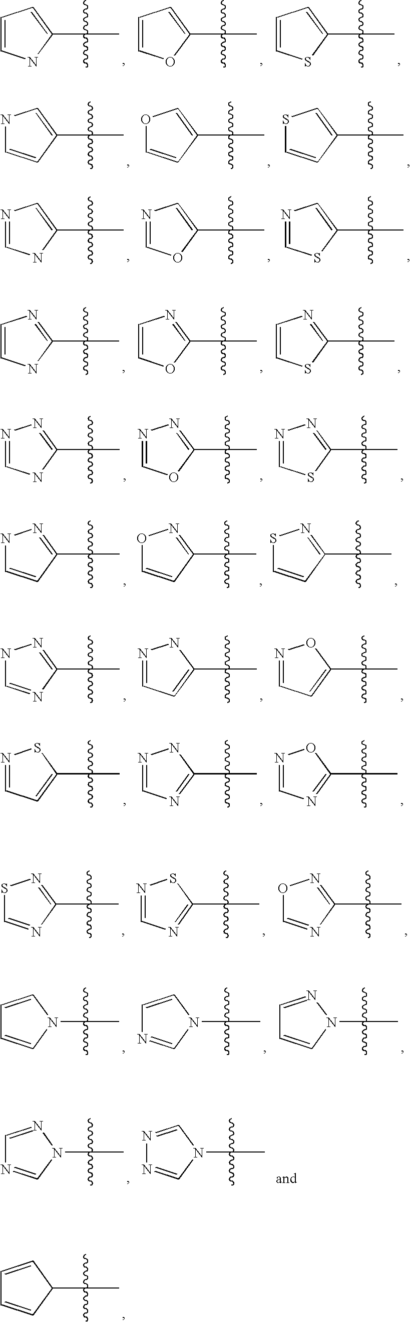

wherein the members of said group are optionally substituted by 1 to 3 R³⁸.

In a preferred embodiment of the compounds according to the present invention, D is selected from the group consisting of

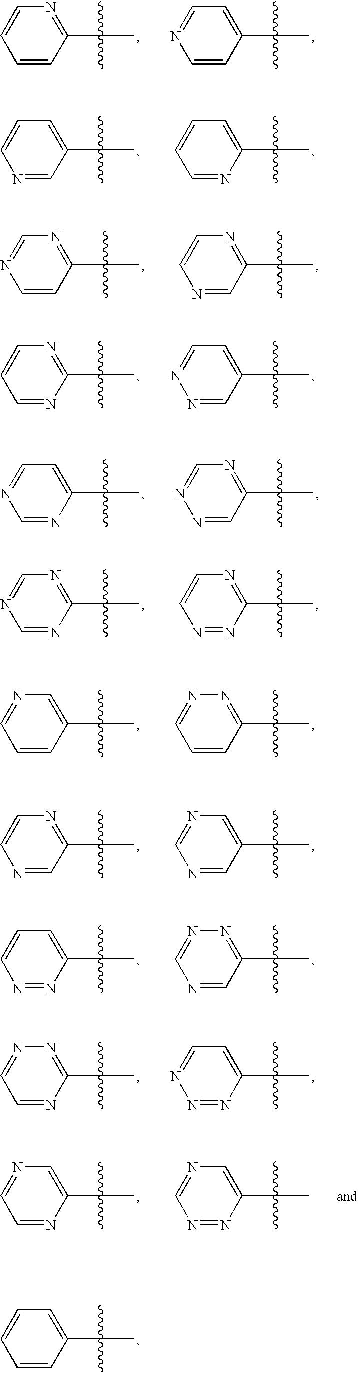

wherein the members of said group are optionally substituted with 1 to 3 R³⁸.

In a preferred embodiment of the compounds according to the present invention, D is selected from the group consisting of

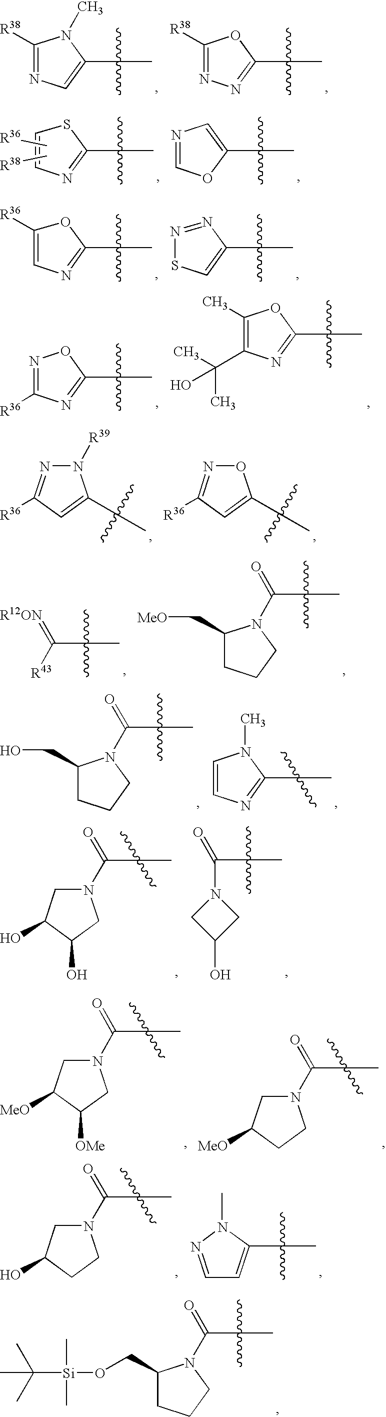

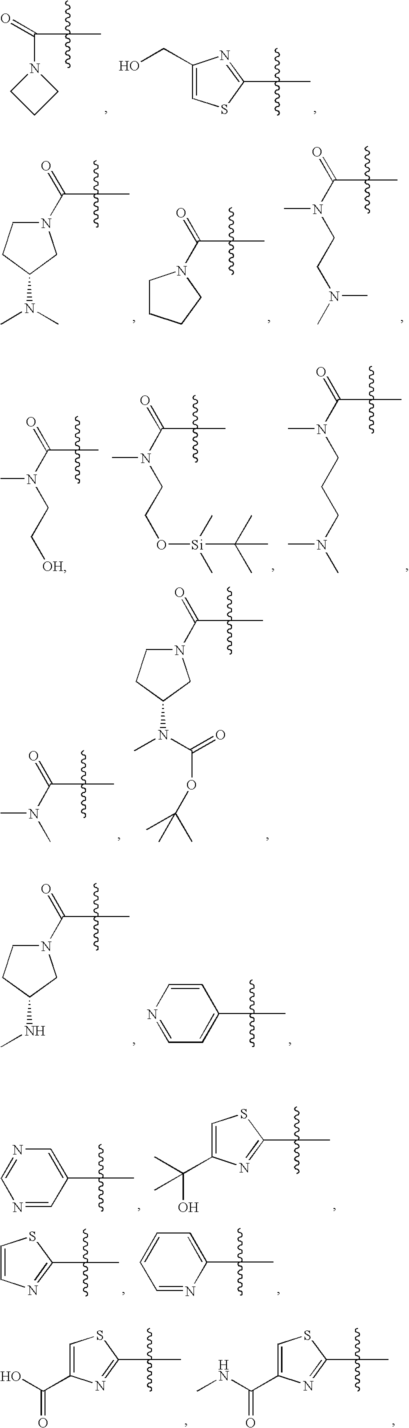

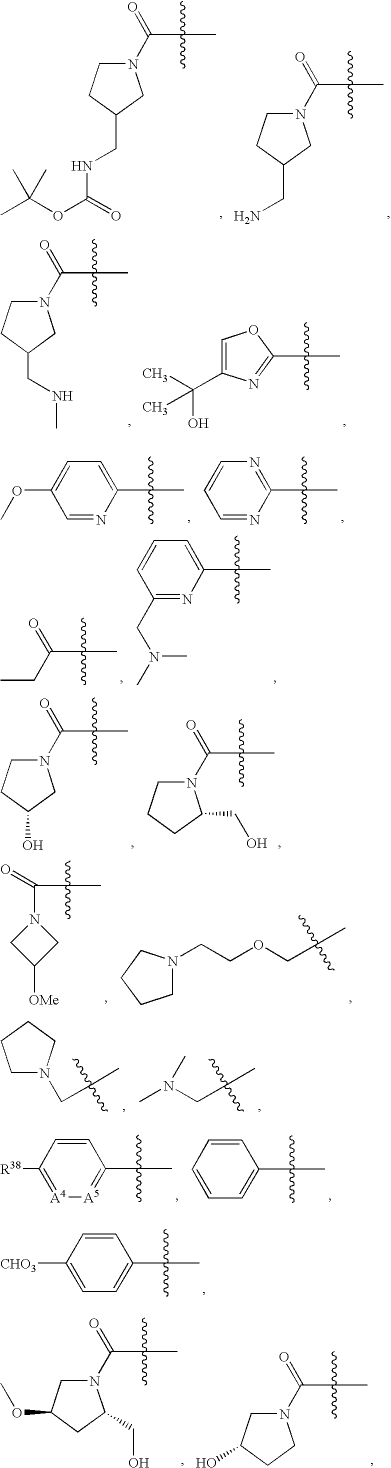

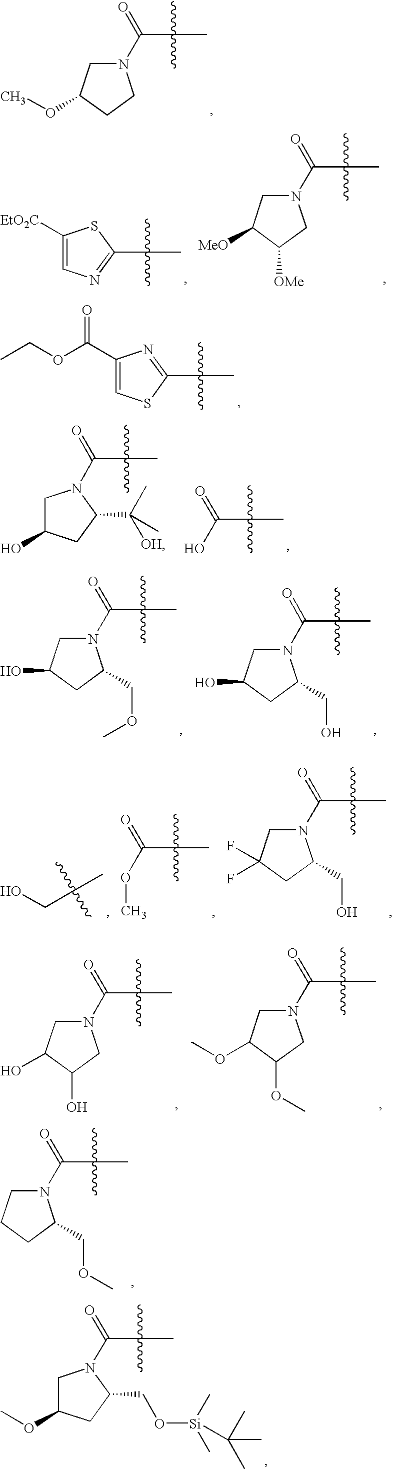

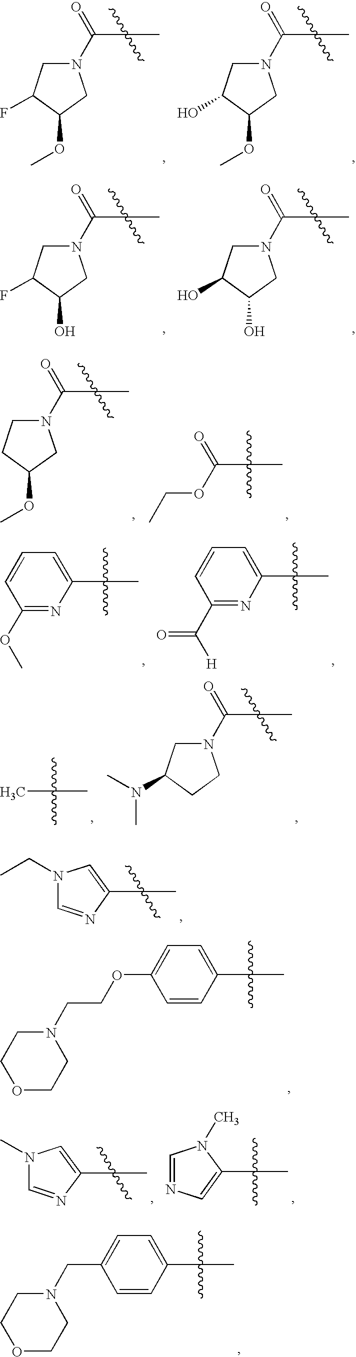

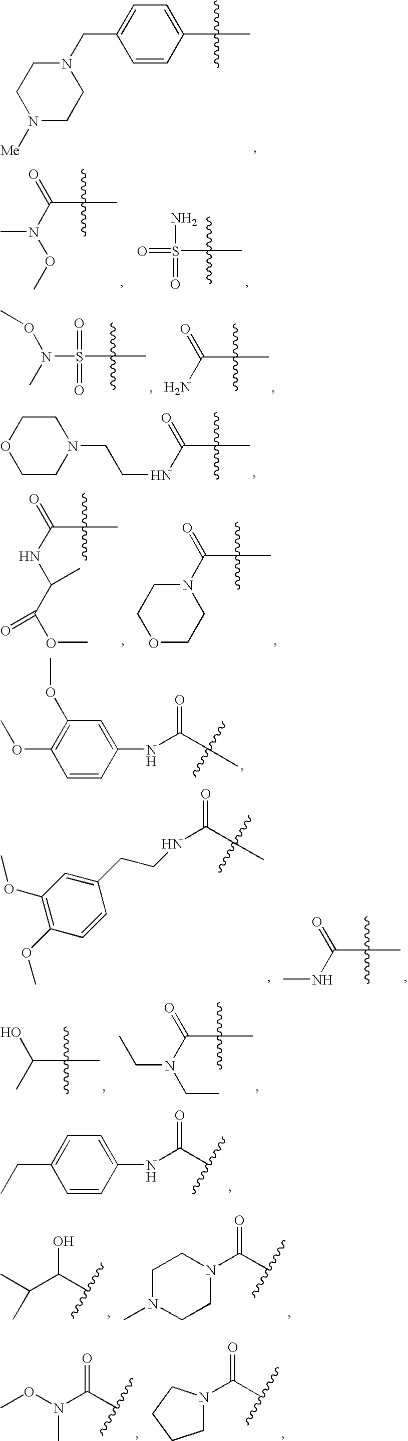

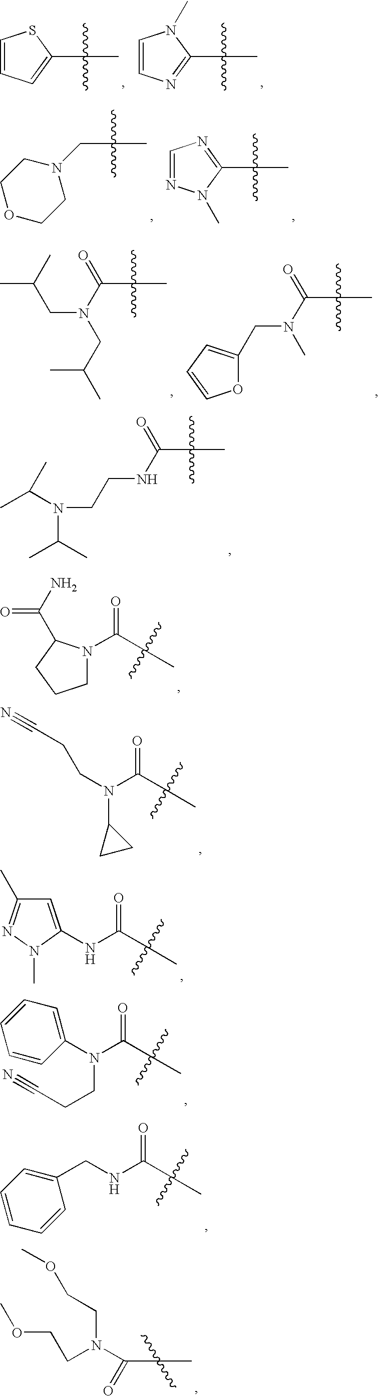

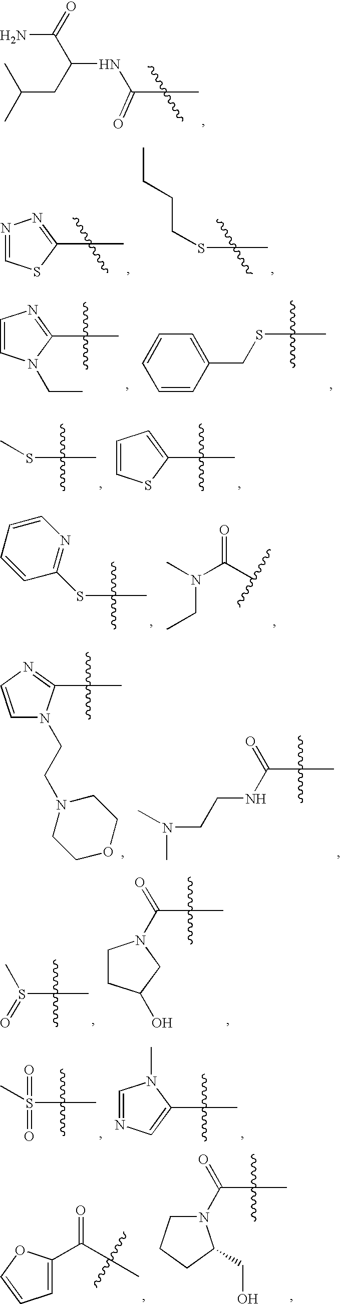

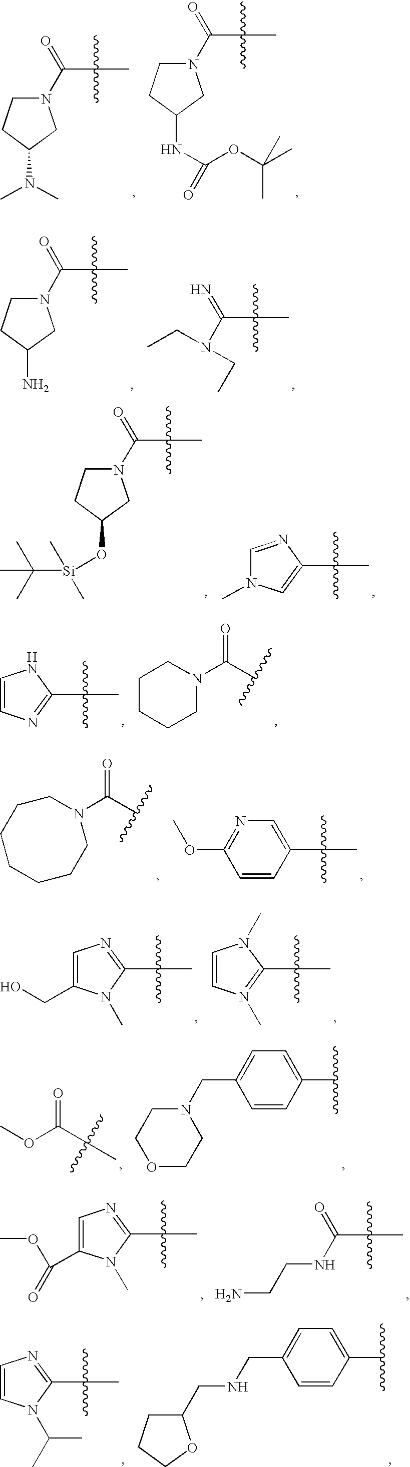

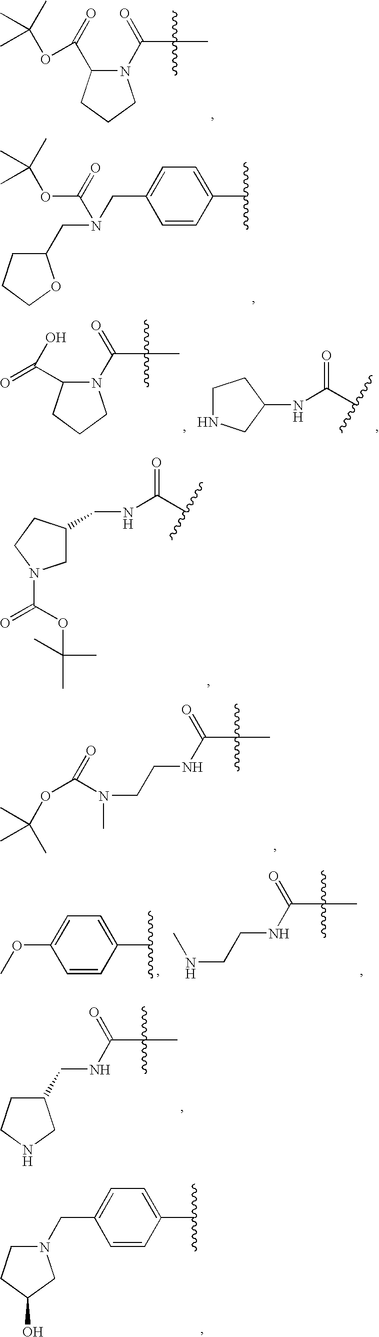

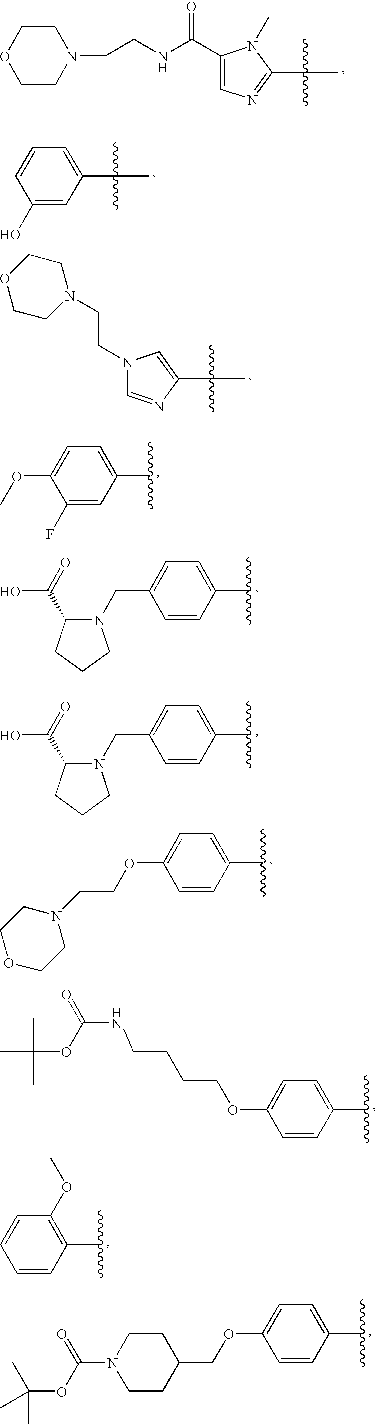

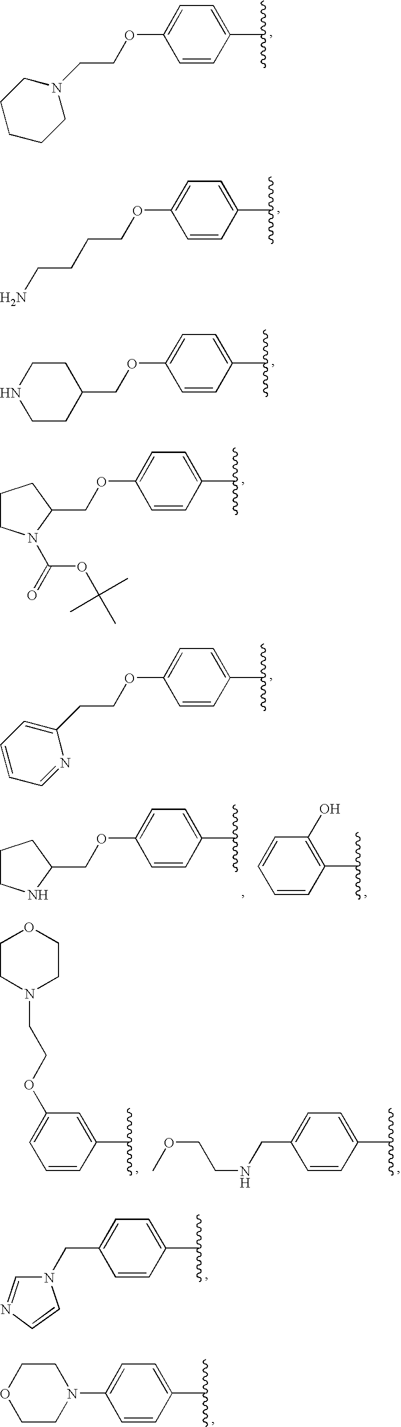

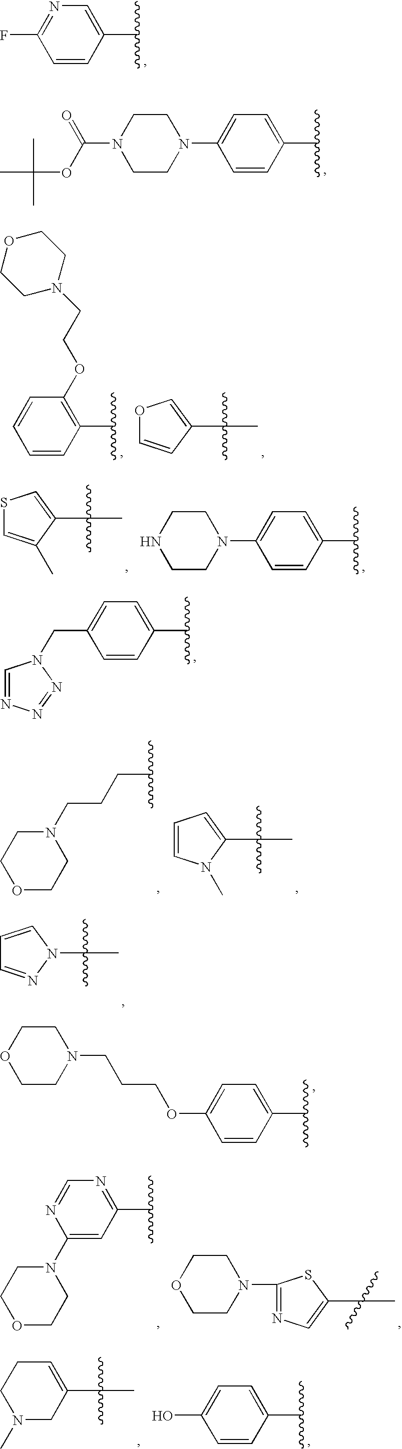

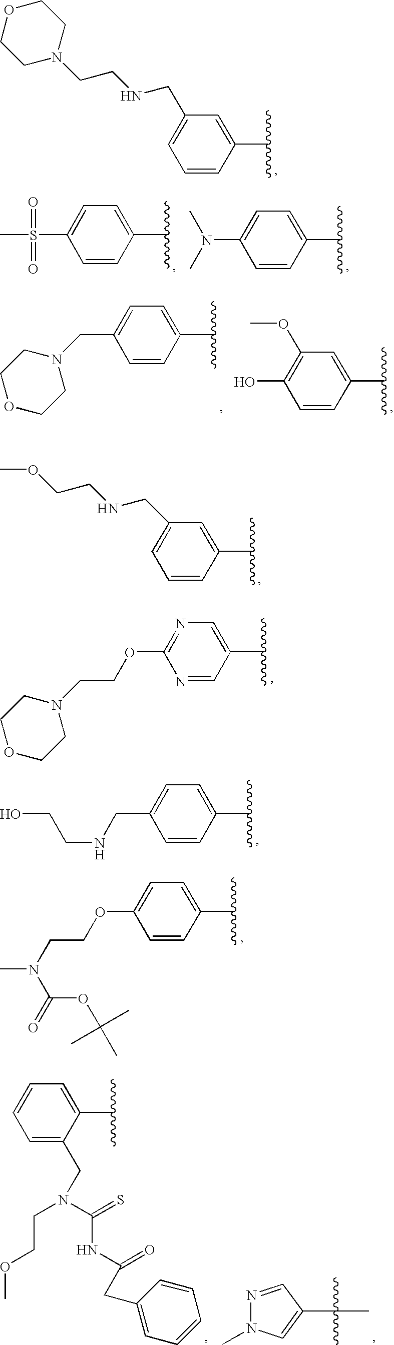

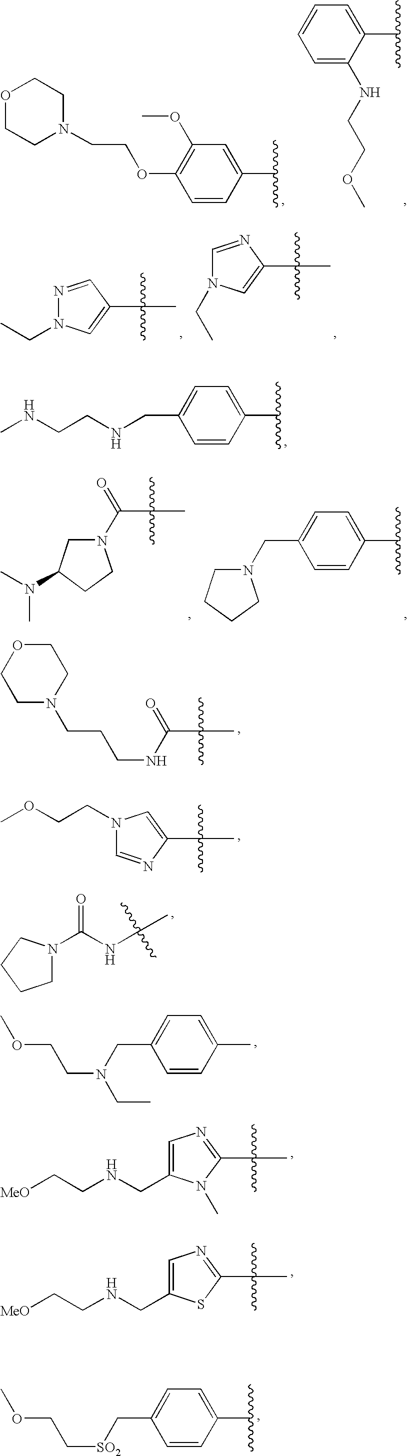

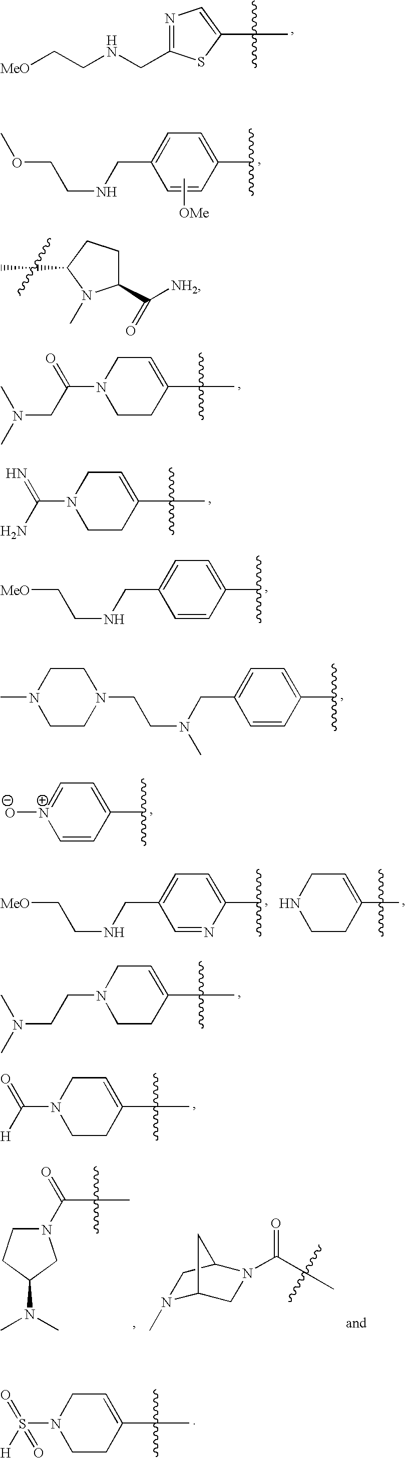

In a preferred embodiment of the compounds according to the present invention, R⁷ is selected from the group consisting of phenyl and pyridyl, which are optionally substituted by 1 to 5 R³⁸.

According to another preferred embodiment of the present invention, D is phenyl, pyridyl, furanyl, imidazolyl, tetrahydropyridyl, thienyl, pyrazolyl, each of which is optionally substituted with 1 to 5 independently selected R³⁸ groups, more preferably 1 to 3 independently selected R³⁸ groups, and more preferably 1 or 2 independently selected R³⁸ groups.

In another preferred embodiment according to the present invention, D is phenyl, optionally substituted with 1 to 5 independently selected R³⁸ groups, more preferably 1 to 3 independently selected R³⁸ groups, and more preferably 1 or 2 independently selected R³⁸ groups.

In another preferred embodiment according to the present invention, D is pyridyl, optionally substituted with 1 to 5 independently selected R³⁸ groups, more preferably 1 to 3 independently selected R³⁸ groups, and more preferably 1 or 2 independently selected R³⁸ groups.

In another preferred embodiment according to the present invention, D is phenyl, optionally substituted with one R³⁸.

In another preferred embodiment according to the present invention, D is pyridyl, optionally substituted with one R³⁸.

In another preferred embodiment according to the present invention, D is phenyl, substituted with one R³⁸.

In another preferred embodiment according to the present invention, D is pyridyl, substituted with one R³⁸.

In another preferred embodiment according to the present invention, D is imidazolyl, substituted with one R³⁸.

In a preferred embodiment of the present invention, each R³⁸ is independently selected from the group consisting of —C(O)NR³⁶R³⁹, —C(O)O—(CH₂)ₙNR³⁶R³⁹, —(CH₂)ⱼNR³⁹(CH₂)ᵢS(O)ⱼ(C₁-C₆ alkyl), —(CH₂)ⱼNR¹⁹(CH₂)R⁶ and —C(O)(CH₂)ⱼNR³⁹(CH₂)ₙR³⁶, and most preferably —(CH₂)ⱼNR³⁹(CH₂)ₙR³⁶.

In a preferred embodiment of the present invention each R³⁸ is independently selected from the group consisting of halo, optionally substituted C₁-C₆ alkyl and —(CH₂)ⱼNR³⁹(CH₂)ₙR³⁶.

In a preferred embodiment of the present invention, R³⁶ is selected from the group consisting of H, —OH, C₁-C₆ alkyl and —(CH₂)ₙA⁴R³⁷, more preferably —(CH₂)ₙOR³⁷ or —(CH₂)ₙSR³⁷, more preferably still —(CH₂)ₙOR³⁷, wherein each n is an integer independently ranging from 0 to 6 (preferably 0 to 4, more preferably 0 to 2, more preferably 1 or 0, most preferably 0), and i is an integer ranging from 2 to 6.

In a preferred embodiment of the present invention, each R³⁸ is independently halo, C₁-C₆alkyl or —(CH₂)ⱼNR³⁹(CH₂)ₙR³⁶.

In a preferred embodiment of the present invention, each R³⁸ is independently —(CH₂)ⱼNR³⁹(CH₂)ₙR³⁶, wherein j is 1 and n is 2.

In a preferred embodiment of the present invention each R³⁸ is independently —(CH₂)ⱼNH(CH₂)ₙA⁴R³⁷.

In a preferred embodiment of the present invention each R³⁸ is independently —(CH₂)ⱼNH(CH₂)ₙOR³⁷, wherein j is 1 or 2 and n is 2.

In a preferred embodiment of the present invention each R³⁸ is independently —(CH₂)NH(CH₂)₂OR³⁷, wherein R³⁷ is optionally substituted C₁-C₆ alkyl, preferably —CH₃.

In a preferred embodiment of the present invention each R³⁸ is independently —(CH₂)NH(CH₂)₃OR³⁷, wherein R³⁷ is optionally substituted C₁-C₆ alkyl, preferably —CH₃.

In a preferred embodiment of the present invention each R³⁸ is independently —(CH₂)₂NH(CH₂)₂OR⁷, wherein R³⁷ is optionally substituted C₁-C₆ alkyl, preferably —CH₃.

In a preferred embodiment of the present invention each R³⁸ is independently —(CH₂)₂NH(CH₂)₃OR³⁷, wherein R³⁷ is optionally substituted C₁-C₆ alkyl, preferably —CH₃.

In a preferred embodiment of the present invention, each R³⁸ is independently —(CH₂)ⱼNR³⁹(CH₂)ᵢS(O)ⱼ(C₁-C₆ alkyl), preferably —(CH₂)NH(CH₂)₂S(O)₂CH₃.

In a preferred embodiment of the present invention, each R³⁸ is independently —C(O)(CH₂)ⱼNR³⁹(CH₂)ₙR³⁶.

In a preferred embodiment of the present invention, each R³⁸ is independently —C(O)NR³⁹(CH₂)₂OR³⁷.

In a preferred embodiment of the present invention, each R³⁸ is independently —C(O)NH(CH₂)₂OR³⁷, wherein R³⁷ is optionally substituted C₁-C₆ alkyl, preferably —CH₃.

In a preferred embodiment of the present invention, each R³⁸ is independently —C(O)O—(CH₂)ₙNR³⁶R³⁹.

In a preferred embodiment of the present invention, each R³⁸ is independently —C(O)O—(CH₂)ₙNR³⁶R³⁹, wherein R³⁶ and R³⁹ are each independently C₁-C₆ alkyl, preferably —CH₃.

In a preferred embodiment of the present invention, each R³⁸ is independently —C(O)O—(CH₂)ₙNHR³⁶R³⁹, wherein R³⁶ and R³⁹ are each independently C₁-C₆ alkyl, preferably —CH₃, and n is preferably 2.

In a preferred embodiment of the present invention each R³⁸ is independently —(CH₂)ⱼNR³⁹(CH₂)ᵢC₃-C₇cycloalkyl, preferably —(CH₂)NHC₃cycloalkyl.

In a preferred embodiment of the present invention each R³⁸ is independently selected from the group consisting of —(CH₂)ₙP(═O)(C₁-C₆alkyl)₂, —(CH₂)ⱼNR³⁹CH₂(CH₂)ₙP(═O)(C₁-C₆alkyl)₂, —NR¹³C(X³)NR¹³—C₁-C₆alkyl-P(═O)(C₁-C₆alkyl)₂, —NR¹³C(X³)NR¹³-arylP(═O)(C₁-C₆alkyl)₂ and —NR¹³C(X³)NR¹³-heteroarylP(═O)(C₁-C₆alkyl)₂, wherein X³ is preferably O or S.

In a preferred embodiment of the present invention each R³⁸ is independently —(CH₂)ₙP(═O)(C₁-C₆alkyl)₂.

In a preferred embodiment of the present invention each R³⁸ is independently —(CH₂)ₙP(═O)(C₁-C₆alkyl)₂.

In a preferred embodiment of the present invention each R³⁸ is independently —(CH₂)₁₋₃P(═O)(C₁-C₃alkyl)₂.

In a preferred embodiment of the present invention each R³⁸ is independently —(CH₂)₁₋₃P(═O)(C₁-C₃alkyl)₂.

In a preferred embodiment of the present invention each R³⁸ is independently —(CH₂)ₙP(═O)(CH₃)₂.

In a preferred embodiment of the present invention each R³⁸ is independently —(CH₂)₁₋₃P(═O)(CH₃)₂.

In a preferred embodiment of the present invention each R³⁸ is independently —(CH₂)ⱼNR³⁹CH₂(CH₂)ₙP(═O)(C₁-C₆alkyl)₂.

In a preferred embodiment of the present invention each R³⁸ is independently —(CH₂)ⱼNHCH₂(CH₂)ₙP(═O)(C₁-C₆alkyl)₂.

In a preferred embodiment of the present invention each R³⁸ is independently —(CH₂)₁₋₂NR³⁹(CH₂)₁₋₃P(═O)(C₁-C₆alkyl)₂.

In a preferred embodiment of the present invention each R³⁸ is independently —(CH₂)₁₋₂NH(CH₂)₁₋₃P(═O)(C₁-C₆alkyl)₂.

In a preferred embodiment of the present invention each R³⁸ is independently —(CH₂)₁₋₂NR³⁹(CH₂)₁₋₃P(═O)(C₁-C₃alkyl)₂.

In a preferred embodiment of the present invention each R³⁸ is independently —(CH₂)₁₋₂NH(CH₂)₁₋₃P(═O)(C₁-C₃alkyl)₂.

In a preferred embodiment of the present invention each R³⁸ is independently —(CH₂)₁₋₂NH(CH₂)₁₋₃P(═O)(CH₃)₂.

In a preferred embodiment of the present invention each R³⁸ is independently —(CH₂)₁NR³⁹(CH₂)₂P(═O)(C₁-C₆alkyl)₂.

In a preferred embodiment of the present invention each R³⁸ is independently —(CH₂)₁NH(CH₂)₂P(═O)(C₁-C₆alkyl)₂.

In a preferred embodiment of the present invention each R³⁸ is independently —(CH₂)₁NH(CH₂)₂P(═O)(C₁-C₃alkyl)₂.

In a preferred embodiment of the present invention each R³⁸ is independently —(CH₂)₁NH(CH₂)₂P(═O)(CH₃)₂.

In a preferred embodiment of the present invention each R³⁸ is independently selected from the group consisting of —NR¹³C(O)NR¹³—C₁-C₆alkyl-P(═O)(C₁-C₆alkyl)₂, —NR¹³C(S)NR¹³—C₁-C₆alkyl-P(═O)(C₁-C₆alkyl)₂, —NR¹³C(O)NR¹³-arylP(═O)(C₁-C₆alkyl)₂, —NR¹³C(S)NR¹³-arylP(═O)(C₁-C₆alkyl)₂, —NR¹³C(O)NR¹³-heteroarylP(═O)(C₁-C₆alkyl)₂ and —NR¹³C(S)NR¹³-heteroarylP(═O)(C₁-C₆alkyl)₂.

In a preferred embodiment of the present invention each R³⁸ is independently selected from the group consisting of —NR¹³C(O)NR¹³-arylP(═O)(C₁-C₃alkyl)₂, —NR¹³C(S)NR¹³-arylP(═O)(C₁-C₃alkyl)₂, —NR¹³C(O)NR¹³-heteroarylP(═O)(C₁-C₃alkyl)₂ and —NR¹³C(S)NR¹³-heteroarylP(═O)(C₁-C₃alkyl)₂.

In a preferred embodiment of the present invention, D is substituted with a preferred R³⁸ as described herein, and further substituted with halo or C₁-C₆alkyl.

In a preferred embodiment of the present invention, D is phenyl or pyridinyl, and R³⁸ is C₁-C₆alkyl, —(CH₂)ⱼNR³⁹(CH₂)ₙR³⁶, —NR¹³C(X³)NR¹³—C₁-C₆alkyl-P(═O)(C₁-C₆alkyl)₂, —(CH₂)ⱼNR³⁹CH₂(CH₂)ₙP(═O)(C₁-C₆alkyl)₂, —NR¹³C(X³)NR¹³-arylP(═O)(C₁-C₆alkyl)₂ or —NR¹³C(X¹)NR¹³-heteroarylP(═O)(C₁-C₆alkyl)₂, wherein X³ is preferably O or S.

In a preferred embodiment of the compounds according to the present invention, D is defined by the group R¹, wherein R¹ is —C≡CH or —C≡C—(CR⁴⁵R⁴⁵)ₙ—R⁴⁶; wherein
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, —(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁹, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙC(O)R⁴⁹, —C(O)₂R⁴⁹, —(CH₂)ₙSR⁴⁹, —(CH₂)ₙS(O)R⁴⁹—(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹, together with the atom to which they are attached, form a 3-8 membered carbo- or hetero-cyclic ring.

In a preferred embodiment of the compounds according to the present invention,
R⁴⁶ is selected from the group consisting of —N(R⁴⁷)—C(O)—N(R⁴⁷)(R⁴⁸), —N(R⁴⁷)—C(O)—(CH₂)ₙ—R⁴⁸ and —(CH₂)ₙNR⁴⁷R⁴⁸; wherein
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, —(CH₂)ₙOR⁵⁰, —(CH₂)ₙS(O)₂R⁴⁹ and —(CH₂)ₙCN, or R⁴⁷ and R⁴⁸, together with the atom to which they are attached, form a 3-8 membered carbo- or hetero-cyclic ring; and
R⁵⁰ and R⁵¹ are independently selected from the group consisting of H and (C₁-C₆)alkyl, or R⁵⁰ and R⁵¹, together with the atom to which they are attached, form a 3-8 membered carbo- or hetero-cyclic ring.

In a preferred embodiment of the compounds according to the present invention, D is selected from the group consisting of

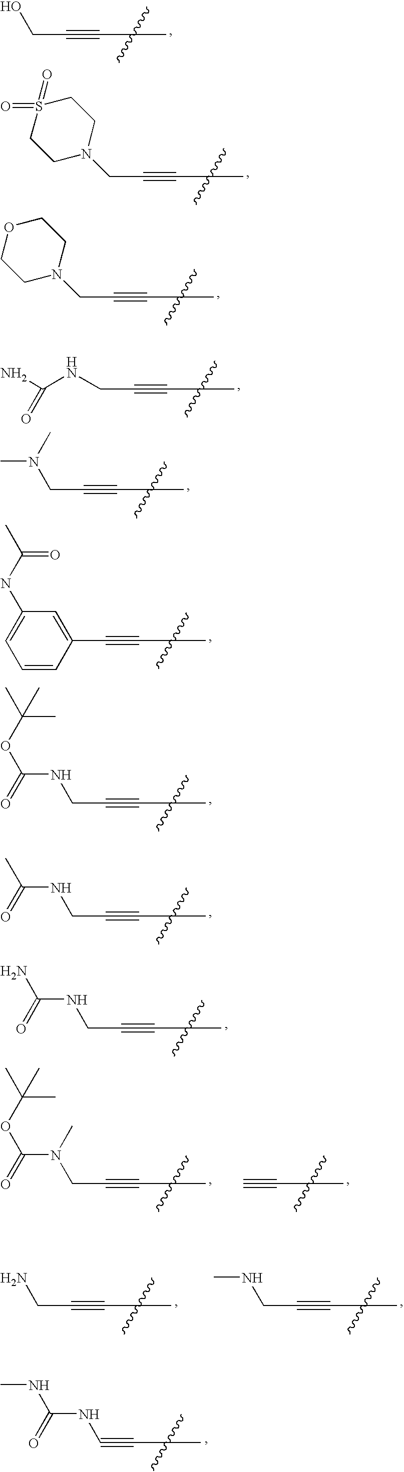

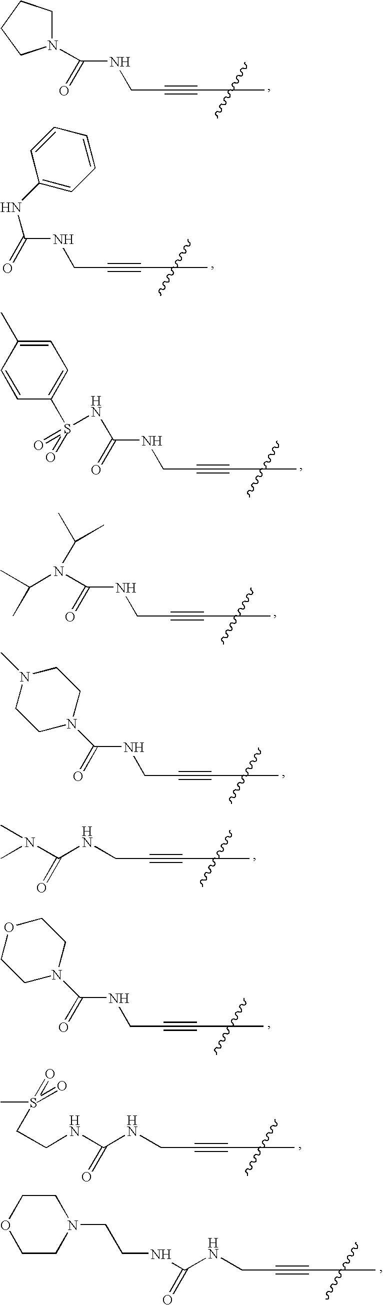

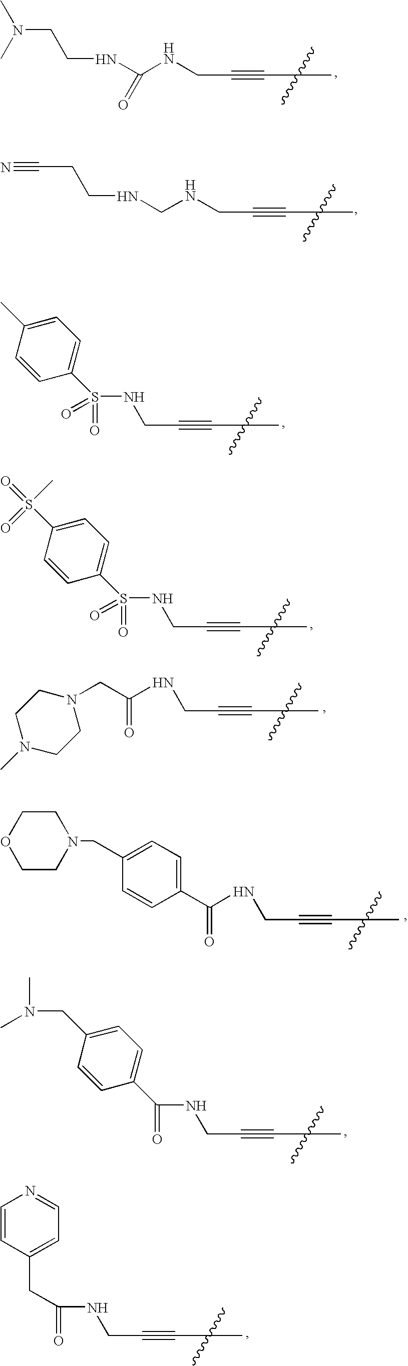

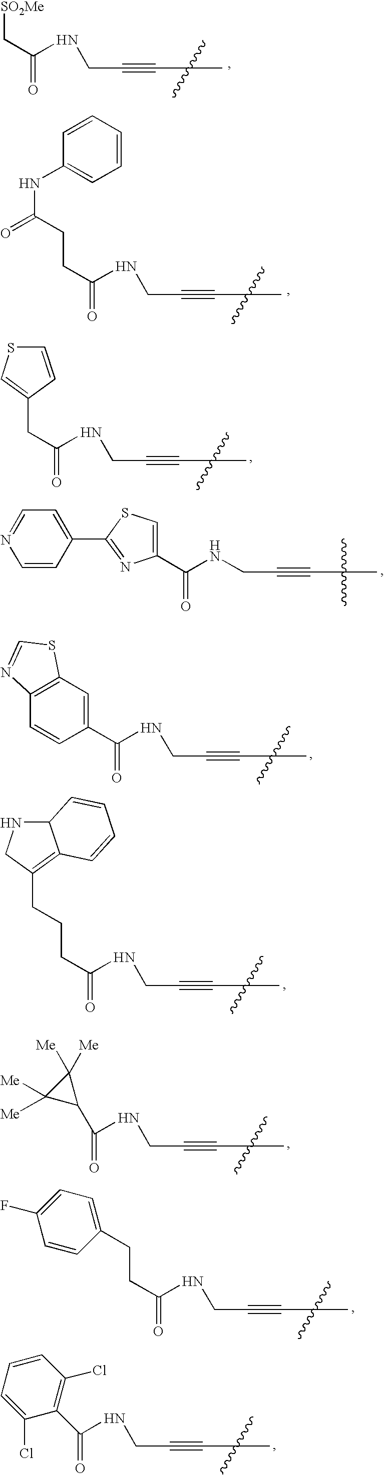

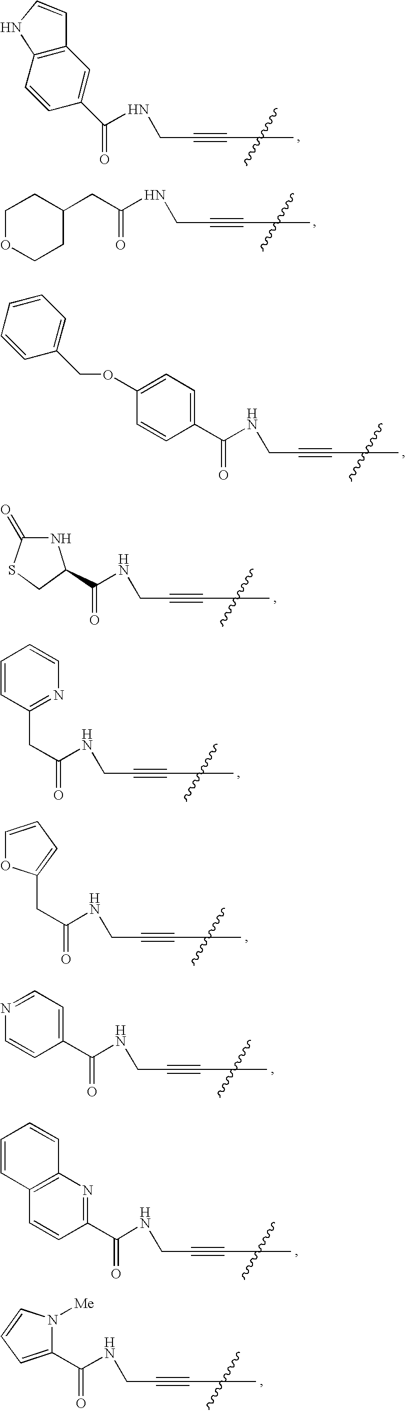

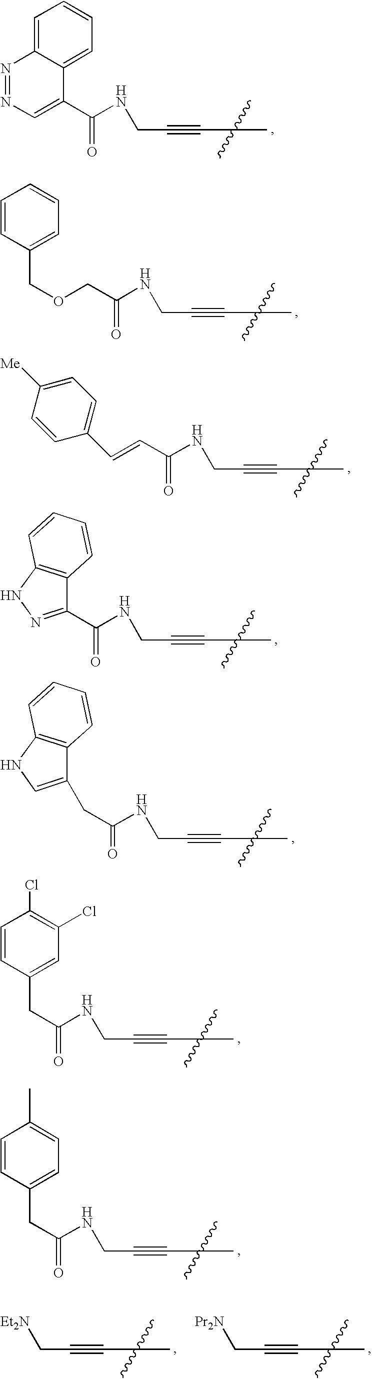

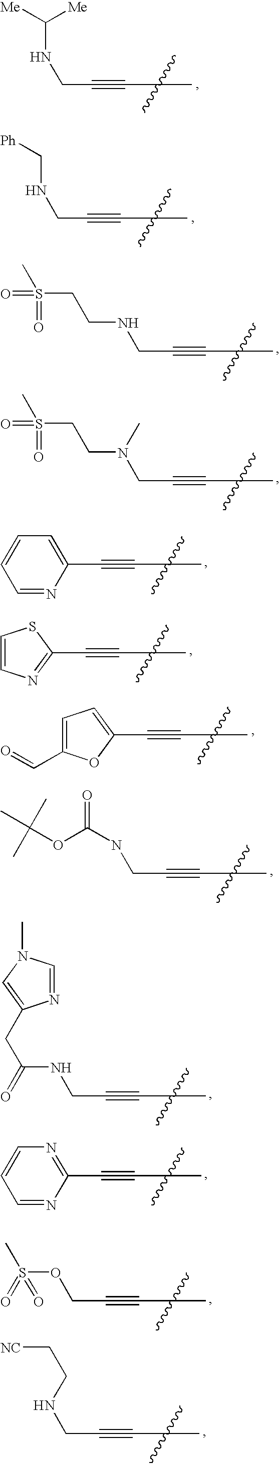

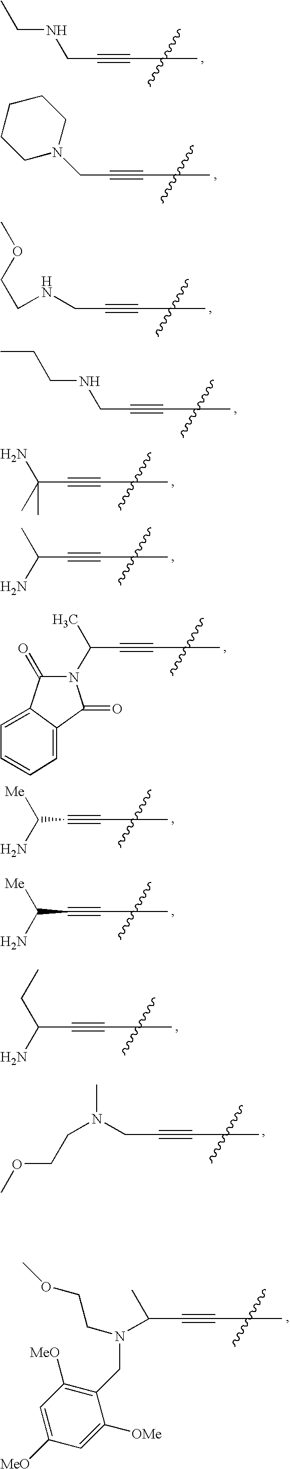

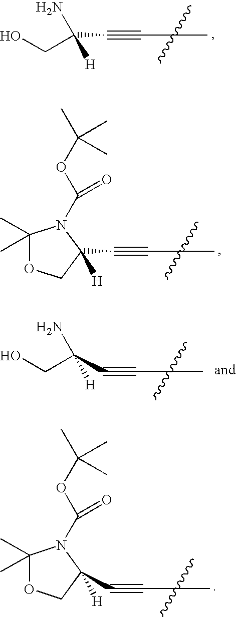

In a preferred embodiment of the compounds according to the present invention, D is defined by the group R²¹, wherein R²¹ is defined by -(Z¹¹)-(Z¹²)ₘ-(Z¹³)ₘ₁, wherein
Z¹¹ is heterocyclyl, when m and m1 are 0, or heterocyclylene, when either m or m1 are 1;
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³, (C₁-C₃)alkyl, —(CH₂)_{q}OR⁵³, —C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³; and
R⁵⁶ is selected from the group consisting of NH₂, (C₁-C₃)alkyl and OR⁵².

In a preferred embodiment of the compounds according to the present invention, Z¹¹ is a heterocyclyl and m and m1 are each 0.

In a preferred embodiment of the compounds according to the present invention, Z¹¹ is a heterocyclyl and m is 0 and n is 0, where the heterocyclyl group is selected from the group consisting of

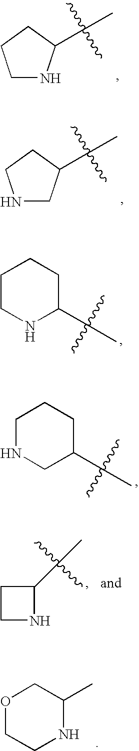

In a preferred embodiment of the compounds according to the present invention, Z¹¹ is heterocyclylene, Z¹² is OC(O), m is 1, m1 is 1 and Z¹³ is heterocyclyl.

In a preferred embodiment of the compounds according to the present invention, Z¹¹ is

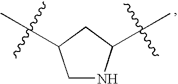

Z is OC(O), and
Z¹³ is

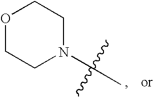

Z¹³ is N(H)R⁵², wherein R⁵² is (C₁-C₃)alkyl.

In a preferred embodiment of the compounds according to the present invention Z¹¹ is heterocyclylene, Z¹² is C(O) and m is 1, m1 is 1 and Z¹³ is (C₁-C₃)haloalkyl.

In a preferred embodiment of the compounds according to the present invention, Z¹¹ is

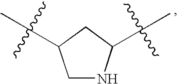

Z is C(O), and
Z¹³ is (C₁-C₃)haloalkyl, preferably —CF₃.

In a preferred embodiment of the compounds according to the present invention, Z¹¹ is heterocyclylene, m is 0, m1 is 1 and Z¹³ is heterocyclyl.

In a preferred embodiment of the compounds according to the present invention, Z¹¹ is

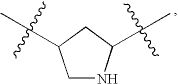

m is 0, and
Z¹³ is

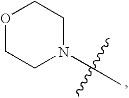

or
Z¹³ is (C₁-C₃)alkyl, or
Z¹³ is —OH, or
Z¹³ is —OR⁵², wherein R⁵² is (C₁-C₃)alkyl, preferably —CH₃ or
Z¹³ is halo, preferably —F, or
Z¹³ is (C₁-C₃)hydroxyalkyl, preferably —CH₃OH.

In a preferred embodiment of the compounds according to the present invention, D is selected from the group consisting of

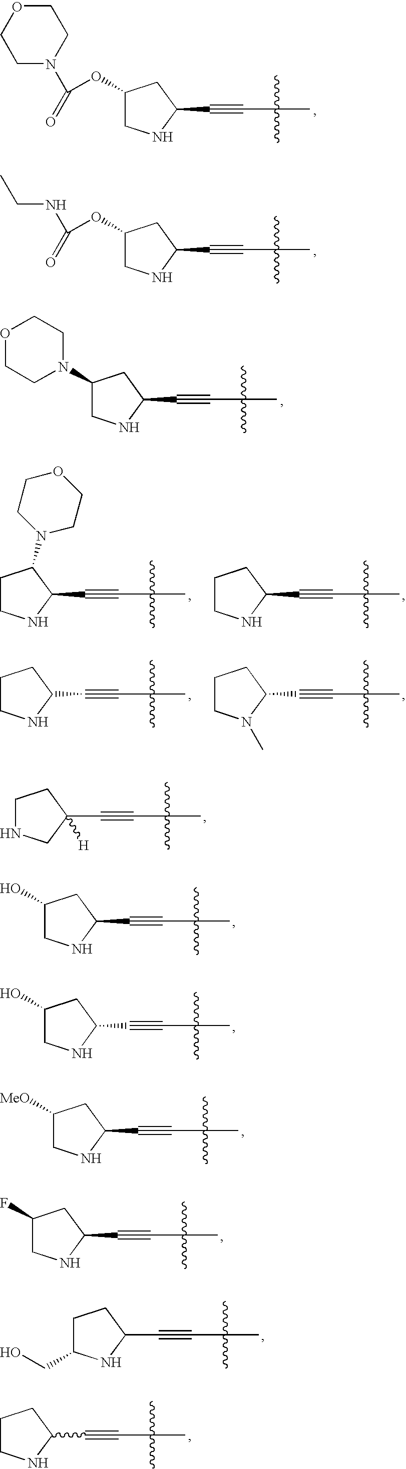

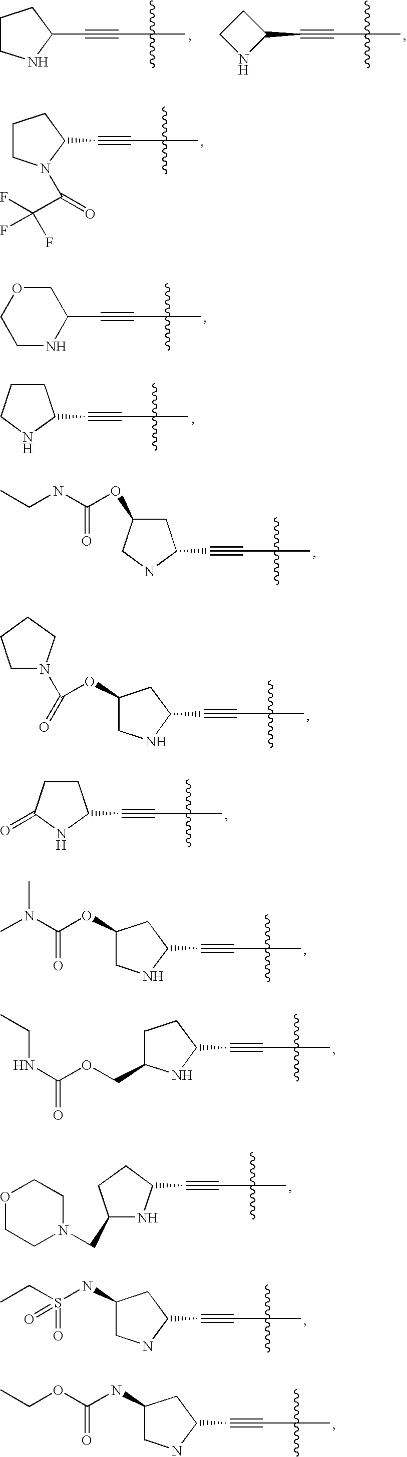

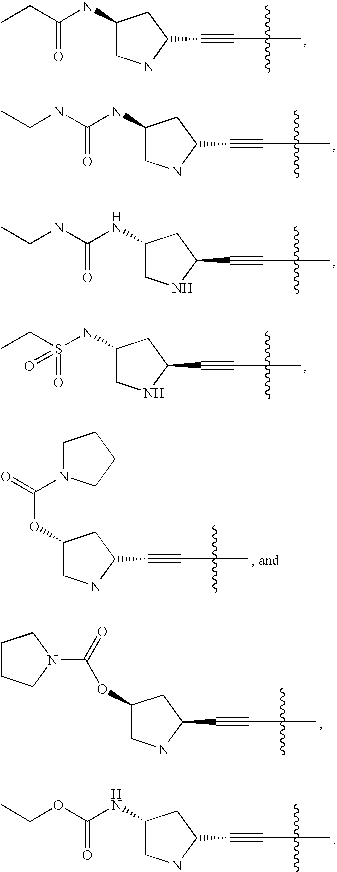

In a preferred embodiment of the compounds according to the present invention wherein D is defined by the group R²¹, the heterocyclic or heterocyclyl group is optionally substituted with a substituent selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo, aryl, aralkyl, heteroaryl and (C₁-C₆)perfluoroalkyl. Such a ring may be optionally fused to one or more other “heterocyclic” ring or cycloalkyl ring. Preferred examples of “heterocyclic” moieties include, but are not limited to, tetrahydrofuranyl, pyranyl, 1,4-dioxaneyl, 1,3-dioxanyl, piperidinyl, piperazinyl, 2,4-piperazinedionyl, pyrrolidinyl, pyrrolidinon-2-yl, pyrrolidinon-3-yl, pyrrolidinon-4-yl, pyrrolidinon-5-yl, imidazolidinyl, pyrazolidinyl, morpholinyl, thiomorpholinyl, tetrahydrothiopyranyl, tetrahydrothiophenyl, and the like

In a preferred embodiment of the compounds according to the present invention wherein D is defined by the group R²¹, the heterocyclylene group is optionally substituted with substituents selected from the group consisting of (C₁-C₆)alkyl, (C₁-C₆)alkoxy, (C₁-C₆)alkylsufanyl, (C₁-C₆)alkylsulfenyl, (C₁-C₆)alkylsulfonyl, oxo, hydroxyl, mercapto, amino optionally substituted by alkyl, carboxy, carbamoyl optionally substituted by alkyl, alkylcarboxyamide, carboxyamide, aminosulfonyl optionally substituted by alkyl, ureido, arylurea, arylthiourea, alkylurea, cycloalkylurea, sulfonylurea, nitro, cyano, halo and (C₁-C₆)perfluoroalkyl, multiple degrees of substitution being allowed. Such a ring may be optionally fused to one or more benzene rings or to one or more of another “heterocyclic” rings or cycloalkyl rings. Preferred examples of “heterocyclylene” include, but are not limited to, tetrahydrofuran-2,5-diyl, morpholine-2,3-diyl, pyran-2,4-diyl, 1,4-dioxane-2,3-diyl, 1,3-dioxane-2,4-diyl, piperidine-2,4-diyl, piperidine-1,4-diyl, pyrrolidine-1,3-diyl, pyrrolidinon-2,3-yl, pyrrolidinon-2,4-yl, pyrrolidinon-2,5-yl, pyrrolidinon-3,4-yl, pyrrolidinon-3,5-yl, pyrrolidinon-4,5-yl, morpholine-2,4-diyl, and the like.

In a preferred embodiment of the present invention, D is selected from the group consisting of

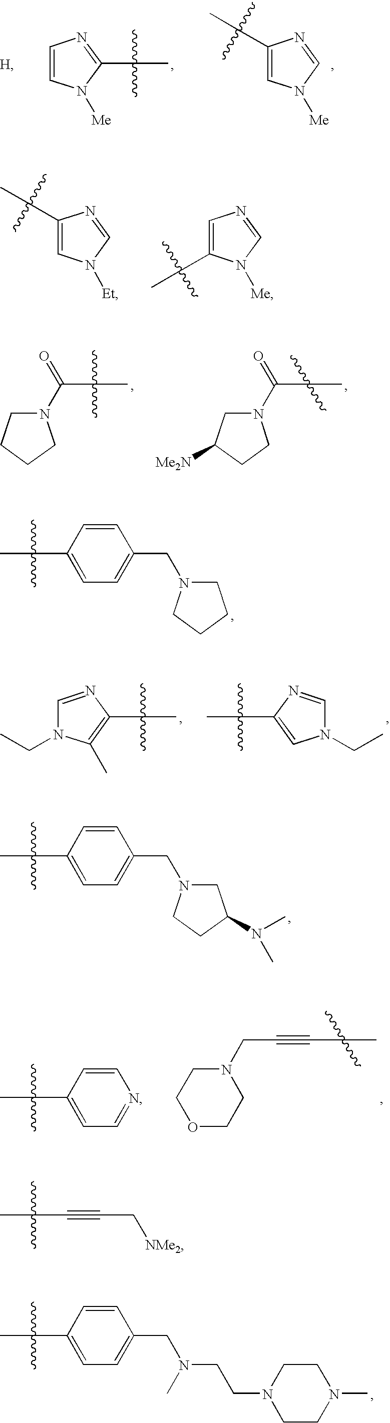

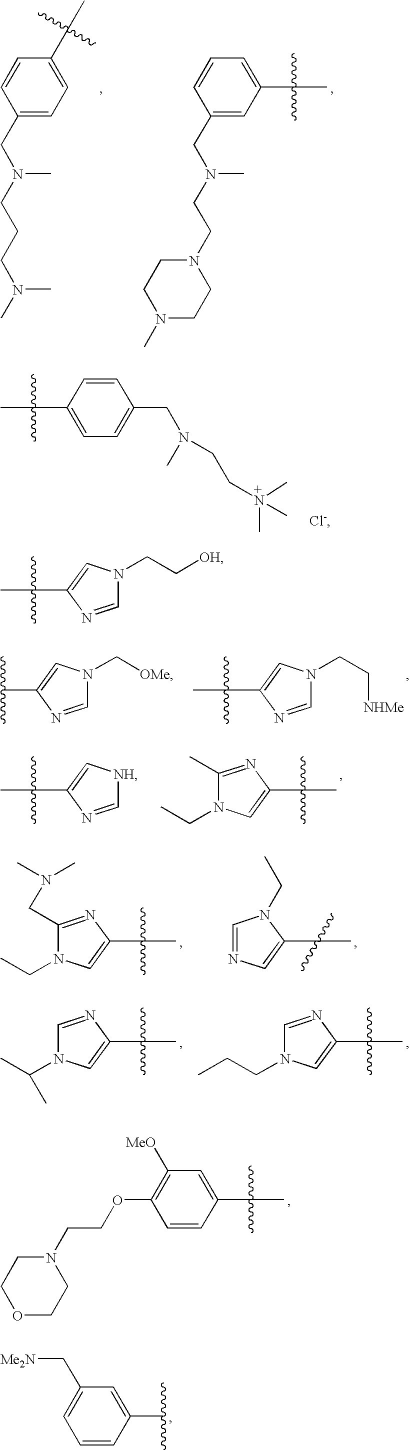

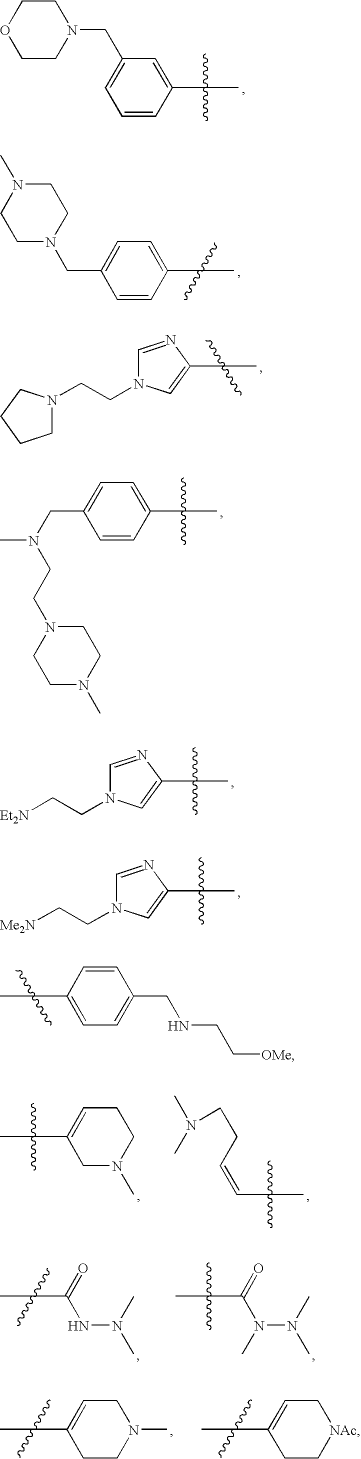

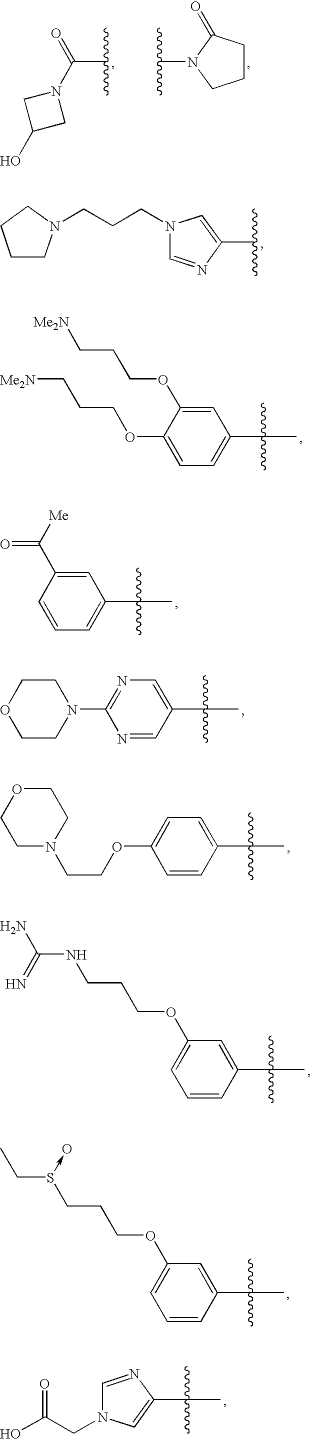

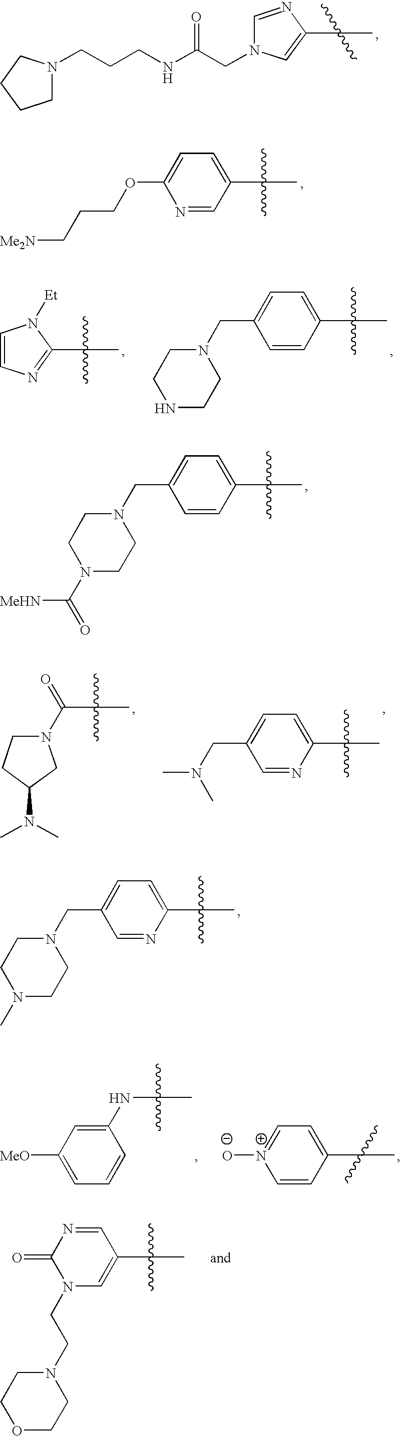

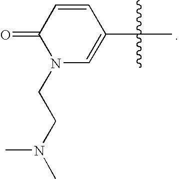

In a preferred embodiment of the present invention,
D is selected from the group consisting of H, —NH₂, —NR⁴²C(═O)R⁴³, —NR⁴²C(═O)NR⁴³—R¹⁰¹, —C≡C—(CR⁴⁵R⁴⁵)ₙ—R⁴⁶, —Y—NR⁴²R⁴³, —NR^{6a}C(O)OR^{6b}, oxo and —C(O)NR⁴²R⁴³.

In a preferred embodiment of the present invention,
D is selected from the group consisting of —NR⁴²C(═O)R⁴³, —NH₂, —NR⁴²C(═O)NR⁴³—R¹⁰¹, —C≡C—(CR⁴⁵R⁴⁵)ₙ—R⁴⁶ and —Y—NR⁴²R⁴³.

In a preferred embodiment of the present invention,
D is —NR⁴²C(═O)-heterocyclyl wherein the heterocyclyl is optionally substituted, preferably with —NR³⁶R³⁹.

In a preferred embodiment of the compounds according to the present invention, M is a monocyclic moiety having the formula:

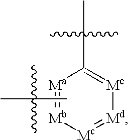

wherein each of M^{a}, M^{b}, M^{c}, M^{d} and M^{e} are independently selected from N and CR¹⁰⁷, with the proviso that no more than 3 of M^{a}, M^{b}, M^{c}, M^{d} and M^{e} are N, wherein
R¹⁰⁷ is selected from the group consisting of hydrogen, halogen, CN, nitro, azido, C₁-C₁₂alkyl, —C₁-C₁₂alkyl-cycloalkyl, —C₁-C₁₂alkyl-aryl, —C₁-C₁₂alkyl-heterocyclyl, —C₁-C₁₂alkyl-heteroaryl, —C₁-C₁₂heteroalkyl-cycloalkyl, —C₁-C₁₂heteroalkyl-aryl, —C₁-C₁₂heteroalkyl-heterocyclyl, —C₁-C₁₂heteroalkyl-heteroaryl, C₂-C₁₂alkenyl, C₂-C₁₂alkynyl, C₃-C₁₂cycloalkyl, C₆-C₁₂aryl, 3-12 membered heteroalicyclic, 5-12 membered heteroaryl, —S(O)₀₋₂R¹⁰⁸, —SO₂NR¹⁰⁸R¹⁰⁹, —S(O)₂OR¹⁰⁸, —NO₂, —NR¹⁰⁸R¹⁰⁹, —(CR¹¹⁰R¹¹¹)₀₋₄OR¹⁰⁸, —CN, —C(O)R¹⁰⁸, —OC(O)R¹⁰⁸, —O(CR110R111)₀₋₄NR¹⁰⁸R¹⁰⁹, —C(═NR¹¹⁰)NR¹⁰⁸R¹⁰⁹, NR¹⁰⁸C(O)NR¹⁰⁹R¹¹⁰, —NR¹⁰⁸S(O)₁₋₂R¹⁰⁹, —C(O)NR¹⁰⁸R¹⁰⁹, —CH═CH—C₆-C₁₂aryl, —CH═CH-(3-12 membered heteroalicyclic), —CH═CH-(5-12 membered heteroaryl), —CH═CH—S(O)₀₋₂R¹⁰⁸, —CH═CH—SO₂NR¹⁰⁸R¹⁰⁹, —CH═CH—S(O)₂OR¹⁰⁸, —CH═CH—NO₂, —CH═CH—NR¹⁰⁸R¹⁰⁹, —CH═CH—(CR¹¹⁰R¹¹¹)₀₋₄OR¹⁰⁸, —CH═CH—CN, —CH═CH—C(O)R¹⁰⁸, —CH═CH—OC(O)R¹⁰, —CH═CH—O(CR110R¹¹¹)₀₋₄R¹⁰⁸, —CH═CH—NR¹⁰⁸C(O)R¹⁰⁹, —CH═CH—(CR¹¹⁰R¹¹¹)₀₋₄C(O)OR¹⁰⁸, —CH═CH—(CR¹¹⁰R¹¹¹)₀₋₄NR¹⁰⁸R¹⁰⁹, —CH═CH—C(═NR¹¹⁰)NR¹⁰⁸R¹⁰⁹, —CH═CH—NR¹⁰⁸C(O)NR¹⁰⁹R¹¹⁰, —CH═CH—NR¹⁰⁸S(O)₁₋₂R¹⁰⁹, —CH═CH—C(O)NR¹⁰⁸R¹⁰⁹, —C≡C—C₆-C₁₂aryl, —C≡C-(3-12 membered heteroalicyclic), —C≡C-(5-12 membered heteroaryl), —C≡C—S(O)₀₋₂R¹⁰⁸, —C≡C—SO₂NR¹⁰⁸R¹⁰⁹, —C≡C—S(O)₂OR¹⁰⁸, —C≡C—NO₂, —C≡C—NR¹⁰⁸R¹⁰⁹, —C≡C—(CR¹¹⁰R¹¹¹)₀₋₄OR¹⁰⁸, —C≡C—CN, —C≡C—C(O)R¹⁰⁸, —C≡C—OC(O)R¹⁰⁸, —C≡C—O(CR110R111)₀₋₄R¹⁰⁸, —C≡C—NR¹⁰⁸C(O)R¹⁰⁹, —C≡C—(CR¹¹⁰R¹¹¹)₀₋₄C(O)OR¹⁰⁸—C≡C—(CR¹¹⁰R¹¹¹)₀₋₄NR¹⁰⁸R¹⁰⁹, —C≡C—C(═NR¹¹⁰)NR¹⁰⁸R¹⁰⁹, —C≡C—NR¹⁰⁸C(O)NR¹⁰⁹R¹¹⁰, —C≡C—NR¹⁰⁸S(O)₁₋₂R¹⁰⁹ and —C≡C—C(O)NR¹⁰⁸R¹⁰⁹,
   wherein each hydrogen of which is optionally substituted by an R¹¹⁷ group;
each R¹⁰⁸, R¹⁰⁹, R¹¹⁰ and R¹¹¹, which may be the same or different, is independently selected from hydrogen, halogen, C₁-C₁₂alkyl, C₂-C₁₂alkenyl, C₂-C₁₂alkynyl, C₃-C₁₂cycloalkyl, C₆-C₁₂aryl, 3-12 membered heteroalicyclic and 5-12 membered heteroaryl, or any two of R¹⁰⁸, R¹⁰⁹, R¹¹⁰ and R¹¹¹ bound to the same nitrogen atom may, together with the nitrogen to which they are bound, be combined to form a 3 to 12 membered heteroalicyclic or 5-12 membered heteroaryl group optionally containing 1 to 3 additional heteroatoms selected from N, O and S, or any two of R¹⁰⁸, R¹⁰⁹, R¹¹⁰ and R¹¹¹ bound to the same carbon atom may be combined to form a C₃-C₁₂ cycloalkyl, C₆-C₁₂ aryl, 3-12 membered heteroalicyclic or 5-12 membered heteroaryl group, and each hydrogen of R¹⁰⁸, R¹⁰⁹, R¹¹⁰ and R¹¹¹ is optionally substituted by from 1 to 6 R¹¹⁷ groups;
each R¹¹⁷, which may be the same or different, is independently selected from halogen, C₁-C₁₂ alkyl, C₂-C₁₂ alkenyl, C₂-C₁₂ alkynyl, C₃-C₁₂ cycloalkyl, C₆-C₁₂ aryl, 3-12 membered heteroalicyclic, 5-12 membered heteroaryl, —CN, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic) and —O—(CH₂)₀₋₄(5 to 12 membered heteroaryl), —C(O)R¹¹⁹, —C(O)OR¹¹⁹ and —C(O)NR¹¹⁹R¹²⁰, and each hydrogen in R¹¹⁷ is optionally substituted by an R¹¹⁸ group;
each R¹¹⁸, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic, 5-12 membered heteroaryl, —O—C₁-C₁₂ alkyl, —O—(CH₂)₀₋₄C₃-C₁₂ cycloalkyl, —O—(CH₂)₀₋₄C₆-C₁₂ aryl, —O—(CH₂)₀₋₄(3-12 membered heteroalicyclic), —O—(CH₂)₀₋₄(5-12 membered heteroaryl) and —CN, and each hydrogen in R¹¹⁸ is optionally substituted by a group selected from halogen, —OH, —CN, —C₁-C₁₂alkyl which may be partially or fully halogenated, —O—C₁-C₁₂ alkyl which may be partially or fully halogenated, —CO, —SO, —SO₂ and —SO₃H;
   each R¹¹⁹ and R¹²⁰, which may be the same or different, is independently selected from hydrogen, halogen, C₁-C₁₂ alkyl, C₁-C₁₂ alkoxy, C₃-C₁₂ cycloalkyl, C₆-C₁₂ aryl, 3-12 membered heteroalicyclic and 5-12 membered heteroaryl, and each R¹¹⁹ and R¹²⁰ is optionally substituted by a group selected from halogen, —OH, —CN, —C₁-C₁₂ alkyl which may be partially or fully halogenated, —O—C₁-C₁₂ alkyl which may be partially or fully halogenated and SO₃H, or R¹¹⁹ and R¹²⁰, taken together with the nitrogen atom to which they are attached, may form a 3-12 membered heteroalicyclic ring optionally substituted by from 1 to 6 R¹¹⁸ groups.

In a preferred embodiment of the present invention, M is an optionally substituted heteroaryl.

In a preferred embodiment of the compounds according to the present invention, M is selected from the group consisting of

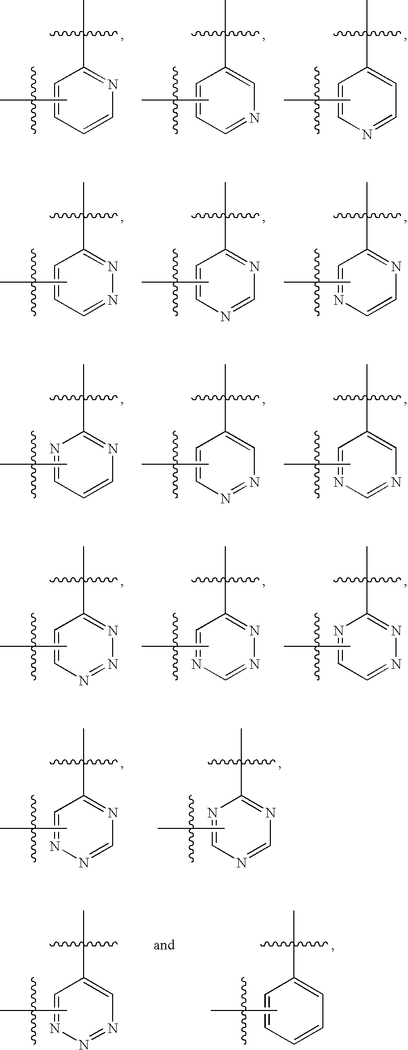

wherein each ring —CH— is optionally substituted with R¹⁰⁷.

In a preferred embodiment of the compounds according to the present invention, M is selected from the group consisting of

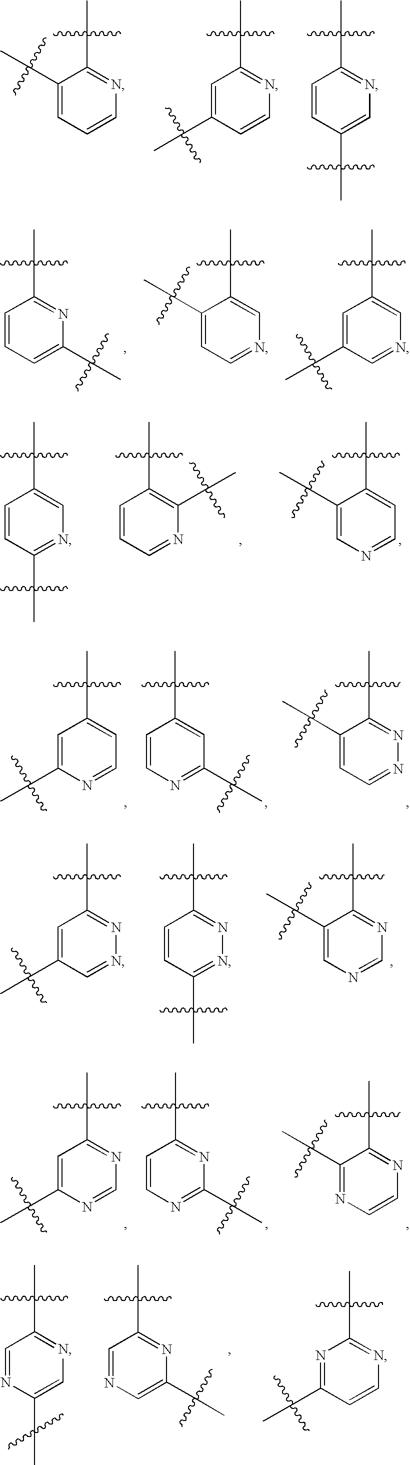

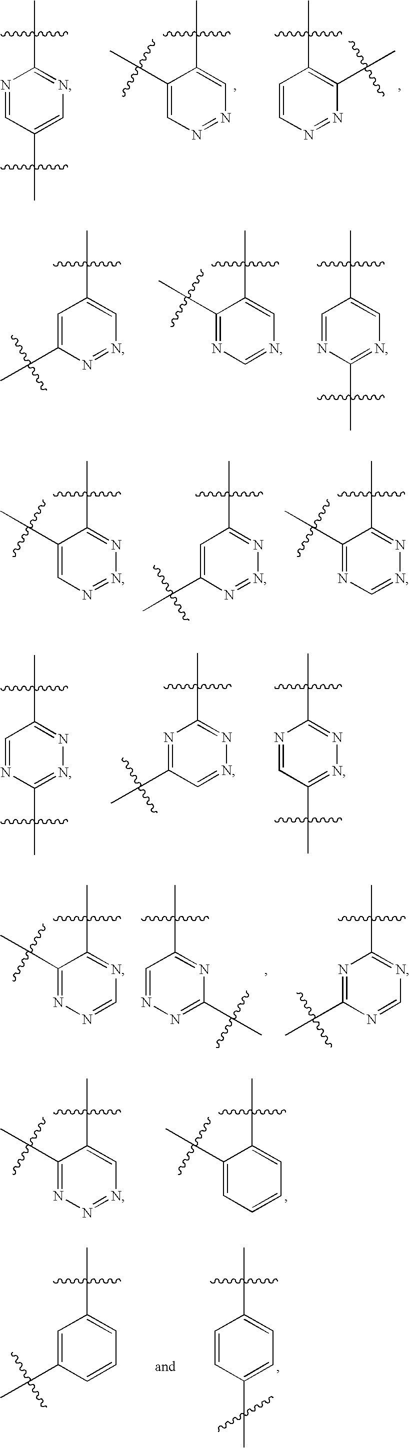

wherein each ring —CH— is optionally substituted with R¹⁰⁷.

In a preferred embodiment of the present invention, M is pyridine or pyrimidine, preferably pyridine.

In a preferred embodiment of the compounds according to the present invention, Z is selected from the group consisting of —O—, —S—, —S(O)₀₋₂ and —NR⁵—, wherein R⁵ is selected from the group consisting of H, an optionally substituted (C₁-C₅)acyl and C₁-C₆ alkyl-O—C(O), wherein C₁-C₆ alkyl is optionally substituted.

In a preferred embodiment of the present invention, Z is selected from the group consisting of —O—, —NH—C(O)—NH—, C₂alkynylene, —NH—, —NH—C(O)— and —NH—SO2-.

In a preferred embodiment of the compounds according to the present invention, Z is —O—.

In a preferred embodiment of the compounds according to the present invention, -M-Z- taken together is selected from the group consisting of

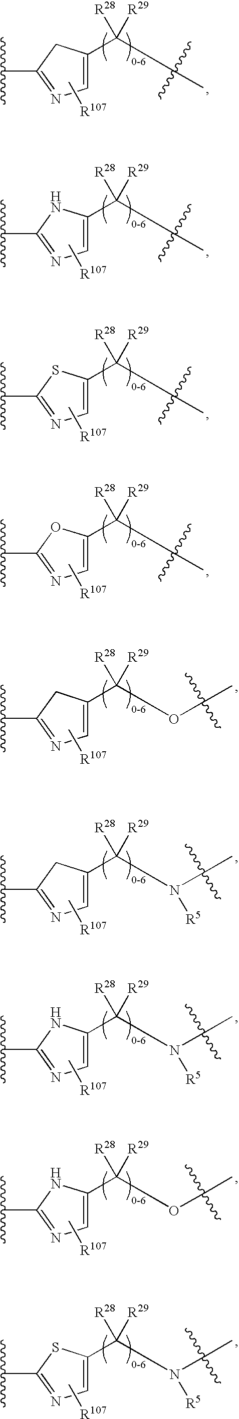

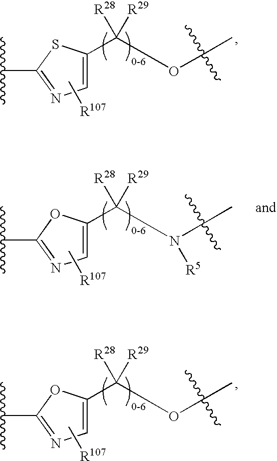

wherein R²⁸ and R²⁹ are independently selected from the group consisting of H, alkyl, substituted alkyl, cycloalkyl, substituted cycloalkyl, aryl, substituted aryl, or taken together form a carbocyclic or heterocyclic ring of 3 to 8 atoms.

In a preferred embodiment of the compounds according to the present invention, Ar is a group of the formula (Z),

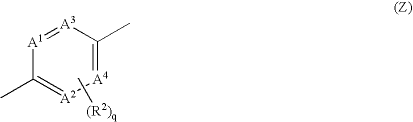

wherein,
A¹, A², A³ and A⁴ are independently selected from the group consisting of N and —CH—, with the proviso that no more than two of A¹, A², A¹ and A⁴ can be N;
R² at each occurrence is independently selected from the group consisting of —H, halogen, trihalomethyl, vinyl, —C≡CH, —CH═CH—, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, —CH═CH-trihalomethyl, —CH═CH—CN, —CH═CH—NO₂, —CH═CH—NH₂, —CH═CH—OR³, —CH═CH—NR³R⁴, —CH═CH—S(O)₀₋₂R³, —CH═CH—S(O)₂NR³R³, —CH═CH—C(O)OR³, —CH═CH—C(O)NR³R³, —CH═CH—N(R³)SO₂R³, —CH═CH—N(R³)C(O)R³, —CH═CH—N(R³)CO₂R³, —CH═CH—C(O)R³, —C≡C-trihalomethyl, —C≡C—CN, —C≡C—NO₂, —C≡C—NH₂, —C≡C—OR³, —C≡C—NR³R⁴, —C≡C—S(O)₀₋₂R³, —C≡C—S(O)₂NR³R³, —C≡C—C(O)OR³, —C≡C—C(O)NR³R³, —C≡C—N(R³)SO₂R³, —C≡C—N(R³)C(O)R³, —C≡C—N(R³)CO₂R³, —C≡C—C(O)R³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T² wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
R³ selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, which optionally contains at least one additional annular heteroatom selected from the group consisting of N, O, S and P; and
q is an integer from 0 to 4.

In a preferred embodiment of the compounds according to the present invention, Ar is selected from the group consisting of phenyl, pyrazine, pyridazine, pyrimidine and pyridine, wherein each of said phenyl, pyrazine, pyridazine, pyrimidine and pyridine are optionally substituted with between zero and four R².

In a preferred embodiment of the compounds according to the present invention, Ar is phenyl, optionally substituted with between zero and four R².

In a preferred embodiment of the present invention, Ar is optionally substituted phenyl, preferably optionally substituted with a substituent selected from the group consisting of F, Cl, C₁-C₆alkyl and C₁-C₆alkoxy, more preferably F; and

In a preferred embodiment of the compounds according to the present invention, Ar is phenyl, substituted with between zero and four halo.

In a preferred embodiment of the compounds according to the present invention, G is the group B-L-T, wherein
B is selected from the group consisting of absent, —N(R¹³)—, —N(SO₂R¹³)—, —O—, —S(O)₀₋₂ and —C(═O)—;
L is selected from the group consisting of absent, —C(═S)N(R¹³)—, —C(═NR¹⁴)N(R¹³)—, —SO₂N(R¹³)—, —SO₂—, —C(═O)N(R¹³)—, —N(R¹³)—, —C(═O)C₁₋₂alkyl-N(R¹³)—, —N(R¹³)C₁₋₂alkyl-C(═O)—, —C(═O)C₀₋₁alkyl-C(═O)N(R¹³)—, —C₀₋₄alkylene, —C(═O)C₀₋₁alkyl-C(═O)OR³—, —C(═NR¹⁴)—C₀₋₁alkyl-C(═O)—, —C(═O)—, —C(═O)C₀₋₁alkyl-C(═O)— and an optionally substituted four to six-membered heterocyclyl containing between one and three annular heteroatoms including at least one nitrogen, wherein an alkyl group of the aforementioned L group is optionally substituted; and
T is selected from the group consisting of —H, —R¹³, —C₀₋₅alkyl, —C₀₋₅alkyl-Q, —O—C₀₋₅alkyl-Q, —C₀₋₅alkyl-O-Q, —N(R¹³)—C₀₋₅alkyl-Q, —C₀₋₅alkyl-SO₂—C₀₋₅alkyl-Q, —C(═O)—C₀₋₅alkyl-Q, —C(═S)—C₀₋₅-alkyl-Q, —C(═NR¹⁴)—C₀₋₅-alkyl-Q, —C₀₋₅alkyl-N(R¹³)-Q, —C(═O)—N(R¹³)—C₀₋₅alkyl-Q, —C(═S)—N(R¹³)—C₀₋₅alkyl-Q, —C(═NR¹⁴)—N(R¹³)—C₀₋₅alkyl-Q, —(C₀₋₅alkyl-C(O))₀₋₁—C₀₋₅alkyl-Q wherein each C₀₋₅alkyl is optionally substituted.

In a preferred embodiment of the compounds according to the present invention, G is a group B-L-T, wherein
B is selected from the group consisting of absent, —N(R¹³)—, —N(SO₂R¹³)—, —O—, —S(O)₀₋₂ and —C(═O)—;
L is selected from the group consisting of absent, —C(═S)N(R¹³)—, —C(═NR¹⁴)N(R¹³)—, —SO₂N(R¹³)—, —SO₂—, —C(═O)N(R¹³)—, —N(R¹³)—, —C(═O)C₁₋₂alkyl-N(R¹³)—, —N(R¹³)C₁₋₂alkyl-C(═O)—, —C(═O)C₀₋₁alkyl-C(═O)N(R¹³)—, —C₀₋₄alkylene, —C(═O)C₀₋₁alkyl-C(═O)OR³—, —C(═NR¹⁴)—C₀₋₁alkyl-C(═O)—, —C(═O)—, —C(═O)C₀₋₁alkyl-C(═O)— and an optionally substituted four to six-membered heterocyclyl containing between one and three annular heteroatoms including at least one nitrogen, wherein an alkyl of the aforementioned L groups is optionally independently substituted with X and X¹, wherein X and X¹ are independently selected from the group consisting of H, (C₁-C₆)alkyl, halo, cyano or nitro, wherein the (C₁-C₆)alkyl is additionally optionally substituted, or X and X¹ together with the atom to which they are attached are a C₃-C₇cycloalkyl; and
T is selected from the group consisting of —H, —R¹³, —C₀₋₅alkyl, —C₀₋₅alkyl-Q, —O—C₀₋₅alkyl-Q, —C₀₋₅alkyl-O-Q, —N(R¹³)—C₀₋₅alkyl-Q, —C₀₋₅alkyl-SO₂—C₀₋₅alkyl-Q, —C(═O)—C₀₋₅alkyl-Q, —C(═S)—C₀₋₅-alkyl-Q, —C(═NR¹⁴)—C₀₋₅-alkyl-Q, —C₀₋₅alkyl-N(R¹³)-Q, —C(═O)—N(R¹³)—C₀₋₅alkyl-Q, —C(═S)—N(R¹³)—C₀₋₅alkyl-Q, —C(═NR¹⁴)—N(R¹³)—C₀₋₅alkyl-Q and —(C₀₋₅alkyl-C(O))₀₋₁—C₀₋₅alkyl-Q, wherein each C₀₋₅alkyl is optionally independently substituted with X and X¹, wherein X and X¹ are independently selected from the group consisting of H, (C₁-C₆)alkyl, halo, cyano or nitro, wherein the (C₁-C₆)alkyl is additionally optionally substituted, or X and X¹ together with the atom to which they are attached are a C₃-C₇cycloalkyl;
R¹³ is selected from the group consisting of —H, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, —C(O)SR³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
two R¹³, together with the atom or atoms to which they are attached, can combine to form a heteroalicyclic optionally substituted with between one and four of R⁶⁰, wherein the heteroalicyclic can have up to four annular heteroatoms, and the heteroalicyclic can have an aryl or heteroaryl fused thereto, in which case the aryl or heteroaryl is optionally substituted with an additional one to four of R⁶⁰;
R¹⁴ is selected from the group —H, —NO₂, —NH₂, —N(R³)R⁴, —CN, —OR³, an optionally substituted (C₁-C₆)alkyl, an optionally substituted heteroalicyclylalkyl, an optionally substituted aryl, an optionally substituted arylalkyl and an optionally substituted heteroalicyclic,
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
R⁶⁰ is selected from the group consisting of —H, halogen, trihalomethyl, —CN, —NO₂, —NH₂, —OR³, —NR³R⁴, —S(O)₀₋₂R³, —SO₂NR³R³, —CO₂R³, —C(O)NR³R³, —N(R³)SO₂R³, —N(R³)C(O)R³, —N(R³)CO₂R³, —C(O)R³, an optionally substituted (C₁-C₆)alkyl, an optionally substituted aryl, an optionally substituted heteroarylalkyl and an optionally substituted arylalkyl;
two R⁶⁰ when attached to a non-aromatic carbon, can be oxo;
Q is a five- to ten-membered ring system, optionally substituted with between zero and four of R²⁰; and
R²⁰ is selected from the group consisting of —H, halogen, trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carbocyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted.

In a preferred embodiment of the compounds according to the present invention, T is selected from the group consisting of —H, —R¹³, —C₀₋₄alkyl, —C₀₋₄alkyl-Q, —O—C₀₋₄alkyl-Q, —C₀₋₄alkyl-O-Q, —N(R¹³)—C₀₋₄alkyl-Q, —C₀₋₄alkyl-SO₂—C₀₋₄alkyl-Q, —C(═O)—C₀₋₄alkyl-Q, —C(═S)—C₀₋₄-alkyl-Q, —C(═NR¹⁴)—C₀₋₄-alkyl-Q, —C₀₋₄alkyl-N(R¹³)-Q, —C(═O)—N(R¹³)—C₀₋₄alkyl-Q, —C(═S)—N(R¹³)—C₀₋₄alkyl-Q, —C(═NR¹⁴)—N(R¹³)—C₀₋₄alkyl-Q and —(C₀₋₄alkyl-C(O))₀₋₁—C₀₋₄alkyl-Q wherein each C₀₋₄alkyl is independently optionally substituted, preferably with X and X¹, wherein X and X¹ are independently selected from the group consisting of H, (C₁-C₆)alkyl, halo, cyano or nitro, wherein the (C₁-C₆)alkyl is additionally optionally substituted, or X and X¹ together with the atom to which they are attached are a C₃-C₇cycloalkyl;

In another preferred embodiment of the compounds according to the present invention, G is selected from the group consisting of

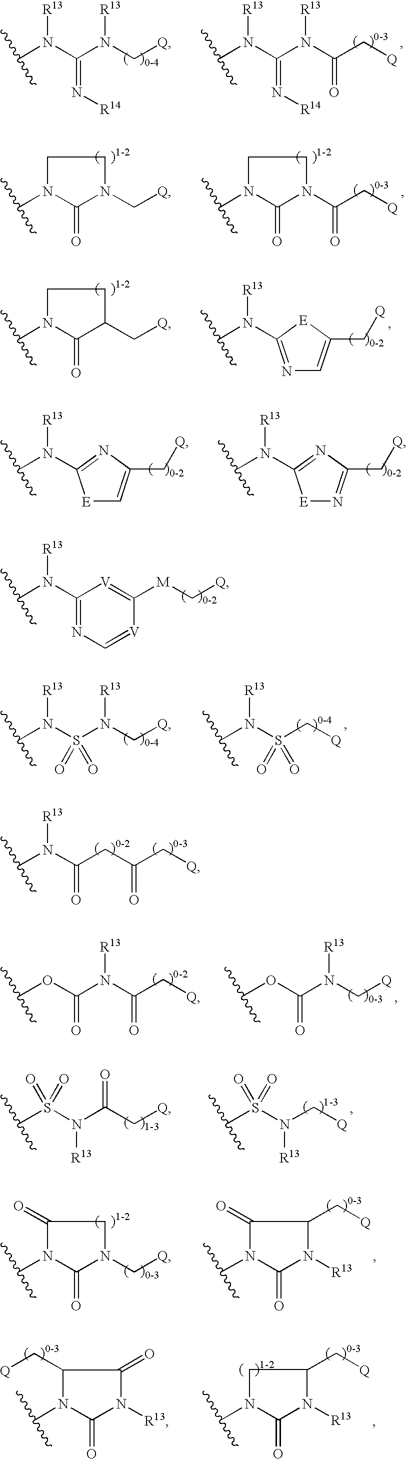

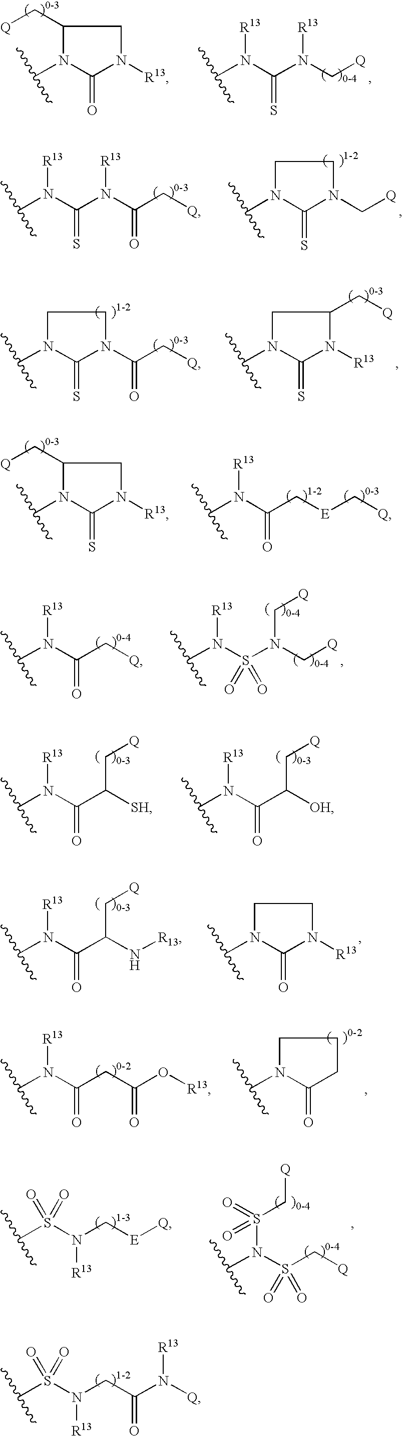

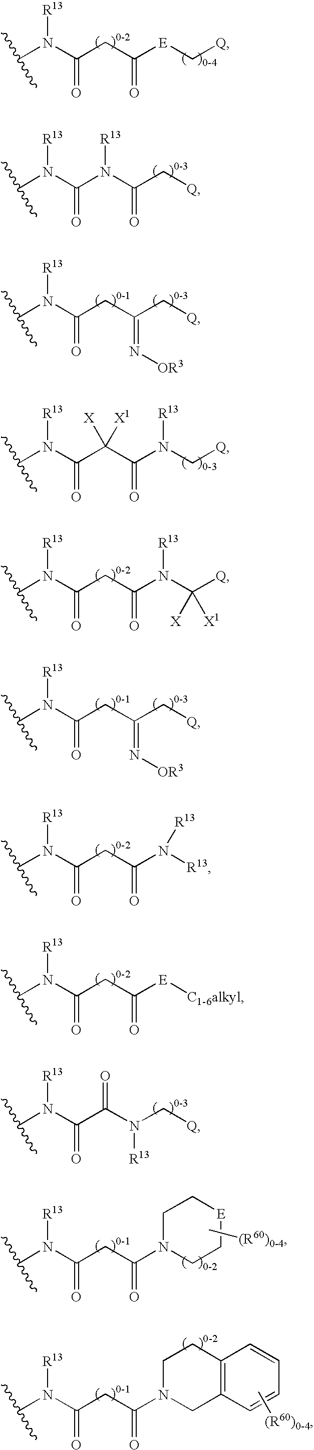

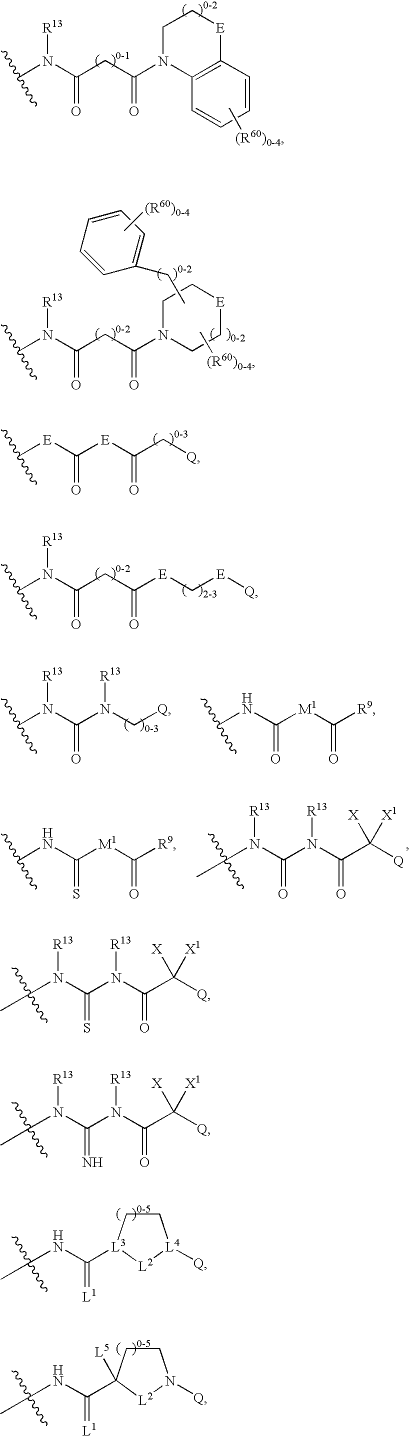

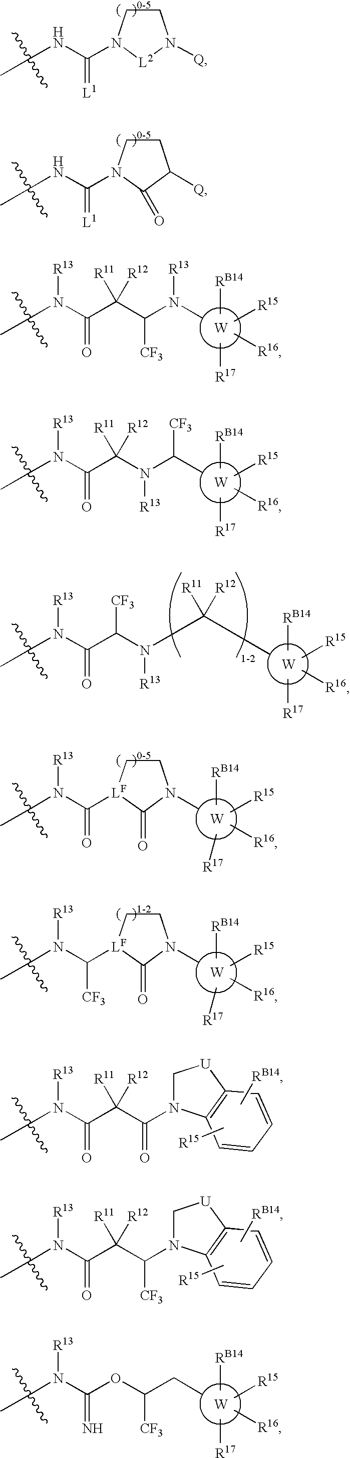

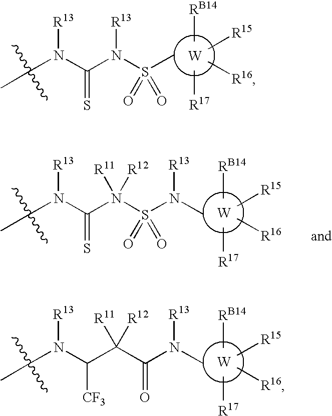

wherein R¹³, R¹⁴, Q, R³, R⁶⁰, L¹, L², L³, L⁴, R^{B14}, R¹⁵, R¹⁶, R¹⁷ and U are as defined above;
any methylene group is independently optionally substituted with R²⁵, wherein
R²⁵ is selected from the group consisting of halogen, trihalomethyl, —CN, —NO₂, —NH₂, —OR³, —NR³, R⁴, —S(O)₀₋₂R³, —SO₂NR³R³, —CO₂R³, —C(O)NR³R³, —N(R³)SO₂R³—N(R³)C(O)R³, —N(R³)CO₂R³, —C(O)R³, an optionally substituted aryl, an optionally substituted arylalkyl, an optionally substituted heteroarylalkyl, and an optionally substituted (C₁-C₆)alkyl,
two R²⁵, together with the carbon or carbons to which they are attached, can combine to form a three- to seven-membered alicyclic or heteroalicyclic, and
two R², on a single carbon can be oxo;
R⁹ is selected from the group consisting of a C₁₋₆ alkyl on which one or more hydrogen atoms are optionally substituted by —R²⁴, -T¹-R^{a}, or —NR^{b}R^{c}, a —N(R^{d})(R^{e}) moiety and a saturated or unsaturated three- to eight-membered carbocyclic or heterocyclic group which is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring wherein, when the three- to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group,
   wherein
T¹ is selected from the group consisting of —O—, —S— and —NH—;
R²⁴ represents a saturated or unsaturated three- to eight-membered carbocyclic or heterocyclic group;
R^{a}, R^{b}, and R^{c}, which may be the same or different, represent a C₁₋₆ alkyl or a saturated or unsaturated three- to eight-membered carbocyclic or heterocyclic group; wherein the three- to eight-membered carbocyclic or heterocyclic group represented by R²⁴, R^{a}, R^{b}, and R^{e} is optionally substituted by a C₁₋₆ alkyl, a C₁₋₆ alkoxy, a halogen atom, nitro, a trifluoromethyl, a C₁₋₆ alkoxy carbonyl, a cyano, a cyano C₁₋₆ alkyl, a C₁₋₆ alkylthio, a phenoxy, an acetyl, or a saturated or unsaturated five- or six-membered heterocyclyl ring; and wherein when the three- to eight-membered carbocyclic or heterocyclic group is substituted by two C₁₋₆ alkyl groups, the two alkyl groups may combine together to form an alkylene chain; and wherein the three- to eight-membered carbocyclic or heterocyclic group may be a bicyclic group condensed with another saturated or unsaturated three- to eight-membered carbocyclic or heterocyclic group; and
R^{d} and R^{e}, which may be the same or different, represent (1) a hydrogen atom, (2) a C₁₋₆ alkyl which is optionally substituted by a C₁₋₆ alkoxy, a C₁₋₆ alkylthio, or a saturated or unsaturated three- to eight-membered carbocyclic or heterocyclic group in which the three- to eight-membered carbocyclic or heterocyclic group is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring and wherein when the three- to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group, or (3) a saturated or unsaturated three- to eight-membered carbocyclic or heterocyclic group which is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring and in which, when the three to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group;
X and X¹ are each independently selected from the group consisting of —H, halogen, cyano, nitro and an optionally substituted C₁-C₆ alkyl, or
X and X¹ together with the atom to which they are attached form a C₃-C₇ cycloalkyl;
E is selected from the group consisting of —O—, —N(R¹³)—, —CH₂— and —S(O)₀₋₂—;
M is selected from the group consisting of —O—, —N(R¹³)—, —CH₂— and —C(═O)N(R¹³);
M¹ represents —C(R²⁶)(R²⁷)—, wherein
R²⁶ and R²⁷ are independently selected from the group consisting of a hydrogen atom, a C₁₋₄ alkyl, a C₁₋₄ alkoxy and —N(R^{f}), wherein
R^{f} is a hydrogen atom or a C₁₋₄ alkyl;
each V is independently selected from the group consisting of ═N— and ═C(H)—; and
L⁵ is selected from the group consisting of H, alkyl, halogen, OMe, —C₀₋₄alkyl-OMe, —C₀₋₄alkylNHMe, —C₀₋₄alkyl-NMe2 and —C₀₋₄alkyl-heterocycle. Preferably, —C₀₋₄alkyl- is —CH2—. Preferably, the heterocycle and —C₀₋₄alkyl- are linked via a N atom in the heterocycle.

In another preferred embodiment, G is selected from the group consisting of:

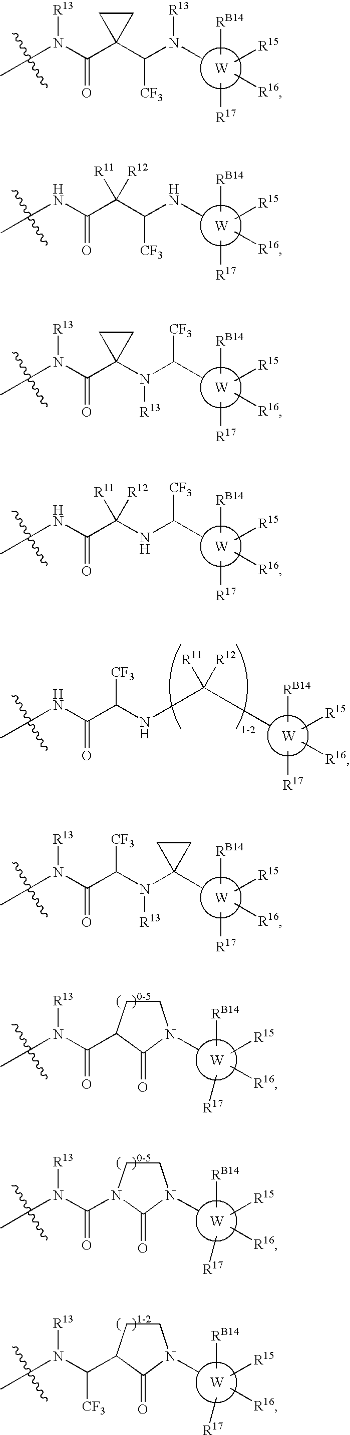

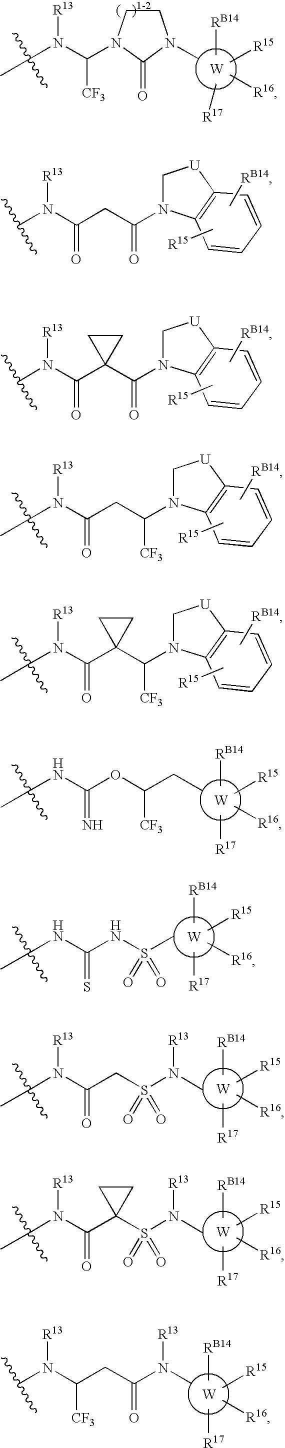

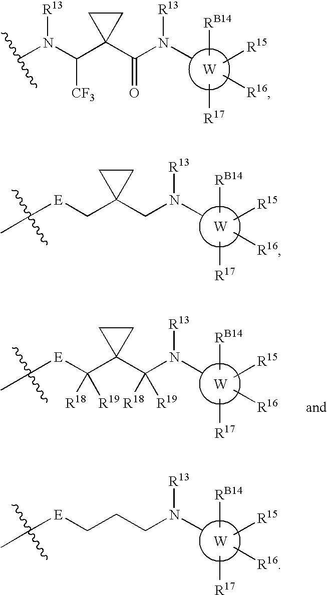

In another preferred embodiment of the present invention, G is

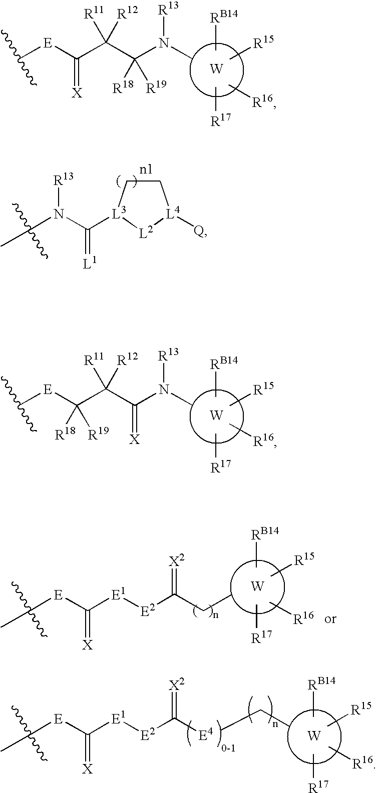

In another preferred embodiment of the present invention, G is

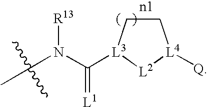

In another preferred embodiment of the compounds according to the present invention, G is selected from the group consisting of

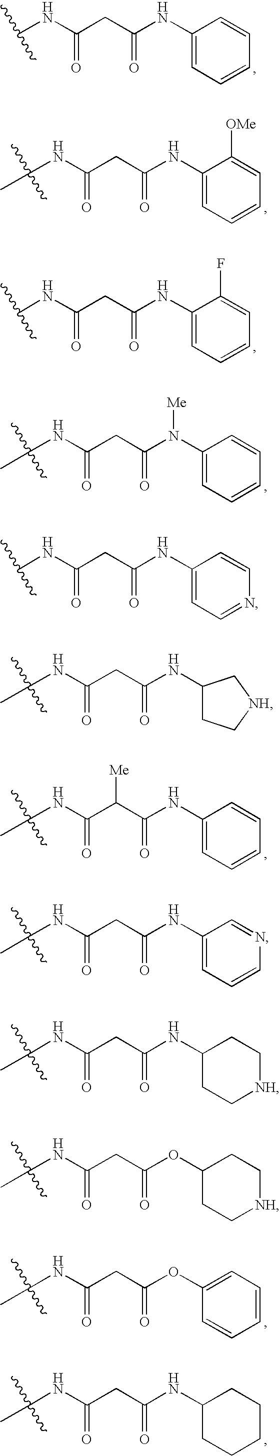

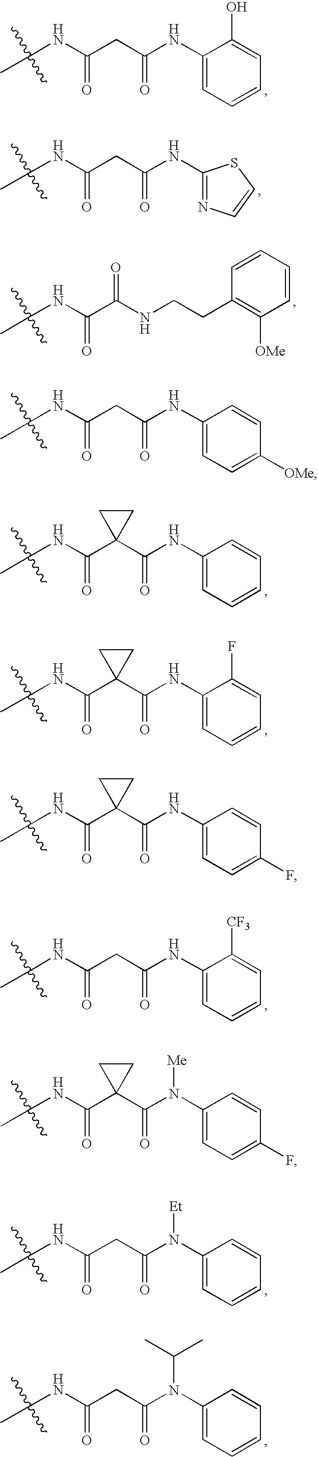

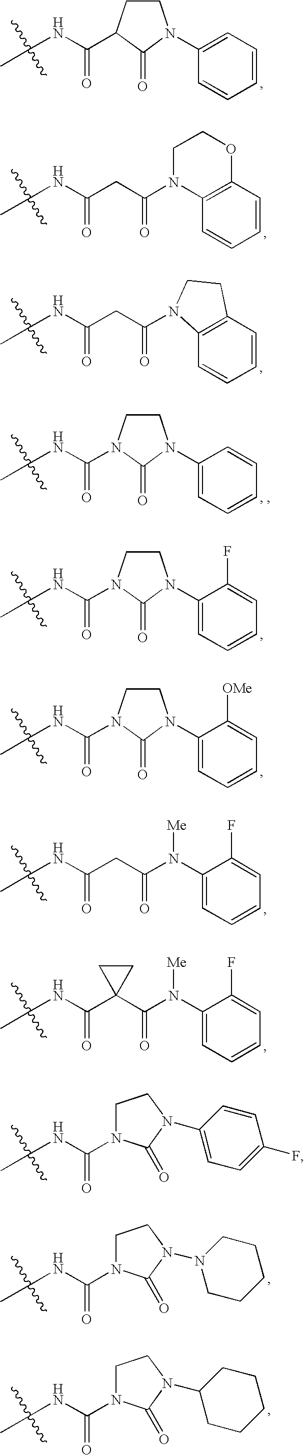

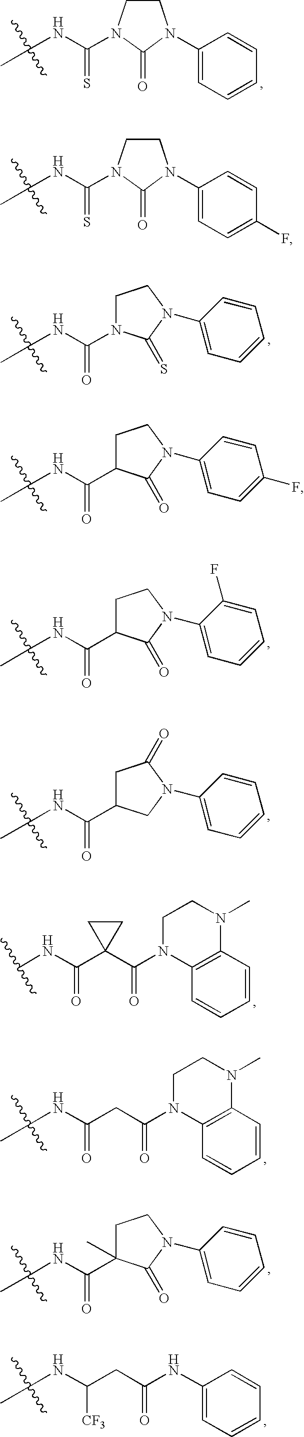

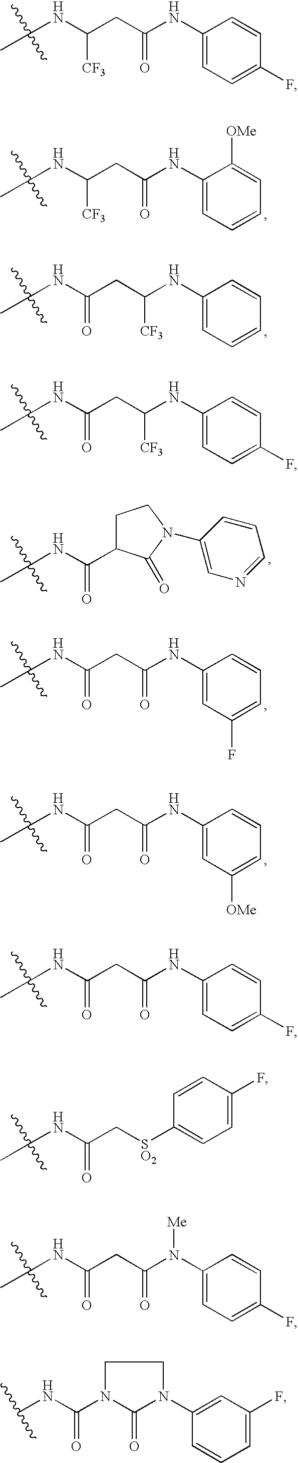

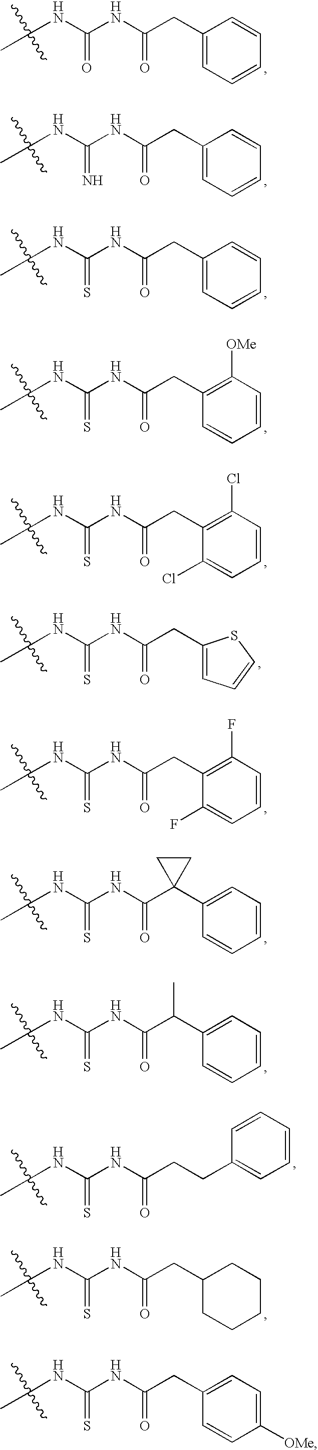

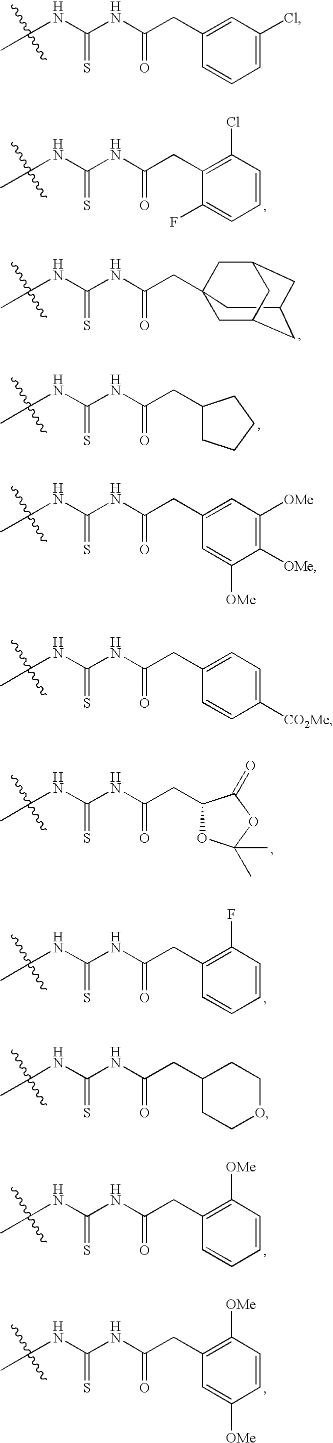

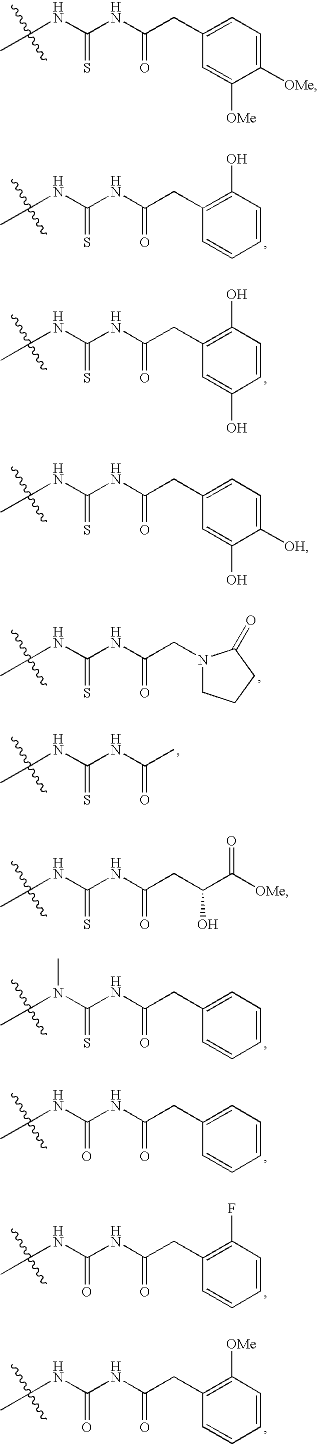

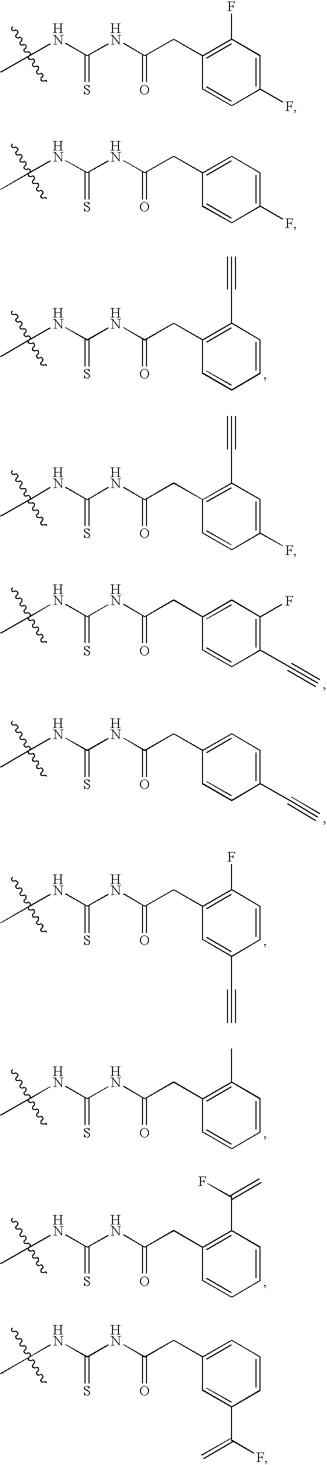

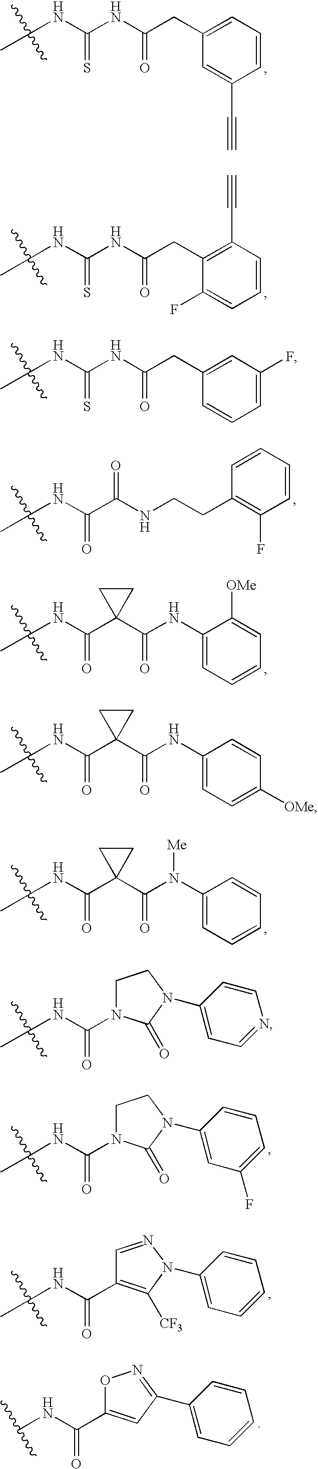

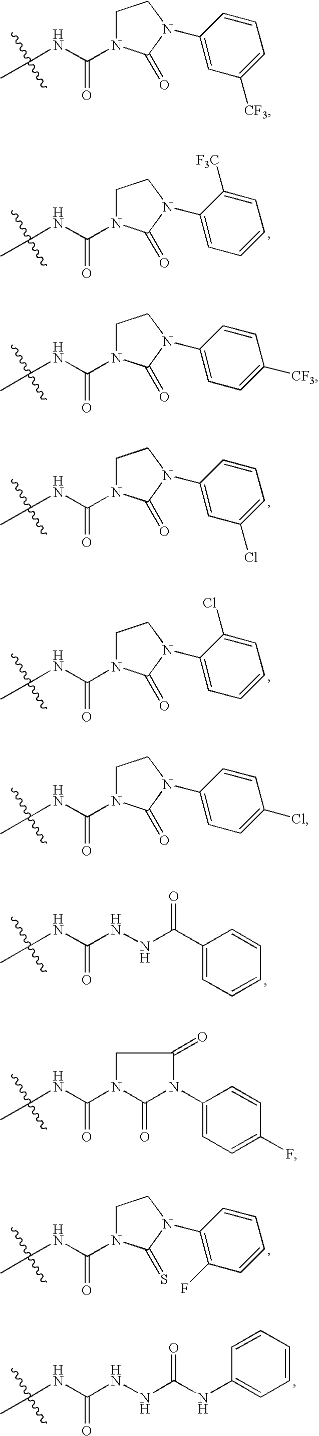

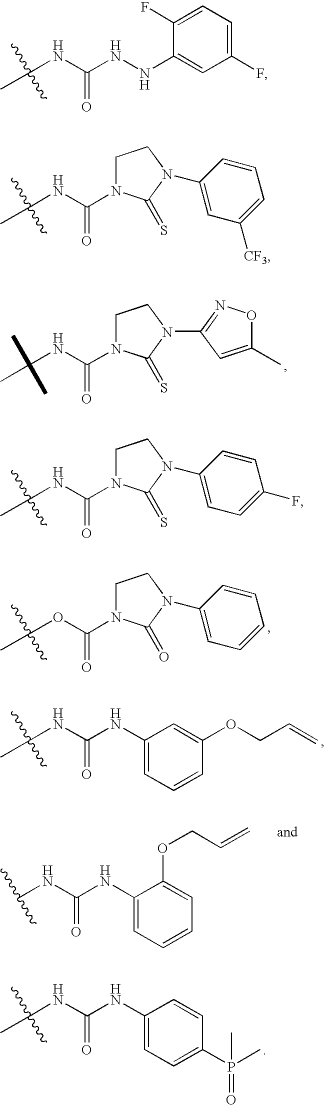

In a preferred embodiment of the compounds according to the present invention, the optionally substituted alkyl group represented by R⁹ preferably represents —(CH₂)p-R²⁴, —(CH₂)p -T-R^{a}, or —(CH₂)p-NR^{b}R^{c} wherein p is an integer of 1 to 6 and R²⁴, R^{a}, R^{b}, and R^{c} are as defined above.

In a preferred embodiment of the compounds according to the present invention in —N(R^{d})(R^{e}) represented by R⁹, preferably, R^{d} represents a hydrogen atom or C₁₋₆ alkyl, and R^{e} represents C₁₋₆ alkyl which is optionally substituted by an optionally substituted saturated or unsaturated five- or six-membered carbocyclic or heterocyclic group; or an optionally substituted saturated or unsaturated five- or six-membered carbocyclic or heterocyclic group.

In a preferred embodiment of the compounds according to the present invention, preferred examples of R⁹ include, but are not limited to, benzyl, fluorobenzyl, difluorobenzyl, chlorobenzyl, methylbenzyl, methoxybenzyl, aniline, fluoroanilino, difluoroanilino, chloroanilino, methylanilino, methoxyanilino, naphthyl, thienyl-2-yl-methyl, and thienyl-3-yl-methyl.

In a preferred embodiment of the compounds according to the present invention, examples of R^{e} include phenyl, fluorophenyl, difluorophenyl, chlorophenyl, methylphenyl, methoxyphenyl, pyridyl, isoxazolyl and quinolyl.

In another preferred embodiment of the compounds according to the present invention, G is selected from the group consisting of:

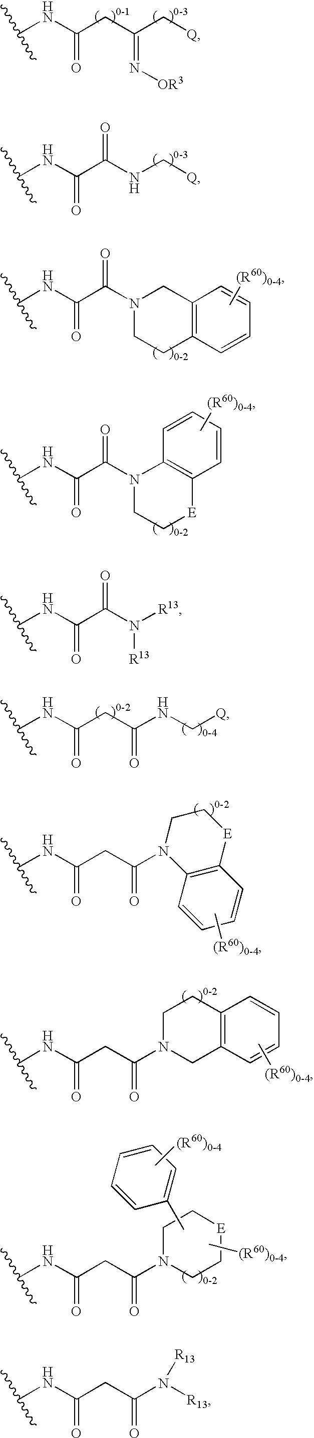

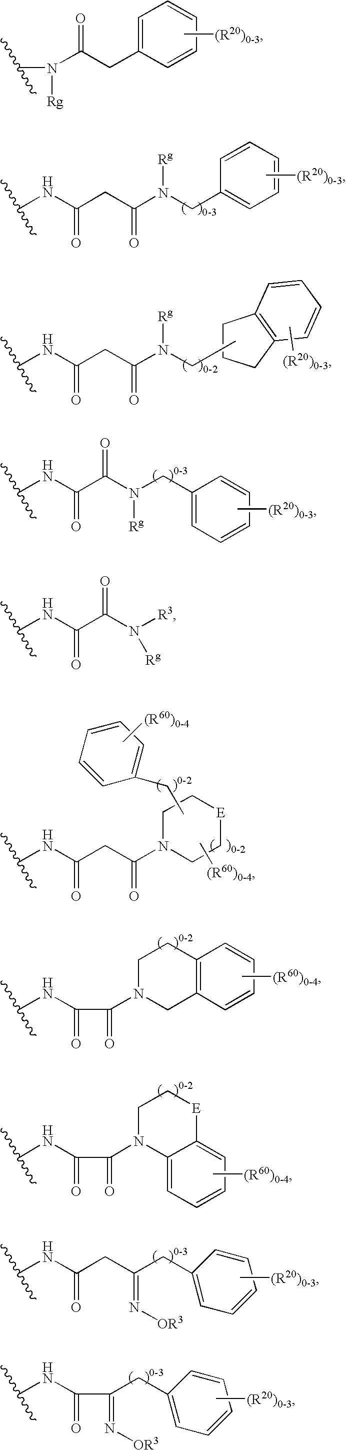

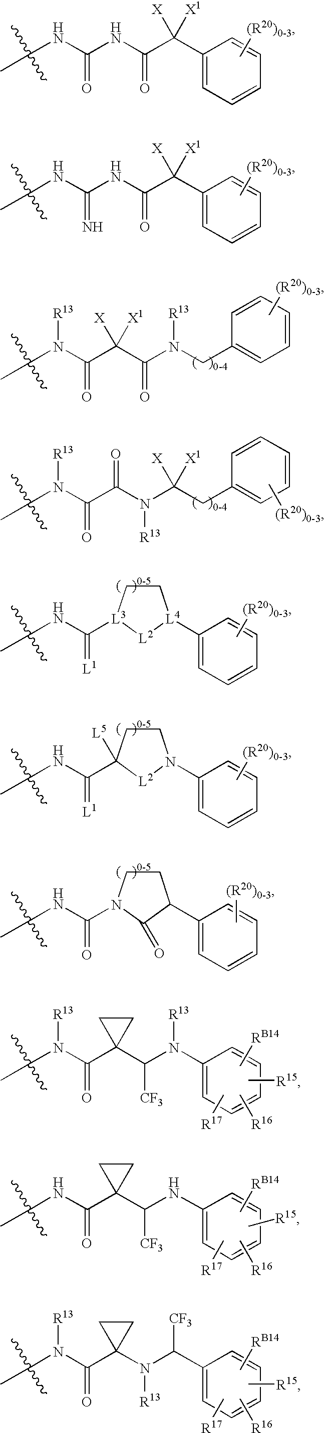

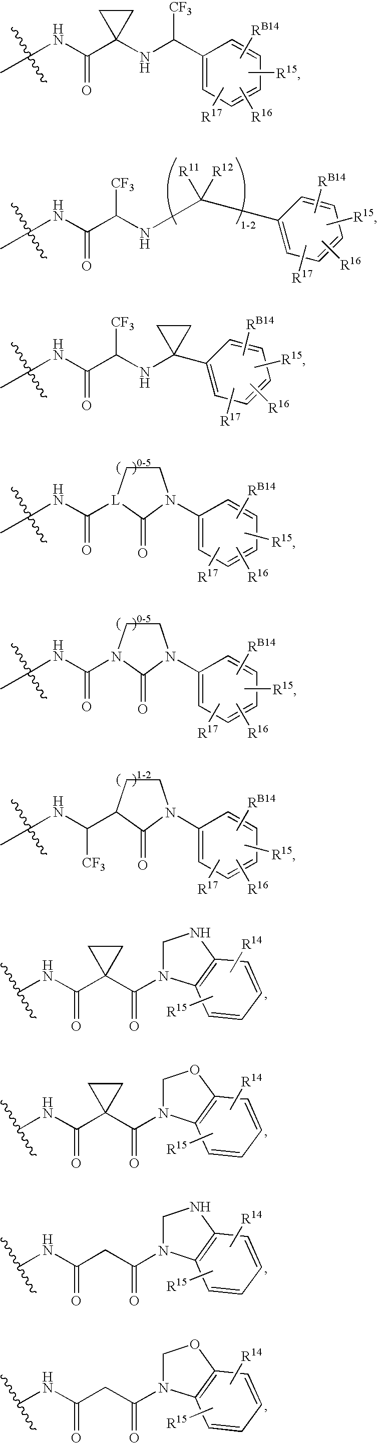

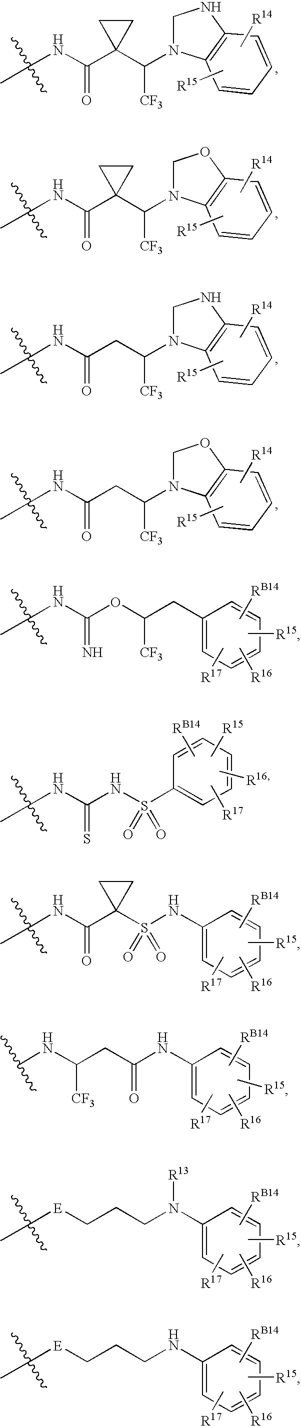

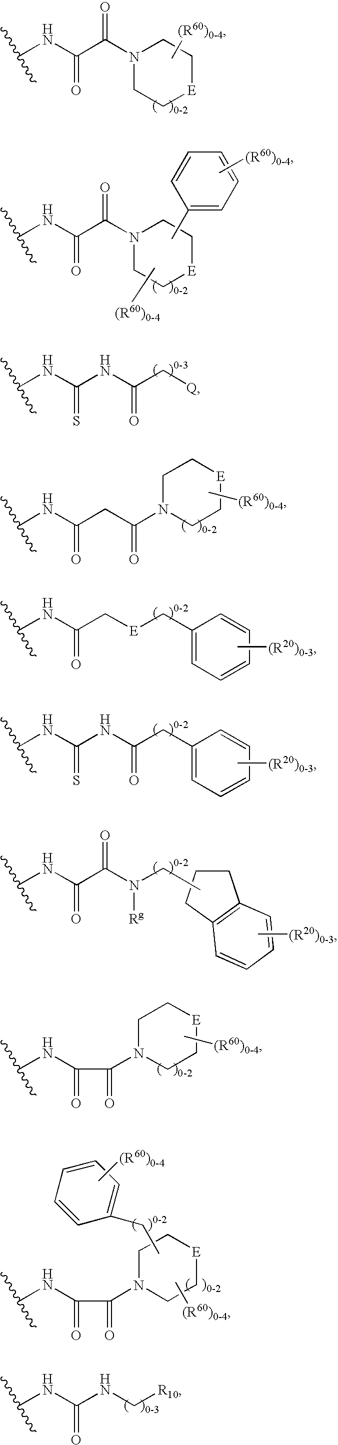

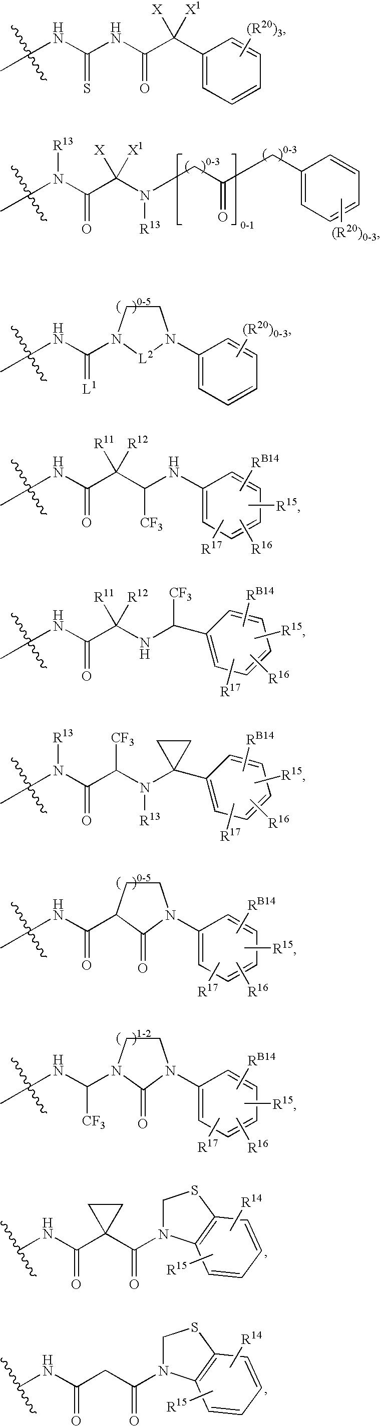

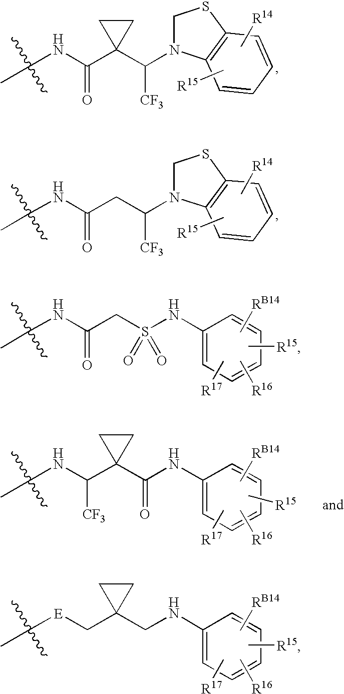

wherein each methylene in any of the above formulae, other than those in a depicted ring, is independently optionally substituted with R²⁵;
R²⁵ is selected from the group consisting of halogen, trihalomethyl, —CN, —NO₂, —NH₂, —OR³, —NR³, R⁴, —S(O)₀₋₂R³, —SO₂NR³R³, —CO₂R³, —C(O)NR³R³, —N(R³)SO₂R³—N(R³)C(O)R³, —N(R³)CO₂R³, —C(O)R³, an optionally substituted aryl, an optionally substituted arylalkyl, an optionally substituted heteroarylalkyl, and an optionally substituted (C₁-C₆)alkyl,
two R²⁵, together with the carbon or carbons to which they are attached, can combine to form a three- to seven-membered alicyclic or heteroalicyclic;
R^{g} is —H or an optionally substituted (C₁-C₆)alkyl;
R¹⁰ is an azolyl, wherein one or more hydrogen atoms are optionally substituted by a moiety selected from the group consisting of a halogen, C₁₋₄ alkyl, C₁₋₄ alkoxy, C₁₋₄ alkylthio, trihalomethyl, nitro, amino optionally independently substituted by one or two of C₁₋₄ alkyl, a C₁₋₄ alkoxycarbonyl C₁₋₄ alkyl, a C₁₋₄ alkylcarbonyl and a C₃₋₅ cyclic alkyl;
X and X¹ are independently selected from the group consisting of —H, halogen, cyano, nitro, C₁-C₆ alkyl, or
X and X¹ taken together with the atom to which they are attached, form a C₃-C₇ cycloalkyl;
E is selected from the group consisting of —O—, —N(R¹³)—, —CH₂— and —S(O)₀₋₂—.

In another preferred embodiment of the present invention, G is

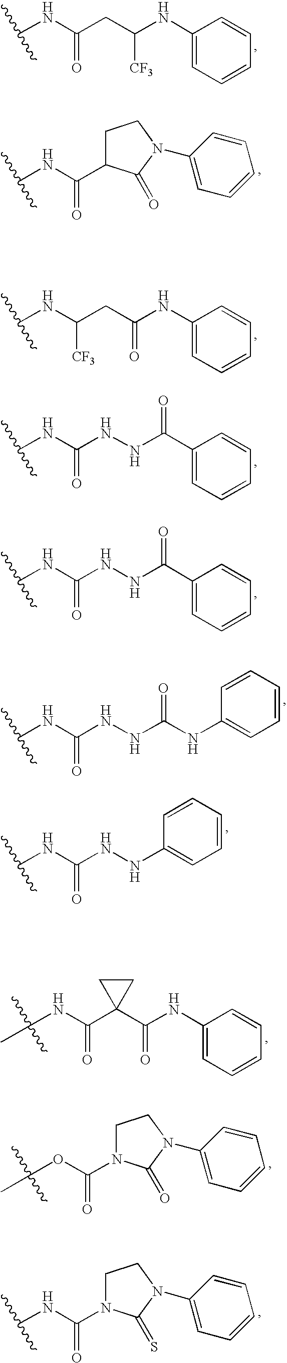

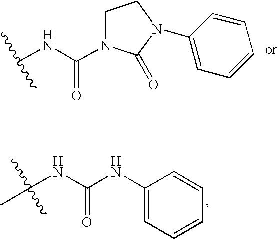

wherein each ring of G is optionally substituted.

In a preferred embodiment of the present invention,

### G is

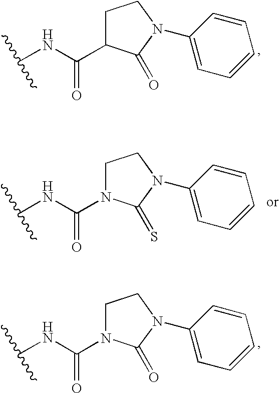

preferably

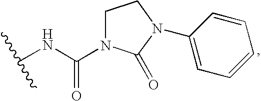

wherein each ring of G is optionally substituted.

In a preferred embodiment of the compounds according to the present invention, a methylene group between two carbonyl groups is mono- or di-substituted with either an optionally substituted (C₁-C₆)alkyl or an optionally substituted spirocycle.

In a preferred embodiment of the compounds according to the present invention, R¹⁰ is selected from the group consisting of

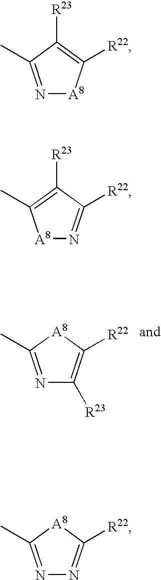

wherein A⁸ is selected from the group consisting of —O—, —S— and —NH—; and
R²² and R²³ are independently selected from the group consisting of —H, halogen, C₁₋₄ alkyl, C₁₋₄ alkoxy, C₁₋₄ alkylthio, trihalomethyl, nitro, amino optionally independently substituted by one or two of C₁₋₄ alkyl, a C₁₋₄ alkoxycarbonyl C₁₋₄ alkyl, a C₁₋₄ alkylcarbonyl and a C₃₋₅ cyclic alkyl.

In a preferred embodiment of the compounds according to the present invention, R¹⁰ is an optionally substituted azolyl selected from the group consisting of imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

In a preferred embodiment of the compounds according to the present invention L¹ is O or S, more preferably O.

In a preferred embodiment of the compounds according to the present invention, L² is —C(O)— or —C(S)—, more preferably —C(O)—.

In a preferred embodiment of the compounds according to the present invention, L³ is N.

In a preferred embodiment of the compounds according to the present invention, L⁴ is N.

In a preferred embodiment of the compounds according to the present invention, L³ is N and L⁴ is CH.

In a preferred embodiment of the compounds according to the present invention, L⁴ is N and L³ is CH.

In a preferred embodiment of the compounds according to the present invention, L³ and L⁴ are N.

In a preferred embodiment of the compounds according to the present invention, Q is selected from the group consisting of arylalkyl, cycloalkyl, heterocyclyl, aryl and heteroaryl, wherein each of said arylalkyl, cycloalkyl, heterocyclyl, aryl and heteroaryl is optionally substituted with 1 to 3 independently selected R²⁰.

In a preferred embodiment of the compounds according to the present invention, Q is selected from the group consisting of

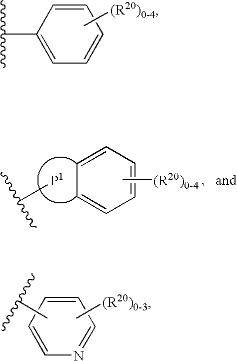

wherein P¹ is a five- to seven-membered ring, including the two shared carbon atoms of the aromatic ring to which P¹ is fused, and wherein P¹ optionally contains between one and three heteroatoms.

In a preferred embodiment of the compounds according to the present invention, Q is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted with between one and four of R²⁰, wherein
each R²⁰ is selected from the group consisting of —H, halogen, trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂, —N(R³)C(O)R³, —N(R³)C(O)OR³, —C(O)R³, —C(O)SR³, C₁-C₄alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₄ alkoxy, an amino optionally substituted by C₁₋₄ alkyl optionally substituted by C₁₋₄ alkoxy and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted

In a preferred embodiment of the present invention, R⁴² is H.

In a preferred embodiment of the present invention, R⁴³ is —Y-(5 to 10 membered heterocyclyl), wherein Y is preferably a bond.

In a preferred embodiment of the present invention, R⁴³ is —Y-(5 to 10 membered heterocyclyl), substituted with —NR³⁶R³⁹ and Y is preferably a bond.

In a preferred embodiment of the present invention R⁴³ is H or alkyl.

In a preferred embodiment of the present invention, R⁴⁶ is heteroaryl, preferably substituted with —(CH₂)ₙNR⁵⁰R⁵¹, wherein preferably n is 0, R⁵⁰ is H, R⁵¹ is —C(O)R⁴⁵ and R⁴⁵ is alkyl.

In a preferred embodiment of the present invention, R⁴⁶ is heteroaryl, preferably substituted with —(CH₂)ₙNR⁵⁰R⁵¹, wherein preferably n is O, R⁵⁰ is H and R⁵¹ is H.

In a preferred embodiment of the present invention R¹⁰¹ is haloalkyl, alkenyl, -alkyl-heterocycle or -alkyl-P(O)(alkyl)₂.

In a preferred embodiment of the present invention,
D is selected from the group consisting of H, —NH₂, —NR⁴²C(═O)R⁴³, —NR⁴²C(═O)NR⁴³—R¹⁰¹, —C≡C—(CR⁴⁵R⁴⁵)ₙ—R⁴⁶, —Y—NR⁴²R⁴³, —NR^{6a}C(O)OR^{6b}, oxo and —C(O)NR⁴²R⁴³;
M is heteroaryl;
Z is selected from the group consisting of —O—, —NH—C(O)—NH—, C₂alkynylene, —NH—, —NH—C(O)— and —NH—SO2-;
Ar is optionally substituted phenyl, preferably optionally substituted with a substituent selected from the group consisting of F, Cl, C₁-C₆alkyl and C₁-C₆alkoxy, more preferably F; and
G is

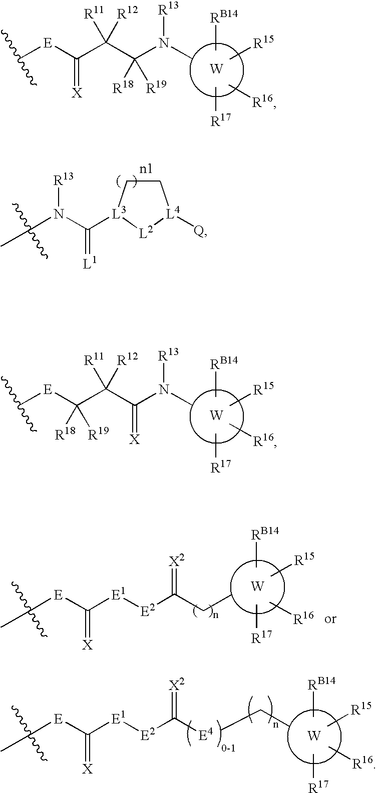

In a preferred embodiment of the present invention,
D is selected from the group consisting of H, —NH₂, —NR⁴²C(═O)R⁴³, —NR⁴²C(═O)NR⁴³—R¹⁰¹, —C≡C—(CR⁴⁵R⁴⁵)ₙ—R⁴⁶, —Y—NR⁴²R⁴³, —NR^{6a}C(O)OR^{6b}, oxo and —C(O)NR⁴²R⁴³;
M is heteroaryl;
Z is selected from the group consisting of —O—, —NH—C(O)—NH—, C₂alkynylene, —NH—, —NH—C(O)— and —NH—SO2-;
Ar is optionally substituted phenyl, preferably optionally substituted with a substituent selected from the group consisting of F, Cl, C₁-C₆alkyl and C₁-C₆alkoxy, more preferably F; and
G is

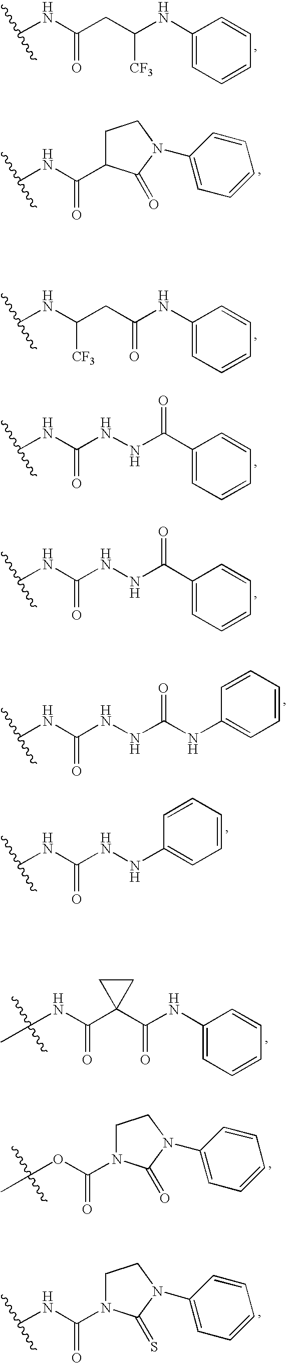

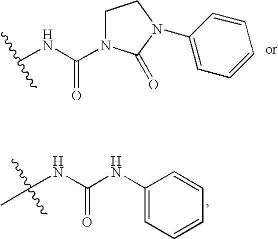

wherein each ring of G is optionally substituted

In a preferred embodiment of the present invention,
D is selected from the group consisting of —NR⁴²C(═O)R⁴³, —NH₂, —NR⁴²C(═O)NR⁴³—R¹⁰¹, —C≡C—(CR⁴⁵R⁴⁵)ₙ—R⁴⁶ and —Y—NR⁴²R⁴³;
M is heteroaryl;
Z is selected from the group consisting of —O—, —NH—C(O)—NH—, C₂alkynylene, —NH—, —NH—C(O)— and —NH—SO₂—;
Ar is optionally substituted phenyl, preferably optionally substituted with a substituent selected from the group consisting of F, Cl, C₁-C₆alkyl and C₁-C₆alkoxy, more preferably F; and
G is

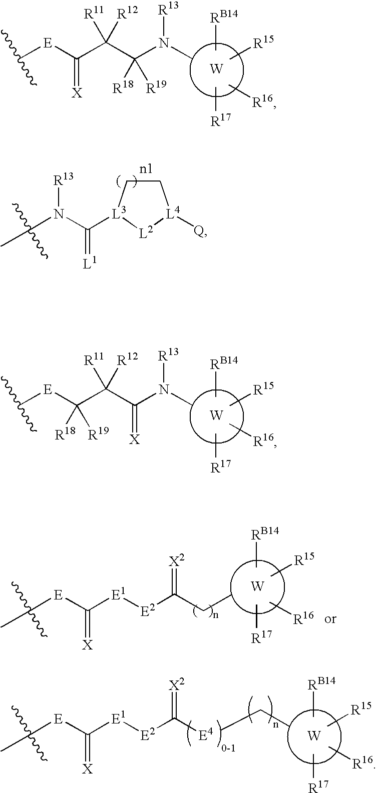

In a preferred embodiment of the present invention,
D is selected from the group consisting of —NR⁴²C(═O)R⁴³, —NH₂, —NR⁴²C(═O)NR⁴³—R¹⁰¹, —C≡C—(CR⁴⁵R⁴⁵)ₙ—R⁴⁶ and —Y—NR⁴²R⁴³;
M is heteroaryl;
Z is selected from the group consisting of —O—, —NH—C(O)—NH—, C₂alkynylene, —NH—, —NH—C(O)— and —NH—SO2-;
Ar is optionally substituted phenyl, preferably optionally substituted with a substituent selected from the group consisting of F, Cl, C₁-C₆alkyl and C₁-C₆alkoxy, more preferably F; and
G is

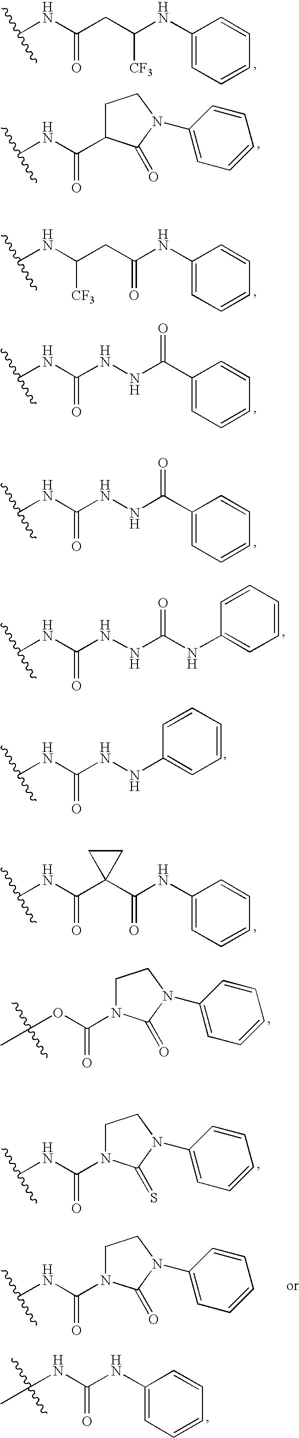

wherein each ring of G is optionally substituted.

In a preferred embodiment of the present invention,
D is selected from the group consisting of H, —NH₂, —NR⁴²C(═O)R⁴³, —NR⁴²C(═O)NR⁴³—R¹⁰¹, —C≡C—(CR⁴⁵R⁴⁵)ₙ—R⁴⁶, —Y—NR⁴²R⁴³, —NR^{6a}C(O)OR^{6b}, oxo and —C(O)NR⁴²R⁴³;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

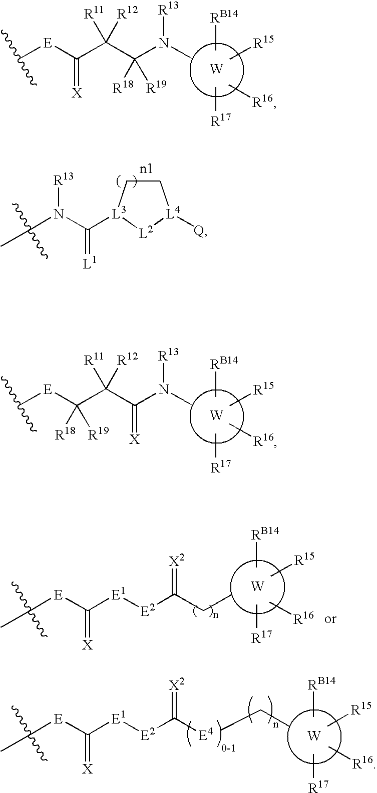

In a preferred embodiment of the present invention,
D is selected from the group consisting of H, —NH₂, —NR⁴²C(═O)R⁴³, —NR⁴²C(═O)NR⁴³—R¹⁰¹, —C≡C—(CR⁴⁵R⁴⁵)—R⁴⁶, —Y—NR⁴²R⁴³, —NR^{6a}C(O)OR^{6b}, oxo and —C(O)NR⁴²R⁴³;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

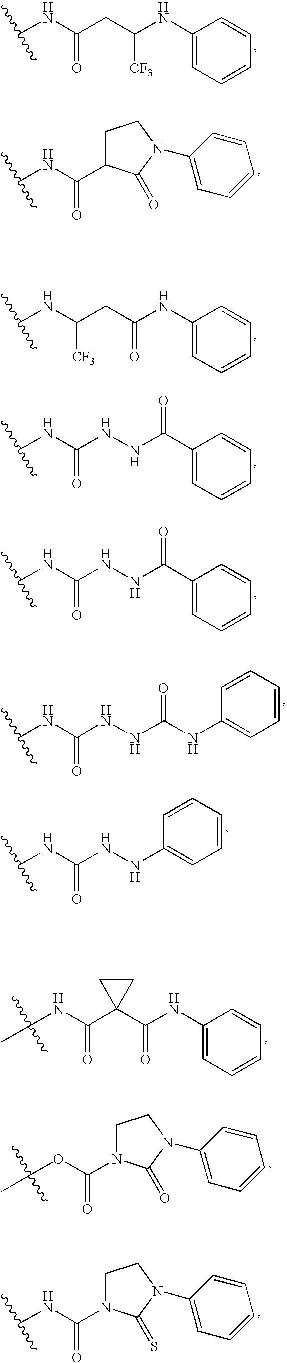

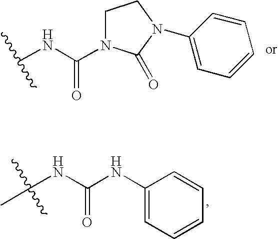

wherein each ring of G is optionally substituted.

In a preferred embodiment of the present invention,
D is selected from the group consisting of —NR⁴²C(═O)R⁴³, —NH₂, —NR⁴²C(═O)NR⁴³—R¹⁰¹, —C≡C—(CR⁴⁵R⁴⁵)ₙ—R⁴⁶ and —Y—NR⁴²R⁴³;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

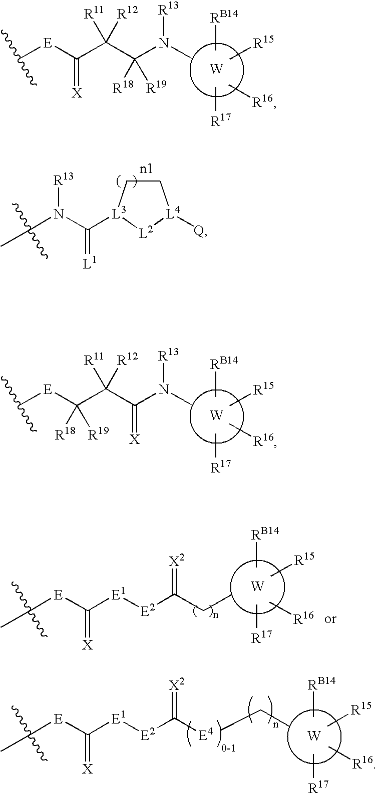

In a preferred embodiment of the present invention,
D is selected from the group consisting of —NR⁴²C(═O)R⁴³, —NH₂, —NR⁴²C(═O)NR⁴³—R¹⁰¹, —C≡C—(CR⁴⁵R⁴⁵)ₙ—R⁴⁶ and —Y—NR⁴²R⁴³;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

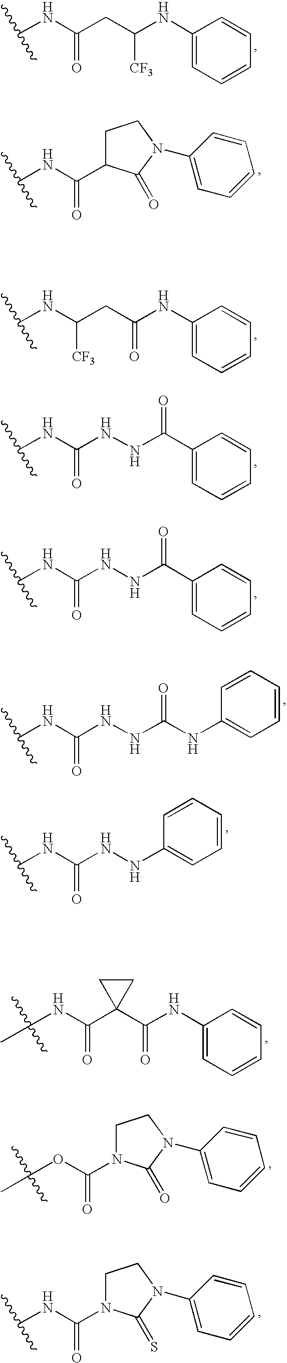

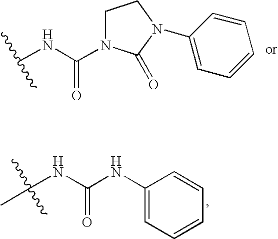

wherein each ring of G is optionally substituted.

In a preferred embodiment of the present invention,
D is —NR⁴²C(═O)-heterocyclyl wherein the heterocyclyl is optionally substituted, preferably with —NR³⁶R³⁹;
M is heteroaryl;
Z is selected from the group consisting of —O—, —NH—C(O)—NH—, C₂alkynylene, —NH—, —NH—C(O)— and —NH—SO2-;
Ar is optionally substituted phenyl, preferably optionally substituted with a substituent selected from the group consisting of F, Cl, C₁-C₆alkyl and C₁-C₆alkoxy, more preferably F; and
G is

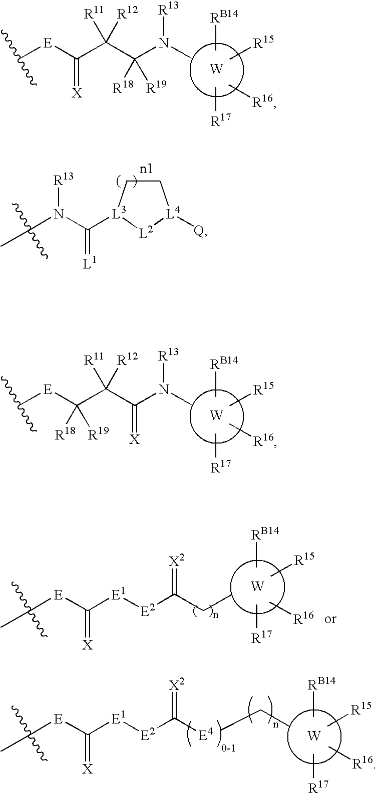

In a preferred embodiment of the present invention,
D is —NR⁴²C(═O)-heterocyclyl wherein the heterocyclyl is optionally substituted, preferably with —NR³⁶R³⁹;
M is heteroaryl;
Z is selected from the group consisting of —O—, —NH—C(O)—NH—, C₂alkynylene, —NH—, —NH—C(O)— and —NH—SO2-;
Ar is optionally substituted phenyl, preferably optionally substituted with a substituent selected from the group consisting of F, Cl, C₁-C₆alkyl and C₁-C₆alkoxy, more preferably F; and
G is

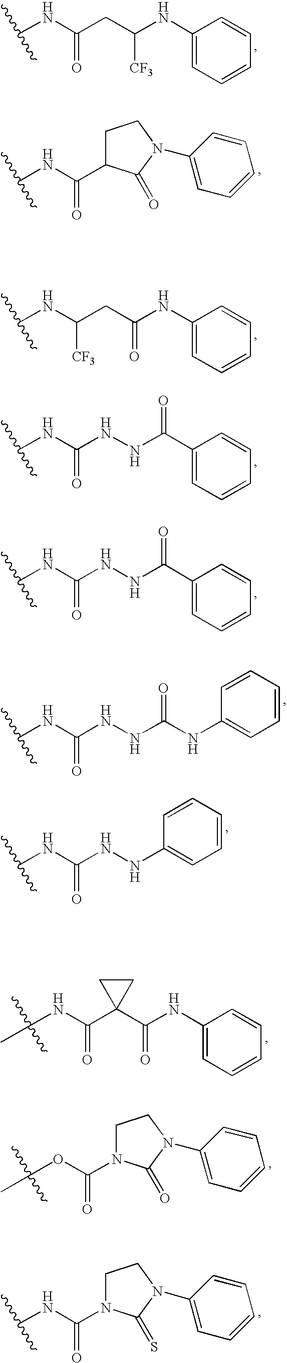

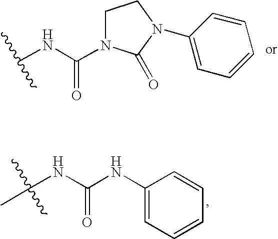

wherein each ring of G is optionally substituted.

In a preferred embodiment of the present invention,
D is —NR⁴²C(═O)-heterocyclyl wherein the heterocyclyl is optionally substituted, preferably with —NR³⁶R³⁹;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

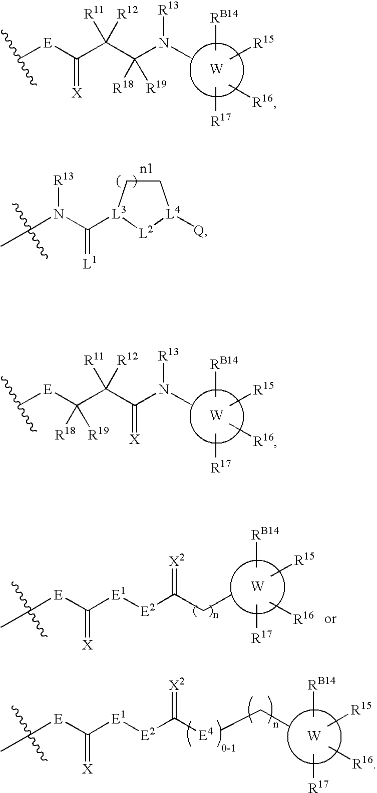

In a preferred embodiment of the present invention,
D is —NR⁴²C(═O)-heterocyclyl wherein the heterocyclyl is optionally substituted, preferably with —NR³⁶R³⁹;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

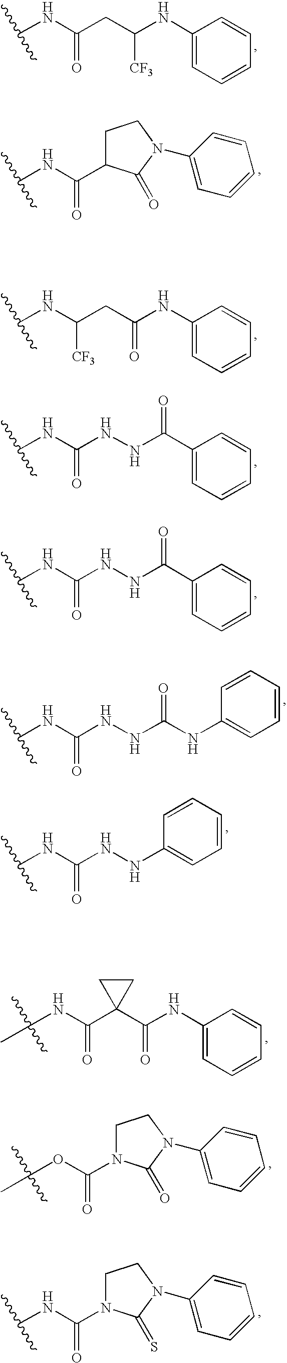

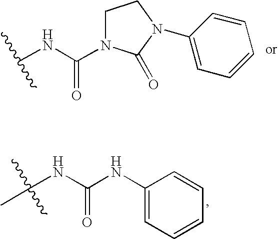

wherein each ring of G is optionally substituted.

In a preferred embodiment of the present invention,
D is —NR⁴²C(═O)-heterocyclyl wherein the heterocyclyl is optionally substituted, preferably with —NR³⁶R³⁹;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

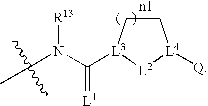

In a preferred embodiment of the present invention,
D is —NR⁴²C(═O)-heterocyclyl wherein the heterocyclyl is optionally substituted, preferably with —NR³⁶R³⁹;
M is pyridine or pyrimidine, preferably pyridine;
Z is —O—;
Ar is optionally substituted phenyl, preferably optionally substituted with a substituent selected from the group consisting of F, Cl, C₁-C₆alkyl and C₁-C₆alkoxy, more preferably F; and
G is

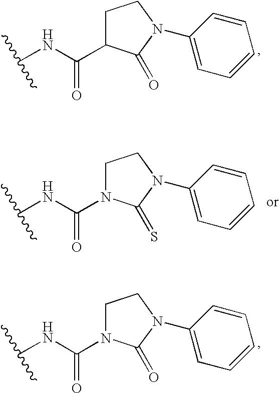

preferably

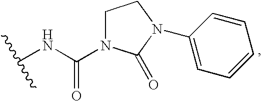

wherein each ring of G is optionally substituted.

Non-limiting examples of preferred compounds according to the invention are shown in Table I below.

TABLE I
Exemplary Compounds

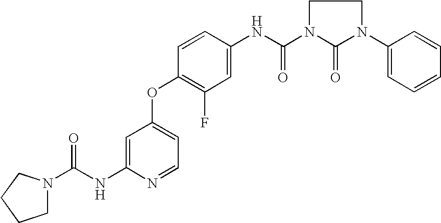

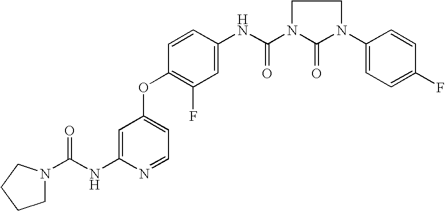

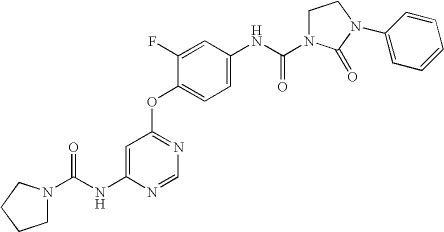

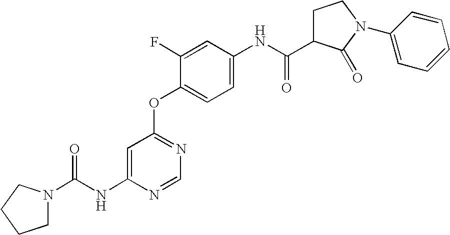

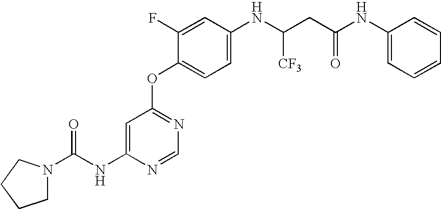

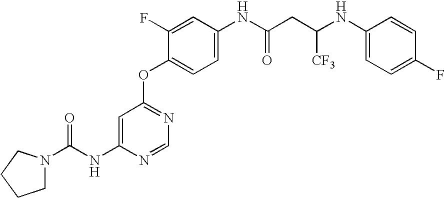

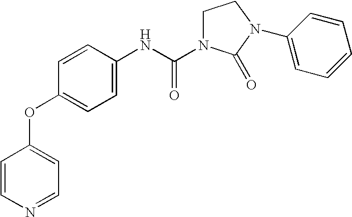

In the second aspect, the invention provides a composition comprising a compound according to the present invention together with a pharmaceutically acceptable excipient. In a preferred embodiment of this aspect, the composition further comprises an additional therapeutic agent.

The third aspect of the invention provides a method of inhibiting kinase activity, preferably protein tyrosine kinase activity, preferably inhibiting VEGF receptor signaling and HGF receptor signaling, the method comprising contacting the kinase with a compound according to the present invention, or with a composition according to the present invention. Inhibition of kinase activity, preferably VEGF and HGF activity, can be in a cell or a multicellular organism. If in a multicellular organism, the method according to this aspect of the invention comprises administering to the organism a compound according to the present invention, or a composition according to the present invention. Preferably the organism is a mammal, more preferably a human.

The data presented herein demonstrate the inhibitory effects of the kinase inhibitors of the invention. These data lead one to reasonably expect that the compounds of the invention are useful not only for inhibition of kinase activity, protein tyrosine kinase activity, or preferred embodiments thereof, such as, VEGF receptor signaling and HGF receptor signaling, but also as therapeutic agents for the treatment of proliferative diseases, including cancer and tumor growth.

Compounds of the present invention show inhibitory activity against at least one of CDK2, Flt1, Flt4, KDR, c-met, Ret, Ron, Tie2, TrkA, Lck, Bmx and Axl.

Preferred compounds according to the invention include those described in the examples below. Compounds were named using Chemdraw Ultra version 10.0, which are available through Cambridgesoft.com, 100 Cambridge Park Drive, Cambridge, Mass. 02140, Namepro version 5.09, which is available from ACD labs, 90 Adelaide Street West, Toronto, Ontario, M5H, 3V9, Canada, or were derived therefrom.

Examples of kinases that are inhibited by the compounds and compositions described herein and against which the methods described herein are useful include, but are not limited to, c-Met and KDR.

Depending on the particular condition, or disease, to be treated, additional therapeutic agents, which could be normally administered to treat that condition, may also be present in the compositions of this invention. In other words, compounds of this invention can be administered as the sole pharmaceutical agent or in combination with one or more other additional therapeutic (pharmaceutical) agents where the combination causes no unacceptable adverse effects. This may be of particular relevance for the treatment of hyper-proliferative diseases such as cancer. In this instance, the compound of this invention can be combined with known cytotoxic agents, signal transduction inhibitors, or with other anti-cancer agents, as well as with admixtures and combinations thereof. As used herein, additional therapeutic agents that are normally administered to treat a particular disease, or condition, are known as “appropriate for the disease, or condition, being treated”. As used herein, “additional therapeutic agents” is meant to include chemotherapeutic agents and other anti-proliferative agents.

For example, chemotherapeutic agents or other anti-proliferative agents may be combined with the compounds of this invention to treat proliferative disease or cancer. Examples of chemotherapeutic agents or other anti-proliferative agents include HDAC inhibitors including, but are not limited to, SAHA, MS-275, MG0103, and those described in WO 2006/010264, WO 03/024448, WO 2004/069823, US 2006/0058298, US 2005/0288282, WO 00/71703, WO 01/38322, WO 01/70675, WO 03/006652, WO 2004/035525, WO 2005/030705, WO 2005/092899, and demethylating agents including, but not limited to, 5-aza-dC, Vidaza and Decitabine and those described in U.S. Pat. No. 6,268,137, U.S. Pat. No. 5,578,716, U.S. Pat. No. 5,919,772, U.S. Pat. No. 6,054,439, U.S. Pat. No. 6,184,211, U.S. Pat. No. 6,020,318, U.S. Pat. No. 6,066,625, U.S. Pat. No. 6,506,735, U.S. Pat. No. 6,221,849, U.S. Pat. No. 6,953,783, U.S. Ser. No. 11/393,380 and PCT/US2006/001791.

In another embodiment of the present invention, for example, chemotherapeutic agents or other anti-proliferative agents may be combined with the compounds of this invention to treat proliferative diseases and cancer. Examples of known chemotherapeutic agents include, but are not limited to, for example, other therapies or anticancer agents that may be used in combination with the inventive anticancer agents of the present invention and include surgery, radiotherapy (in but a few examples, gamma-radiation, neutron beam radiotherapy, electron beam radiotherapy, proton therapy, brachytherapy, and systemic radioactive isotopes, to name a few), endocrine therapy, taxanes (taxol, taxotere etc), platinum derivatives, biologic response modifiers (interferons, interleukins, and tumor necrosis factor (TNF), TRAIL receptor targeting agents, to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate, Pemetrexed etc), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), Cell cycle inhibitors (KSP mitotic kinesin inhibitors, CENP-E and CDK inhibitors), enzymes (Asparaginase), and hormones (Tamoxifen, Leuprolide, Flutamide, and Megestrol), Gleevec™, adriamycin, dexamethasone, and cyclophosphamide. Antiangiogenic agents (Avastin and others). Kinase inhibitors (Imatinib (Gleevec), Sutent, Nexavar, Erbitux, Herceptin, Tarceva, Iressa and others). Agents inhibiting or activating cancer pathways such as the mTOR, HIF (hypoxia induced factor) pathways and others. For a more comprehensive discussion of updated cancer therapies see, http://www.nci.nih.gov/, a list of the FDA approved oncology drugs at http://www.fda.gov/cder/cancer/druglistframe.htm, and The Merck Manual, Eighteenth Ed. 2006, the entire contents of which are hereby incorporated by reference.

In another embodiment, the compounds of the present invention can be combined with cytotoxic anti-cancer agents. Examples of such agents can be found in the 13th Edition of the Merck Index (2001) These agents include, by no way of limitation, asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other cytotoxic drugs suitable for use with the compounds of the invention include, but are not limited to, those compounds acknowledged to be used in the treatment of neoplastic diseases, such as those for example in Goodman and Gilman's The Pharmacological Basis of Therapeutics (Ninth Edition, 1996, McGraw-Hill). These agents include, by no way of limitation, aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other cytotoxic anti-cancer agents suitable for use in combination with the compounds of the invention also include newly discovered cytotoxic principles such as oxaliplatin, gemcitabine, capecitabine, epothilone and its natural or synthetic derivatives, temozolomide (Quinn et al., J. Clin. Oncology 2003, 21(4), 646-651), tositumomab (Bexxar), trabedectin (Vidal et al., Proceedings of the American Society for Clinical Oncology 2004, 23, abstract 3181), and the inhibitors of the kinesin spindle protein Eg5 (Wood et al., Curr. Opin. Pharmacol. 2001, 1, 370-377).

In another embodiment, the compounds of the present invention can be combined with other signal transduction inhibitors. Of particular interest are signal transduction inhibitors which target the EGFR family, such as EGFR, HER-2, and HER-4 (Raymond et al., Drugs 2000, 60 (Suppl.1), 15-23; Harari et al., Oncogene 2000, 19 (53), 6102-6114), and their respective ligands. Examples of such agents include, by no way of limitation, antibody therapies such as Herceptin (trastuzumab), Erbitux (cetuximab), and pertuzumab. Examples of such therapies also include, by no way of limitation, small-molecule kinase inhibitors such as ZD-1839/Iressa (Baselga et al., Drugs 2000, 60 (Suppl. 1), 33-40), OSI-774/Tarceva (Pollack et al. J. Pharm. Exp. Ther. 1999, 291(2), 739-748), CI-1033 (Bridges, Curr. Med. Chem. 1999, 6, 825-843), GW-2016 (Lackey et al., 92nd AACR Meeting, New Orleans, Mar. 24-28, 2001, abstract 4582), CP-724,714 (Jani et al., Proceedings of the American Society for Clinical Oncology 2004, 23, abstract 3122), HKI-272 (Rabindran et al., Cancer Res. 2004, 64, 3958-3965), and EKB-569 (Greenberger et al., 11th NCI-EORTC-AACR Symposium on New Drugs in Cancer Therapy, Amsterdam, Nov. 7-10, 2000, abstract 388).

In another embodiment, the compounds of the present invention can be combined with other signal transduction inhibitors targeting receptor kinases of the split-kinase domain families (VEGFR, FGFR, PDGFR, flt-3, c-kit, c-fms, and the like), and their respective ligands. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, Mar. 24-28, 2001, abstract 3152), AG-13736 (Herbst et al., Clin. Cancer Res. 2003, 9, 16 (suppl 1), abstract C253), KRN-951 (Taguchi et al., 95<th> AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539).

In another embodiment, the compounds of the present invention can be combined with inhibitors of the Raf/MEK/ERK transduction pathway (Avruch et al., Recent Prog. Horm. Res. 2001, 56, 127-155), or the PKB (akt) pathway (Lawlor et al., J. Cell Sci. 2001, 114, 2903-2910). These include, by no way of limitation, PD-325901 (Sebolt-Leopold et al., Proceedings of the American Association of Cancer Research 2004, 45, abstract 4003), and ARRY-142886 (Wallace et al., Proceedings of the American Association of Cancer Research 2004, 45, abstract 3891).

In another embodiment, the compounds of the present invention can be combined with inhibitors of histone deacetylase. Examples of such agents include, by no way of limitation, suberoylanilide hydroxamic acid (SAHA), LAQ-824 (Ottmann et al., Proceedings of the American Society for Clinical Oncology 2004, 23, abstract 3024), LBH-589 (Beck et al., Proceedings of the American Society for Clinical Oncology 2004, 23, abstract 3025), MS-275 (Ryan et al., Proceedings of the American Association of Cancer Research 2004, 45, abstract 2452), FR-901228 (Piekarz et al., Proceedings of the American Society for Clinical Oncology 2004, 23, abstract 3028) and MGCD0103 (U.S. Pat. No. 6,897,220).

In another embodiment, the compounds of the present invention can be combined with other anti-cancer agents such as proteasome inhibitors, and m-TOR inhibitors. These include, by no way of limitation, bortezomib (Mackay et al., Proceedings of the American Society for Clinical Oncology 2004, 23, Abstract 3109), and CCI-779 (Wu et al., Proceedings of the American Association of Cancer Research 2004, 45, abstract 3849). The compounds of the present invention can be combined with other anti-cancer agents such as topoisomerase inhibitors, including but not limited to camptothecin.

Those additional agents may be administered separately from the compound-containing composition, as part of a multiple dosage regimen. Alternatively, those agents may be part of a single dosage form, mixed together with the compound of this invention in a single composition. If administered as part of a multiple dosage regimen, the two active agents may be submitted simultaneously, sequentially or within a period of time from one another which would result in the desired activity of the agents.

The amount of both the compound and the additional therapeutic agent (in those compositions which comprise an additional therapeutic agent as described above) that may be combined with the carrier materials to produce a single dosage form will vary depending upon the host treated and the particular mode of administration.

In those compositions which comprise an additional therapeutic agent, that additional therapeutic agent and the compound of this invention may act synergistically.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

### General Procedures

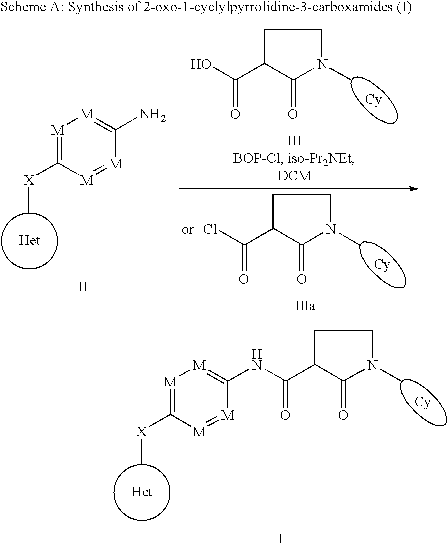 [0626] X=O, S, NH, N-alkyl;
Het=a monocyclic heteroaromatic ring systems mentioned in the specification and optionally substituted;
Cy=carbocyclic, heterocyclic, aromatic and heteroaromatic ring systems mentioned in the specification;
M independently selected from CH, N, and C—Y where Y are the substituents mentioned in the specification;

2-Oxo-1-cyclylpyrrolidine-3-carboxamides of a general formula I could be prepared via a coupling reaction between amines II and 2-oxo-1-cyclylpyrrolidine-3-carboxylic acids of a general formula III (scheme A), whereas amines II represent appropriately substituted various scaffolds suitable for the synthesis of kinase inhibitors or other compounds of pharmaceutical interest. Coupling of amines II with the acids III could be achieved in aprotic solvents such as DCM, CHCl₃, toluene, ethylene glycol dimethyl ether, MeCN, DMF, DMSO, THF, dioxane and like, using activating agents used in peptide chemistry and known to the skilled in the art, in the presence of organic bases such as DIPEA, Et₃N, DBU, DMAP, N-methylmorpholine, N-methylpiperidine, and like. Alternatively, acyl chlorides IIIa could be used instead of the acids III in the same types of solvents and in the presence of above mentioned bases. In these cases no activating agents are needed.

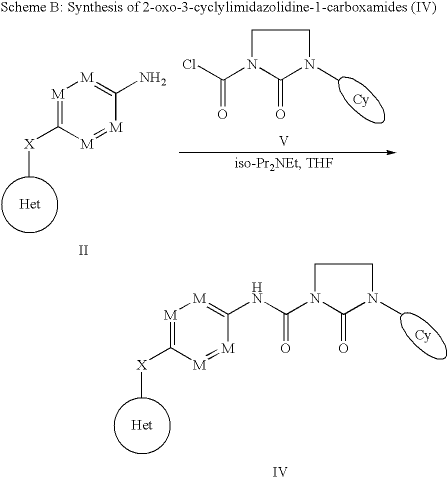

X=O, S, NH, N-alkyl;
Het=a monocyclic heteroaromatic ring systems mentioned in the specification and optionally substituted;
Cy=carbocyclic, heterocyclic, aromatic and heteroaromatic ring systems mentioned in the specification;
M independently selected from CH, N, and C—Y where Y are the substituents mentioned in the specification;

2-Oxo-3-cyclylimidazolidine-1-carboxamides of a general formula IV could be prepared via a condensation reaction between amines II and 2-oxo-3-cyclylimidazolidine-1-carbonyl chlorides of a general formula V (scheme B), whereas amines II represent appropriately substituted various scaffolds suitable for the synthesis of kinase inhibitors or other compounds of pharmaceutical interest. Coupling of amines II with the carbonyl chlorides V could be achieved in aprotic solvents such as DCM, CHCl₃, toluene, ethylene glycol dimethyl ether, MeCN, DMF, DMSO, THF, dioxane and like, in the presence of organic bases such as DIPEA, Et₃N, DBU, DMAP, N-methylmorpholine, N-methylpiperidine, and like.

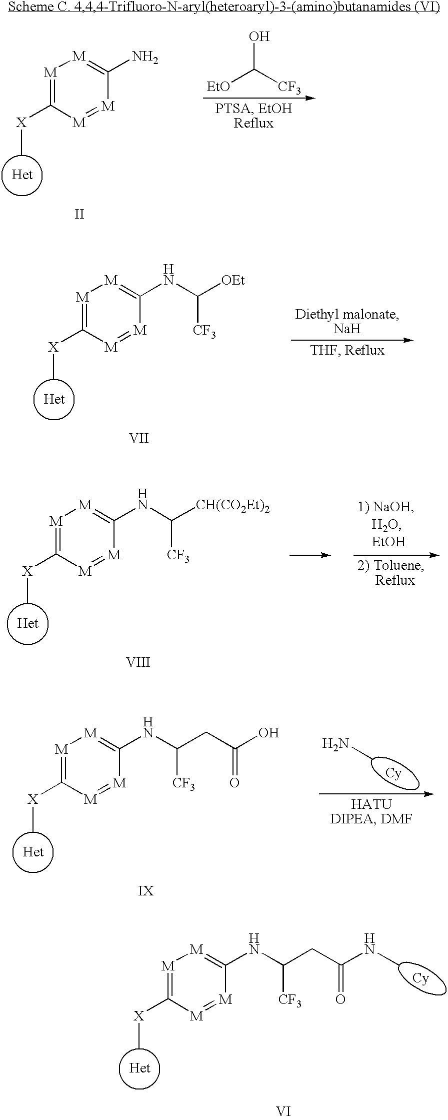

X=O, S, NH, N-alkyl;
Het=a monocyclic heteroaromatic ring systems mentioned in the specification and optionally substituted;
Cy=carbocyclic, heterocyclic, aromatic and heteroaromatic ring systems mentioned in the specification;
M independently selected from CH, N, and C—Y where Y are the substituents mentioned in the specification;

4,4,4-Trifluoro-N-aryl(heteroaryl)-3-(amino)butanamides of the general formula VI may be obtained via a short reaction sequence starting from the amines II. Amines II upon treatment with trifluoroacetaldehyde ethyl hemiacetal under acidic conditions (e.g. in the presence of 4-toluenesulfonic acid) in polar solvents such as ethanol are transformed into N-(1-ethoxy-2,2,2-trifluoroethyl)amines of the general structure VII. Compounds VII reacting with malonates under basic conditions form 2-(2,2,2-trifluoro-1-(amino)ethyl)malonates such as VIII. The amino di-esters VIII undergo alkaline hydrolysis to form the intermediate malonic acids (not shown in the scheme C), which are further decarboxylated, to afford 4,4,4-trifluoro-3-(amino)butanoic acids IX. Acids IX are coupled to different primary or secondary amines using standard techniques (for example, the ones described in the Scheme A), to produce title compounds VI.

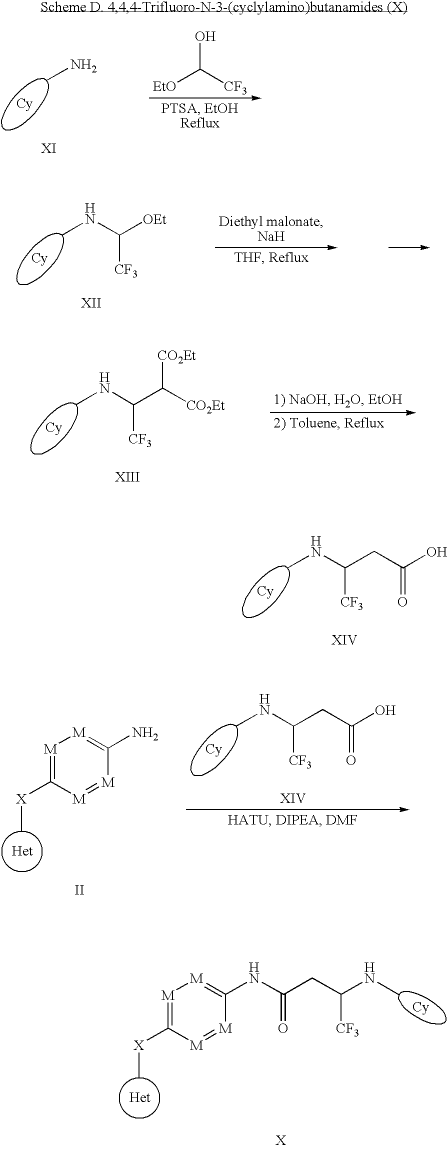

X=O, S, NH, N-alkyl;
Het=a monocyclic heteroaromatic ring systems mentioned in the specification and optionally substituted;
Cy=carbocyclic, heterocyclic, aromatic and heteroaromatic ring systems mentioned in the specification;
M independently selected from CH, N, and C—Y where Y are the substituents mentioned in the specification;

4,4,4-Trifluoro-N-3-(cyclylamino)butanamides of the general formula X may be obtained via a similar short reaction sequence as in Scheme C using the same sets of amines II and amines XI. Amines XI upon treatment with trifluoroacetaldehyde ethyl hemiacetal under acidic conditions (e.g. in the presence of 4-toluenesulfonic acid) in polar solvents such as ethanol are transformed into N-(1-ethoxy-2,2,2-trifluoroethyl)arylamines of the general structure XII. Compounds XII reacting with malonates under basic conditions form diethyl 2-(2,2,2-trifluoro-1-(cyclylamino)ethyl)malonates such as XIII. The amino di-esters XIII undergo alkaline hydrolysis to form the intermediate malonic acids (not shown in the scheme D), which are further decarboxylated, to afford 4,4,4-trifluoro-3-(cyclylamino)butanoic acids XIV. Acids XIV are coupled to various amines of the general structure II, using standard techniques (for example, the ones described in the Scheme A), to produce title compounds X.

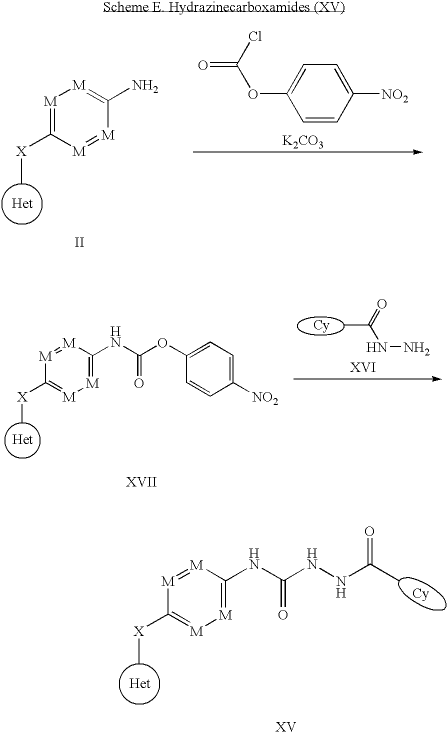

X=O, S, NH, N-alkyl;
Het=a monocyclic heteroaromatic ring systems mentioned in the specification and optionally substituted;
Cy=carbocyclic, heterocyclic, aromatic and heteroaromatic ring systems mentioned in the specification;
M independently selected from CH, N, and C—Y where Y are the substituents mentioned in the specification;

Hydrazinecarboxamides of general formula XV may be obtained using amines II and hydrazides XVI (Scheme E). Amines II upon treatment with 4-nitrophenyl chloroformate could be converted to the intermediates such as XVII (not isolated from the reaction mixtures), which further react with hydrazides XVI to form target molecules XV. Reagents such as triphosgene, carbonyl di-imidazole, etc. to form intermediate species capable to react with hydrazides XVI, may be used instead of 4-nitrophenyl chloroformate.

### PARTICULAR EXAMPLES

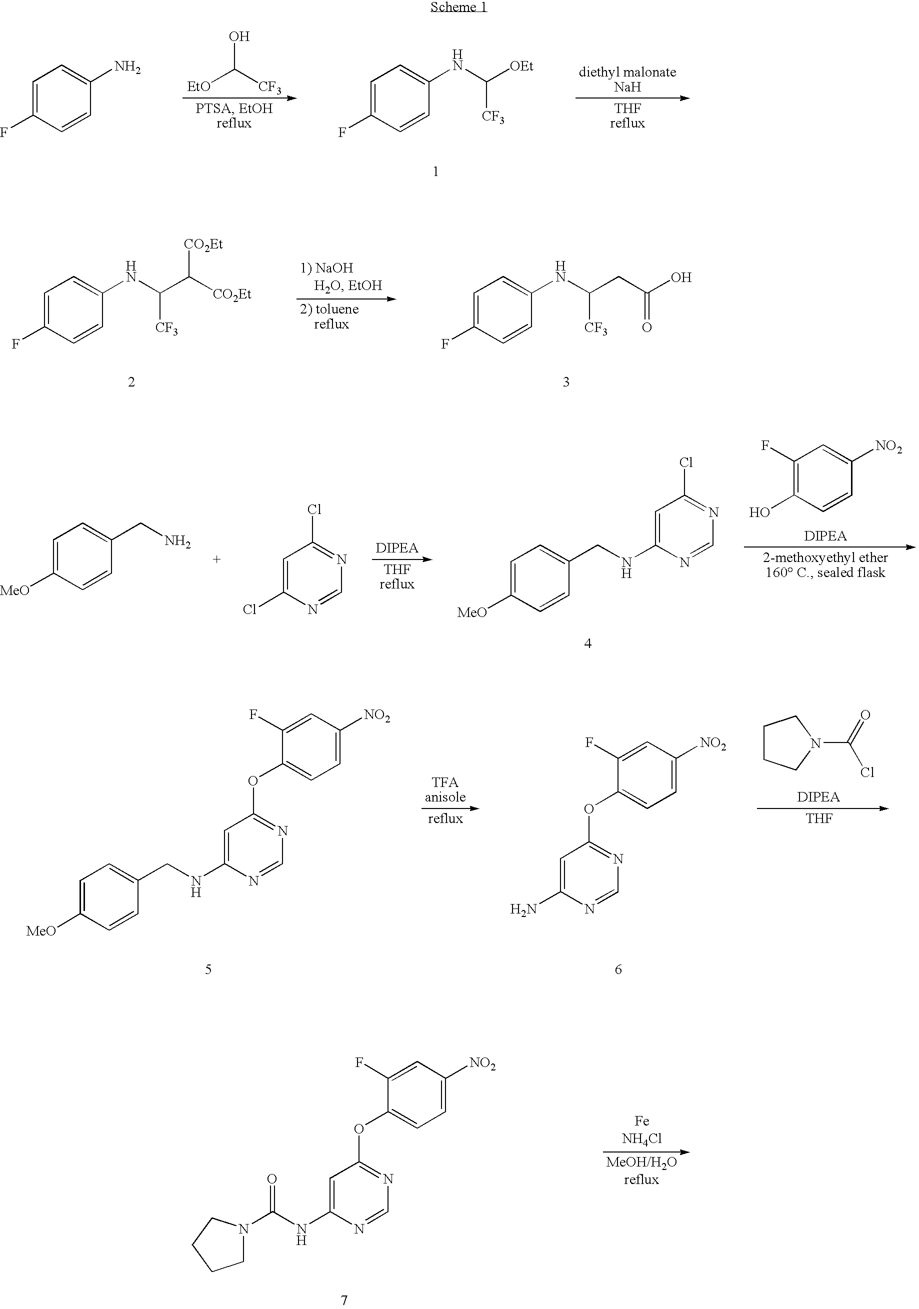

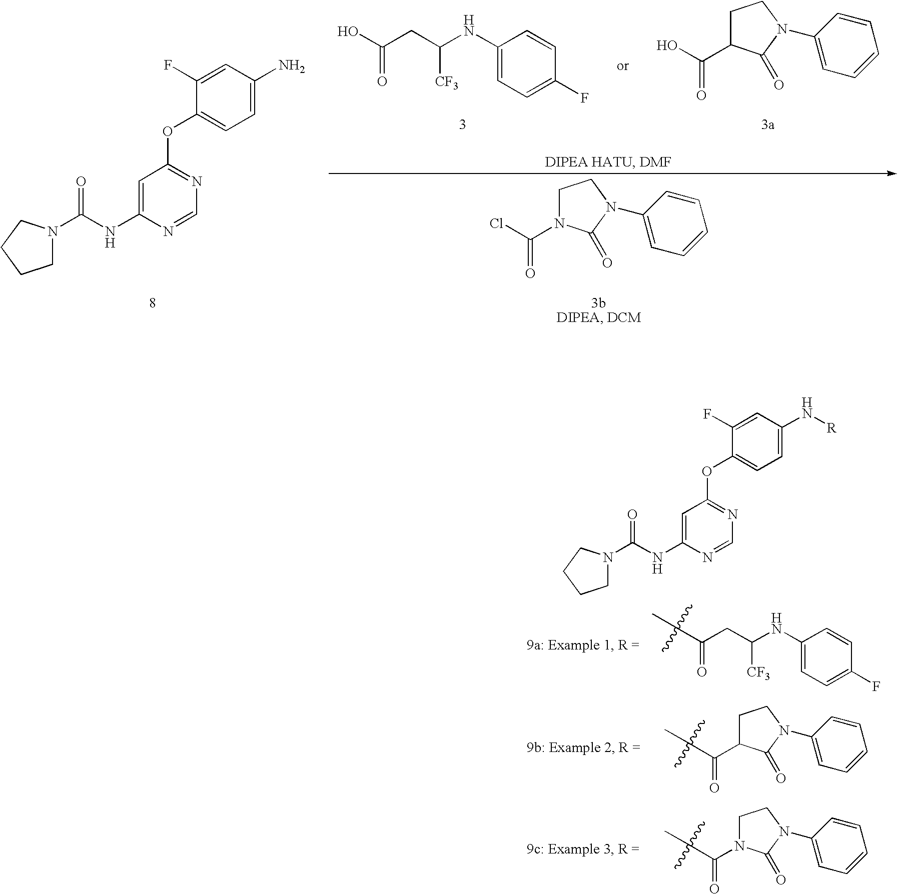

### Example 1

### N-(6-(2-Fluoro-4-(4,4,4-trifluoro-3-(4-fluorophenylamino)butanamido)phenoxy)pyrimidin-4-yl)pyrrolidine-1-carboxamide (9a)

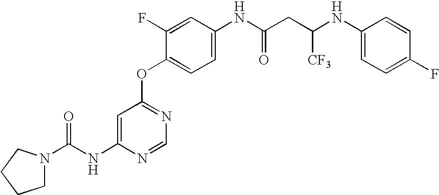

### Step 1. N-(1-Ethoxy-2,2,2-trifluoroethyl)-4-fluoroaniline (1)

A solution of 4-fluoroaniline (5 mL, 53.0 mmol), trifluoroacetaldehyde ethyl hemiacetal (6.2 mL, 53.0 mmol) and p-toluenesulfonic acid monohydrate (502 mg, 2.6 mmol) in ethanol (53 mL) was heated to reflux overnight under continuous stirring [Y. Gong, K. Kato. Journal of Fluorine Chem., 125 (2004), 767-773]. The reaction mixture was cooled, the solvent was removed under reduced pressure, the residue was dissolved in EtOAc and was washed with a saturated aqueous solution of sodium bicarbonate, dried over anhydrous magnesium sulfate, filtered and evaporated under reduced pressure to afford title compound 1 (4.16 g, not pure) as a yellow oil which was used directly for next step.

### Step 2. Diethyl 2-(2,2,2-trifluoro-1-(4-fluorophenylamino)ethyl)malonate (2)

A solution of diethyl malonate (6.85 mL, 45 mmol) in anhydrous tetrahydrofuran (25 mL) was added drop wise at 0° C. over 20 min into a dispersion of sodium hydride (60% in oil, 1.80 g, 45 mmol) in dry tetrahydrofuran (100 mL) [Y. Gong, K. Kato. Journal of Fluorine Chem., 125 (2004), 767-773]. To the resultant solution 1 (8.51, 39 mmol) in dry tetrahydrofuran (10 mL) was added and the reaction mixture was heated to reflux under vigorous stirring for 24 h. The reaction mixture was then cooled, acidified to pH 3 using a 1N HCl solution and extracted with EtOAc. The extract was dried over anhydrous sodium sulfate, filtered and evaporated under reduced pressure. The residue was purified by column chromatography on silica gel (eluent a gradient of ethyl acetate-hexane, from 0:100 to 20:80) to afford title compound 2 (14.22 g, 40.5 mmol, 98% yield) as a yellow-orange oil (not completely pure). MS: 352.1 (M+1).

### Step 3. 4,4,4-Trifluoro-3-(4-fluorophenylamino)butanoic acid (3)

A solution of 2 (14.22 g, 40.0 mmol) and sodium hydroxide (16.19 g, 40.5 mmol) in a mixture of water (34 mL) and ethanol (150 mL) was stirred at room temperature for 24 h [Y. Gong, K. Kato. Journal of Fluorine Chem., 125 (2004), 767-773]. The solvents were removed under reduced pressure leaving a white solid which was triturated in ether, collected by filtration, rinsed with ether and dried under high vacuum. This material was then re-dissolved in water. The solution was neutralized to pH 4 with a 3N HCl solution and extracted with EtOAc. The organic extract was dried over anhydrous magnesium sulfate, filtered and evaporated under reduced pressure. The solid residue was dissolved in dry toluene (130 mL), heated to reflux for 1 h under continuous stirring, and the solvent was removed under reduced pressure. The residue was purified by column chromatography on silica gel (eluent a gradient of EtOAc-hexane, from 0:100 to 40:60). Fractions containing the product were evaporated and the residue was partitioned between dichloromethane and a saturated aqueous solution of sodium bicarbonate. The two layers were separated (the organic extract was discarded) and the aqueous layer was acidified to pH=3. This acidic solution was extracted twice with dichloromethane. The combined organic layers were dried with magnesium sulfate, filtered and evaporated to afford title compound 3 (1.608 mg, 6.4 mmol, 15% yield) as a yellow solid. ¹H NMR (400 MHz, CDCl₃) δ ppm: 12.61 (s, 1H), 6.97-6.90 (m, 2H), 6.74-6.69 (m, 2H), 6.06 (d, J=9.2 Hz, 1H), 4.52-4.46 (m, 1H), 2.77 (dd, J=16.2, 3.4 Hz, 1H), 2.55 (dd, J=16.2, 10.0 Hz, 1H). MS: 252.1 (M+1).

### Step 4. 6-Chloro-N-(4-methoxybenzyl)pyrimidin-4-amine (4)

A mixture of 4,6-dichloropyrimidine (1 g, 6.71 mmol), 4-methoxybenzylamine (0.96 mL, 7.38 mmol) and N,N-diisopropylethylamine (2.92 mL, 16.8 mmol) in dry tetrahydrofuran (30 mL) was heated to reflux for 16 h under vigorous stirring. The reaction mixture was cooled, diluted with ethyl acetate, successively washed with saturated aqueous sodium bicarbonate solution and a saturated aqueous ammonium chloride solution, dried over anhydrous sodium sulfate, filtered and evaporated under reduced pressure. The yellow residue was purified by column chromatography on silica gel (eluent a gradient of EtOAc-hexane, from 30:70 to 50:50) to afford title compound 4 (1.47 g, 5.89 mmol, 88% yield) as an off-white solid. ¹H NMR (400 MHz, DMSO-d₆) δ ppm: 8.35-8.20 (bs, 1H), 8.18-8.07 (bs, 1H), 7.24 (d, J=8.4 Hz, 2H), 6.89 (d, J=8.4 Hz, 2H), 6.55 (bs, 1H), 4.52-4.26 (m, 2H), 3.72 (s, 3H). MS: 250.1 (M+1).

### Step 5. 6-(2-Fluoro-4-nitrophenoxy)-N-(4-methoxybenzyl)pyrimidin-4-amine (5)

A mixture of compound 4 (10 g, 40.0 mmol), 2-fluoro-4-nitrophenol (7.55 g, 48.1 mmol), N,N-diisopropylethylamine (70 mL, 400 mmol) in 2-methoxyethyl ether (200 mL) was heated at 160° C. in a sealed pressure bottle for 90 h. The mixture was cooled, diluted with ethyl acetate. The organic layer was successively washed with saturated aqueous solution of ammonium chloride and saturated aqueous solution of sodium bicarbonate, dried over anhydrous sodium sulfate and the solvent was removed under reduced pressure. The residue was triturated with hexane and ethyl acetate to give the title compound 5 (8.59 g, 23.2 mmol, 58%) as a brown solid. MS: 371.2 (M+1).

### Step 6. 6-(2-Fluoro-4-nitrophenoxy)pyrimidin-4-amine (6)

A mixture of compound 5 (8.59 g, 23.2 mmol), and anisole (2.53 mL, 23.2 mmol) in TFA (100 mL) was heated to reflux for 6 h. The TFA was removed under reduced pressure and the residue was partitioned between ethyl acetate and a saturated aqueous solution of sodium bicarbonate. The layers were separated, the organic layer was dried over anhydrous sodium sulfate and the solvent was removed under reduced pressure. The residue was purified by column chromatography on silica gel (eluent ethyl acetate-dichloromethane, 50:50) and trituration in a mixture of ethyl acetate and hexane to afford title compound 6 (4.9 g, 19.6 mmol, 84% yield) as a beige solid. MS: 251.0 (M+1).

### Step 7. N-(6-(2-Fluoro-4-nitrophenoxy)pyrimidin-4-yl)pyrrolidine-1-carboxamide (7)

To a stirred mixture of compound 6 (2 g, 8.0 mmol) and N,N-diisopropylethylamine (4.2 mL, 24 mmol) in tetrahydrofuran (20 mL) was added 1-pyrrolidinecarbonyl chloride (3 mL, 27.2 mmol). The mixture was heated to reflux for 20 h, cooled and partitioned between ethyl acetate and water. The layers were separated and the aqueous phase was extracted a second time with ethyl acetate. The organic layers were combined, dried over anhydrous sodium sulfate, filtered and evaporated under reduced pressure. The residue was purified by column chromatography on silica gel (Biotage 25M column, linear gradient 0-80% ethyl acetate in dichloromethane) to afford title compound 7 (900 mg, 2.59 mmol, 32% yield) as a yellow solid. MS: 348.1 (M+1).

### Step 8. N-(6-(4-Amino-2-fluorophenoxy)pyrimidin-4-yl)pyrrolidine-1-carboxamide (8)

To a suspension of compound 7 (900 mg, 2.59 mmol) in a mixture of methanol (26 mL) and water (13 mL) was added ammonium chloride (120 mg, 2.23 mmol) followed by iron powder (1.23 g, 22.0 mmol). The mixture was heated to reflux under nitrogen for 40 min, diluted with methanol and dichloromethane, filtered and the solvents were removed under reduced pressure. The residue was purified by column chromatography on silica gel (Biotage 25M column, linear gradient 0-5% methanol in dichloromethane) to afford title compound 8 (620 mg, 1.95 mmol, 75% yield) as a yellow solid. ¹H NMR (400 MHz, DMSO-d₆) δ ppm: 9.30 (s, 1H), 8.38 (s, 1H), 7.34 (s, 1H), 6.94 (t, J=8.8 Hz, 1H), 6.46 (dd, J=12.8, 2.4 Hz, 1H), 6.37 (ddd, J=8.8, 2.4, 0.8 Hz, 1H), 5.39 (s, 2H), 3.39 (bs, 4H), 1.82 (bs, 4H). MS: 318.2 (M+1).

### Step 9. N-(6-(2-fluoro-4-(4,4,4-trifluoro-3-(4-fluorophenylamino)butanamido)phenoxy)pyrimidin-4-yl)pyrrolidine-1-carboxamide (9a)

To a stirred solution of compound 8 (60 mg, 0.189 mmol), carboxylic acid 3 (90 mg, 0.38 mmol) and N,N-diisopropylethylamine (0.12 mL, 0.66 mmol) in dry N,N-dimethylformamide (5 mL) was added the HATU reagent (215 mg, 0.57 mmol). The mixture was stirred at room temperature for 16 h, quenched with saturated aqueous sodium bicarbonate solution and extracted with ethyl acetate. The extract was dried over anhydrous sodium sulfate, filtered and evaporated under reduced pressure. The residue was purified by column chromatography on silica gel (Biotage 12M column, linear gradient 0-10% methanol in dichloromethane) followed by trituration with a mixture ethyl acetate-hexane to afford title compound 9a (59 mg, 0.107 mmol, 57% yield) as an off-white solid. ¹H NMR (400 MHz, DMSO-d₆) δ ppm: 10.38 (s, 1H), 9.39 (s, 1H), 8.37 (d, J=1.2 Hz, 1H), 7.69 (dd, J=12.8, 2.0 Hz, 1H), 7.43 (d, J=0.8 Hz, 1H), 7.33-7.26 (m, 2H), 6.94 (t, J=8.8 Hz, 2H), 6.77-6.71 (m, 2H), 6.04 (d, J=5.2 Hz, 1H), 4.70-4.58 (m, 1H), 3.50-3.30 (m, 4H), 3.90 (dd, J=16.0, 4.0 Hz, 1H), 2.73 (dd, J=16.0, 9.2 Hz, 1H), 1.82 (bs, 4H). MS: 551.2 (M+1).

### Example 2

### N-(6-(2-Fluoro-4-(2-oxo-1-phenylpyrrolidine-3-carboxamido)phenoxy)pyrimidin-4-yl)pyrrolidine-1-carboxamide (9b)

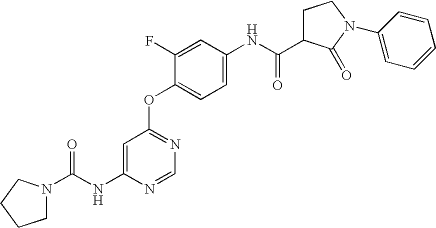

Starting from compound 8 and following the same procedure as described for the synthesis of compound 9a (scheme 1, example 1, step 9) but substituting carboxylic acid 3 by 2-oxo-1-phenylpyrrolidine-3-carboxylic acid (3a), title compound 9b was obtained in 33% yield as a beige solid. ¹H NMR (400 MHz, DMSO-d₆) δ ppm: 10.61 (s, 1H), 9.41 (s, 1H), 8.40 (s, 1H), 7.81 (dd, J=12.8, 2.4 Hz, 1H), 7.70-7.65 (m, 2H), 7.46 (d, J=1.2 Hz, 1H), 7.43-7.37 (m, 3H), 7.34 (t, J=8.4 Hz, 1H), 7.20-7.15 (m, 1H), 3.99-3.87 (m, 2H), 3.77 (t, J=8.4 Hz, 1H), 3.40 (bs, 4H), 2.50-2.34 (m, 2H), 1.83 (bs, 4H). MS: 505.3 (M+1).

### Example 3

### N-(3-Fluoro-4-(6-(pyrrolidine-1-carboxamido)pyrimidin-4-yloxy)phenyl)-2-oxo-3-phenylimidazolidine-1-carboxamide (9c)

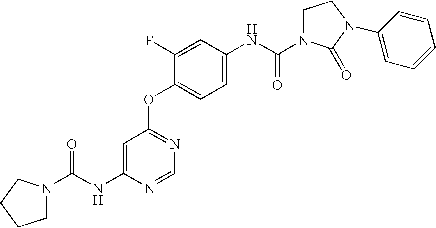

To a solution of the amine 8 (example 1, scheme 1) (40 mg, 0.13 mmol) and N,N-diisopropylethylamine (68 μL, 0.39 mmol) in dry dichloromethane (5 mL) at 0° C. under nitrogen was added 2-oxo-3-phenylimidazolidine-1-carbonyl chloride (3b) (0.1 M solution in tetrahydrofuran, 2 mL, 0.20 mmol) {This solution was prepared by heating a mixture of 1-phenylimidazolidine-2-one (175 mg, 1.08 mmol) and triphosgene (112 mg, 0.378 mmol) in dry tetrahydrofuran (11 mL) at 70° C. for 3 h [Mayer et al, J. Med. Chem. 2000, 43, 3653-3664 J. A. Maclaren, Aust. J. Chem. 1977, 30, 455-457 and J. Chem. Res. Synop. 2000, 9, 440-441} The reaction mixture was allowed to warm to room temperature and stirring was continued for an additional 16 h. Methanol (5 mL) was then added to the reaction mixture and the solvents were removed under reduced pressure. The residue was diluted with ethyl acetate and the organic phase was washed with saturated aqueous sodium bicarbonate solution, saturated aqueous ammonium chloride solution and brine, dried over anhydrous sodium sulfate, filtered and evaporated under reduced pressure. The residue was purified by column chromatography on silica gel (Biotage 12M column, linear gradient 0-20% methanol in dichloromethane and linear gradient 0-100% ethyl acetate in dichloromethane) followed by purification by preparative HPLC (Aquasil C-18 column, linear gradient: MeOH/water [0.05% formic acid in both] 40% to 90%) and trituration with a mixture ethyl acetate-hexane, to afford title compound 9c (14.6 mg, 0.029 mmol, 22% yield) as a beige solid. ¹H NMR (400 MHz, DMSO-d₆) δ ppm: 10.51 s, 1H), 9.41 (s, 1H), 8.40 (s, 1H), 7.74 (dd, J=13.2, 2.4 Hz, 1H), 7.63 (dd, J=8.4, 1.0 Hz, 2H), 7.47 (d, J=1.0 Hz, 1H), 7.46-7.40 (m, 2H), 7.38-7.30 (m, 2H), 7.18 (t, J=7.2 Hz, 1H), 4.00-3.91 (m, 4H), 3.41 (bs, 4H), 1.83 (bs, 4H). MS: 506.3 (M+1).

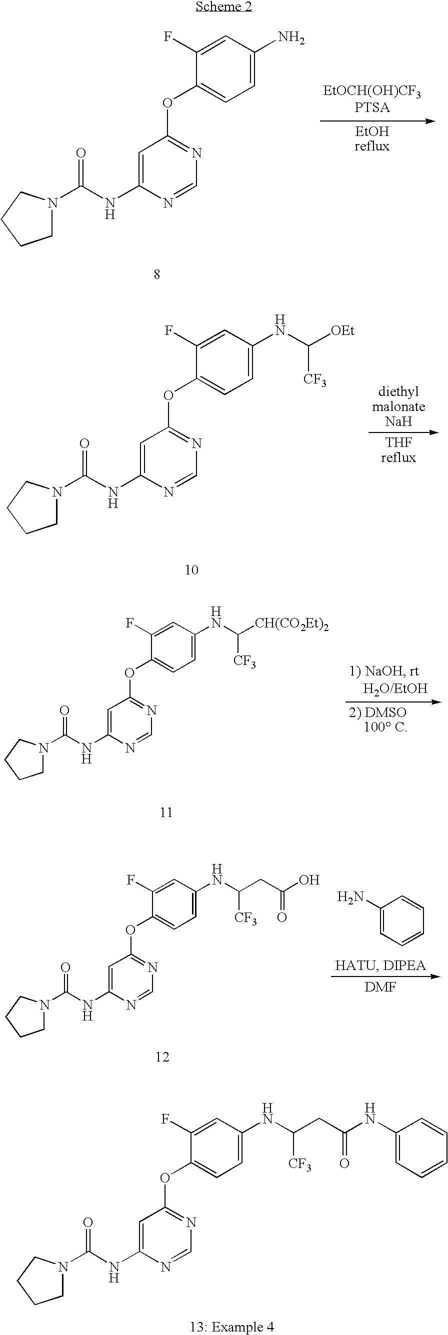

### Example 4

### N-(6-(2-Fluoro-4-(1,1,1-trifluoro-4-oxo-4-(phenylamino)butan-2-ylamino)phenoxy)pyrimidin-4-yl)pyrrolidine-1-carboxamide (13)

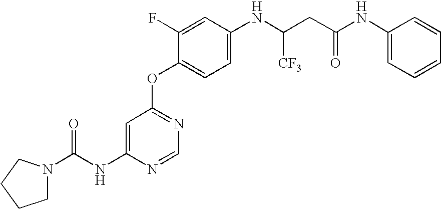

### Step 1. N-(6-(4-(1-Ethoxy-2,2,2-trifluoroethylamino)-2-fluorophenoxy)pyrimidin-4-yl)pyrrolidine-1-carboxamide (10)

A mixture of amine 8 (260 mg, 0.819 mmol), trifluoroacetaldehyde ethyl hemiacetal (0.29 mL, 2.46 mmol) and 4-toluenesulfonic acid monohydrate (171 mg, 0.901 mmol) in ethanol (35 mL) was heated to reflux for 24 h under nitrogen. The reaction mixture was concentrated, diluted with ethyl acetate, washed with saturated aqueous sodium bicarbonate solution, dried over anhydrous sodium sulfate, filtered and evaporated under reduced pressure to afford title compound 10 (360 mg, 0.812 mmol, 99% yield) which was used without purification. MS: 444.2 (M+1).

### Step 2. Diethyl 2-(2,2,2-trifluoro-1-(3-fluoro-4-(6-(pyrrolidine-1-carboxamido)pyrimidin-4-yloxy)phenylamino)ethyl)malonate (11)

To a solution of compound 10 (360 mg, 0.812 mmol) and diethyl malonate (0.136 mL, 0.893 mmol) in anhydrous tetrahydrofuran (10 mL) under nitrogen was added sodium hydride (60% in oil, 71 mg, 1.79 mmol). The mixture was heated to reflux for 5 h, cooled, diluted with water, acidified to pH 3 using 1N HCl solution and extracted with ethyl acetate. The extract was dried over anhydrous sodium sulfate, filtered and evaporated under reduced pressure. The residue was purified by column chromatography on silica gel (Biotage 25M, linear gradient 20-60% ethyl acetate-dichloromethane) to afford compound 11 (100 mg, 0.179 mmol, 22% yield). MS: 558.3 (M+1).

### Step 3. 4,4,4-Trifluoro-3-(3-fluoro-4-(6-(pyrrolidine-1-carboxamido)pyrimidin-4-yloxy)phenylamino)butanoic acid (12)

A solution of compound 11 (100 mg, 0.179 mmol) and sodium hydroxide (72 mg, 1.79 mmol) in water (0.5 mL) and ethanol (2.5 mL) was stirred at room temperature for 24 h. The solvents were removed under reduced pressure and the residue was re-dissolved in water (20 mL). The solution was neutralized to pH 3 with a 3N HCl solution and extracted with ethyl acetate. The organic extract was dried over anhydrous sodium sulfate and the solvent was removed under reduced pressure. The remained solid was dissolved in DMSO (4 mL) and the solution was heated at 100° C. for 1 h, cooled, diluted with water, acidified to pH 3 using a 3N HCl solution and extracted three times with ethyl acetate. The combined organic extracts were dried over anhydrous sodium sulfate, filtered and evaporated under reduced pressure. The residue was purified by column chromatography on silica gel (Biotage 12M, linear gradient 0-20% methanol-dichloromethane) to afford compound 12 (76 mg, 0.17 mmol, 92% yield) as a solid material. MS: 458.2 (M+1).

### Step 4. N-(6-(2-Fluoro-4-(1,1,1-trifluoro-4-oxo-4-(phenylamino)butan-2-ylamino) phenoxy)pyrimidin-4-yl)pyrrolidine-1-carboxamide (13)

Following the same procedure as described for compound 9a, scheme 1, example 1, step 9, but substituting carboxylic acid 3 by compound 12 and compound 8 by aniline, title compound 13 was obtained in 19% yield as a white solid. ¹H NMR (400 MHz, DMSO-d₆) δ ppm: 10.12 (s, 1H), 9.33 (s, 1H), 8.38 (d, J=1.2 Hz, 1H), 7.56 (dd, J=8.4, 1.2 Hz, 2H), 7.40 (d, J=1.2 Hz, 1H), 7.29 (t, J=8.0 Hz, 2H), 7.08-7.01 (m, 2H), 6.74 (dd, J=13.6, 2.4 Hz, 1H), 6.58 (dd, J=8.8, 2.4 Hz, 1H), 6.46 (d, J=9.2 Hz, 1H), 4.56-4.53 (m, 1H), 3.50-3.30 (m, 4H), 2.90 (dd, J=15.6, 3.6 Hz, 1H), 2.75 (dd, J=15.6, 9.2 Hz, 1H), 1.82 (bs, 4H). MS: 533.2 (M+1).

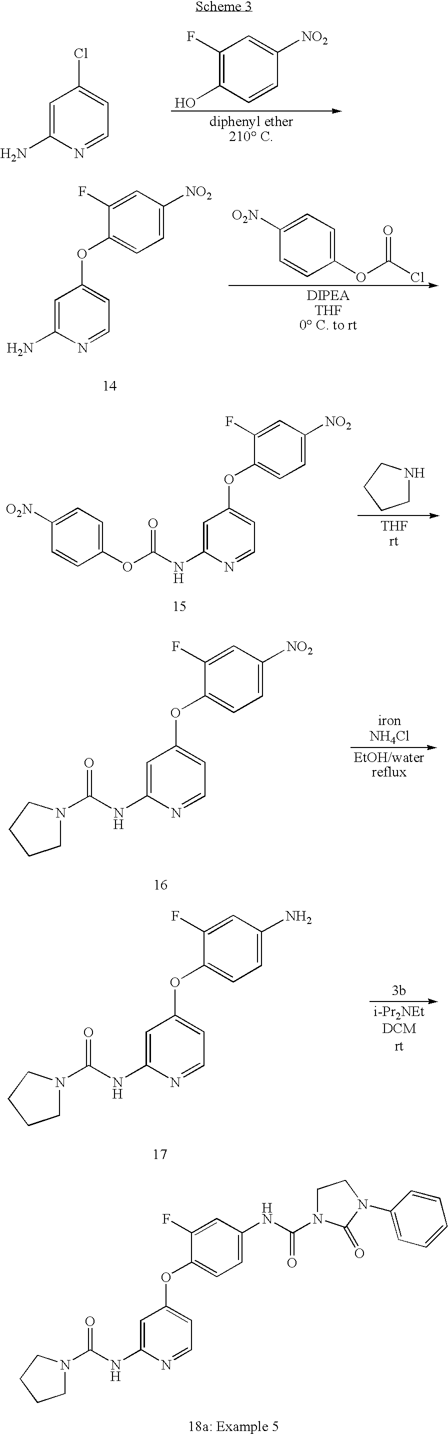

### Example 5

### N-(3-Fluoro-4-(2-(pyrrolidine-1-carboxamido)pyridin-4-yloxy)phenyl)-2-oxo-3-phenylimidazolidine-1-carboxamide (18a)

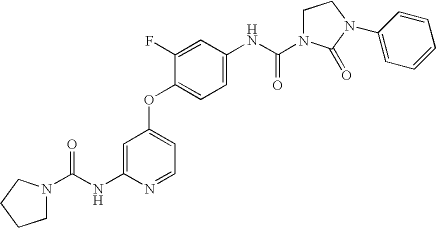

### Step 1. 4-(2-Fluoro-4-nitrophenoxy)pyridin-2-amine (14)

A mixture of 4-chloropyridin-2-amine (0.232 g, 1.805 mmol) [Wachi, K. and Terada, A. Chem. Pharm. Bull. 28(2) 465-472 (1980)], 2-fluoro-4-nitrophenol (0.567 g, 3.61 mmol) and potassium carbonate (0.748 g, 5.41 mmol) in diphenyl ether (2.406 ml) was stirred at 210° C. for 1 day. It was then cooled down to room temperature and partitioned between DCM and water. The organic phase was collected, dried over anhydrous sodium sulfate, filtered and concentrated under reduced pressure. The residue was purified by flash chromatography (50% to 75% EtOAc in hexanes followed by pure EtOAc) to afford the title compound 14 (0.245 g, 0.983 mmol, 54% yield) as an orange solid. MS: 350.1 (M+1).

### Step 2. 4-Nitrophenyl 4-(2-fluoro-4-nitrophenoxy)pyridin-2-ylcarbamate (15)

4-Nitrophenyl carbamate (0.297 g, 1.475 mmol) was added to a solution of 4-(2-fluoro-4-nitrophenoxy)pyridin-2-amine (14) (0.245 g, 0.983 mmol) and DIPEA (0.275 ml, 1.573 mmol) in THF (10 ml) at 0° C. The reaction mixture was stirred for 3 hrs and allowed to warm to room temperature over that period of time. It was then diluted with EtOAc, washed with 5% aqueous sodium bicarbonate and brine; dried over anhydrous sodium sulfate, filtered and concentrated under reduced pressure to afford the title compound 15 (0.4 g, 0.965 mmol, 98% yield). The material was used in the next step without further purification. MS: 415.1 (M+1).

### Step 3. N-(4-(2-Fluoro-4-nitrophenoxy)pyridin-2-yl)pyrrolidine-1-carboxamide (16)

A mixture of 4-nitrophenyl 4-(2-fluoro-4-nitrophenoxy)pyridin-2-ylcarbamate (15) (0.406 g, 0.98 mmol) and pyrrolidine (0.245 mL, 2.94 mmol) in THF (5 mL) was stirred overnight at room temperature. It was then diluted with EtOAc, washed with 5% ammonium chloride solution, dried over anhydrous sodium sulfate, filtered and concentrated under reduced pressure. The residue was purified by flash chromatography (80% to 100% EtOAc in hexanes) to afford the title compound 19 (0.284 g, 0.820 mmol, 84% yield) as a white solid. MS: 347.1 (M+1).

### Step 4. N-(4-(4-Amino-2-fluorophenoxy)pyridin-2-yl)pyrrolidine-1-carboxamide (17)

Iron powder (0.366 g, 6.56 mmol) was added to mixture of N-(4-(2-fluoro-4-nitrophenoxy)pyridin-2-yl)pyrrolidine-1-carboxamide (19) (0.284 g, 0.820 mmol) and ammonium chloride (0.037 g, 0.697 mmol) in ethanol (5.47 ml)/water (2.73 ml) and was heated to reflux under vigorous stirring for 40 min. The mixture was then cooled and filtered through a Celite® pad. The filtrate was collected and concentrated under reduced pressure. The residue was re-dissolved in DCM, the organic solution was washed with water; dried over anhydrous Na₂SO₄, filtered and concentrated under reduced pressure to afford the title compound 20 (0.255 g, 0.806 mmol, 98% yield) as creamy solid. MS: 317.1 (M+1).

### Step 5. N-(3-Fluoro-4-(2-(pyrrolidine-1-carboxamido)pyridin-4-yloxy)phenyl)-2-oxo-3-phenylimidazolidine-1-carboxamide (18a)

2-Oxo-3-phenylimidazolidine-1-carbonyl chloride (3b, scheme 1, example 3) (0.182 g, 0.811 mmol) was added to a solution of N-(4-(4-amino-2-fluorophenoxy)pyridin-2-yl)pyrrolidine-1-carboxamide 17 (0.171 g, 0.541 mmol) and DIPEA (0.378 ml, 2.162 mmol) in THF (2 ml) and the mixture was stirred at room temperature for 2 h. The crude mixture was concentrated and the residue purified by flash chromatography (eluent 5% to 10% MeOH in EtOAc) to afford the title compound 18a (0.020 g, 0.040 mmol, 7% yield) as white solid. ¹H NMR (400 MHz, DMSO-d₆) 10.51 (s, 1H), 8.03 (d, 1H, J=3.7 Hz), 7.71 (d, 1H, J=2.4 Hz), 6.68 (dd, 1H, J=1.3 Hz, J=12.1 Hz), 7.55 (m, 2H), 7.42 (m, 2H), 6.9-7.0 (m, 3H), 7.00 (s, 1H), 6.52 (dd, 1H, J=2.3 Hz, J=5.8 Hz). MS: 505.2 (M+1).

TABLE 1
Characterization of compounds 18b-18i (examples 6-13) prepared according to
Scheme 3

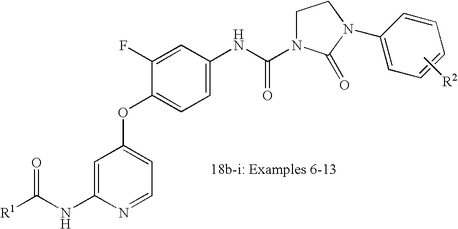

| Cpd | Ex. | R¹ | R² | Name | Characterization |
|---|---|---|---|---|---|
| 18b | 6 | 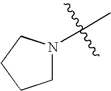 | 4-F | N-(3-fluoro-4-(2-(pyrrolidine-1-carboxamido)pyridin-4-yloxy)phenyl)-3-(4-fluorophenyl)-2-oxoimidazolidine-1-carboxamide | ¹H NMR (400 MHz DMSO-d₆) 10.49(s, 1H), 8.69 (s, 1H), 8.09 (d, 1H, J =5.7 Hz), 7.76 (d, 1H, J = 10.8 Hz),7.62 (m, 2H), 7.45 (m, 1H), 7.2-7.4(m, 4H), 6.58 (m, 1H), 3.93 (s, 4H),1.78 (s, 4H). MS (m/z): (M + 1)⁺ 523.3 |
| 18c | 7 | 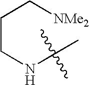 | H | N-(4-(2-(3-(3-(dimethylamino)propyl)ureido)pyridin-4-yloxy)-3-fluorophenyl)-2-oxo-3-phenylimidazolidine-1-carboxamide | ¹H NMR (400 MHz DMSO-d₆) δ(ppm): 10.54 (s, 1H), 9.15 (s, 1H),8.21 (s, 0.5H), 8.07 (dd, J = 5.9 Hz,1H), 8.03 (br, 1H), 7.81 (dd, J =2.4 Hz, J = 12.9 Hz, 1H), 7.65-7.61 (m,2H), 7.45-7.40 (m, 3H), 7.34 (t, J =8.9 Hz, 1H), 7.20-7.16 (m, 1H), 6.94 (s, 1H), 6.57 (dd, J = 2.4 Hz, J = 5.9 Hz,1H), 3.99-3.92 (m, 4H), 3.14 (m, 2H),2.36 (t, J = 7.2 Hz, 2H), 2.22 (s, 6H),1.61-1.57 (m, 2H). MS (m/z): (M + 1)536.3. |
| 18d | 8 | 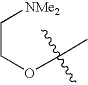 | H | 2-(dimethylamino)ethyl4-(2-fluoro-4-(2-oxo-3-phenylimidazolidine-1-carboxamido)phenoxy)pyridin-2-ylcarbamate | ¹H NMR (400 MHz, DMSO-d₆) δ(ppm): 10.55 (s, 1H), 10.25 (s, 1H),8.29 (s, 0.6H), 8.15 (d, J = 5.7 Hz, 1H),7.82 (dd, J = 12.9 Hz, J = 2.4 Hz, 1H),7.65-7.62 (m, 2H), 7.46-7.41 (m, 3H),7.38-7.20 (m, 2H), 7.20-7.16 (m, 1H),6.67 (dd, J = 2.4 Hz, J = 5.7 Hz, 1H), 4.11 (t, J = 5.9 Hz, 2H), 3.97-3.95 (m,4H), 2.46 (t, J = 5.9 Hz, 2H), 2.16 (s,6H). MS (m/z): (M + 1) 523.3. |
| 18e | 9 | 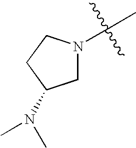 | H | (R)-N-(4-(2-(3-(dimethylamino)pyrrolidine-1-carboxamido)pyridin-4-yloxy)-3-fluorophenyl)-2-oxo-3-phenylimidazolidine-1-carboxamide | ¹H NMR (400 MHz, DMSO-d₆) δ(ppm): 10.54 (s, 1H), 8.76 (s, 1H),8.11 (d, J = 5.6 Hz, 1H), 7.80 (dd, J =2.5 Hz, J = 12.9 Hz, 1H), 7.64-7.62 (m,2H), 7.47-7.39 (m, 4H), 7.32 (t, J =8.8 Hz, 1H), 7.18 (t, J = 7.4 Hz, 1H), 6.62 (dd, J = 2.3 Hz, J = 5.7 Hz, 1H),4.00-3.91 (m, 4H), 3.65-3.45 (m, 3H),3.08 (m, 1H), 2.64 (m, 1H), 2.15 (s,6H), 2.02 (m, 1H), 1.65 (m, 1H) MS(m/z): (M + 1) 548.3. |
| 18f | 10 | 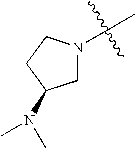 | H | (S)-N-(4-(2-(3-(dimethylamino)pyrrolidine-1-carboxamido)pyridin-4-yloxy)-3-fluorophenyl)-2-oxo-3-phenylimidazolidine-1-carboxamide | ¹H NMR (400 MHz, DMSO-d₆) δ(ppm): 10.54 (s, 1H), 8.76 (s, 1H),8.11 (d, J = 5.6 Hz, 1H), 7.80 (dd, J =2.5 Hz, J = 12.9 Hz, 1H), 7.64-7.62 (m,2H), 7.47-7.39 (m, 4H), 7.32 (t, J =8.8 Hz, 1H), 7.18 (t, J = 7.4 Hz, 1H), 6.62 (dd, J = 2.3 Hz, J = 5.7 Hz, 1H),4.00-3.91 (m, 4H), 3.65-3.45 (m, 3H),3.08 (m, 1H), 2.64 (m, 1H), 2.15 (s,6H), 2.02 (m, 1H), 1.65 (m, 1H) MS(m/z): (M + 1) 548.3. |
| 18g | 11 | 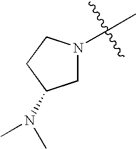 | 4-F | (R)-N-(4-(2-(3-(dimethylamino)pyrrolidine-1-carboxamido)pyridin-4-yloxy)-3-fluorophenyl)-3-(4-fluorophenyl)-2-oxoimidazolidine-1-carboxamide | ¹H NMR (400 MHz, DMSO-d₆) δ(ppm): 10.462 (s, 1H), 8.02 (d, J =5.7 Hz, 1H), 7.69-7.65 (m, 2H), 7.51-7.48 (m, 2H), 7.18-7.08 (m, 5H), 6.54(dd, J = 2.2 Hz, J = 5.7 Hz, 1H), 4.10-4.06 (m, 2H), 3.96-3.92 (m, 2H), 3.76 (t, J = 8.2 Hz, 1H), 3.66 (t, J = 8.8 Hz,1H), 3.45-3.38 (m, 1H), 3.32-3.27 (m,1H), 2.91-2.83 (m, 1H), 2.33 (s, 6H),2.22-2.16 (m, 1H), 2.04-1.94 (m,1H) MS (m/z): (M + 1) 566.2. |
| 18h | 12 | 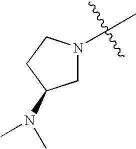 | 4-F | (S)-N-(4-(2-(3-(dimethylamino)pyrrolidine-1-carboxamido)pyridin-4-yloxy)-3-fluorophenyl)-3-(4-fluorophenyl)-2-oxoimidazolidine-1-carboxamide | ¹H NMR (400 MHz, DMSO-d₆) δ(ppm): 10.462 (s, 1H), 8.02 (d, J =5.7 Hz, 1H), 7.69-7.65 (m, 2H), 7.51-7.48 (m, 2H), 7.18-7.08 (m, 5H), 6.54(dd, J = 2.2 Hz, J = 5.7 Hz, 1H), 4.10-4.06 (m, 2H), 3.96-3.92 (m, 2H), 3.76 (t, J = 8.2 Hz, 1H), 3.66 (t, J = 8.8 Hz,1H), 3.45-3.38 (m, 1H), 3.32-3.27 (m,1H), 2.91-2.83 (m, 1H), 2.33 (s, 6H),2.22-2.16 (m, 1H), 2.04-1.94 (m,1H) MS (m/z): (M + 1) 566.2. |
| 18i | 13 | 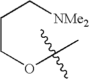 | H | 3-(dimethylamino)propyl4-(2-fluoro-4-(2-oxo-3-phenylimidazolidine-1-carboxamido)phenoxy),pyridin-2-ylcarbamate | ¹H NMR (400 MHz, DMSO-d₆) δ(ppm): 10.55 (s, 1H), 10.21 (s, 1H),8.15 (d, J = 5.7 Hz, 1H), 7.82 (dd, J =11.9 Hz, J = 2.1 Hz, 1H), 7.63 (m, 2H),7-45-7.33 (m, 5H), 7.18 (t, J = 7.3 Hz,1H) 6.67 (dd, J = 5.8 Hz, 2.3 Hz, 1H),4.05 (t, J = 6.7 Hz, 2H), 3.96-3.95 (m,4H), 2.25 (t, J = 7 Hz, 2H), 1.69 (t, J =6.8 Hz, 2H) MS (m/z): (M + 1) 537.3. |

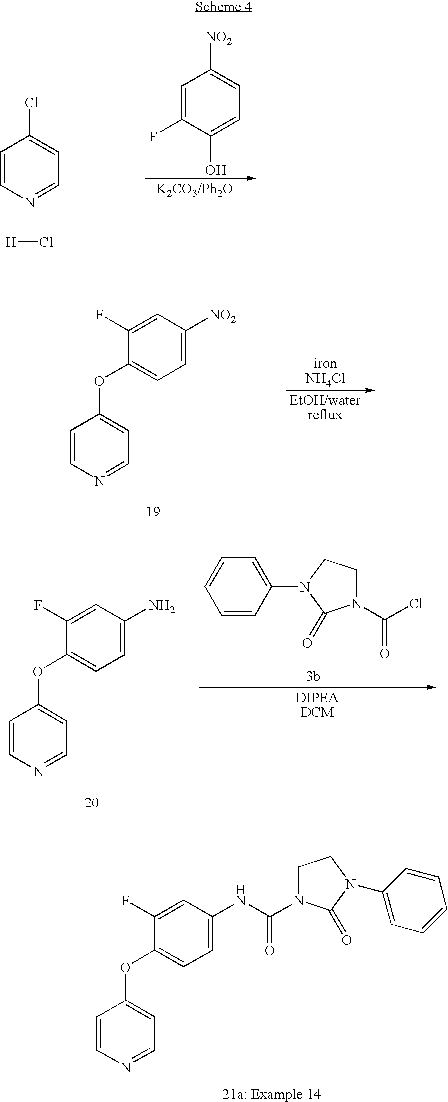

### Example 14

### N-(3-Fluoro-4-(pyridin-4-yloxy)phenyl)-2-oxo-3-phenylimidazolidine-1-carboxamide (21a)

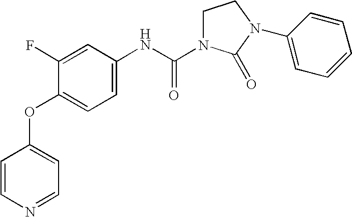

### Step 1. 4-(2-Fluoro-4-nitrophenoxy)pyridine (19)

A mixture of 4-chloropyridine hydrochloride (5 g, 33.3 mmol), 2-fluoro-4-nitrophenol (10.47 g, 66.7 mmol) and potassium carbonate (9.21 g, 66.7 mmol) in diphenyl ether (44.4 ml) was stirred at 150° C. for 1 h. It was then cooled to room temperature, suspended in ether and filtered. The solid residue was suspended in aqueous sodium bicarbonate and extracted with EtOAc; the organic phase was washed with water and brine, and put aside (solution 1). The ether filtrate was extracted with 1N HCl; the acidic aqueous extract was collected, basified to pH˜11 by addition of 1N NaOH and extracted with EtOAc (solution 2). Both solutions were combined, dried over anhydrous sodium sulfate, filtered and concentrated under reduced pressure. The resultant syrup was treated with hexanes to form a precipitate which was collected by filtration and dried under reduced pressure to afford the title compound 19 (2.79 g, 11.91 mmol, 35% yield) as light cream solid. MS: 235.1 (M+1).

### Step 2. 3-Fluoro-4-(pyridin-4-yloxy)aniline (20)

Iron powder (5.32 g, 95 mmol) was added to a mixture of 4-(2-fluoro-4-nitrophenoxy)pyridine (19) (2.79 g, 11.91 mmol) and ammonium chloride (0.542 g, 10.13 mmol) in a mixture of ethanol (11.85 ml) and water (5.93 ml) and was heated to reflux under vigorous stirring for 40 min. The reaction mixture was then filtered through a Celite® pad, and the filtrate was concentrated under reduced pressure. The residue was dissolved in DCM, extracted with water; the organic phase was dried over anhydrous Na₂SO₄, filtered and concentrated under reduced pressure to afford the title compound 20 (2.13 g, 10.43 mmol, 88% yield) as creamy solid. MS: 205.1 (M+1).

### Step 3. N-(3-Fluoro-4-(pyridin-4-yloxy)phenyl)-2-oxo-3-phenylimidazolidine-1-carboxamide (21a)

Starting from compound 20 and following the same procedure as described for the synthesis of compound 18a (scheme 1, step 5, example 5), title compound 21a was obtained in 49% yield as white solid. ¹H NMR (400 MHz, DMSO-d₆) 10.54 (s, 1H), 8.47 (d, J=6.1 Hz, 2H), 7.81 (dd, J=2.3 Hz, J=12.3 Hz, 1H), 7.64-7.61 (m, 2H), 7.45-7.34 (m, 4H), 7.20-7.16 (m, 1H), 6.94 (dd, J=1.4 Hz, J=4.7 Hz, 2H), 4.0-3.91 (m, 4H). MS (M+1) 393.2.

TABLE 2
Characterization of compounds 21c-21h (examples 15-21) prepared according to
Scheme 4.

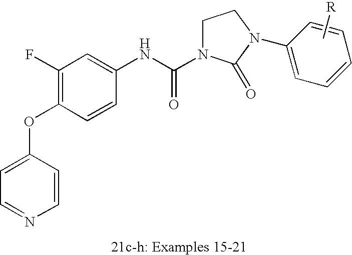

| Cpd | Ex. | R | Name | Characterization |
|---|---|---|---|---|
| 21b | 15 | H | 2-oxo-3-phenyl-N-(4- | ¹H NMR (DMSO-d₆, 400 MHz) 10.43 (s, |
| | | | (pyridin-4- | 1H), 8.45 (dd, J = 1.6 Hz, J = 4.7 Hz, 2H), |
| | | | yloxy)phenyl)imidazolidine- | 7.66-7.61 (m, 4H), 7.45-7.40 (m, 2H), |
| | | | 1-carboxamide | 7.19-7.15 (m, 3H), 6.90 (m, 2H), 3.96 |
| | | | | (m, 4H). m/z: (M + 1)⁺ 375.2 (100%). |
| 21c | 16 | 3-CF₃ | N-(3-fluoro-4-(pyridin-4- | ¹H NMR (400 MHz, DMSO-d₆) δ |
| | | | yloxy)phenyl)-2-oxo-3-(3- | (ppm): 10.43 (s, 1H), 8.48-8.46 (m, 2H), |
| | | | (trifluoromethyl)phenyl) | 8.17 (s, 1H), 7.83 (dd, J = 2.4 Hz, J = |
| | | | imidazolidine-1- | 12.9 Hz, 1H), 7.77 (dd, J = 2.4 Hz, J = |
| | | | carboxamide | 8.4 Hz, 1H), 7.67 (t, J = 7.8 Hz, 1H), 7.53 |
| | | | | (dd, J = 0.8 Hz, J = 7.8 Hz, 1H), 7.48-7.45 |
| | | | | (m, 1H), 7.35 (t, J = 8.8 Hz, 1H), 6.94- |
| | | | | 6.92 (m, 2H). MS (m/z): (M + 1) 461.1. |
| 21d | 17 | 2-CF₃ | N-(3-fluoro-4-(pyridin-4- | ¹H NMR (400 MHz, DMSO-d₆) δ |
| | | | yloxy)phenyl)-2-oxo-3-(2- | (ppm): 10.43 (s, 1H), 8.78-8.44 (m, 2H), |
| | | | (trifluoromethyl)phenyl) | 8.17 (s, 1H), 7.83 (dd, J = 2.2 Hz, J = |
| | | | imidazolidine-1- | 12.9 Hz, 1H), 7.77 (dd, J = 2.2 Jz, J = |
| | | | carboxamide | 8.4 Hz, 1H), 7.67 (t, J = 7.8 Hz, 1H), 7.53 |
| | | | | (J = 0.8 Hz, J = 7.8 Hz, 1H), 7.48-7.49 (m, |
| | | | | 1H), 7.35 (t, J = 8.8 Hz, 1H), 6.94-6.92 |
| | | | | (m, 2H), 4.05. MS (m/z): (M + 1) 461.1. |
| 21e | 18 | H | N-(3-chloro-4-(pyridin-4- | ¹H NMR (400 MHz, DMSO-d₆) δ |
| | | | yloxy)phenyl)-2-oxo-3- | (ppm): 10.52 (s, 1H), 8.47-8.46 (m, 2H), |
| | | | phenylimidazolidine-1- | 7.99 (d, J = 2.5 Hz, 1H), 7.64-7.62 (m, |
| | | | carboxamide | 2H), 7.57 (dd, J = 2.5 Hz, J = 8.8 Hz, 1H), |
| | | | | 7.45-7.41 (m, 2H), 7.36 (d, J = 8.8 Hz, |
| | | | | 1H), 7.20-7.16 (m, 1H), 7.16-6.87 (m, |
| | | | | 2H), 4.00-3.94 (m, 4H). MS (m/z): |
| | | | | (M + 1) 409.1. |
| 21f | 19 | 4-Cl | 3-(4-chlorophenyl)-N-(3- | ¹H NMR (400 MHz, DMSO-d₆) δ |
| | | | fluoro-4-(pyridin-4- | (ppm): 10.48 (s, 1H), 8.47-8.46 (m, 2H), |
| | | | yloxy)phenyl)-2- | 7.81 (dd, J = 2.4 Hz, J = 12.9 Hz, 1H), |
| | | | oxoimidazolidine-1- | 7.67-7.64 (m, 2H), 7.51-7.48 (m, 2H), |
| | | | carboxamide | 7.43-7.40 (m, 1H), 7.35 (t, J = 8.8 Hz, |
| | | | | 1H), 6.94-6.93 (m, 2H), 3.96-3.93 (m, |
| | | | | 4H). MS (m/z): (M + 1) 427.1. |
| 21g | 20 | 3-Cl | 3-(3-chlorophenyl)-N-(3- | ¹H NMR (400 MHz, DMSO-d₆) δ |
| | | | fluoro-4-(pyridin-4- | (ppm): 10.46 (s, 1H), 8.45-8.46 (m, 2H), |
| | | | yloxy)phenyl)-2- | 7.85-7.80 (m, 2H), 7.52-7.43 (m, 3H), |
| | | | oxoimidazolidine-1- | 7.36 (t, J = 9 Hz, 1H), 7.25-7.22 (m, 1H), |
| | | | carboxamide | 6.94-6.93 (m, 2H), 3.99-3.92 (m, 4H). |
| | | | | MS (m/z): (M + 1) 427.1. |
| 21h | 21 | 2-Cl | 3-(2-chlorophenyl)-N-(3- | ¹H NMR (400 MHz, DMSO-d₆) δ |
| | | | fluoro-4-(pyridin-4- | (ppm): 10.45 (s, 1H), 8.47 (d, J = 4.7 Hz, |
| | | | yloxy)phenyl)-2- | 1H), 7.84-7.79 (m, 2H), 7.51-7.42 (m, |
| | | | oxoimidazolidine-1- | 4H), 7.35 (t, J = 8.8 Hz, 1H), 7.24-7.21 |
| | | | carboxamide | (m, 1H), 6.89 (m, 2H), 3.99-3.89 (m, 4H) |
| | | | | MS (m/z): (M + 1) 427.1. |

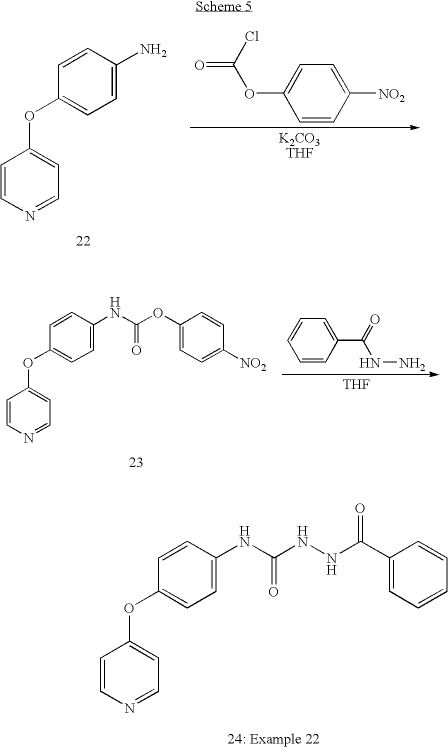

### Example 9

### 2-Benzoyl-N-(4-(pyridin-4-yloxy)phenyl)hydrazinecarboxamide (24)

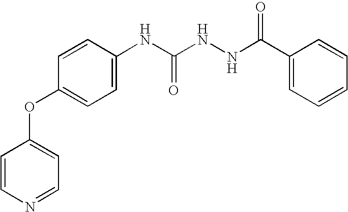

### Step 1. 4-Nitrophenyl 4-(pyridin-4-yloxy)phenylcarbamate (23)

4-Nitrophenyl chloroformate (0.541 g, 2.69 mmol) was added to a mixture of 4-(pyridin-4-yloxy)aniline (22, 0.25 g, 1.343 mmol) and potassium carbonate (0.371 g, 2.69 mmol) in THF (13.43 ml) at 0° C. The mixture was stirred at 0° C. for 5 h, allowed to gradually warm to room temperature and stirred overnight. The crude reaction mixture containing the title compound 23 was used in the next step without further purification. MS: 352.1 (M+1).

### Step 2. 2-Benzoyl-N-(4-(pyridin-4-yloxy)phenyl)hydrazinecarboxamide (24)

Benzohydrazide (0.549 g, 4.03 mmol) was added to the above mentioned solution of 23 and the reaction mixture was heated to reflux overnight. It was then concentrated under reduced pressure, the residue was purified by preparative HPLC (column: Luna C18 (2), 5 cm ID; gradient: 60% MeOH to 95% of MeOH in water, 60 min) affording title compound 24 (0.06 g, 0.164 mmol, 12% yield) as white fluffy solid. ¹H NMR (400 MHz, DMSO-d₆) 10.31 (s, 1H), 9.1 (s, 1H), 8.44-8.42 (m, 2H), 8.31 (s, 1H), 8.22 (s, 0.6H), 7.94-7.92 (m, 2H), 7.61-7.56 (m, 3H), 7.53-7.49 (m, 2H), 7.12-7.08 (m, 2H), 6.89-6.87 (m, 2H). MS: 349.1 (M+1).

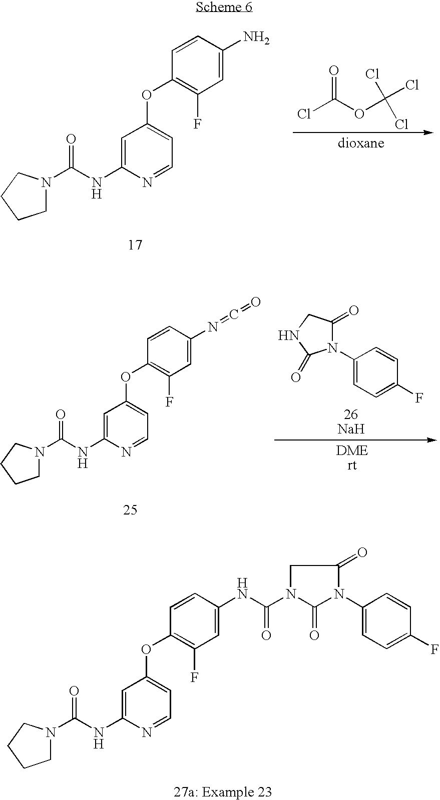

### Example 23

### N-(3-fluoro-4-(2-(pyrrolidine-1-carboxamido)pyridin-4-yloxy)phenyl)-3-(4-fluorophenyl)-2,4-dioxoimidazolidine-1-carboxamide (27a)

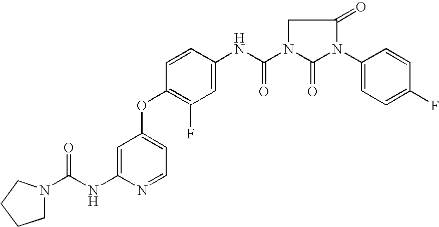

### Step 1. N-(4-(2-fluoro-4-isocyanatophenoxy)pyridin-2-yl)pyrrolidine-1-carboxamide (25)

A solution of 17 (100 mg, 0.316 mmol) in dioxane (2 mL) was treated with trichloromethyl chloroformate (0.191 mL, 1.581 mmol) and stirred at room temperature for 18 h. The reaction mixture was diluted with DCM then the precipitate was filtered under an atmosphere of argon to afford the title compound 25, and used in the next step without further purification.

### Step 2. N-(3-fluoro-4-(2-(pyrrolidine-1-carboxamido)pyridin-4-yloxy)phenyl)-3-(4-fluorophenyl)-2,4-dioxoimidazolidine-1-carboxamide (27a)

A solution of 25 (350 mg, 1.022 mmol) and 3-(4-fluorophenyl)imidazolidine-2,4-dione 26 (397 mg, 2.045 mmol) (prepared similarly to 3b, Scheme 1, example 3) in anhydrous DME (8 mL) was treated with sodium hydride (123 mg, 3.07 mmol) under argon atmosphere. The reaction mixture was cooled to 0° C. and stirred for 1 hr then concentrated and purified by flash chromatography using 50-60-70-80-100% EtOAc/hex. Subsequent purification with Gilson using 50-95% MeOH/water (aquasil column) afforded title compound 27a (21 mg, 4% yield). ¹H NMR (400 MHz, DMSO-d6) δ ppm: 10.43 (s, 1H), 8.48-8.46 (m, 2H), 8.17 (s, 1H), 7.83 (dd, J=2.4 Hz, J=12.9 Hz, 1H), 7.77 (dd, J=2.4 Hz, J=8.4 Hz, 1H), 7.67 (t, J=7.8 Hz, 1H), 7.53 (dd, J=0.8 Hz, J=7.8 Hz, 1H), 7.48-7.45 (m, 1H), 7.35 (t, J=8.8 Hz, 1H), 6.94-6.92 (m, 2H). MS: 461.1 (M+1). Compounds 27d-h (Examples 26-30) were prepared in one step from 17 (Scheme 6) similarly to 24 (example 22, Scheme 5).

TABLE 3
Characterization of compounds 27b-27h (examples 24-30) prepared according
to Schemes 5 and 6.

| Cpd | Ex. | Structure | Name | Characterization |
|---|---|---|---|---|
| 27b | 24 | 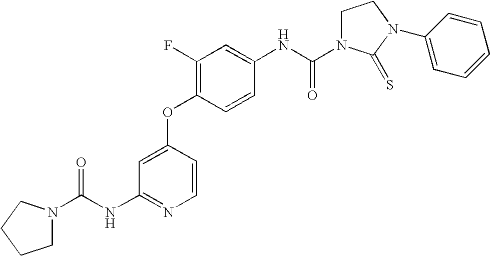 | N-(3-fluoro-4-(2-(pyrrolidine-1-carboxamido)pyridin-4-yloxy)phenyl)-3-phenyl-2-thioxoimidazolidine-1-carboxamide | ¹H NMR (400 MHz,DMSO-d₆) δ (ppm): 12.50(s, 1H), 8.67 (s, 1h), 8.10 (d,1H, J = 5.7 Hz), 7.75 (dd,1H, J = 2.3 Hz, J = 13.6 Hz),7.49 (m, 4H), 7.45 (d, 1H, J =2.3 Hz), 7.3-7.4 (m, 3H),6.61 (d, 1H, J = 2.3 Hz, J =5.6 Hz), 4.22 (dd, 2H, J = 6.2 Hz, J = 11.8 Hz), 4.08(dd, 2H). MS (m/z): 521.3(M + 1). |
| 27c | 25 | 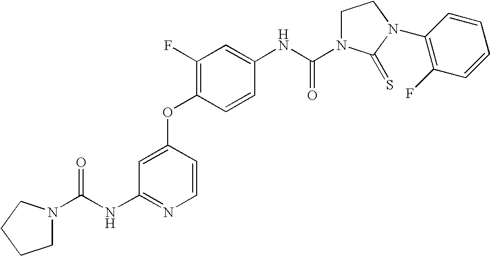 | N-(3-fluoro-4-(2-(pyrrolidine-1-carboxamido)pyridin-4-yloxy)phenyl)-3-(2-fluorophenyl)-2-thioxoimidazolidine-1-carboxamide | ¹H NMR (400 MHz, DMSO-d₆) δ (ppm): 12.9 (s, 1H),8.69 (s, 1H), 8.10 (d, 1H, J =5.7 Hz), 8.75 (dd, 1H, J =2.3 Hz, J = 12.9 Hz), 7.46(d, 1H, J = 2.3 Hz), 7.1-7.4(m, 6H), 6.59 (dd, 1H, J =2.3 Hz, J = 5.6 Hz), 4.30 (t,2H, J = 7.0 Hz), [3.3 (6H)], 1.79 (s, 4H). MS (m/z):(M + 1) 539.3. |
| 27d | 26 | 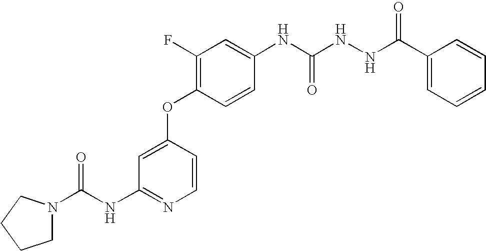 | N-(3-chloro-4-(pyridin-4-yloxy)phenyl)-2-oxo-3-phenylimidazolidine-1-carboxamide | ¹H NMR (400 MHz,DMSO-d₆) δ (ppm): 10.33(s, 1H), 9.32 (br.s, 1H), 8.68(s, 1H), 8.49 (s, 1H), 8.09(d, 1H, J = 5.6 Hz), 7.92 (d,2H, 7.1 Hz), 7.68 (dd, 1H, J =2.1 Hz, J = 7.3 Hz), 7.58(t, 1H, J = 7.2 Hz), 7.50 (t,2H, J = 7.6 Hz), 7.45 (d, 1H, J = 2.3 Hz), 7.31 (m, 1H),7.24 (t, 1H, J = 8.8 Hz), 6.58(dd, 1H, J = 2.5 Hz, J = 5.9Hz), [3.37 4H], 1.79 (s,4H)) MS (m/z): (M + 1)479.3. |
| 27e | 27 | 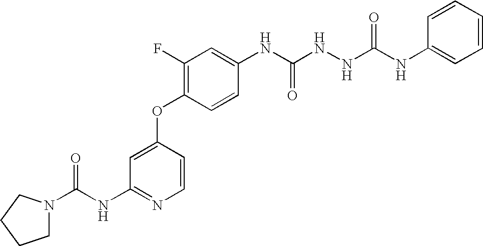 | N1-(3-fluoro-4-(2-(pyrrolidine-1-carboxamido)pyridin-4-yloxy)phenyl)-N2-phenylhydrazine-1,2-dicarboxamide | ¹H NMR (400 MHz,DMSO-d₆) δ (ppm): 9.26(br.s, 1H), 8.86 (s, 1H), 8.67(s, 1H), 8.33 (s, 0.15H), 8.27(s, 1H), 8.08 (d, 2H, J = 5.7Hz), 7.72 (d, 1H, J = 12.9Hz), 7.50 (d, 2H, J = 7.8Hz), 7.44 (d, 1H, J = 2.3Hz), 7.33 (m, 1H), 7.25 (m,3H), 6.94 (t, 1H, J = 7.2 Hz), 6.58 (dd, 1H, J = 2.3 Hz, J =5.6 Hz) MS (m/z): (M + 1)494.3. |
| 27f | 28 | 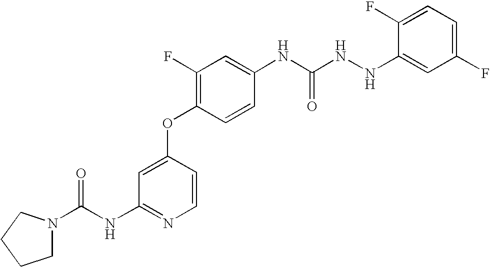 | N-(4-(4-(2-(2,5-difluorophenyl)hydrazinecarboxamido)-2-fluorophenoxy)pyridin-2-yl)pyrrolidine-1-carboxamide | ¹H NMR (400 MHz,DMSO-d₆) δ (ppm): 9.26(br.s, 1H), 8.67 (s, 1H), 8.51(s, 1H), 8.24 (s, 0.ssH), 8.09(d, 2H, J = 5.4 Hz), 7.74 (d,1H, J = 12.9 Hz), 7.44 (s,1H), 7.40 (m, 1H), 7.23 (t,1H, J = 9.0 Hz), 7.14 (m,1H), 6.59 (m, 3H), [3.4, 4H], 1.79 (s, 4H) MS (m/z):(M + 1) 487.3. |
| 27g | 29 | 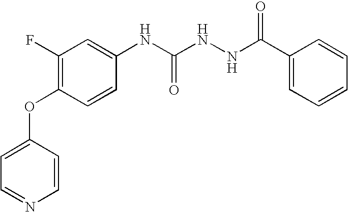 | N-(3-fluoro-4-(pyridin-4-yloxy)phenyl)-2-oxo-3-(3-(trifluoromethyl)phenyl)imidazolidine-1-carboxamide | ¹H NMR (400 MHz,DMSO-d₆) δ (ppm): 10.34(d, J = 1.4 Hz, 1H), 9.23 (br,1H), 8.46 (br, 2H), 8.40 (s,1H), 7.93 (m, 2H), 7.72 (dd,J = 11 Hz, J = 2.3 Hz, 1H),7.61-7.57 (m, 1H), 7.53-7.50 (m, 2H), 7.36-7.26(m, 2H), 6.93 (m, 2H) MS(m/z): (M + 1) 367.2. |
| 27h | 30 | 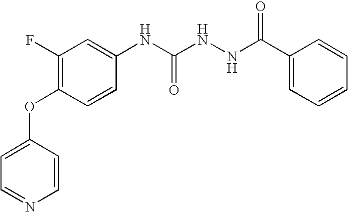 | N-(3-fluoro-4-(pyridin-4-yloxy)phenyl)-2-oxo-3-(2-(trifluoromethyl)phenyl)imidazolidine-1-carboxamide | ¹H NMR (400 MHz,DMSO-d₆) δ (ppm): 10.61(br, 1H), 9.30 (br, 1H), 8.55(s, 1H), 8.46-8.44 (m, 2H),7.98 (dd, J = 7.6 Hz, 1H),7.78 (t, J = 7.83 Hz, 1H),7.73-7.69 (m, 1H0, 7.34-7.26 (m, 2H), 6.92-6.91 (m,2H) MS (m/z): (M + 1)435.2. |
| 27i | 74 | 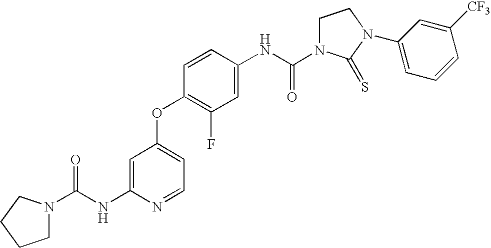 | N-(3-fluoro-4-(2-(pyrrolidine-1-carboxamido)pyridin-4-yloxy)phenyl)-2-thioxo-3-(3-(trifluoromethyl)phenyl)imidazolidine-1-carboxamide | ¹H NMR (400 MHz,DMSO-d₆) δ (ppm): 11.86(s, 1H), 8.69 (s, 1H), 8.09(d, 1H, J = 5.7 Hz), 7.76 (d,1H, J = 2.2 Hz, J = 12.9 Hz),7.62 (t, 1H, J = 8.0 Hz), 7.52(d, 1H, J = 8.0 Hz), 7.45 (m,2H), 7.3-7.4 (m, 3H), 6.58 (dd, 1H, J = 2.6 Hz, J = 5.6Hz), 4.27 (t, 2H, J = 7.2 Hz),1.78 (s, 4H).) MS (m/z):589.3 (M + 1). |
| 27j | 75 | 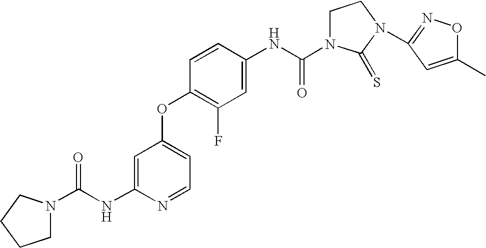 | N-(3-fluoro-4-(2-(pyrrolidine-1-carboxamido)pyridin-4-yloxy)phenyl)-3-(5-methylisoxazol-3-yl)-2-thioxoimidazolidine-1-carboxamide | ¹H NMR (400 MHz,DMSO-d₆) δ (ppm): 12.11(s, 1H), 8.70 (s, 1H), 8.10(d, 1H, J = 5.7 Hz), 7.76 (dd,1H, J = 2.5 Hz, J = 12.6 Hz),7.46 (d, 1H, J = 2.6 Hz), 7.3-7.4 (m, 2H), 6.61 (dd, 1J =2.5 Hz, J = 5.8 Hz), 6.40 (s,1H), 4.27 (t, 2H, J = 7.2 Hz), 2.39 (s, 3H), 1.79 (s, 4H).MS (m/z): 526.3 (M + 1). |
| 27k | 76 | 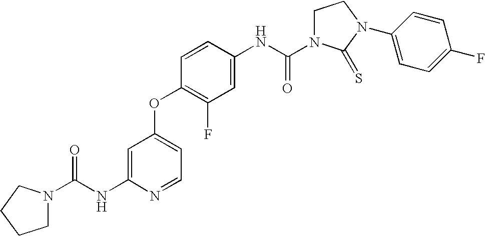 | N-(3-fluoro-4-(2-(pyrrolidine-1-carboxamido)pyridin-4-yloxy)phenyl)-3-(4-fluorophenyl)-2-thioxoimidazolidine-1-carboxamide | ¹H NMR (400 MHz,DMSO-d₆) δ (ppm): 12.08(s, 1H), 8.69 (s, 1H), 8.10(d, 1H, J = 5.7 Hz), 7.75 (dd,1H, J = 2.3 Hz, J = 11.6 Hz),7.44 (d, 1H, J = 8.2 Hz),7.29 (m, 2H), 7.21 (t, 2H, J =8.7 Hz), 7.12 (m, 2H),6.60 (dd, 1H, J = 2.4 Hz, J = 5.7 Hz), 4.25 (t, 2H, J = 7.1Hz), 1.79 (s, 4H). MS (m/z):539.3 (M + 1). |

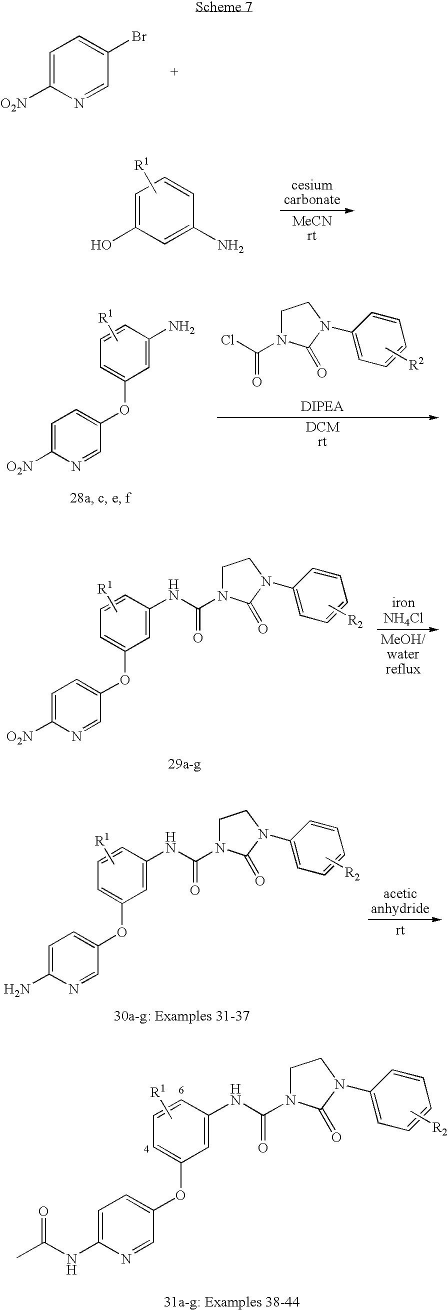

a, R¹=R²⁼H
b, R¹=H, R²=4-F
c, R¹=4-Me, R²=H
d, R¹=4-Me, R²=4-F
e, R¹=4-OMe, R²=H
f, R¹=6-Cl, R²=H
g, R¹=6-Cl, R²=4-F

### Example 31

### N-(3-(6-aminopyridine-3-yloxy)phenyl)-2-oxo-3-phenylimidazolidine-1-carboxamide (30a)

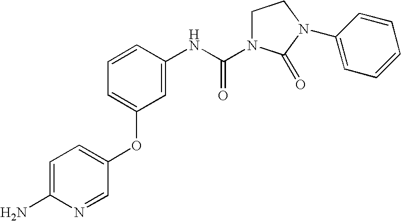

Step 1. 3-(6-nitropyridin-3-yloxy)aniline (28a)

To a stirred solution of 5-bromo-2-nitropyridine (2.00 g, 9.85 mmol) and 3-aminophenol (1.29 g, 11.82 mmol) in acetonitrile (150 mL) under nitrogen was added cesium carbonate (7.36 g, 22.59 mmol). The reaction mixture (suspension) was stirred at room temperature for one week, and concentrated. The crude residue was partitioned between ethyl acetate and water. The organic layer was collected and successively washed with water and brine, dried over magnesium sulfate, filtered, and concentrated. The crude residue was purified by flash column chromatography on silica gel (AcOEt/hexanes:30/70 to 50/50) to afford the title compound 28a (1.03 g, 4.45 mmol, 45% yield, slightly contaminated) as a bright yellow sticky solid. ¹H NMR (400 MHz, DMSO-d₆) δ (ppm): 8.38 (d, J=2.9 Hz, 1H), 8.33 (d, J=9.0 Hz, 1H), 7.58 (dd, J=9.0, 2.9 Hz, 1H), 7.11 (t, J=8.0 Hz, 1H), 6.52-6.45 (m, 1H), 6.37-6.25 (m, 2H), 5.43 (s, 2H). MS (m/z): 232.1 (M+H)⁺.

### Step 2. N-(3-(6-nitropyridin-3-yloxy)phenyl)-2-oxo-3-phenylimidazolidine-1-carboxamide (29a)

To a stirred solution of 28a (490 mg, 2.12 mmol) and diisopropylethylamine (1.11 mL, 6.36 mmol) in dichloromethane (20 mL) under nitrogen at room temperature was slowly added 3b (519 mg, 2.33 mmol) in DCM (2 mL). The reaction mixture was stirred overnight, quenched with methanol, concentrated, and suspended in a minimum of DCM in MeOH. The suspension was triturated for 30 min, filtered, rinsed with MeOH, air-dried and dried under high vacuum to afford the title compound 29a (805 mg, 1.92 mmol, 90% yield) as an off-white solid (soluble in acetonitrile). ¹H NMR (400 MHz, DMSO-d₆) δ (ppm): 10.52 (s, 1H), 8.45 (d, J=2.7 Hz, 1H), 8.36 (d, J=9.0 Hz, 1H), 7.67 (dd, J=9.0, 2.7 Hz, 1H), 7.65-7.56 (m, 3H), 7.50-7.37 (m, 4H), 7.17 (t, J=7.3 Hz, 1H), 6.98-6.92 (m, 1H), 3.98-3.87 (m, 4H). MS (m/z): 420.2 (M+H)⁺.

### Step 3. N-(3-(6-aminopyridine-3-yloxy)phenyl)-2-oxo-3-phenylimidazolidine-1-carboxamide (30a)

A stirred suspension of 29a (667 mg, 1.59 mmol), iron powder (355 mg, 6.36 mmol) and ammonium chloride (128 mg, 2.39 mmol) in a mixture of methanol/water (30 mL/6 mL) was heated to reflux for 5 hrs, then at room temperature. The reaction mixture was filtered through celite, rinsed with MeOH and acetonitrile, and concentrated. The crude residue was adsorbed on silica gel and purified by flash column chromatography on silica gel (2% of ammonium hydroxide in MeOH/DCM: 2/98 to 5/95) to afford the title compound 30a (427 mg, 1.10 mmol, 69% yield) as an off-white solid. ¹H NMR (400 MHz, DMSO-d₆) δ (ppm): 10.38 (s, 1H), 7.76 (d, J=2.9 Hz, 1H), 7.61 (d, J=7.8 Hz, 2H), 7.41 (t, J=8.0 Hz, 2H), 7.29-7.19 (m, 3H), 7.16 (t, J=7.3 Hz, 1H), 7.06 (dd, J=8.1, 1.1 Hz, 1H), 6.61 (dd, J=8.3, 1.7 Hz, 1H), 6.50 (d, J=9.0 Hz, 1H), 5.91 (s, 2H), 3.98-3.85 (m, 4H). MS (m/z): 390.2 (M+H)⁺.

Compounds 30b-30g (examples 32-37) were prepared in three steps from 5-bromo-2-nitropyridine and the appropriately substituted 3-aminophenol (Scheme 7) similarly to compound 30a (example 31, Scheme 7).

TABLE 4
Characterization of compounds 30b-30g (examples 32-37) prepared according to
Scheme 7

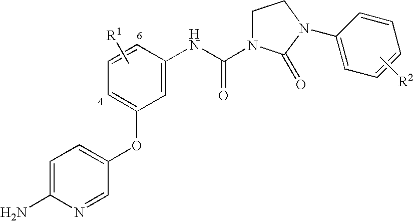

| Cpd | Ex. | R¹ | R² | Name | Characterization |
|---|---|---|---|---|---|
| 30b | 32 | H | 4-F | N-(3-(6-aminopyridin-3- | ¹H NMR (400 MHz, DMSO-d₆) δ |
| | | | | yloxy)phenyl)-3-(4- | (ppm): mixture of rotamers, 10.35 (s, |
| | | | | fluorophenyl)-2- | 1H), 7.75 (d, J = 2.9 Hz, 1H), 7.66- |
| | | | | oxoimidazolidine-1- | 7.59 (m, 2H), 7.30-7.19 (m, 5H), |
| | | | | carboxamide | 7.08-7.03 (m, 1H), 6.64-6.59 (m, 1H), |
| | | | | | 6.50 (dd, J = 8.9, 0.5 Hz, 1H), 5.90 (s, |
| | | | | | 2H), 3.96-3.85 (m, 4H). MS (m/z): |
| | | | | | (M + 1408.3. |
| 30c | 33 | 4-Me | H | N-(3-(6-aminopyridin-3- | ¹H NMR (400 MHz, DMSO-d₆) δ |
| | | | | yloxy)-4-methylphenyl)- | (ppm): mixture of rotamers, 10.25 (s, |
| | | | | 2-oxo-3- | 1H), 7.71 (d, J = 2.9 Hz, 1H), 7.61- |
| | | | | phenylimidazolidine-1- | 7.56 (m, 2H), 7.44-7.36 (m, 2H), |
| | | | | carboxamide | 7.21-7.12 (m, 3H), 7.04 (d, J = 2.0 |
| | | | | | Hz, 1H), 6.98 (dd, J = 8.1, 2.0 Hz, |
| | | | | | 1H), 6.50 (d, J = 8.8 Hz, 1H), 5.85 (s, |
| | | | | | 2H), 3.95-3.81 (m, 4H), 2.21 (s, 3H). |
| | | | | | MS (m/z): (M + 1) 404.2. |
| 30d | 34 | 4-Me | 4-F | N-(3-(6-aminopyridin-3- | ¹H NMR (400 MHz, DMSO-d₆) δ |
| | | | | yloxy)-4-methylphenyl)- | (ppm): mixture of rotamers, 10.22 (s, |
| | | | | 3-(4-fluorophenyl)-2- | 1H), 7.71 (d, J = 2.7 Hz, 1H), 7.64- |
| | | | | oxoimidazolidine-1- | 7.57 (m, 2H), 7.29-7.21 (m, 2H), |
| | | | | carboxamide | 7.20-7.14 (m, 2H), 7.02 (d, J = 2.2 |
| | | | | | Hz, 1H), 6.98 (dd, J = 8.1, 2.1 Hz, |
| | | | | | 1H), 6.49 (d, J = 8.8 Hz, 1H), 5.87 (s, |
| | | | | | 2H), 3.94-3.81 (m, 4H), 2.21 (s, 3H). |
| | | | | | MS (m/z): (M + 1) 422.2. |
| 30^{e} | 35 | 4-Ome | H | N-(3-(6-aminopyridin-3- | ¹H NMR (400 MHz, DMSO-d₆) δ |
| | | | | yloxy)-4-methoxyphenyl)- | (ppm): mixture of rotamers, 10.16 (s, |
| | | | | 2-oxo-3- | 1H) 7.68 (d, J = 2.9 Hz, 1H), 7.62- |
| | | | | phenylimidazolidine-1- | 7.56 (m, 2H), 7.44-7.37 (m, 2H), |
| | | | | carboxamide | 7.19-7.03 (m, 5H), 6.47 (dd, J = 9.0, |
| | | | | | 0.6 Hz, 1H), 5.80 (s, 2H), 3.96-3.81 |
| | | | | | (m, 4H), 3.77 (s, 3H). MS (m/z): |
| | | | | | (M + 1) 420.2. |
| 30f | 36 | 6-Cl | H | N-(5-(6-aminopyridin-3- | ¹H NMR (400 MHz, DMSO-d₆) δ |
| | | | | yloxy)-2-chlorophenyl)-2- | (ppm): mixture of rotamers, 10.97 (s, |
| | | | | oxo-3- | 1H), 7.92 (d, J = 2.9 Hz, 1H), 7.76 (d, |
| | | | | phenylimidazolidine-1- | J = 2.9 Hz, 1H), 7.63-7.57 (m, 2H), |
| | | | | carboxamide | 7.47-7.38 (m, 3H), 7.23 (dd, J = 9.0, |
| | | | | | 2.9 Hz, 1H), 7.17 (tt, J = 7.4, 1.0 Hz, |
| | | | | | 1H), 6.65 (dd, J = 8.8, 2.9 Hz, 1H), |
| | | | | | 6.51 (d, J = 8.8 Hz, 1H), 5.95 (s, 2H), |
| | | | | | 3.99-3.84 (m, 4H). MS (m/z): (M + 1) |
| | | | | | 424.2. |
| 30g | 37 | 6-Cl | 4-F | N-(5-(6-aminopyridin-3- | ¹H NMR (400 MHz, DMSO-d₆) δ |
| | | | | yloxy)-2-chlorophenyl)-3- | (ppm): mixture of rotamers, 10.95 (s, |
| | | | | (4-fluorophenyl)-2- | 1H), 7.93 (d, J = 2.9 Hz, 1H), 7.77 (d, |
| | | | | oxoimidazolidine-1- | J = 2.7 Hz, 1H), 7.67-7.59 (m, 2H), |
| | | | | carboxamide | 7.44 (d, J = 8.8 Hz, 1H), 7.32-7.24 |
| | | | | | (m, 2H), 7.23 (dd, J = 9.0, 2.9 Hz, |
| | | | | | 1H), 6.65 (dd, J = 8.8, 2.9 Hz, 1H), |
| | | | | | 6.50 (d, J = 8.8 Hz, 1H), 5.95 (s, 2H), |
| | | | | | 3.99-3.84 (m, 4H). MS (m/z): (M + 1) |
| | | | | | 442.2. |

### Example 38

### N-(3-(6-acetamidopyridin-3-yloxy)phenyl)-2-oxo-3-phenylimidazolidine-1′-carboxamide (31a)

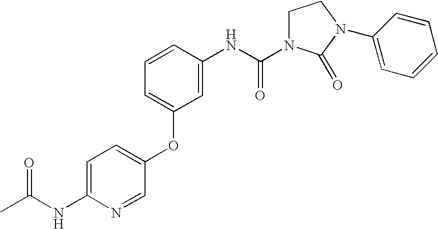

30a (50 mg, 0.13 mmol) was dissolved in anhydrous acetic anhydride (2 mL) and stirred for three days. The reaction mixture (suspension) was quenched with a 10% solution of NaHCO₃ and stirred for 1 h. The suspension was collected by filtration, rinsed with water and air-dried. The crude solid was purified by flash column chromatography on silica gel (MeOH/DCM 05/95) and coprecipitated in AcOEt/hexanes to afford the title compound 31a (52 mg, 0.12 mmol, 94% yield) as a white fluffy solid. ¹H NMR (400 MHz, DMSO-d₆) δ (ppm): mixture of rotamers, 10.57 (s, 1H), 10.43 (s, 1H), 8.15 (d, J=3.1 Hz, 1H), 8.12 (d, J=9.2 Hz, 1H), 7.61 (dd, J=8.7, 0.9 Hz, 2H), 7.55 (dd, J=9.0, 2.9 Hz, 1H), 7.41 (t, J=8.0 Hz, 2H), 7.37 (t, J=2.2 Hz, 1H), 7.33 (t, J=8.1 Hz, 1H), 7.20-7.13 (m, 2H), 6.72 (dd, J=8.2, 1.6 Hz, 1H), 3.99-3.85 (m, 4H), 2.09 (s, 3H). MS (m/z): 432.2 (M+H)⁺.

Examples 39-44 (compounds 31b-31g) were prepared in one step from the appropriately substituted compound 30 and acetic anhydride (Scheme 7) similarly to compounds 31a (example 38, Scheme 7).

TABLE 5
Characterization of compounds 31b-31g (examples 39-44) prepared according to
Scheme 7

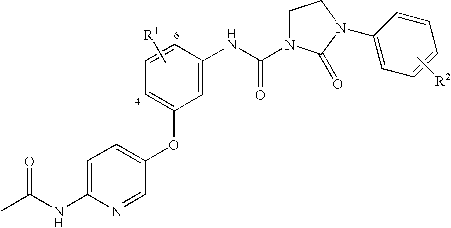

| Cpd | Ex. | R¹ | R² | Name | Characterization |
|---|---|---|---|---|---|
| 31b | 39 | H | 4-F | N-(3-(6-acetamidopyridin- | ¹H NMR (400 MHz, DMSO-d₆) δ |
| | | | | 3-yloxy)phenyl)-3-(4- | (ppm): mixture of rotamers, 10.56 |
| | | | | fluorophenyl)-2- | (s, 1H), 10.40 (s, 1H), 8.15 (dd, J = |
| | | | | oxoimidazolidine-1- | 3.0, 0.7 Hz, 1H), 8.12 (d, J = 9.0 |
| | | | | carboxamide | Hz, 1H), 7.66-7.58 (m, 2H), 7.55 |
| | | | | | (dd, J = 9.0, 2.9 Hz, 1H), 7.36 (t, J = |
| | | | | | 2.2 Hz, 1H), 7.32 (t, J = 8.2 Hz, |
| | | | | | 1H), 7.30-7.22 (m, 2H), 7.18-7.13 |
| | | | | | (m, 1H), 6.75-6.69 (m, 1H), 3.97- |
| | | | | | 3.84 (m, 4H), 2.09 (s, 3H). MS |
| | | | | | (m/z): (M + 1) 450.2. |
| 31c | 40 | 4-Me | H | N-(3-(6-acetamidopyridin- | ¹H NMR (400 MHz, DMSO-d₆) δ |
| | | | | 3-yloxy)-4- | (ppm): mixture of rotamers, 10.53 |
| | | | | methylphenyl)-2-oxo-3- | (s, 1H), 10.32 (s, 1H), 8.13-8.05 |
| | | | | phenylimidazolidine-1- | (m, 2H), 7.63-7.56 (m, 2H), 7.44 |
| | | | | carboxamide | (dd, J = 9.0, 2.9 Hz, 1H), 7.43-7.36 |
| | | | | | (m, 2H), 7.25 (d, J = 8.6 Hz, 1H), |
| | | | | | 7.23 (d, J = 2.0 Hz, 1H), 7.15 (t, J = |
| | | | | | 7.4 Hz, 1H), 7.11 (dd, J = 8.2, 2.2 |
| | | | | | Hz, 1H), 3.96-3.82 (m, 4H), 2.19 |
| | | | | | (s, 3H), 2.08 (s, 3H). MS (m/z): |
| | | | | | (M + 1) 446.3. |
| 31d | 41 | 4-Me | 4-F | N-(3-(6-acetamidopyridin- | ¹H NMR (400 MHz, DMSO-d₆) δ |
| | | | | 3-yloxy)-4- | (ppm): mixture of rotamers, 10.54 |
| | | | | methylphenyl)-3-(4- | (s, 1H), 10.29 (s, 1H), 8.13-8.05 |
| | | | | fluorophenyl)-2- | (m, 2H), 7.65-7.57 (m, 2H), 7.44 |
| | | | | oxoimidazolidine-1- | (dd, J = 9.0, 2.9 Hz, 1H), 7.29-7.19 |
| | | | | carboxamide | (m, 4H), 7.10 (dd, J = 8.2, 2.2 Hz, |
| | | | | | 1H), 3.95-3.81 (m, 4H), 2.19 (s, |
| | | | | | 3H), 2.08 (s, 3H). MS (m/z): |
| | | | | | (M + 1) 464.3. |
| 31e | 42 | 4-OMe | H | N-(3-(6-acetamidopyridin- | ¹H NMR (400 MHz, DMSO-d₆) δ |
| | | | | 3-yloxy)-4- | (ppm): mixture of rotamers, 10.48 |
| | | | | methoxyphenyl)-2-oxo-3- | (s, 1H), 10.24 (s, 1H), 8.04 (bd, J = |
| | | | | phenylimidazolidine-1- | 9.2 Hz, 1H), 8.02 (dd, J = 3.0, 0.5 |
| | | | | carboxamide | Hz, 1H), 7.63-7.57 (m, 2H), 7.45- |
| | | | | | 7.32 (m, 4H), 7.24 (dd, J = 8.8, 2.5 |
| | | | | | Hz, 1H), 7.16 (tt, J = 7.3, 1.0 Hz, |
| | | | | | 1H), 7.13 (d, J = 9.0 Hz, 1H), 3.97- |
| | | | | | 3.83 (m, 4H), 3.75 (s, 3H), 2.07 (s, |
| | | | | | 3H). MS (m/z): (M + 1) 462.3. |
| 31f | 43 | 6-Cl | H | N-(5-(6-acetamidopyridin- | ¹H NMR (400 MHz, DMSO-d₆) δ |
| | | | | 3-yloxy)-2-chlorophenyl)- | (ppm): mixture of rotamers, 11.03 |
| | | | | 2-oxo-3- | (s, 1H), 10.59 (s, 1H), 8.18 (dd, J = |
| | | | | phenylimidazolidine-1- | 2.9, 0.6 Hz, 1H), 8.13 (d, J = 8.8 |
| | | | | carboxamide | Hz, 1H), 8.01 (d, J = 2.9 Hz, 1H), |
| | | | | | 7.64-7.56 (m, 3H), 7.52 (d, J = 8.8 |
| | | | | | Hz, 1H), 7.46-7.39 (m, 2H), 7.18 |
| | | | | | (tt, J = 7.4, 1.1 Hz, 1H), 6.77 (dd, J = |
| | | | | | 8.8, 2.9 Hz, 1H), 3.99-3.86 (m, |
| | | | | | 4H), 2.10 (s, 3H). MS (m/z): |
| | | | | | (M + 1) 466.2. |
| 31g | 44 | 6-Cl | 4-F | N-(5-(6-acetamidopyridin- | ¹H NMR (400 MHz, DMSO-d₆) δ |
| | | | | 3-yloxy)-2-chlorophenyl)- | (ppm): mixture of rotamers, 11.01 |
| | | | | 3-(4-fluorophenyl)-2- | (s, 1H), 10.59 (s, 1H), 8.18 (d, J = |
| | | | | oxoimidazolidine-1- | 2.7 Hz, 1H), 8.13 (d, J = 9.0 Hz, |
| | | | | carboxamide | 1H), 8.00 (d, J = 2.9 Hz, 1H), 7.66- |
| | | | | | 7.59 (m, 2H), 7.58 (dd, J = 9.0, 2.9 |
| | | | | | Hz, 1H), 7.51 (d, J = 8.8 Hz, 1H), |
| | | | | | 7.32-7.24 (m, 2H), 6.77 (dd, J = |
| | | | | | 8.8, 2.9 Hz, 1H), 3.99-3.85 (m, |
| | | | | | 4H), 2.10 (s, 3H). MS (m/z): |
| | | | | | (M + 1) 484.2. |

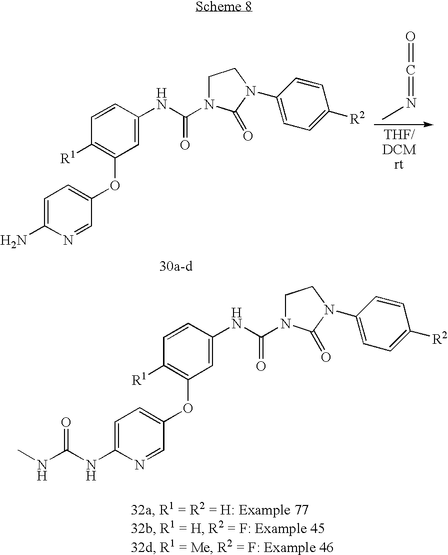

### Example 45

### 3-(4-fluorophenyl)-N-(3-(6-(3-methylureido)pyridin-3-yloxy)phenyl)-2-oxoimidazolidine-1-carboxamide (32b)

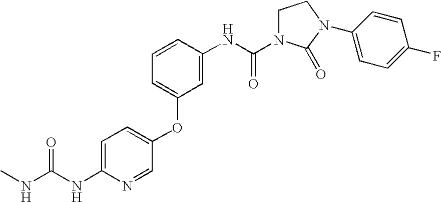

To a stirred solution of 30b (50 mg, 0.12 mmol) in a mixture of THF/DCM (5 mL/5 mL) at room temperature was added a large excess of methyl isocyanate (0.6 g, 10.52 mmol) over few days. The reaction mixture was stirred at room temperature for one week. Then it was quenched with methanol and stirred for 30 min. The suspension was collected by filtration, rinsed with methanol, air-dried, and dried under high vacuum to afford the title compound 32b (43 mg, 0.09 mmol, 75% yield) as a white solid. ¹H NMR (400 MHz, DMSO-d₆) δ (ppm): mixture of rotamers, 10.39 (s, 1H), 9.29 (s, 1H), 8.03 (d, J=2.9 Hz, 1H), 7.79-7.70 (m, 1H), 7.66-7.59 (m, 2H), 7.52 (dd, J=9.0, 2.9 Hz, 1H), 7.46 (d, J=9.2 Hz, 1H), 7.34-7.22 (m, 4H), 7.13 (dd, J=8.0, 1.2 Hz, 1H), 6.71-6.66 (m, 1H), 3.97-3.84 (m, 4H), 2.72 (d, J=4.5 Hz, 3H). MS (m/z): 465.3 (M+H)⁺.

Examples 46 and 77 (compounds 32d and 32a) were prepared in one step from the appropriate 6-aminopyridine 30 and methyl isocyanate (Scheme 8) similarly to compound 32b (example 45, Scheme 8).

TABLE 6
Characterization of compounds 32a and 32d (examples 46 and 77) prepared
according to Scheme 8

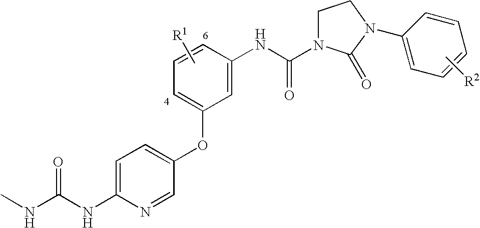

| Cpd | Ex. | R¹ | R² | Name | Characterization |
|---|---|---|---|---|---|
| 32a | 77 | H | H | N-(3-(6-(3- | ¹H NMR (400 MHz, DMSO-d₆) δ |
| | | | | methylureido)pyridin-3- | (ppm): mixture of rotamers, 10.41 (s, |
| | | | | yloxy)phenyl)-2-oxo-3- | 1H), 9.28 (s, 1H), 8.03 (d, J = 2.5 Hz, |
| | | | | phenylimidazolidine-1- | 1H), 7.78-7.70 (m, 1H), 7.61 (d, J = 8.0 |
| | | | | carboxamide | Hz, 2H), 7.52 (dd, J = 9.0, 2.7 Hz, 1H), |
| | | | | | 7.49-7.37 (m, 3H), 7.35-7.27 (m, 2H), |
| | | | | | 7.19-7.10 (m, 2H), 6.69 (dd, J = 8.0, 2.0 |
| | | | | | Hz, 1H), 3.98-3.85 (m, 4H), 2.72 (d, J = |
| | | | | | 4.5 Hz, 3H). MS (m/z): 447.3 (M + 1). |
| 32d | 46 | 4-Me | 4-F | 3-(4-fluorophenyl)-N-(4- | ¹H NMR (400 MHz, DMSO-d₆) δ |
| | | | | methyl-3-(6-(3- | (ppm): mixture of rotamers, 10.26 (s, |
| | | | | methylureido)pyridin-3- | 1H), 9.25 (s, 1H), 7.95 (t, J = 1.8 Hz, |
| | | | | yloxy)phenyl)-2- | 1H), 7.78-7.69 (m, 1H), 7.64-7.57 (m, |
| | | | | oxoimidazolidine-1- | 2H), 7.47-7.40 (m, 2H), 7.29-7.20 (m, |
| | | | | carboxamide | 3H), 7.15 (d, J = 2.2 Hz, 1H), 7.07 (dd, J = |
| | | | | | 8.2, 2.2 Hz, 1H), 3.94-3.81 (m, 4H), |
| | | | | | 2.71 (d, J = 4.7 Hz, 3H), 2.20 (s, 3H). |
| | | | | | MS (m/z): 479.3 (M + H)^{+.}. |

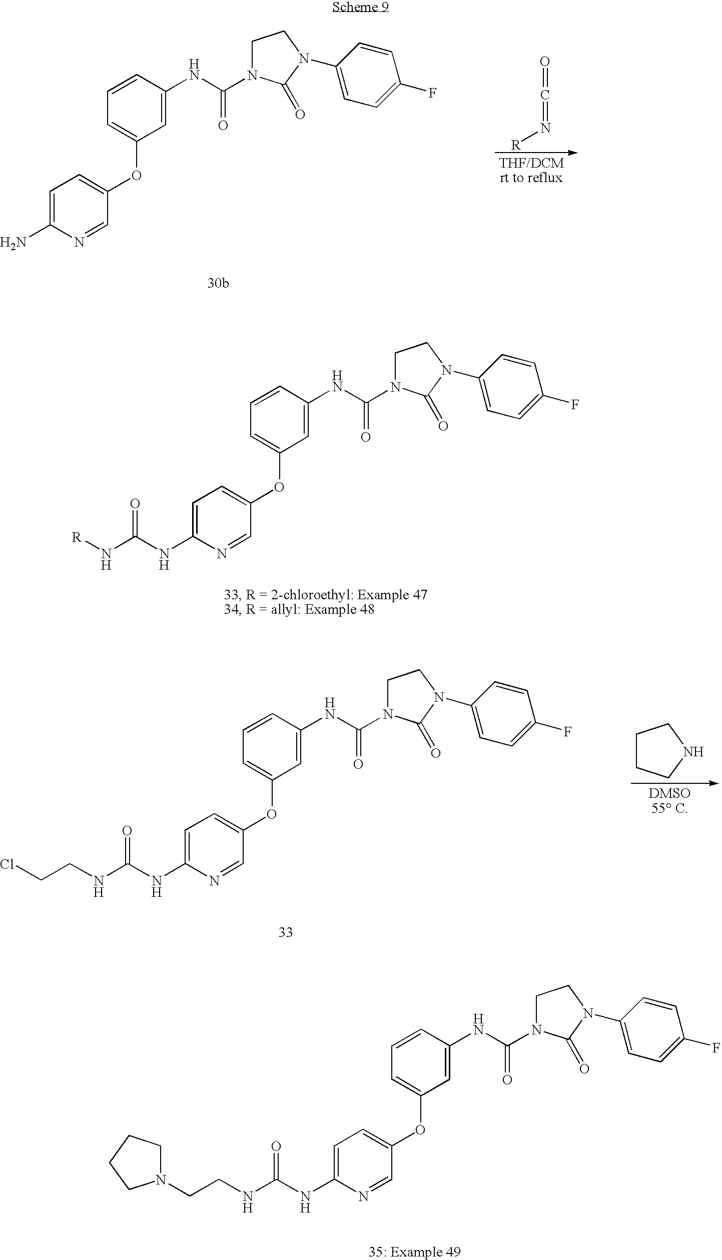

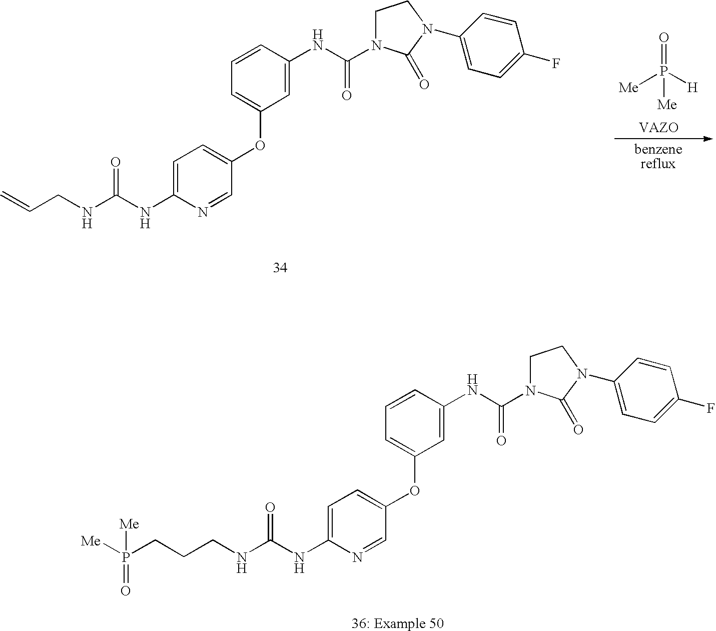

### Example 47

### N-(3-(6-(3-(2-chloroethyl)ureido)pyridin-3-yloxy)phenyl)-3-(4-fluorophenyl)-2-oxoimidazolidine-1-carboxamide (33)

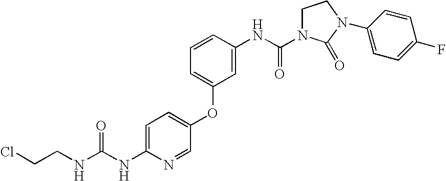

To a stirred solution of 30b (100 mg, 0.25 mmol) in a mixture of THF/DCM (5 mL/10 mL) at room temperature under nitrogen was added a large excess of 2-chloroethyl isocyante (0.5 mL, 5.86 mmol) over two days. The reaction mixture was stirred at rt for two days, then refluxed for 8 h. The reaction mixture (white suspension) was quenched at rt with methanol and shaken for 30 min. The suspension was collected by filtration, rinsed with methanol, air-dried, and dried under high vacuum to afford the title compound 33 (71 mg, 0.14 mmol, 56% yield) as a white fluffy solid. ¹H NMR (400 MHz, DMSO-d₆) δ (ppm): mixture of rotamers, 10.39 (s, 1H), 9.39 (s, 1H), 8.14-8.04 (m, 1H), 8.04 (dd, J=2.7, 1.0 Hz, 1H), 7.66-7.59 (m, 2H), 7.56-7.48 (m, 2H), 7.33 (t, J=2.3 Hz, 1H), 7.32-7.22 (m, 3H), 7.16-7.11 (m, 1H), 6.72-6.67 (m, 1H), 3.96-3.85 (m, 4H), 3.70 (t, J=6.1 Hz, 2H), 3.50 (q, J=5.9 Hz, 2H). MS (m/z): 513.3 (M+H)⁺.

### Example 48

### N-(3-(6-(3-allylureido)pyridin-3-yloxy)phenyl)-3-(4-fluorophenyl)-2-oxoimidazolidine-1-carboxamide (34)

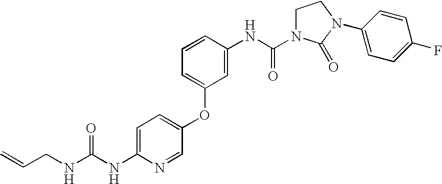

The title compound 34 (example 48) was prepared in one step starting from 30b and allyl isocyanate as an off-white fluffy solid similarly to compound 33 (example 47, Scheme 9). ¹H NMR (400 MHz, DMSO-d₆) δ (ppm): mixture of rotamers, 10.39 (s, 1H), 9.31 (s, 1H), 8.05 (dd, J=2.4, 1.1 Hz, 1H), 7.96-7.87 (m, 1H), 7.66-7.59 (m, 2H), 7.55-7.47 (m, 2H), 7.35-7.22 (m, 4H), 7.16-7.10 (m, 1H), 6.72-6.66 (m, 1H), 5.95-5.84 (m, 1H), 5.22-5.13 (m, 1H), 5.12-5.05 (m, 1H), 3.97-3.78 (m, 6H). MS (m/z): 491.3 (M+H)⁺.

### Example 49

### 3-(4-fluorophenyl)-2-oxo-N-(3-(6-(3-(2-(pyrrolidin-1-yl)ethyl)ureido)pyridin-3-yloxy)phenyl)imidazolidine-1-carboxamide (35)

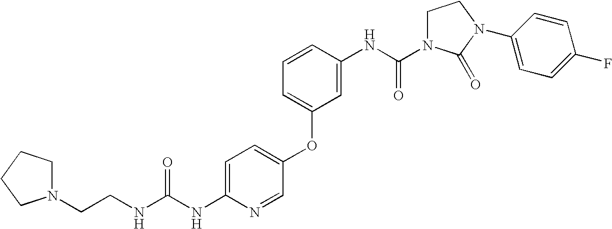

To a stirred solution of 33 (37.8 mg, 0.07 mmol) in DMSO (2 mL) at room temperature under nitrogen was added an excess of pyrrolidine (60 μL, 0.74 mmol). The reaction mixture was heated at 55° C. for 2 h, then rt for overnight. It was quenched with a small amount of methanol and coprecipitated in water. The resulting suspension was shaken for 1 h, filtered-off, rinsed with water and air-dried. The crude solid was purified by flash column chromatography on silica gel (2% of ammonium hydroxide in MeOH/DCM:05/95 to 20/80), coprecipitated in MeOH/water, filtered-off, rinsed with water, air-dried and dried under high vacuum to afford the title compound 35 (23 mg, 0.04 mmol, 57% yield) as a white fluffy solid. ¹H NMR (400 MHz, DMSO-d₆) δ (ppm): mixture of rotamers, 10.39 (s, 1H), 9.26 (bs, 1H), 8.01 (d, J=3.5 Hz, 1H), 7.82-7.72 (m, 1H), 7.66-7.59 (m, 2H), 7.55 (d, J=9.2 Hz, 1H), 7.51 (dd, J=9.1, 2.8 Hz, 1H), 7.34-7.22 (m, 4H), 7.15-7.10 (m, 1H), 6.71-6.66 (m, 1H), 3.98-3.84 (m, 4H), 8H are masked by water and DMSO, 1.71-1.64 (m, 4H). MS (m/z): 548.4 (M+H)⁺.

### Example 50

### N-(3-(6-(3-(3-(dimethylphosphoryl)propyl)ureido)pyridin-3-yloxy)phenyl)-3-(4-fluorophenyl)-2-oxoimidazolidine-1-carboxamide (36)

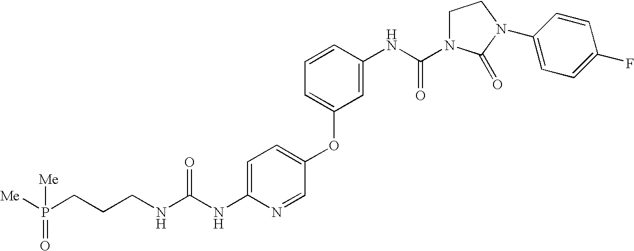

A stirred solution of 34 (25 mg, 0.05 mmol), dimethylphosphine oxide (39 mg, 0.5 mmol, prepared according to WO 2005/009348 A2) and VAZO [1-1′-azobis(cyclohexane-carbonitrile), 5 mg, 0.02 mmol) in benzene (10 mL) under nitrogen was heated to reflux for 10 hrs, then rt. The reaction mixture was quenched with MeOH and concentrated. The crude residue was purified by flash column chromatography on silica gel (2% of ammonium hydroxide in MeOH/DCM:05/95 to 20/80) and by Gilson (Thermo, aquasil C18, 250×21.2 mm, 5 μm, 0.05% of formic acid in both MeOH/water: 70/30 to 95/5 over 30 min) to afford the title compound 36 (5.5 mg, 0.01 mmol, 19% yield) as a colorless film. ¹H NMR (400 MHz, MeOH-d₄) δ (ppm): mixture of rotamers, 10.53 (s, 0.2H), 8.02 (bs, 1H), 7.66-7.58 (m, 2H), 7.45 (bd, J=8.2 Hz, 1H), 7.37 (bs, 1H), 7.30 (t, J=8.1 Hz, 1H), 7.19-7.09 (m, 4H), 6.70 (dd, J=8.3, 1.7 Hz, 1H), 4.02-3.90 (m, 4H), 3.46-3.35 (m, 2H), 1.93-1.79 (m, 4H), 1.53 (d, J²_{P-H}=12.9 Hz, 6H), 2 NH urea are missing. MS (m/z): 569.4 (M+H)⁺.

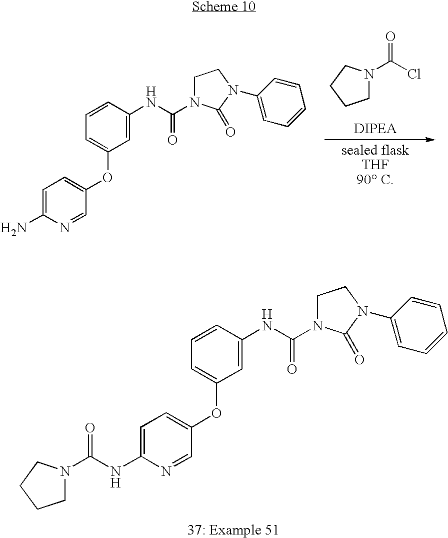

### Example 51

### 2-oxo-3-phenyl-N-(3-(6-(pyrrolidine-1-carboxamido)pyridin-3-yloxy)phenyl) imidazolidine-1-carboxamide (37)

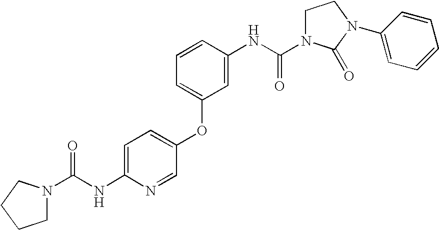

In a sealed flask, a stirred solution of 30a (100 mg, 0.26 mmol), DIPEA (134 μL, 0.77 mmol) and 1-pyrrolidinecarbonyl chloride (284 μL, 2.57 mmol) was heated at 90° C. overnight. The reaction mixture was quenched at rt with methanol and concentrated. The crude residue was purified by flash column chromatography on silica gel (2% of ammonium hydroxide in MeOH/DCM:05/95) and twice by Gilson (Thermo, aquasil C18, 250×21.2 mm, 5 μm, 0.05% of formic acid in both MeOH/water:30/70 to 85/15 over 30 min) to afford the title compound 37 (1.7 mg, 0.003 mmol, 1% yield) as an yellow sticky solid. ¹H NMR (400 MHz, MeOH-d₄) δ (ppm): 2 NH are missing, 8.80-7.30 (3 bumps, 3H), 7.65 (d, J=7.8 Hz, 2H), 7.43 (t, J=8.1 Hz, 3H), 7.34 (t, J=7.8 Hz, 1H), 7.26-7.15 (m, 2H), 6.80-6.70 (m, 1H), 4.64 (bs, 2H), 4.02 (s, 4H), 3.70-3.42 (m, 2H), 2.20-1.90 (m, 4H). MS (m/z): 487.3 (M+H)⁺.

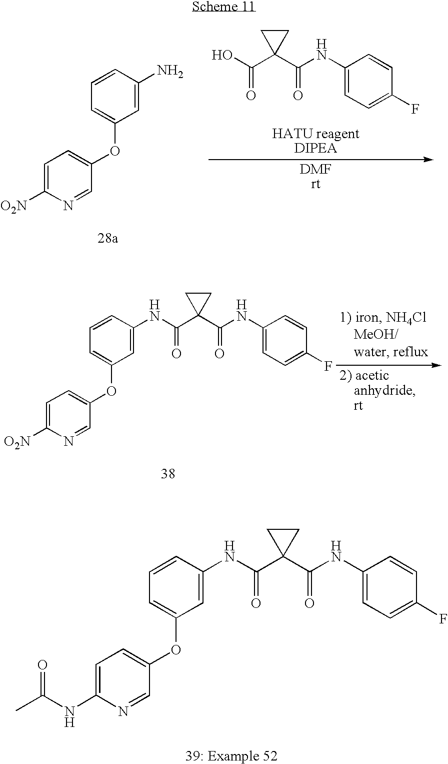

### Example 52

### N-(3-(6-acetamidopyridin-3-yloxy)phenyl)-N-(4-fluorophenyl)cyclopropane-1,1-dicarboxamide (39)

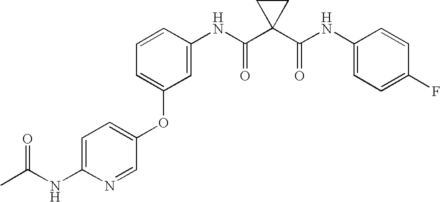

### Step 1. N-(4-fluorophenyl)-N-(3-(6-nitropyridin-3-yloxy)phenyl)cyclopropane-1,1-dicarboxamide (38)

To a stirred solution of 28a (280 mg, 1.21 mmol), 1-(4-fluorophenylcarbamoyl)cyclopropanecarboxylic acid (542 mg, 2.43 mmol, prepared according to US 2007/0004675 A1, compound 181), DIPEA (0.63 mL, 3.63 mmol) in anhydrous DMF (10 mL) under nitrogen was added HATU reagent (1.151 g, 3.03 mmol). The reaction mixture was stirred at room temperature overnight, quenched by addition of a saturated aqueous solution of ammonium chloride followed by the addition of ethyl acetate. After separation, the organic layer was successively washed with a saturated aqueous solution of ammonium chloride, water and brine, and concentrated. The crude residue was purified by flash column chromatography on silica gel (AcOEt/hexanes: 30/70 to 50/50) and coprecipitated in AcOEt/hexanes to afford the title compound 38 (465 mg, 1.07 mmol, 88% yield) as a white fluffy solid. MS (m/z): 437.1 (M+H)⁺.

### Step 2. N-(3-(6-acetamidopyridin-3-yloxy)phenyl)-N-(4-fluorophenyl)cyclopropane-1,1-dicarboxamide (39)

The title compound 39 (example 52) was prepared in one step starting from 38 as an off-white fluffy solid (Scheme 11) following the same procedure as in example 38, steps 3-4 (Scheme 7). ¹H NMR (400 MHz, DMSO-d₆) δ (ppm): mixture of rotamers, 10.56 (s, 1H), 10.14 (s, 1H), 9.95 (s, 1H), 8.15-8.08 (m, 2H), 7.64-7.56 (m, 2H), 7.52 (dd, J=9.1, 3.0 Hz, 1H), 7.40-7.32 (m, 2H), 7.29 (t, J=8.0 Hz, 1H), 7.17-7.09 (m, 2H), 6.74 (ddd, J=7.9, 2.4, 1.2 Hz, 1H), 2.08 (s, 3H), 1.44-1.36 (m, 4H). MS (m/z): 449.2 (M+H)⁺.

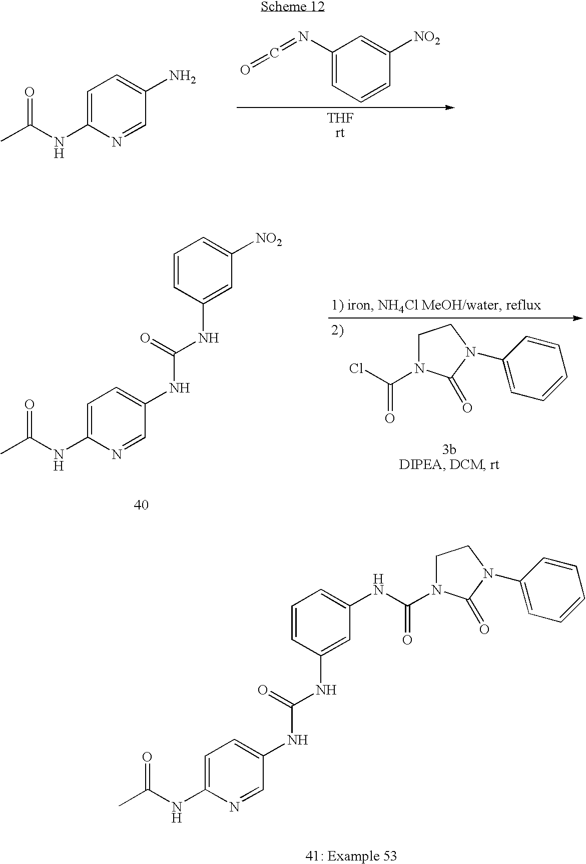

### Example 53

### N-(3-(3-(6-acetamidopyridin-3-yl)ureido)phenyl)-2-oxo-3-phenylimidazolidine-1-carboxamide (41)

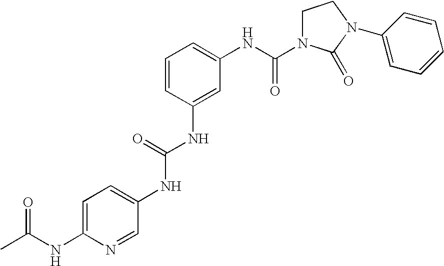

### Step 1. N-(5-(3-(3-nitrophenyl)ureido)pyridin-2-yl)acetamide (40)

To a stirred solution under nitrogen of 2-acetamido-5-aminopyridine (1.00 g, 6.62 mmol) in anhydrous THF (50 mL) was added 3-nitrophenyl isocyanate (1.194 g, 7.28 mmol). The reaction mixture was stirred at room temperature for one day, and quenched with MeOH. The suspension was stirred for 30 min, collected by filtration, rinsed with MeOH, air-dried and dried under high vacuum to afford the title compound 40 (1.84 g, 5.83 mmol, 88% yield) as a pinky solid). MS (m/z): 316.2 (M+H)⁺.

### Step 2. N-(3-(3-(6-acetamidopyridin-3-yl)ureido)phenyl)-2-oxo-3-phenylimidazolidine-1-carboxamide (41)

The title compound 41 (example 53) was prepared in one step starting from 40 as a pale pinky solid (Scheme 12) following the same procedure as in example 31, steps 3 and 2 (Scheme 7). ¹H NMR (400 MHz, DMSO-d₆) δ (ppm): mixture of rotamers, 10.39 (s, 2H), 8.86 (s, 1H), 8.68 (s, 1H), 8.42 (d, J=2.5 Hz, 1H), 8.01 (bd, J=9.0 Hz, 1H), 7.86-7.76 (m, 2H), 7.63 (d, J=8.0 Hz, 2H), 7.42 (t, J=8.0 Hz, 2H), 7.27-7.08 (m, 4H), 4.01-3.87 (m, 4H), 2.06 (s, 3H). MS (m/z): 474.2 (M+H)⁺.

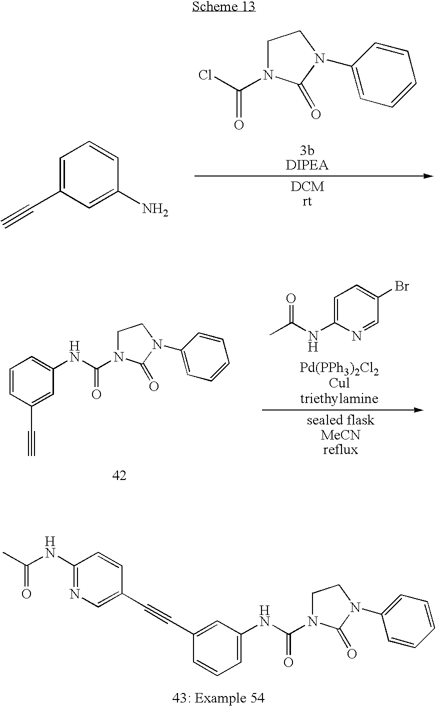

### Example 54

### N-(3-((6-acetamidopyridin-3-yl)ethynyl)phenyl)-2-oxo-3-phenylimidazolidine-1-carboxamide (43)

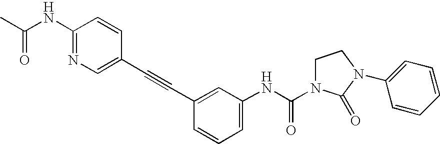

### Step 1. N-(3-ethynylphenyl)-2-oxo-3-phenylimidazolidine-1-carboxamide (42)

To a stirred solution of 3-aminophenylacetylene (250 mg, 2.13 mmol) and diisopropylethylamine (1.12 mL, 6.40 mmol) in dichloromethane (20 mL) under nitrogen at room temperature was added 3b (527 mg, 2.35 mmol). After overnight, the reaction mixture was quenched with methanol, concentrated, and suspended in MeOH. The suspension was stirred for 30 min, filtered-off, rinsed with MeOH, air-dried and dried under high vacuum to afford the title compound 42 (594 mg, 1.94 mmol, 91% yield) as a white fluffy solid. ¹H NMR (400 MHz, DMSO-d₆) δ (ppm): 10.43 (s, 1H), 7.76 (t, J=1.8 Hz, 1H), 7.65-7.59 (m, 2H), 7.49 (ddd, J=8.2, 2.2, 1.0 Hz, 1H), 7.45-7.39 (m, 2H), 7.34 (t, J=7.9 Hz, 1H), 7.21-7.14 (m, 2H), 4.21 (s, 1H), 3.99-3.88 (m, 4H). MS (m/z): 306.2 (M+H)⁺.

### Step 2. N-(3-((6-acetamidopyridin-3-yl)ethynyl)phenyl)-2-oxo-3-phenylimidazolidine-1-carboxamide (43)

In a sealed flask, a stirred suspension of 42 (170 mg, 0.56 mmol), 2-acetamido-5-bromopyridine (100 mg, 0.46 mmol), Pd(PPh₃)₂Cl₂ (33 mg, 0.047 mmol), and CuI (18 mg, 0.093 mmol) in anhydrous acetonitrile (20 mL) was degassed with nitrogen for 10 min before the addition of triethylamine (324 μL, 2.33 mmol). The reaction mixture was heated to reflux for 4 h, then rt. It was quenched with a saturated aqueous solution of ammonium chloride, followed by the addition of ethyl acetate. After separation the organic layer (presence of a solid) was washed with a saturated aqueous solution of ammonium chloride, water and brine, filtered, and concentrated. The crude residue was adsorbed on silica gel, purified by flash chromatography on silica gel (AcOEt/DCM:20/80 to 30/70) and triturated in ethyl acetate to afford the title compound 43 (20 mg, 0.045 mmol, 10% yield) as a pale brown solid. ¹H NMR (400 MHz, DMSO-d₆) δ (ppm): 10.73 (s, 1H), 10.47 (s, 1H), 8.52 (dd, J=2.3, 0.9 Hz, 1H), 8.13 (d, J=8.6 Hz, 1H), 7.96 (dd, J=8.6, 2.3 Hz, 1H), 7.86 (t, J=1.8 Hz, 1H), 7.66-7.60 (m, 2H), 7.51 (ddd, J=8.2, 2.2, 1.0 Hz, 1H), 7.47-7.36 (m, 3H), 7.26 (dt, J=7.8, 1.3 Hz, 1H), 7.18 (tt, J=7.3, 1.0 Hz, 1H), 4.01-3.89 (m, 4H), 2.12 (s, 3H). MS (m/z): 440.3 (M+H)⁺.

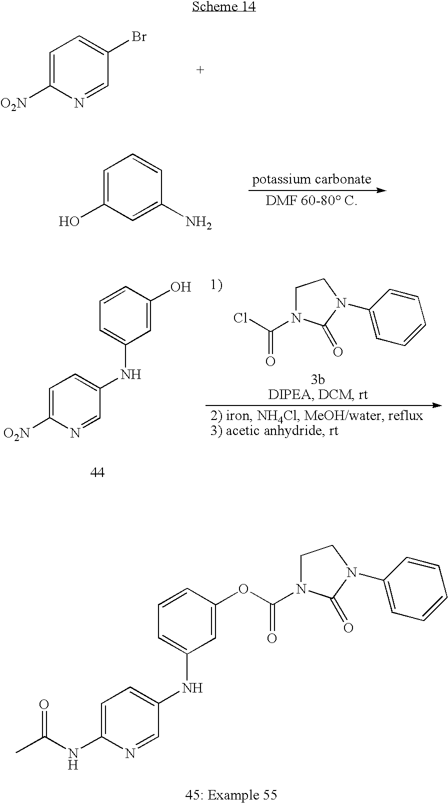

### Example 55

### 3-(6-acetamidopyridin-3-ylamino)phenyl 2-oxo-3-phenylimidazolidine-1-carboxylate (45)

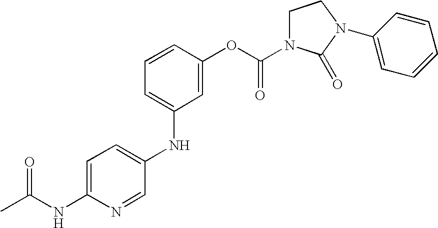

### Step 1. 3-(6-nitropyridin-3-ylamino)phenol (44)

To a stirred solution of 5-bromo-2-nitropyridine (3.00 g, 9.85 mmol) and 3-aminophenol (1.77 g, 16.26 mmol) in anhydrous DMF (50 mL) under nitrogen was added potassium carbonate (3.60 g, 26.05 mmol). The reaction mixture was heated to 60-80° C. for two days, then at room temperature. The reaction mixture was partitioned between ethyl acetate and water. After separation, the organic layer was successively washed with water, a saturated aqueous solution of ammonium chloride, water, a saturated aqueous solution of sodium bicarbonate, water and brine, and concentrated. The crude residue was purified by flash column chromatography on silica gel (AcOEt/hexanes:30/70 to 50/50) and coprecipitated in AcOEt/hexanes to afford the title compound 44 (1.52 g, 6.59 mmol, 45% yield, slightly contaminated with the regioisomer 28a) as an yellow-orange crystalline solid. ¹H NMR (400 MHz, DMSO-d₆) δ (ppm): mixture of rotamers, 9.05 (dd, J=2.8, 0.5 Hz, 1H), 8.59 (dd, J=9.2, 2.9 Hz, 1H), 7.13 (dd, J=9.0, 0.6 Hz, 1H), 7.08 (t, J=7.9 Hz, 1H), 6.50-6.45 (m, 1H), 6.34 (t, J=2.2 Hz, 1H), 6.31-6.26 (m, 1H), 5.35 (s, 2H). MS (m/z): 232.2 (M+H)⁺.

### Step 2. 3-(6-acetamidopyridin-3-ylamino)phenyl 2-oxo-3-phenylimidazolidine-1-carboxylate (45)

The title compound 45 (example 55) was prepared in one step starting from 44 as a white fluffy solid (Scheme 14) following the same procedure as in example 38, steps 2-4 (Scheme 7). ¹H NMR (400 MHz, DMSO-d₆) δ (ppm): mixture of rotamers, 10.43 (s, 1H), 10.13 (s, 1H), 8.33 (dd, J=2.7, 0.4 Hz, 1H), 8.07 (dd, J=8.9, 2.8 Hz, 1H), 7.65-7.58 (m, 2H), 7.45-7.38 (m, 3H), 7.34 (t, J=8.1 Hz, 1H), 7.22 (ddd, J=8.2, 2.0, 1.0 Hz, 1H), 7.17 (tt, J=7.4, 1.0 Hz, 1H), 7.03 (d, J=8.6 Hz, 1H), 6.78 (ddd, J=8.0, 2.3, 1.0 Hz, 1H), 3.99-3.85 (m, 4H), 2.05 (s, 3H). MS (m/z): 432.2 (M+H)⁺.

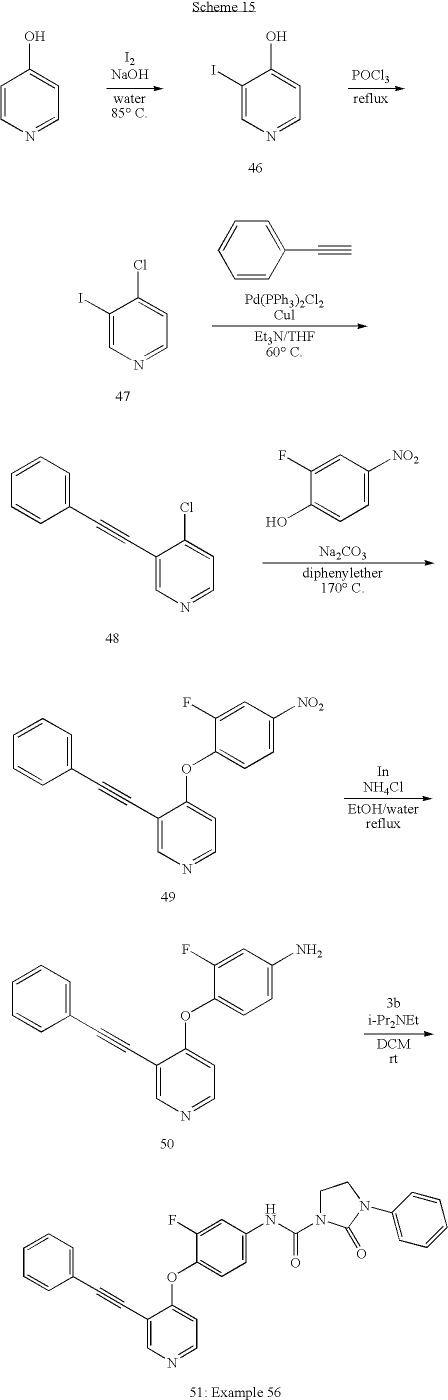

### Example 56

### N-(3-fluoro-4-(3-(phenylethynyl)pyridin-4-yloxy)phenyl)-2-oxo-3phenylimidazolidine-1-carboxamide (51)

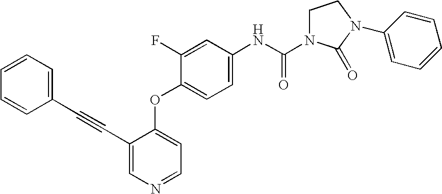

### Step 1. 3-iodopyridin-4-ol (46)

To a stirred solution of 4-hydroxypyridine (5.0 g, 52.6 mmol) in water (90 ml) were successively added sodium hydroxide (5.4 g, 135 mmol) and iodine (28.0 g, 110 mmol). The reaction mixture was heated 85° C. for 16 hours then cooled-down to room temperature. The product was collected by filtration and dry under high vacuum to afford the title compound 46 (7.56 g, 34.2 mmol, 65%) as a white solid. MS: 222.0 (M+1).

### Step 2. 4-chloro-3-iodopyridine (47)

A stirred solution under nitrogen of 46 (2.00 g, 9.05 mmol) in POCl₃ (20 ml) was heated to reflux for four hours, then rt. The reaction mixture was poured slowly into ice and the pH was adjusted to 10-11 with an aqueous solution of ammonium hydroxide. The aqueous layer was extracted twice with dichloromethane. The combined organic layer was washed with brine, dried over anhydrous Na₂SO₄, filtered and concentrated to afford the title compound 47 (1.27 g, 5.30 mmol, 58%) as a brown solid. MS: 239.9 (M+1).

### Step 3. 4-chloro-3-(phenylethynyl)pyridine (48)

To a stirred solution under nitrogen of 47 (212 mg, 0.885 mmol) in anhydrous THF (4.4 ml) was added phenylacetylene (0.097 ml, 0.885 mmol), copper iodide (8.4 mg, 0.044 mmol), dichlorobis(triphenylphosphine)palladium(II) (16 mg, 0.022 mmol) and triethylamine (0.370 ml, 2.66 mmol). The reaction mixture was heated at 60° C. for 48 hours. The reaction mixture was diluted with a saturated solution of NaHCO₃ and extracted twice with dichloromethane. The combined organic layer was washed with brine, dried over anhydrous MgSO₄, filtered and concentrated. The crude residue was purified by flash column chromatography on silica gel (0% to 30% EtOAc in hexanes) to afford the title compound 48 (34.0 mg, 0.159 mmol, 18%) as a solid. MS: 214.0 (M+1).

### Step 4. 4-(2-fluoro-4-nitrophenoxy)-3-(phenylethynyl)pyridine (49)

To a stirred solution of 48 (35 mg, 0.164 mmol) in diphenyl ether (0.204 ml) was added 2-fluoro-4-nitrophenol (77 mg, 0.491 mmol) and sodium carbonate (52 mg, 0.491 mmol). The reaction mixture was heated at 170° C. for four hours, then rt. It was diluted with dichloromethane and filtered. The mother liquid was concentrated and the crude residue was purified by flash column chromatography on silica gel (0% to 50% EtOAc in hexanes) to afford the title compound 49 (40.0 mg, 0.120 mmol, 73.0%) as a yellow oil. MS: 335.0 (M+1).

### Step 5. 3-fluoro-4-(3-(phenylethynyl)pyridin-4-yloxy)aniline (50)

To a stirred solution of 49 (40 mg, 0.120 mmol) in EtOH (2.0 ml) and water (1.0 ml) was added ammonium chloride (64 mg, 1.20 mmol) and indium (55 mg, 0.48 mmol). The reaction mixture was heated to reflux for six hours, then rt. It was filtered, concentrated, dissolved in dichloromethane and washed with a lot of water. The organic layer was dried over anhydrous MgSO₄, filtered and concentrated. The crude residue was purified by flash column chromatography on silica gel (0% to 60% EtOAc in hexanes) to afford the title compound 50 (19.4 mg, 0.064 mmol, 53%) as an orange solid. Ms: 305.0 (M+1).

### Step 6. N-(3-fluoro-4-(3-(phenylethynyl)pyridin-4-yloxy)phenyl)-2-oxo-3-phenylimidazolidine-1-carboxamide (51)

The title compound 51 (example 56) was prepared in one step from 50 and 3b as a yellow solid (Scheme 15) following the same procedure as in example 5, step 5 (Scheme 3). ¹H NMR (400 MHz, DMSO-d₆) δ ppm: 10.55 (s, 1H), 8.80 (s, 1H), 8.46 (d, J=5.8 Hz, 1H), 7.84 (d, J=12.9 Hz, 1H), 7.62 (d, J=8.8 Hz, 2H), 7.58-7.54 (m, 3H), 7.46-7.40 (m, 7H), 7.35 (t, J=7.4 Hz, 1H), 7.17 (t, J=7.4 Hz, 1H), 7.09 (t, J=7.6 Hz, 1H), 3.99-3.90 (m, 6H). MS: 493.0 (M+1).

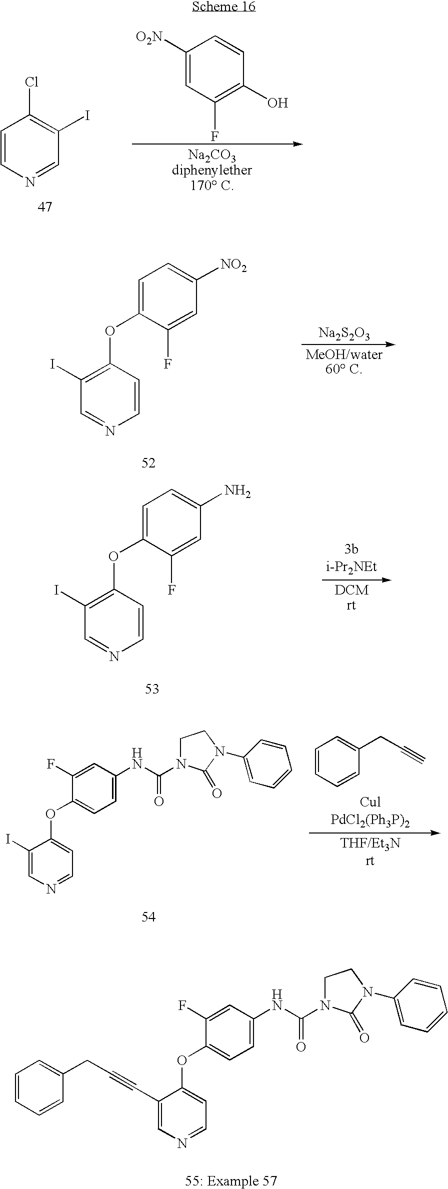

### Example 57

### N-(3-fluoro-4-(3-(3-phenylprop-1-ynyl)pyridin-4-yloxy)phenyl)-2-oxo-3-phenyl-imidazolidine-1-carboxamide (55)

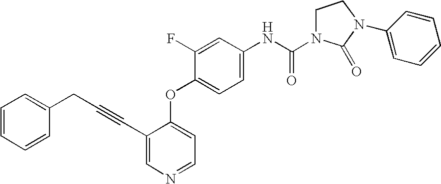

### Step 1. 4-(2-fluoro-4-nitrophenoxy)-3-iodopyridine (52)

To a stirred solution of 47 (346 mg, 1.445 mmol) in diphenyl ether (6 ml) was added sodium carbonate (459 mg, 4.34 mmol) and 2-fluoro-4-nitrophenol (681 mg, 4.34 mmol). The reaction mixture was heated to 170° C. for four hours then cooled-down to room temperature. The reaction mixture was diluted with dichloromethane, filtered and concentrated. The crude residue was purified by flash column chromatography on silica gel (0% to 35% EtOAc in hexanes) to afford the title compound 52 (400 mg, 1.111 mmol, 77%) as a yellow solid. MS: 361.0 (M+1).

### Step 2. 3-fluoro-4-(3-iodopyridin-4-yloxy)aniline (53)

To a stirred solution at 60° C. of 52 (382 mg, 1.061 mmol) in MeOH (2.60 ml) was added a solution of sodium hydrosulfite (646 mg, 3.71 mmol) in water (2.60 ml). The reaction mixture was stirred for 15 minutes at 60° C. and one more hour at room temperature. The reaction mixture was extracted with EtOAc, washed with brine, dried over anhydrous MgSO₄, filtered and concentrated to afford the title compound 53 (164 mg, 0.497 mmol, 46%) as a yellow solid. MS: 331.0 (M+1).

### Step 3. N-(3-fluoro-4-(3-iodopyridin-4-yloxy)phenyl)-2-oxo-3-phenylimidazolidine-1-carboxamide (54)

To a stirred solution under nitrogen of 53 (164 mg, 0.497 mmol) and diisopropylethylamine (0.173 ml, 0.994 mmol) in dichloromethane (3.6 ml) was added 3b (167 mg, 0.745 mmol). The reaction mixture was stirred at room temperature for 48 hours, diluted with EtOAc, and successively washed with water, a saturated solution of sodium bicarbonate and brine, dried over anhydrous MgSO₄, filtered and concentrated to afford the title compound 54 (214 mg, 0.413 mmol, 83%) as a solid.

### Step 4. N-(3-fluoro-4-(3-(3-phenylprop-1-ynyl)pyridin-4-yloxy)phenyl)-2-oxo-3-phenylimidazolidine-1-carboxamide (55)

To a stirred solution under nitrogen of 54 (214 mg, 0.413 mmol) in THF (2.0 ml) was added copper iodide (4 mg, 0.021 mmol), dichlorobis(triphenylphosphine)palladium(II) (7.4 mg, 10.32 μmol), triethylamine (0.172 ml, 1.24 mmol) and benzylacetylene (53 mg, 0.454 mmol). The reaction mixture was stirred at room temperature for 24 hours. The solvent was removed and the residue was directly purified by flash column chromatography on silica gel (0% to 40% EtOAc in hexanes) to afford the title compound 55 (5.0 mg, 9.87 μmol, 2.4%) as a red oil. ¹H NMR (400 MHz, DMSO-d₆) δ ppm: 10.53 (s, 1H), 8.62 (s, 1H), 8.37 (d, J=5.8 Hz, 1H), 7.80 (d, J=12.7 Hz, 1H), 7.62 (d, J=8.9 Hz, 2H), 7.44-7.31 (m, 7H), 7.24 (t, J=7.2 Hz, 1H), 7.17 (t, J=7.4 Hz, 1H), 6.72 (d, J=5.2 Hz, 1H), 3.99-3.90 (m, 6H). MS: 507.1 (M+1).

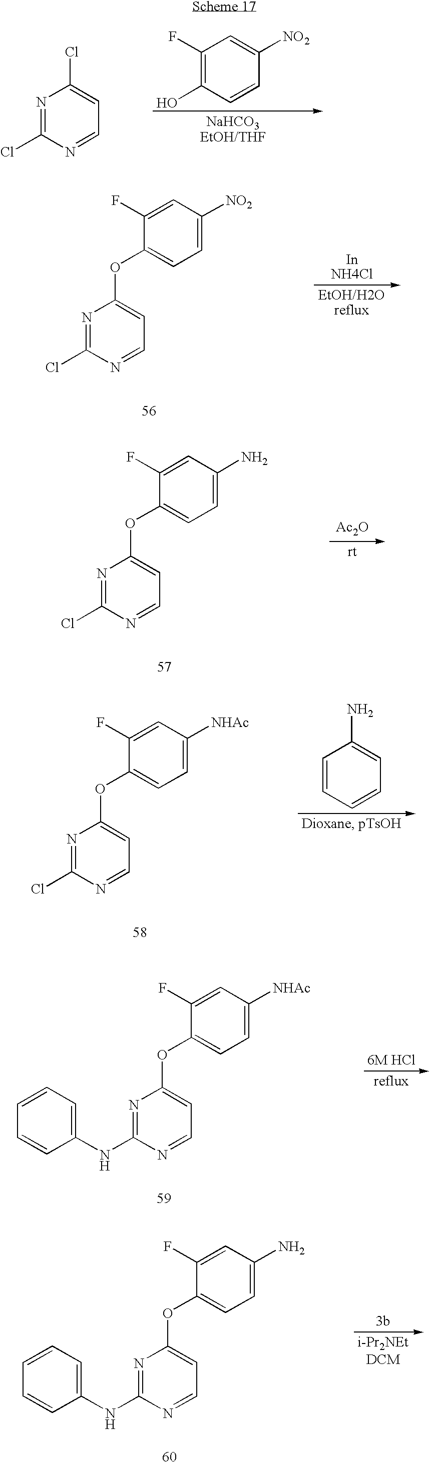

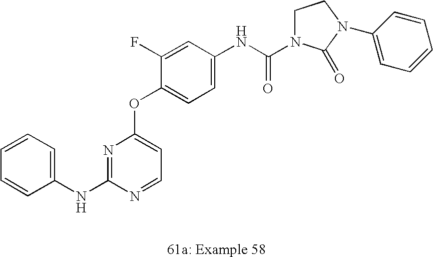

### Example 58

### N-(3-fluoro-4-(2-(phenylamino)pyrimidin-4-yloxy)phenyl)-2-oxo-3-phenyl-imidazolidine-1-carboxamide (61a)

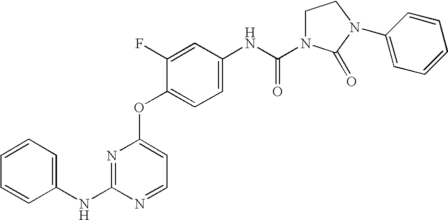

### Step 1: 2-chloro-4-(2-fluoro-4-nitrophenoxy)pyrimidine (56)

To a solution of 2,4-dichloropyrimidine (1.1 g, 7.38 mmol) in EtOH (7 ml) and THF (3 ml) was added 2-fluoro-4-nitrophenol (0.58 g, 3.69 mmol) and NaHCO₃ (930 mg, 11.08 mmol) and the reaction mixture was heated to reflux for 24 hours. The solvents were removed and the crude mixture was dissolved in EtOAc and washed well with an aqueous solution of NaHCO₃. The organic phase was collected, dried over anhydrous Na₂SO₄, filtered and concentrated. The crude residue was purified by flash column chromatography on silica gel (10% EtOAc in hexanes) to afford the title compound 56 (700 mg, 70% yield) as a white solid. MS (m/z): 293.1-295.1 (M+Na).

### Step 2: 4-(2-chloropyrimidin-4-yloxy)-3-fluoroaniline (57)

To a solution of 56 (590 mg, 2.188 mmol) in EtOH (40 ml) and water (20 ml) was added ammonium chloride (1171 mg, 2.6 mmol) and indium metal (1005 mg, 1.039 mmol) and the reaction mixture was heated to reflux for 6 hours. The mixture was cooled to rt and filtered. The solvents were removed and the crude amine was dissolved in DCM and washed well with water. The organic layer was collected, dried over Na₂SO₄, filtered and concentrated. The crude residue was purified by flash column chromatography on silica gel (30% EtOAc in hexane) to afford the title compound 57 (500 mg, 95%) as a brown oil. MS (m/z)=240.1/242.1 (M+H).

### Step 3: N-(4-(2-chloropyrimidin-4-yloxy)-3-fluorophenyl)acetamide (58)

A solution of 57 (500 mg, 2.087 mmol) was dissolved in Ac₂O (10 ml) at room temperature for 24 hours. The solvent was removed and the crude acetate was adsorbed onto silica gel and purified by flash column chromatography on silica gel (50% EtOAc in hexanes) to afford the title compound 58 (359 mg, 61%) as a white solid. MS (m/z)=282.1-284.1 (M+H).

### Step 4: N-(3-fluoro-4-(2-(phenylamino)pyrimidin-4-yloxy)phenyl)acetamide (59)

To a solution of 58 (359 mg, 1.275 mmol) in dioxane (12.7 ml) was added aniline (142 mg, 1.529 mmol) and p-TsOH (194 mg, 1.02 mmol) and the reaction mixture was heated to reflux for 2 hours. The solvent was removed and the residue was dissolved in EtOAc and washed well with water and satd. NaHCO₃ soln. The organic phase was collected, dried over Na₂SO₄, filtered and concentrated. The crude residue was triturated in ethyl ether to afford the title compound 59 (367 mg, 85%) as a white solid. MS (m/z): 339.2 (M+H).

### Step 5: 4-(4-amino-2-fluorophenoxy)-N-phenylpyrimidin-2-amine (60)

A suspension of 59 (367 mg, 1.085 mmol) in 6M HCl (25 ml) was heated to reflux for one hour. The mixture was cooled to room temperature and made basic with an aqueous solution of ammonium hydroxide. The mixture was extracted with EtOAc, and the organic phase was dried over Na₂SO₄, filtered and concentrated. The crude residue was purified by flash column chromatography on silica gel (EtOAc) to afford the title compound 60 (300 mg, 93%) a brown oil. MS (m/z): 297.1 (M+H).

### Step 6: N-(3-fluoro-4-(2-(phenylamino)pyrimidin-4-yloxy)phenyl)-2-oxo-3-phenyl-imidazolidine-1-carboxamide (61a)

The title compound 61a (example 58) was prepared in one step from 60 and 3b as white solid (Scheme 17) following the same procedure as in example 5, step 5 (Scheme 3). ¹H NMR (400 MHz, DMSO-d₆) 10.53 (s, 1H), 9.62 (s, 1H), 8.37 (d, J=5.48 Hz, 1H), 7.76 (m, 1H), 7.62 (m, 2H) 7.45-7.33 (m, 7H), 7.16 (t, J=7.43 Hz, 1H), 7.07 (t, J=7.43 Hz, 2H), 6.84 (t, J=7.43 Hz, 1H), 6.55 (d, J=5.48 Hz, 1H), 3.94 (m, 4H). MS (m/z): 485.3 (M+H).

### Example 59

### N-(3-fluoro-4-(2-(4-methoxyphenylamino)pyrimidin-4-yloxy)phenyl)-2-oxo-3-phenylimidazolidine-1-carboxamide (61b)

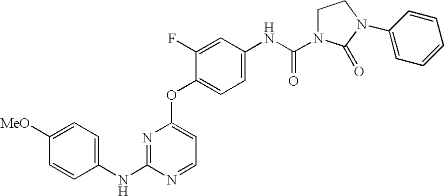

The title compound 61b (example 59) was prepared in three steps starting from 58 and 4-methoxyaniline as a white solid similarly to compound 61a (example 58, Scheme 17). ¹H NMR (400 MHz, DMSO-d₆): 10.51 (s, 1H), 9.43 (s, 1H), 8.32 (m, 1H), 7.77 (m, 1H), 7.63-7.60 (m, 2H), 7.44-7.313 (m, 6H), 7.18 (m, 1H), 6.67 (m, 2H), 6.48 (m, 1H), 3.95 (m, 4H), 3.64 (s, 3H). MS (m/z): 515.2 (M+H).

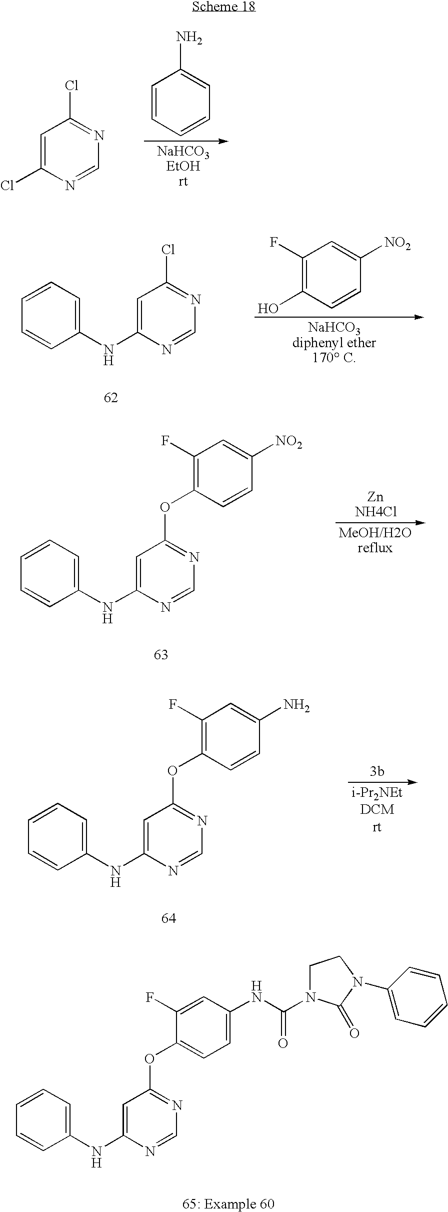

### Example 60

### N-(3-fluoro-4-(6-(phenylamino)pyrimidin-4-yloxy)phenyl)-2-oxo-3-phenyl-imidazolidine-1-carboxamide (65)

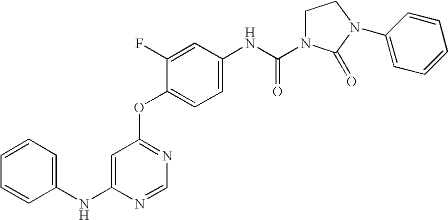

### Step 1: 6-chloro-N-phenylpyrimidin-4-amine (62)

To a solution of 4,6-dichloropyrimidine (2 g, 13.42 mmol) in EtOH (15 ml) was added aniline (1.094 g, 11.75 mmol) and NaHCO₃ (1.692 g, 20.14 mmol) and the reaction mixture was stirred at room temperature for three hours. The solvent was removed and the crude mixture was suspended in EtOAc and washed well with water. The organic phase was collected, dried over sodium sulfate, filtered and concentrated. The crude residue was purified by flash column chromatography on silica gel (20% EtOAc in hexanes) to afford the title compound 62 (690 mg, 50%) as a white solid. MS (m/z): 206.1-208.1 (M+H).

### Step 2: 6-(2-fluoro-4-nitrophenoxy)-N-phenylpyrimidin-4-amine (63)

A suspension of 62 (370 mg, 1.80 mmol), 2-fluoro-4-nitrophenol (848 mg, 5.40 mmol) and sodium bicarbonate (453 mg, 5.40 mmol) in Ph₂O (5 ml) was heated to 170° C. for three hours. The reaction mixture was cooled-down to room temperature and diluted with DCM. The mixture was filtered and concentrated. The crude residue was purified by flash column chromatography on silica gel (20% EtOAc in hexanes) to afford the title compound 63 (571 mg, 97%) as a white solid. MS (m/z): 327.2 (M+H).

### Step 3: 6-(4-amino-2-fluorophenoxy)-N-phenylpyrimidin-4-amine (64)

To a solution of 63 (571 mg, 1.75 mmol) in MeOH (7 ml) and water (3 ml) was added ammonium chloride (187 mg, 3.5 mmol) and zinc dust (1.03 g, 15.75 mmol) and the reaction mixture was heated to reflux for 15 hours. The reaction mixture was cooled-down to rt and filtered. The filtrate was concentrated and dissolved in EtOAc then washed with water. The organic phase was collected, dried over anhydrous Na₂SO₄, filtered and concentrated to afford the title compound 64 (252 mg, 49%) as brown solid. MS (m/z): 297.1 (M+H).

### Step 4: N-(3-fluoro-4-(6-(phenylamino)pyrimidin-4-yloxy)phenyl)-2-oxo-3-phenyl-imidazolidine-1-carboxamide (65)

The title compound 65 (example 60) was prepared in one step from 64 and 3b as yellow solid (Scheme 18) following the same procedure as in example 5, step 5 (Scheme 3). ¹H NMR (400 MHz, DMSO-d₆): 10.49 (s, 1H), 9.63 (s, 1H), 8.30 (s, 1H), 7.72 (m, 1H), 7.62-7.57 (m, 4H), 7.39-7.28 (m, 6H), 7.18 (m, 1H), 7.03 (m, 1H), 6.21 (s, 1H), 3.94 (s, 4H). MS (m/z): 485.3 (M+H).

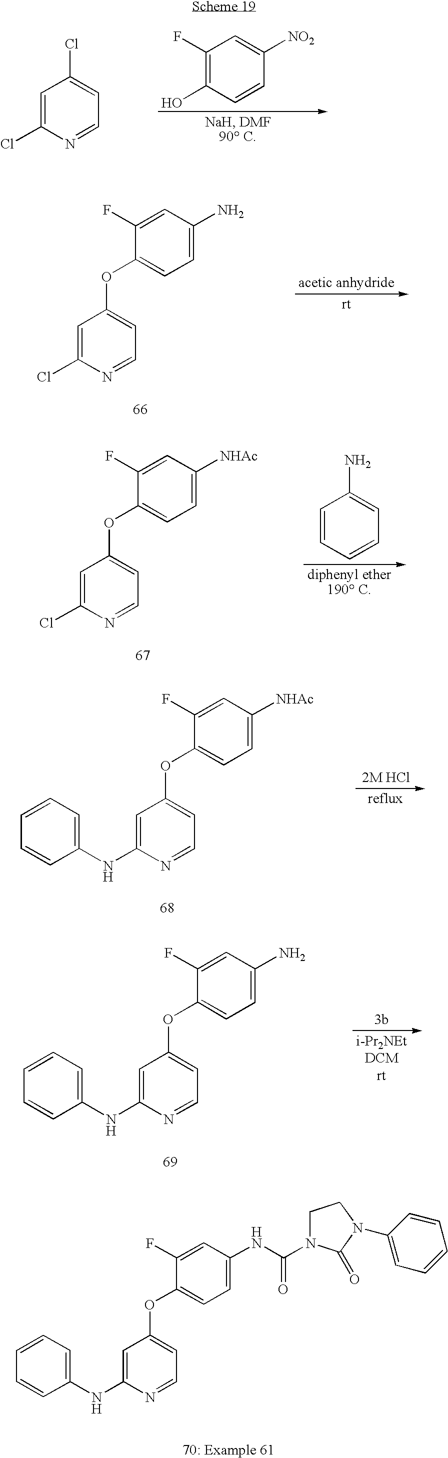

### Example 61

### N-(3-fluoro-4-(2-(phenylamino)pyridin-4-yloxy)phenyl)-2-oxo-3-phenylimidazolidine-1-carboxamide (70)

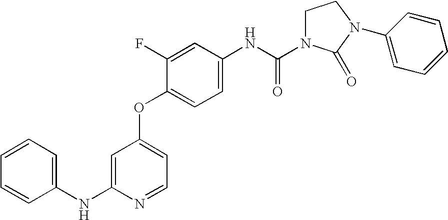

### Step 1: 4-(2-chloropyridin-4-yloxy)-3-fluoroaniline (66)

To a solution of 4-amino-2-fluorophenol (500 mg, 3.93 mmol) in DMF (10 ml) was added NaH (173 mg, 60% on mineral oil, 4.33 mmol) and the reaction mixture was stirred at room temperature for 30 min. The reaction mixture was heated to 90° C. and stirred for 4 hours. The mixture was cooled-down to rt, diluted with water and EtOAc. The organic phase was washed well with water, dried over anhydrous Na₂SO₄, filtered and concentrated. The crude residue was purified by flash column chromatography on silica gel (40% EtOAc in hexanes) to afford the title compound 66 (650 mg, 70%) as a beige solid. MS (m/z): 239.1 (M+H)

### Step 2: N-(4-(2-chloropyridin-4-yloxy)-3-fluorophenyl)acetamide (67)

A solution of 66 (650 mg, 2.672 mmol) in Ac₂O (10 ml) was stirred at room temperature for 3 hours. The solvent was removed and the resultant oil was dissolved in EtOAc and washed well with sat NaHCO₃ soln. The organic phase was dried over anhydrous Na₂SO₄, filtered and concentrated to give the title compound 67 (760 mg, 99%) as a brown solid. MS (m/z): 281.2 (M+H).

### Step 3: N-(3-fluoro-4-(2-(phenylamino)pyridin-4-yloxy)phenyl)acetamide (68)

To a solution of 67 (760 mg, 2.71 mmol) in diphenyl ether (10 ml) was added aniline (504 mg, 5.42 mmol) and the reaction mixture was stirred at 190° C. for 3 hours. The reaction mixture was cooled-down to rt, diluted with DCM, filtered and concentrated. The crude residue was purified by flash column chromatography on silica gel (60% EtOAc in hexanes) to afford the title compound 68 (685 mg, 75%) an oil. MS (m/z): 338.1 (M+H).

### Step 4: 4-(4-amino-2-fluorophenoxy)-N-phenylpyridin-2-amine (69)

A mixture of 68 (685 mg, 2.031 mmol) in 2M HCl (10 ml) was heated to reflux for 4 hours. The water was removed and the residue was dissolved in a mixture of sodium bicarbonate and DCM. The organic phase was collected, dried over anhydrous Na₂SO₄, filtered and concentrated to afford the title compound 69 (400 mg, 67%) as beige solid. MS (m/z): 296.2 (M+H).

### Step 5: N-(3-fluoro-4-(2-(phenylamino)pyridin-4-yloxy)phenyl)-2-oxo-3-phenylimidazolidine-1-carboxamide (70)

The title compound 70 (example 61) was prepared in one step from 69 and 3b as yellow solid (Scheme 19) following the same procedure as in example 5, step 5 (Scheme 3). ¹H NMR (400 MHz, DMSO-d₆): 10.53 (s, 1H), 9.0 (s, 1H), 8.02 (m, 1H), 7.85 (m, 1H), 7.62 (m, 4H), 7.35 (m, 4H), 7.20 (m, 3H), 6.86 (m, 1H), 6.43 (m, 1H), 6.17 (m, 1H), 3.95 (m, 4H). MS (m/z): 484.2 (M+H).

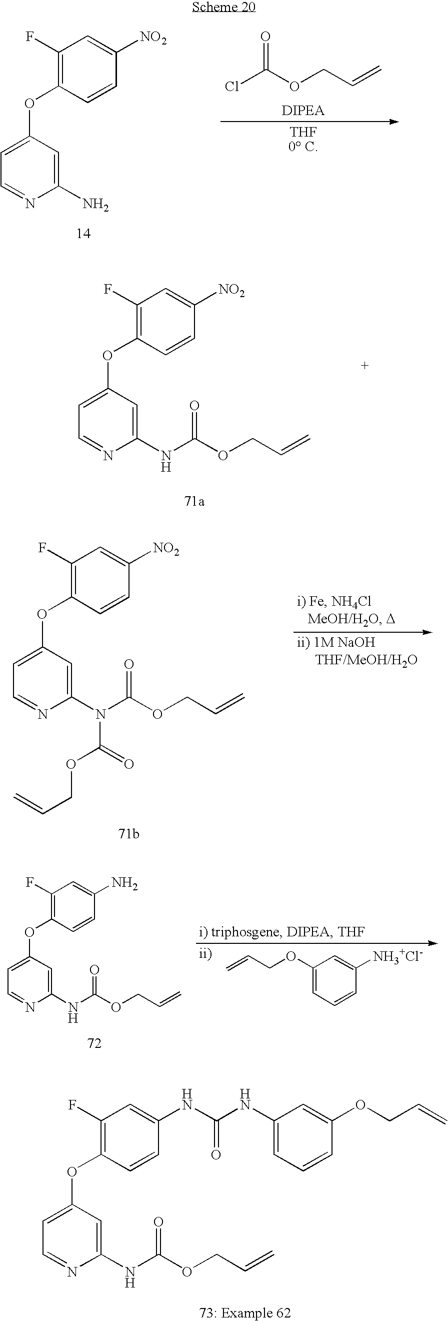

### Example 62

### Allyl 4-(4-(3-(3-(allyloxy)phenyl)ureido)-2-fluorophenoxy)pyridin-2-ylcarbamate (73)

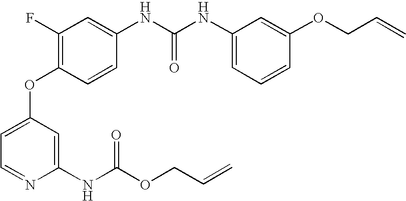

### Step 1: Allyl 4-(2-fluoro-4-nitrophenoxy)pyridin-2-ylcarbamate (71a) and allyl N-4-(2-fluoro-4-nitrophenoxy)pyridin-2-yl-N-allyloxycarbonyl-carbamate (71b)

To a stirred solution at room temperature of 14 (0.83 g, 3.3 mmol) in THF (40 mL) was added DIPEA (2.0 mL, 11 mmol) and allyl chloroformate (1.0 mL, 9.4 mmol) and the resulting solution was stirred at room temperature for 3 h. The reaction mixture was concentrated, partitioned between ethyl acetate and water, washed with water, dried over magnesium sulfate, filtered and concentrated to afford a mixture (approx. 1:1) of mono-Alloc product 71a and bis-Alloc product 71b (0.86 g, ˜70% combined yield). This mixture was used crude in the next step. 71a, MS: 334.1 (M+H) and 71b, MS: 418.2 (M+H).

### Step 2: Allyl 4-(4-amino-2-fluorophenoxy)pyridin-2-ylcarbamate (72)

To a stirred solution of 71a-b (0.86 g, 2.3 mmol) in MeOH (75 mL) was added iron powder (2.3 g, 41 mmol) and ammonium chloride (0.17 g, 3.1 mmol) in water (5 mL). The resulting suspension was heated to reflux for 2 h, then cooled, filtered through celite, and concentrated. Silica gel chromatography (50% ethyl acetate/hexanes) of the residue yielded 72 contaminated with the corresponding bis-Alloc product. The impure product was dissolved in THF (40 mL), and 1M aqueous NaOH (5 mL, 5.0 mmol) and methanol (5 mL) were added. The solution was stirred at r.t. for 4 h, and partially concentrated. The resulting solid was isolated by suction filtration and washed with water and methanol. The solid was dissolved in formic acid and concentrated. The residue was dissolved in 3M aqueous HCl (50 mL) and heated to 80° C. for 3 h. The solution was cooled to r.t., neutralized with solid sodium bicarbonate, and extracted with ethyl acetate. The organic phase was washed with brine, dried over magnesium sulfate, filtered and concentrated. The crude residue was purified by silica gel chromatography (50% ethyl acetate/hexanes) to afford the title compound 72 (0.40 g, 64%). ¹H NMR (400 MHz, DMSO-d₆) δ (ppm): 10.27 (s, 1H); 8.10 (d, J=5.7, 2H); 7.31 (d, J=2.3, 1H); 6.96 (t, J=9.0, 1H); 6.60 (dd, J=5.7, 2.4, 1H); 6.49 (dd, J=13.3, 2.5, 1H); 6.42-6.38 (m, 1H); 5.95-5.88 (m, 1H); 5.46 (s, 2H); 5.35-5.30 (m, 1H); 5.22-5.18 (m, 1H); 4.58-4.55 (m, 2H). MS: 304.2 (M+H).

### Step 3: Allyl 4-(4-(3-(3-(allyloxy)phenyl)ureido)-2-fluorophenoxy)pyridin-2-ylcarbamate (73)

To a stirred solution at 0° C. of 72 (15 mg, 0.049 mmol) in THF (40 mL) was added DIPEA (0.05 mL, 0.25 mmol) and triphosgene (5 mg, 0.016 mmol) and the resulting solution was stirred for 3 h. 3-Allyloxyaniline hydrochloride (14 mg, 0.074 mmol) was added and the solution was allowed to warm to room temperature and stirred for 18 h. The mixture was then concentrated and the residue purified by Gilson reverse phase HPLC (Aquasil C-18 column, 45-100% MeOH/H₂O+0.05% of HCO₂H, 30 min. linear gradient elution) and lyophilization to yield the title compound 73 (15 mg, 63%). ¹H NMR (400 MHz, DMSO-d₆) δ (ppm): 10.36 (s, 1H); 9.05 (s, 1H); 8.83 (s, 1H); 8.15 (d, J=5.9, 1H); 7.70 (dd, J=13.3, 2.3, 1H); 7.35-7.27 (m, 2H); 7.22-7.15 (m, 3H); 6.94 (d, J=8.0, 1H); 6.68 (dd, J=5.6, 2.3), 1H); 6.60-6.57 (m, 1H); 6.08-6.01 (m, 1H); 5.95-5.88 (m, 1H); 5.42-5.30 (m, 2H); 5.27-5.18 (m, 2H); 4.57-4.52 (m, 4H). MS: 479.3 (M+H).

### Example 63

### Allyl 4-(4-(3-(2-(allyloxy)phenyl)ureido)-2-fluorophenoxy)pyridin-2-ylcarbamate (74)

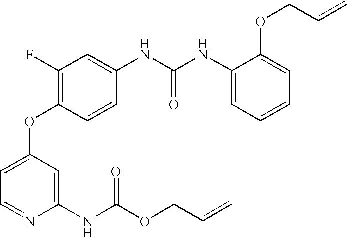

The title compound 74 (example 63) was prepared in one step from 72 and 2-allyloxyaniline (Scheme 20) following the same procedure as in example 62, step 3 (Scheme 20). ¹H NMR (400 MHz, DMSO-d₆) δ (ppm): 10.34 (s, 1H); 9.72 (s, 1H); 8.23 (s, 1H); 8.15 (d, J=5.9, 1H); 8.11 (dd, J=7.6, 2.0, 1H); 7.77-7.71 (m, 1H); 7.36 (d, J=2.4, 1H); 7.31 (t, J=9.0, 1H); 7.20-7.16 (m, 1H); 7.05-7.00 (m, 1H); 6.98-6.88 (m, 2H); 6.68 (dd, J=5.6, 2.3, 1H); 6.16-6.06 (m, 1H); 5.97-5.86 (m, 1H); 5.47-5.43 (m, 1H); 5.35-5.31 (m, 1H); 5.32-5.30 (m, 1H); 5.23-5.18 (m, 1H); 4.72-4.68 (m, 2H); 4.58-4.54 (m, 2H). MS: 479.3 (M+H).

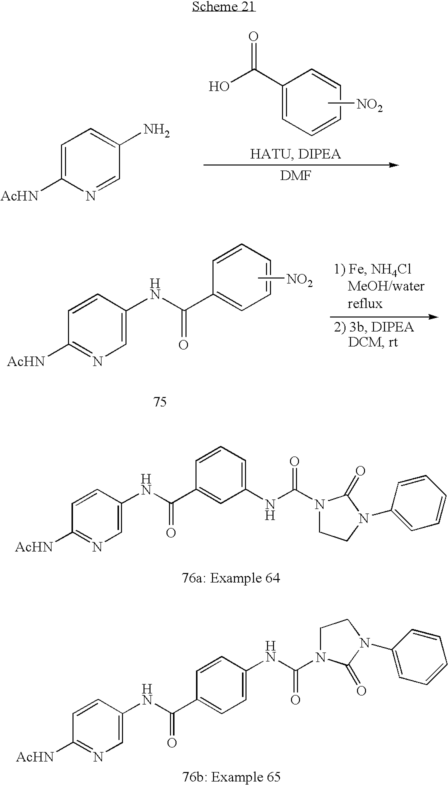

### Example 35

### N-(3-(6-acetamidopyridin-3-ylcarbamoyl)phenyl)-2-oxo-3-phenylimidazolidine-1-carboxamide (76a)

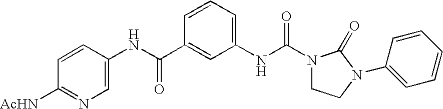

### Step 1. N-(6-acetamidopyridin-3-yl)-3-nitrobenzamide (75a)

To a solution of 2-acetamido-5-aminopyridine (0.5 g, 3.31 mmol) in DMF (7 ml) under nitrogen at room temperature was added 3-nitrobenzoic acid (0.663 g, 3.97 mmol), DIPEA (1.73 ml, 9.92 mmol) and HATU (1.89 g, 4.96 mmol). The reaction mixture was stirred for 18 h, diluted with ethyl acetate, washed with a saturated aqueous solution of sodium bicarbonate and a saturated aqueous solution of ammonium chloride. The organic phase was dried over anhydrous sodium sulfate, filtered and concentrated. The crude residue was purified via Biotage (linear gradient 0-10%, methanol/dichloromethane; 25M column). Trituration in a mixture ethyl acetate-hexane afforded the title compound 75a (0.60 g, 2.00 mmol, 60%) as a pink solid. ¹H NMR (400 MHz, CDCl₃) δ ppm: 10.72 (s, 1H), 10.52 (s, 1H), 8.82 (t, J=2.0 Hz, 1H), 8.72 (t, J=2.0 Hz, 1H), 8.48-8.40 (m, 2H), 8.12-8.08 (m, 2H), 7.86 (t, J=8.0 Hz, 1H), 2.09 (s, 3H).

### Step 2. N-(3-(6-acetamidopyridin-3-ylcarbamoyl)phenyl)-2-oxo-3-phenylimidazolidine-1-carboxamide (76a)

The title compound 76a (example 64) was prepared in one step from compound 75a following the same procedure as in example 3, steps 8 and 9 (Scheme 1). Final purification by Gilson Prep-HPLC (Phenomenex C18 column, linear gradient 30-95%, eluent methanol-water (0.05% formic acid in both), 30 ml/min over 60 min) afforded compound 76a as a white solid (formate salt). ¹H NMR (400 MHz, CDCl₃) δ ppm: 10.57 (s, 1H), 10.49 (s, 1H), 10.42 (s, 1H), 8.71 (dd, J=1.6, 1.6 Hz, 1H), 8.44 (s, 0.6H, formate), 8.12-8.05 (m, 3H), 7.87-7.83 (m, 1H), 7.68 (d, J=8.0 Hz, 1H), 7.64 (dd, J=8.8, 0.8 Hz, 2H), 7.51 (t, J=8.0 Hz, 1H), 7.46-7.40 (m, 2H), 7.18 (t, J=7.6 Hz, 1H), 4.02-3.92 (m, 4H), 2.08 (s, 3H). MS: 459.3 (M+1).

### Example 65

### N-(4-(6-acetamidopyridin-3-ylcarbamoyl)phenyl)-2-oxo-3-phenylimidazolidine-1-carboxamide (76b)

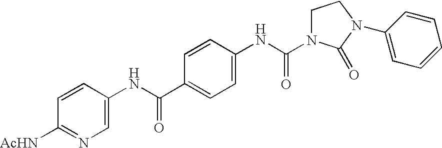

The title compound 76b (example 65) was prepared in three steps from 2-acetamido-5-aminopyridine and 4-nitrobenzoic acid as a beige solid (formate salt) following the same procedure as in example 64, steps 1 and 2 (Scheme 21). ¹H NMR (400 MHz, CDCl₃) δ ppm: 10.65 (s, 1H), 10.46 (s, 1H), 10.28 (s, 1H), 8.73-8.69 (m, 1H), 8.12-8.02 (m, 2H), 7.99 (d, J=8.8 Hz, 2H), 7.72 (d, J=8.8 Hz, 2H), 7.64 (dd, J=8.8, 1.2 Hz, 2H), 7.43 (t, J=8.0 Hz, 2H), 7.18 (t, J=7.6 Hz, 1H), 4.02-3.91 (m, 4H), 2.08 (s, 3H). MS: 459.3 (M+1).

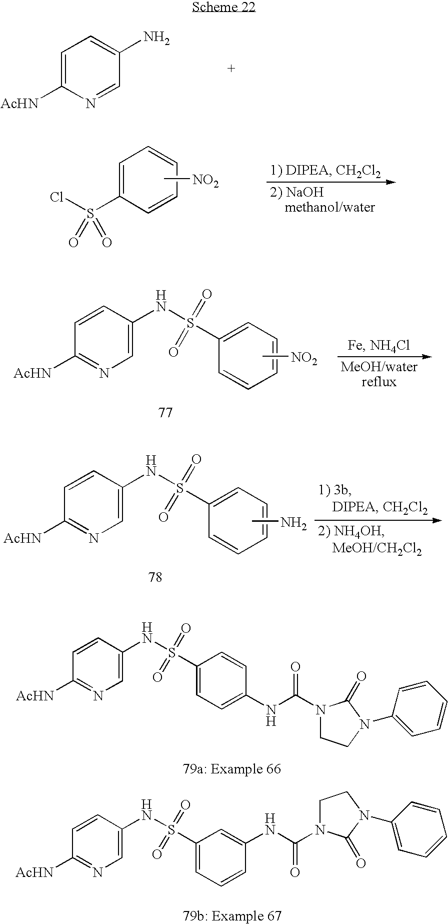

### Example 66

### N-(4-(N-(6-acetamidopyridin-3-yl)sulfamoyl)phenyl)-2-oxo-3-phenylimidazolidine-1-carboxamide (79a)

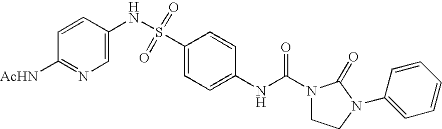

### Step 1. N-(5-(4-nitrophenylsulfonamido)pyridin-2-yl)acetamide (77a)

To a solution of 2-acetamido-5-aminopyridine (1 g, 6.62 mmol) in dichloromethane (33 ml) under nitrogen at room temperature was added 4-nitrobenzenesulfonyl chloride (3.23 g, 14.6 mmol) and triethylamine (2.77 ml, 19.9 mmol). The reaction mixture was stirred for 18 h. The solid suspension was filtered off, rinsed with dichloromethane and dried. The solid was suspended in a mixture of MeOH (34 ml) and a 1N aqueous solution of NaOH (13.5 ml, 13.5 mmol) and stirred for 18 h. The mixture was concentrated, diluted with water and acidified to pH 3 using a 1N aqueous solution of HCl. The suspension was collected by filtration, rinsed with water and dried to afford the title compound 77a (2.22 g, 6.62 mmol, quantitative) as a beige solid. MS: 337.1 (M+1).

### Step 2. N-(5-(4-aminophenylsulfonamido)pyridin-2-yl)acetamide (78a)

Starting from compound 77a and following a procedure similar to the one described for compound 8 (example 1, step 8), title compound 78a was obtained as a brown solid. MS: 307.2 (M+1).

### Step 3. N-(4-(N-(6-acetamidopyridin-3-yl)sulfamoyl)phenyl)-2-oxo-3-phenyl-imidazolidine-1-carboxamide (79a)

To a solution of compound 78a (100 mg, 0.326 mmol) in dichloromethane (4 ml) under nitrogen at room temperature was added 2-oxo-3-phenylimidazolidine-1-carbonyl chloride 3b (88 mg, 0.39 mmol) and DIPEA (0.17 ml, 0.98 mmol). The reaction mixture was stirred for 18 h and methanol (1 ml) was added. The solid suspension was filtered off, rinsed with methanol and suspended in dichloromethane (5 ml) and methanol (5 ml). Ammonium hydroxide (0.64 ml, 16 mmol) was added and the mixture was stirred for 18 h. The solvents were removed under reduced pressure and the residue purified by trituration in a mixture of methanol/dichloromethane to afford the title compound 79a (55 mg, 0.11 mmol, 34%) as a white solid. ¹H NMR (400 MHz, CDCl₃) δ ppm: 10.66 (s, 1H), 10.43 (s, 1H), 10.15 (s, 1H), 7.97-7.92 (m, 2H), 7.73-7.68 (m, 2H), 7.68-7.59 (m, 4H), 7.47-7.39 (m, 3H), 7.17 (t, J=7.6 Hz, 1H), 4.00-3.88 (m, 4H), 2.03 (s, 3H). MS: 495.2 (M+1).

### Example 67

### N-(3-(N-(6-acetamidopyridin-3-yl)sulfamoyl)phenyl)-2-oxo-3-phenylimidazolidine-1-carboxamide (79b)

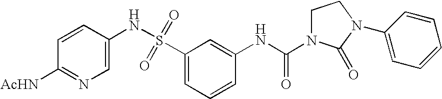

The title compound 79b (example 67) was prepared in three steps from 2-acetamido-5-aminopyridine and 3-nitrobenzenesulfonyl chloride as a white solid following the same procedure as in example 66, steps 1-3 (Scheme 22). ¹H NMR (400 MHz, CDCl₃) δ ppm: 10.57 (s, 1H), 10.44 (s, 1H), 10.32 (s, 1H), 8.16 (t, J=2.0 Hz, 1H), 7.98-7.92 (m, 2H), 7.65-7.60 (m, 3H), 7.52-7.39 (m, 4H), 7.38-7.34 (m, 1H), 7.18 (t, J=7.6 Hz, 1H), 4.00-3.88 (m, 4H), 2.03 (s, 3H). MS: (495.3M+1).

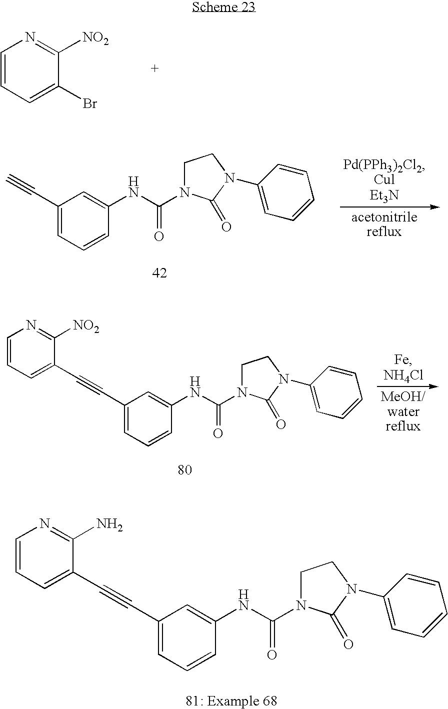

### Example 68

### N-(3-((2-aminopyridin-3-yl)ethynyl)phenyl)-2-oxo-3-phenylimidazolidine-1-carboxamide (81)

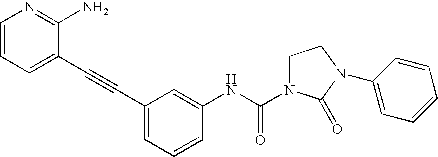

### Step 1. N-(3-((2-nitropyridin-3-yl)ethynyl)phenyl)-2-oxo-3-phenylimidazolidine-1-carboxamide (80)

Starting from compound 42 and following the same procedure as described to prepare compound 43 (scheme 13, example 54, step 2) but replacing 2-acetamido-5-bromopyridine by 3-bromo-2-nitropyridine, compound 80 was obtained as a brown solid. MS: 428.2 (M+1).

Step 2. N-(3-((2-aminopyridin-3-yl)ethynyl)phenyl)-2-oxo-3-phenylimidazolidine-1-carboxamide formate (81)

Compound 81 (example 68) was prepared starting from compound 80 and following a procedure similar to the one described for compound 8 (example 1, step 8). Purification by Gilson Prep-HPLC (Phenomenex C18 column, linear gradient 40-95% Methanol/water (0.05% formic acid in both), 30 mL/min over 60 min) afforded compound 81 as a beige solid (formate salt). ¹H NMR (400 MHz, CDCl₃) δ ppm: 10.47 (s, 1H), 8.49 (bs, 2H), 7.98 (dd, J=4.8, 1.6 Hz, 1H), 7.84 (bs, 1H), 7.66-7.58 (m, 4H), 7.46-7.32 (m, 4H), 7.18 (t, J=7.2 Hz, 1H), 6.57 (dd, J=7.2, 5.2 Hz, 1H), 6.34 (bs, 2H), 4.10-3.89 (m, 4H). MS: 398.3 (M+1).

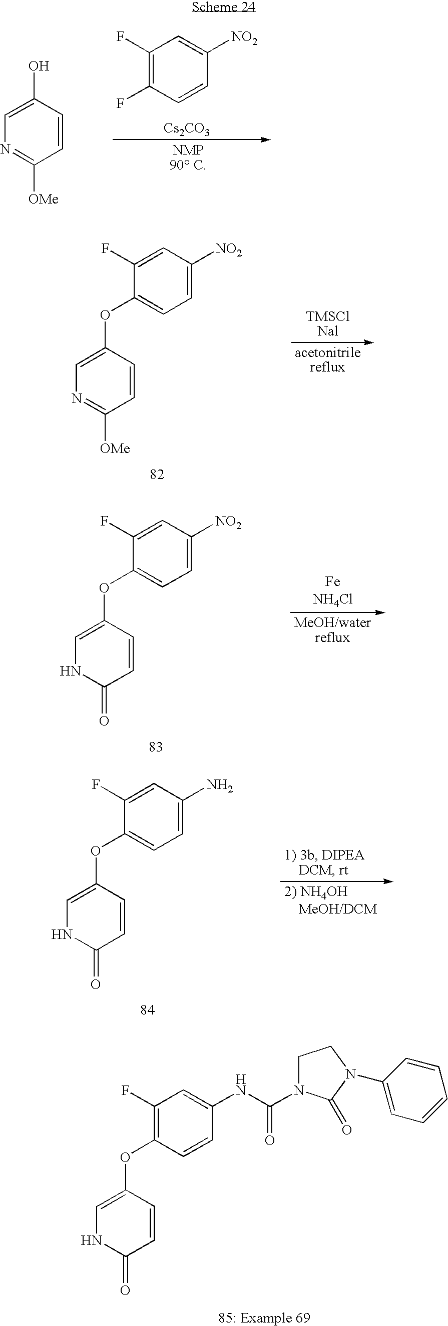

### Example 69

### N-(3-fluoro-4-(6-oxo-1,6-dihydropyridin-3-yloxy)phenyl)-2-oxo-3-phenylimidazolidine-1-carboxamide (85)

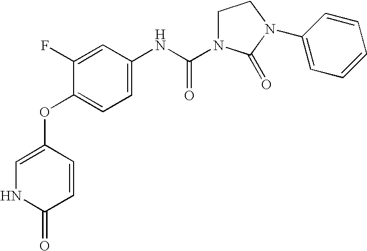

### Step 1. 5-(2-fluoro-4-nitrophenoxy)-2-methoxypyridine (82)

A suspension of 6-methoxypyridin-3-ol (0.620 g, 4.96 mmol) [WO 98/25920, Bioorg. Med. Chem. Lett. 8 (1998) 2797-2802], 3,4-difluoronitrobenzene (0.55 ml, 4.96 mmol) and cesium carbonate (3.23 g, 9.91 mmol) in N-methyl-2-pyrrolidinone (7 ml) was stirred overnight at 90° C. Water was added and the aqueous phase was extracted twice with ethyl acetate. The combined organic extracts were washed with a saturated aqueous solution of sodium bicarbonate, dried over anhydrous sodium sulfate and the solvent was removed under reduced pressure. The crude residue was purified via Biotage (0-2%, methanol/dichloromethane; 25M column) to afford the title compound 82 (0.74 g, 2.8 mmol, 57%) as a yellow solid. MS: 265.1 (M+1).

### Step 2. 5-(2-fluoro-4-nitrophenoxy)pyridin-2(1H)-one (83)

To a solution of compound 82 (0.24 g, 0.91 mmol) in acetonitrile (9 ml) under nitrogen at room temperature was added chlorotrimethylsilane (1.16 ml, 9.1 mmol) and sodium iodide (0.34 g, 2.27 mmol). The reaction mixture was heated to reflux for 2 h. It was cooled-down and quenched by adding ammonium hydroxide 20%, and extracted twice with ethyl acetate. The combined organic layers were dried over anhydrous sodium sulfate and concentrated. The crude residue was purified via Biotage (linear gradient 0-10%, methanol/dichloromethane; 25M column) to afford the title compound 83 (0.13 g, 0.52 mmol, 57%) as a beige solid. MS: (251.0M+1).

### Step 3. 5-(4-amino-2-fluorophenoxy)pyridin-2(1H)-one (84)

To a suspension of compound 83 (0.13 g, 0.52 mmol) in methanol (10 ml) and water (5 ml) at room temperature was added ammonium chloride (24 mg, 0.45 mmol) and iron powder (247 mg, 4.42 mmol). The reaction mixture was heated to reflux for 40 min. The solvents were removed under reduced pressure and the residue was purified by flash column chromatography on silica gel (10% methanol/dichloromethane) to afford the title compound 84 (100 mg, 0.454 mmol, 87%) as a beige solid. MS: 221.0 (M+1).

### Step 4. N-(3-fluoro-4-(6-oxo-1,6-dihydropyridin-3-yloxy)phenyl)-2-oxo-3-phenylimidazolidine-1-carboxamide (85)

To a solution under nitrogen at room temperature of compound 84 (100 mg, 0.454 mmol) in dichloromethane (5 ml) was slowly added compound 3b (204 mg, 0.91 mmol) followed by DIPEA (317 μl, 1.817 mmol). The mixture was stirred for 18 h, diluted with ethyl acetate, washed with a saturated aqueous solution of sodium bicarbonate, a saturated aqueous solution of ammonium chloride and brine. The organic phase was dried over anhydrous sodium sulfate, filtered and the solvent was removed under reduced pressure. The residue was purified via Biotage (linear gradient 0-10%, methanol/dichloromethane; 12M column). The white solid obtained was dissolved in dichloromethane (10 mL) and a mixture of 2% ammonium hydroxide in methanol (10 mL) was added. The mixture was stirred for 18 h and the solvents were removed under reduced pressure. The residue was purified via Biotage (linear gradient 0-20%, methanol/dichloromethane; 25M column) and triturated in methanol to afford the title compound 85 (36 mg, 0.088 mmol, 19%) as a white solid. ¹H NMR (400 MHz, CDCl₃) δ ppm: 11.40 (bs, 1H), 10.41 (s, 1H), 7.71 (dd, J=13.2, 2.4 Hz, 1H), 7.62 (d, J=8.0 Hz, 2H), 7.46-7.36 (m, 4H), 7.24 (d, J=7.6 Hz, 1H), 7.17 (t, J=7.6 Hz, 1H), 7.04 (t, J=8.8 Hz, 1H), 6.42 (d, J=10.4 Hz, 1H), 4.00-3.88 (m, 4H). MS: 409.1 (M+1).

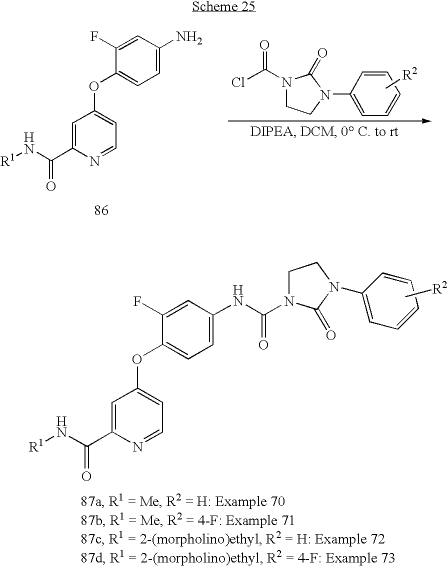

Examples 70-73 (compounds 87a-87d) were prepared in one step from the appropriate anilines 86 (prepared according to Organic Process Research & Development 2002, 6, 777 and Bioorg. Med. Chem. Lett. 2004, 14, 783) using the same procedure as described to prepare compound 9c (example 3, scheme 1).

TABLE 7
Characterization of compounds 87a-87d (examples 70-73) prepared according to
Scheme 25

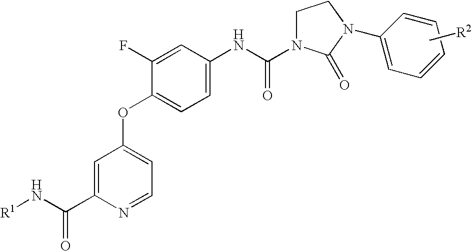

| Cpd | Ex. | R¹ | R² | Name | Characterization |
|---|---|---|---|---|---|
| 87a | 70 | Me | H | 4-(2-fluoro-4-(2-oxo-3- | ¹H NMR (400 MHz, DMSO-d₆) δ (ppm): |
| | | | | phenylimidazolidine-1- | 10.57 (s, 1H), 8.81 (q, J = 4.8 Hz, 1H), 8.54 |
| | | | | carboxamido)phenoxy)-N- | (d, J = 6.0 Hz, 1H), 7.85 (dd, J = 12.8 Hz, |
| | | | | methylpicolinamide | 1H), 7.64 (dd, J = 8.8, 1.2 Hz, 2H), 7.48-7.38 |
| | | | | | (m, 5H), 7.23-7.15 (m, 2H), 4.02-3.91 (m, |
| | | | | | 4H), 2.79 (d, J = 4.8 Hz, 3H). MS: 450.2 |
| | | | | | (M = H). |
| 87b | 71 | Me | 4-F | 4-(2-fluoro-4-(3-(4- | ¹H NMR (400 MHz, DMSO-d₆) δ (ppm): |
| | | | | fluorophenyl)-2- | 10.54 (s, 1H), 8.84-8.77 (m, 1H), 8.54 (d, J = |
| | | | | oxoimidazolidine-1- | 5.6 Hz, 1H), 7.84 (d, J = 12.4 Hz, 1H), 7.68- |
| | | | | carboxamido)phenoxy)-N- | 7.62 (m, 2H), 7.47-7.37 (m, 3H), 7.29 (t, J = |
| | | | | methylpicolinamide | 8.8 Hz, 2H), 7.21 (dd, J = 5.6, 2.4 Hz, 1H), |
| | | | | | 4.01-3.90 (m, 4H), 2.79 (d, J = 4.4 Hz, 3H). |
| | | | | | MS: 468.2 (M + H). |
| 87c | 72 | 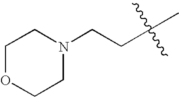 | H | 4-(2-fluoro-4-(2-oxo-3-phenylimidazolidine-1-carboxamido)phenoxy-N-(2-morpholinoethyl)picolinamide | ¹H NMR (400 MHz, DMSO-d₆) δ (ppm):10.57 (s, 1H), 9.20 (m, 1H), 8.59 (d, J = 5.6Hz, 1H), 7.85 (dd, J = 13.2, 2.4 Hz, 1H), 7.63(dt, J = 8.8, 1.4 Hz, 2H), 7.50-7.38 (m, 5H),7.27 (dd, J = 5.6, 2.8 Hz, 1H), 7.18 (t, J = 7.2Hz, 1H), 4.02-3.91 (m, 6H), 3.76-3.62 (m,4H). MS: 549.2(M + H). |
| 87d | 73 | 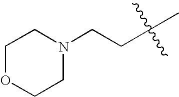 | 4-F | 4-(2-fluoro-4-(3-(4-fluorophenyl)-2-oxoimidazolidine-1-carboxamido)phenoxy)-N-(2-morpholinoethyl)picolinamide | ¹H NMR (400 MHz, DMSO-d₆) δ (ppm):10.54 (s, 1H), 8.75 (t, J = 6.0 Hz, 1H), 8.56(d, J = 5.6 Hz, 1H), 7.84 (dd, J = 12.8, 1.6Hz, 1H), 7.68-7.62 (m, 2H), 7.47-7.38 (m,3H), 7.32-7.25 (m, 2H), 7.23 (dd, J = 5.6, 2.4Hz, 1H), 4.00-3.90 (m, 4H), 3.55 (t, J = 4.8 Hz, 4H). MS: 567.3 (M + H). |

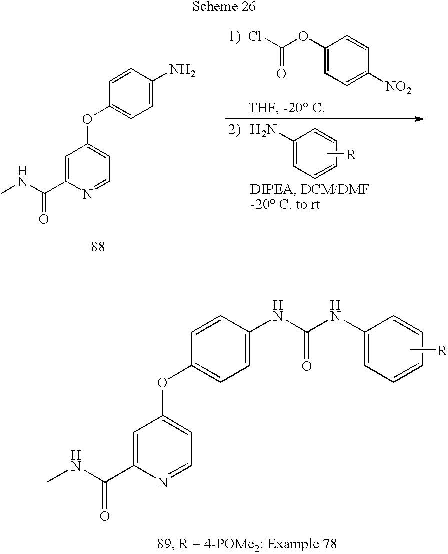

### Example 78

### 4-(4-(3-(4-(dimethylphosphoryl)phenyl)ureido)phenoxy)-N-methylpicolinamide (89)

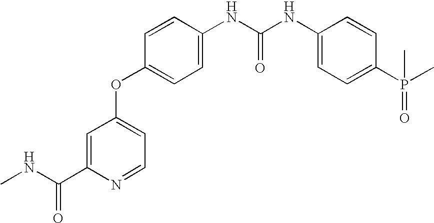

To a solution of 88 (272 mg, 1.118 mmol, prepared according to Organic Process Research & Development 2002, 6, 777), in THF under nitrogen at −20° C. was added 4-nitrophenyl chloroformate (338 mg, 1.677 mmol). The reaction mixture was stirred at −20° C. for 1 h and a solution of 4-(dimethylphosphoryl)aniline (378 mg, 2.236 mmol, prepared according to WO 2005/009348 A2) in THF/DMF (1 mL/0.5 mL) and diisopropylethylamine (1 mL) were added, respectively. Then, the temperature was allowed to warm-up slowly to room temperature and the reaction mixture was stirred for overnight. It was concentrated, diluted with ethyl acetate and washed with a saturated aqueous solution of NH₄Cl. After separation, the aqueous layer was extracted with ethyl acetate. The combined organic layers were washed twice with brine and concentrated. The crude residue was purified via Biotage (linear gradient 0-20%, MeOH/DCM) to afford the title compound 89 (116 mg, 24% yield) as an off-white solid. ¹H NMR (400 MHz, DMSO-d₆) δ (ppm): 9.05 (s, 1H), 8.97 (s, 1H), 8.81-8.75 (m, 1H), 8.50 (d, J=5.2 Hz, 1H), 7.70-7.56 (m, 6H), 7.37 (d, J=2.4 Hz, 1H), 7.20-7.13 (m, 3H), 2.78 (d, J=4.8 Hz, 3H), 1.61 (d, J=13.6 Hz, 6H). MS: 439.3 (M+H).

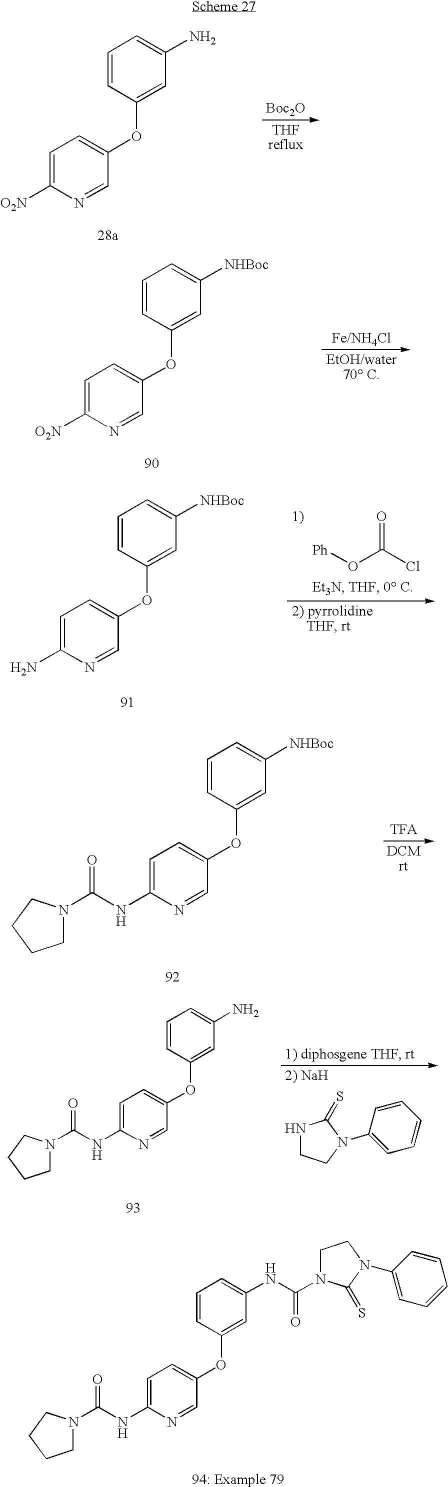

### Example 79

### 3-phenyl-N-(3-(6-(pyrrolidine-1-carboxamido)pyridin-3-yloxy)phenyl)-2-thioxoimidazolidine-1-carboxamide (94)

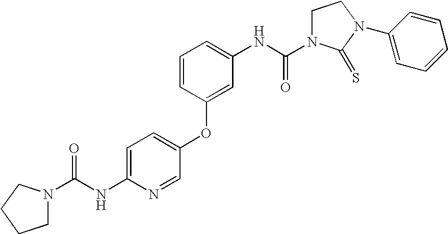

### Step 1. tert-butyl 3-(6-nitropyridin-3-yloxy)phenylcarbamate (90)

A solution of 28a (385 mg, 1.665 mmol) and Boc₂O (0.773 mL, 3.33 mmol) in THF (5 mL) was heated to reflux overnight. The reaction mixture was concentrated and purified by flash column chromatography on silica gel (EtOAc/hexanes:10/90 to 30/70) to afford the title compound 90 (530 mg, 96% yield). MS (m/z): 354.2 (M+Na).

### Step 2. tert-butyl 3-(6-aminopyridin-3-yloxy)phenylcarbamate (91)

A suspension of 90 (530 mg, 1.6 mmol), iron (536 mg, 9.60 mmol) and NH₄Cl (86 mg, 1.600 mmol) in Ethanol (10 mL)/Water (5 mL) was stirred at 70° C. for 2 hrs. The reaction mixture was filtered through celite and concentrated to afford the title compound 91 (573 mg, quantitative yield). MS (m/z): 302.2 (M+1).

### Step 3. tert-butyl 3-(6-(pyrrolidine-1-carboxamido)pyridin-3-yloxy)phenylcarbamate (92)

A solution of 91 (353 mg, 1.171 mmol) and Et₃N (0.261 mL, 1.874 mmol) in THF (6 mL) were cooled to 0° C. and phenylchloroformate (0.220 mL, 1.757 mmol) was added dropwise. The reaction mixture was stirred at rt for 2 hrs, diluted in EtOAc and successively washed with a saturated aqueous solution of NaHCO₃ and brine. The organic phase was dried over anhydrous sodium sulfate, filtered and concentrated. To a solution of the crude material in THF (5 mL) was added pyrrolidine (0.196 mL, 2.373 mmol), and the reaction mixture was stirred at rt overnight. It was then diluted in EtOAc and washed with a saturated aqueous solution of NH₄Cl. The organic phase was dried over anhydrous sodium sulfate, filtered and concentrated. The crude residue was purified by flash column chromatography on silica gel (EtOAc/hexanes:50/50 to 70/30) to afford the title compound 92 (130 mg, 55% yield). MS (m/z): 399.3 (M+1).

### Step 4. N-(5-(3-aminophenoxy)pyridin-2-yl)pyrrolidine-1-carboxamide (93)

A solution of 92 (120 mg, 0.301 mmol) and TFA (1 mL, 12.98 mmol) in DCM (5 mL) was stirred at rt overnight. The reaction mixture was concentrated, diluted in DCM and neutralized with 1M NaOH solution. The organic phase was separated, dried over anhydrous sodium sulfate, filtered and concentrated to afford the title compound 93 (77 mg, 86% yield). MS (m/z): 299.3 (M+1).

### Step 5. 3-phenyl-N-(3-(6-(pyrrolidine-1-carboxamido)pyridin-3-yloxy)phenyl)-2-thioxoimidazolidine-1-carboxamide (94)

A solution of 93 (77 mg, 0.25 mmol) in THF (5 mL) was treated with diphosgene (0.016 mL, 0.129 mmol) and stirred at rt for 30 min. The reaction mixture was then treated with 1-phenylimidazolidine-2-thione (69.5 mg, 0.390 mmol) and 60% NaH mineral oil suspension (15.6 mg, 0.390 mmol) and stirred at rt for 1 hr. The reaction mixture was quenched by a saturated aqueous solution NaHCO₃ and extracted with EtOAc. The organic phase was dried over anhydrous sodium sulfate, filtered and concentrated. The crude residue was purified by flash column chromatography on silica gel (EtOAc/hexanes:50/50 to 60/40) to afford the title compound 94 (20 mg, 20% yield). ¹H NMR (400 MHz, DMSO-d₆) δ (ppm): 12.05 (s, 1H), 8.72 (m, 1H), 8.06 (m, 1H), 7.91 (m, 1H), 7.0-7.6 (m, 9H), 6.68 (m, 1H), 4.20 (m, 2H), 1.83 (m, 4H). MS (m/z): 503.3 (M+1).

### Example 80

### N-(3-(6-acetamidopyridin-3-yloxy)phenyl)-3-phenyl-2-thioxoimidazolidine-1-carboxamide (95)

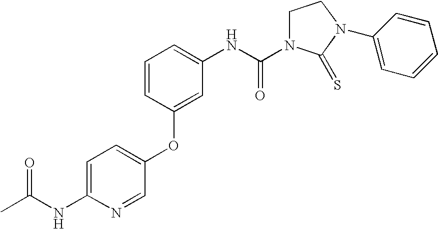

The title compound 95 (example 80) was prepared in three steps from 91 and acetic anhydride following the same procedure as in example 38, step 4 (Scheme 7) and in example 79, steps 4 and 5 (Scheme 27). ¹H NMR (400 MHz, DMSO-d₆) δ (ppm): 12.36 (s, 1H), 10.55 (s, 1H), 8.13 (d, J=2.8 Hz, 1H), 8.10 (d, J=9.0 Hz, 1H), 7.53 (dd, J=9.0, 2.9 Hz, 1H), 7.46 (m, 4H), 7.3-7.4 (m, 3H), 7.07 (d, J=9.2 Hz, 1H), 6.72 (dd, J=8.3, 1.6 Hz, 1H), 4.15 (m, 2H), 4.04 (m, 2H), 2.07 (s, 3H). MS (m/z): 448.4 (M+1).

| | | | | |
|---|---|---|---|---|
| 27l | 81 | 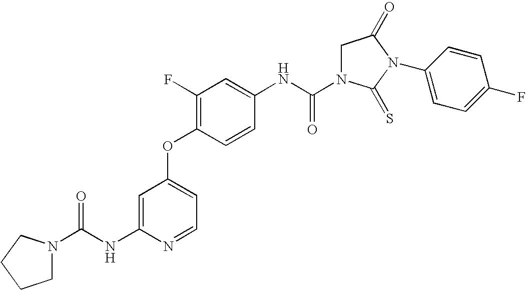 | N-(3-fluoro-4-(2-(pyrrolidine-1-carboxamido)pyridin-4-yloxy)phenyl)-3-(4-fluorophenyl)-4-oxo-2-thioxoimidazolidine-1-carboxamide | ¹H NMR (400 MHz,DMSO-d₆) δ (ppm): 11.96(s, 1H), 8.72 (s, 1H), 8.10(d, J = 5.6 Hz, 1H), 7.77(dd, J = 12.6, 1.6 Hz, 1H),7.45 (d, J = 14.4 Hz, 1H),7.40 (m, 5H), 7.30 (d, J =6.4 Hz, 1H), 6.60 (dd, J =5.7, 2.4 Hz, 1H), 4.69 (s,2H), 1.78 (bs, 4H). MS (m/z): 553.3 (M + 1). |

### Pharmaceutical Compositions

In one embodiment, the invention provides pharmaceutical compositions comprising an inhibitor of VEGF receptor signaling and HGF receptor signaling according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In certain preferred embodiments, compositions of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term “pharmaceutically acceptable salt(s)” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

### Inhibition of VEGF Receptor Signaling and HGF Receptor Signaling

In another embodiment the invention provides a method of inhibiting VEGF receptor signaling and HGF receptor signaling in a cell, comprising contacting a cell in which inhibition of VEGF receptor signaling and HGF receptor signaling is desired with an inhibitor of VEGF receptor signaling and HGF receptor signaling according to the invention. Because compounds of the invention inhibit VEGF receptor signaling and HGF receptor signaling, they are useful research tools for in vitro study of the role of VEGF receptor signaling and HGF receptor signaling in biological processes.

Preferably, the method according to this embodiment of the invention causes an inhibition of cell proliferation of the contacted cells. The phrase “inhibiting cell proliferation” is used to denote an ability of an inhibitor of VEGF receptor signaling and HGF receptor signaling to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth with calipers and comparing the size of the growth of contacted cells with non-contacted cells.

Preferably, growth of cells contacted with the inhibitor is retarded by at least 50% as compared to growth of non-contacted cells. More preferably, cell proliferation is inhibited by 100% (i.e., the contacted cells do not increase in number). Most preferably, the phrase “inhibiting cell proliferation” includes a reduction in the number or size of contacted cells, as compared to non-contacted cells. Thus, an inhibitor of VEGF receptor signaling and HGF receptor signaling according to the invention that inhibits cell proliferation in a contacted cell may induce the contacted cell to undergo growth retardation, to undergo growth arrest, to undergo programmed cell death (i.e., to apoptose), or to undergo necrotic cell death.

In some preferred embodiments, the contacted cell is a neoplastic cell. The term “neoplastic cell” is used to denote a cell that shows aberrant cell growth. Preferably, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some preferred embodiments, the contacted cell is in an animal. Thus, the invention provides a method for treating a cell proliferative disease or condition in an animal, comprising administering to an animal in need of such treatment a therapeutically effective amount of a VEGF receptor signaling and HGF receptor signaling inhibitor of the invention. Preferably, the animal is a mammal, more preferably a domesticated mammal. Most preferably, the animal is a human.

The term “cell proliferative disease or condition” is meant to refer to any condition characterized by aberrant cell growth, preferably abnormally increased cellular proliferation. Examples of such cell proliferative diseases or conditions amenable to inhibition and treatment include, but are not limited to, cancer. Examples of particular types of cancer include, but are not limited to, breast cancer, lung cancer, colon cancer, rectal cancer, bladder cancer, leukemia and renal cancer. In particularly preferred embodiments, the invention provides a method for inhibiting neoplastic cell proliferation in an animal comprising administering to an animal having at least one neoplastic cell present in its body a therapeutically effective amount of a VEGF receptor signaling and HGF receptor signaling inhibitor of the invention.

### ASSAY EXAMPLES

### Assay Example 1

### Inhibition of c-met and VEGF Activity

The following protocols were used to assay the compounds of the invention.

In Vitro Receptor Tyrosine Kinase Assays (c-Met/HGF receptor and VEGF receptor KDR)

These tests measure the ability of compounds to inhibit the enzymatic activity of recombinant human c-Met/HGF receptor and VEGF receptor enzymatic activity.

A 1.3-kb cDNA corresponding to the intracellular domain of c-Met or c-Met IC (Genbank accession number NP000236-1 amino acid 1078 to 1337) is cloned into the BamHI/XhoI sites of the pBlueBacHis2A vector (Invitrogen) for the production of a histidine-tagged version of that enzyme. This construct is used to generate recombinant baculovirus using the Bac-N-Blue™ system according to the manufacturer's instructions (Invitrogen).

The c-Met IC protein is expressed in Hi-5 cells (Trichoplusia Ni) upon infection with recombinant baculovirus construct. Briefly, Hi-5 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.2 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Hi-5 cell pellets infected with the C-Met IC recombinant baculovirus are thawed and gently resuspended in Buffer A (20 mM Tris pH 8.0, 10% glycerol, 1 μg/ml pepstatin, 2 μg/ml Aprotinin and leupeptin, 50 μg/ml PMSF, 50 μg/ml TLCK and 10 μM E64, 0.5 mM DTT and 1 mM Levamisole) using 3 ml of buffer per gram of cells. The suspension is Dounce homogenized after which it is centrifuged at 22500 g, 30 min., 4° C. The supernatant (cell extract) is used as starting material for purification of c-Met IC.

The supernatant is loaded onto a QsepharoseFF column (Amersham Biosciences) equilibrated with Buffer B (20 mM Tris pH 8.0, 10% glycerol) supplemented with 0.05M NaCl. Following a ten column volume (CV) wash with equilibration buffer, bound proteins are eluted with a 5 CV salt linear gradient spanning from 0.05 to 1M NaCl in Buffer B. Typically, the conductivity of selected fractions rank between 6.5 and 37 mS/cm. This Qsepharose eluate has an estimated NaCl concentration of 0.33M and is supplemented with a 5M NaCl solution in order to increase NaCl concentration at 0.5M and also with a 5M Imidazole (pH 8.0) solution to achieve a final imidazole concentration of 15 mM. This material is loaded onto a HisTrap affinity column (GE Healthcare) equilibrated with Buffer C (50 mM NaPO₄ pH 8.0, 0.5M NaCl, 10% glycerol) supplemented with 15 mM imidazole. After a 10 CV wash with equilibration buffer and an 8 CV wash with buffer C+40 mM imidazole, bound proteins are eluted with an 8 CV linear gradient (15 to 500 mM) of imidazole in buffer C. C-Met IC enriched fractions from this chromatography step are pooled based on SDS-PAGE analysis. This pool of enzyme undergoes buffer exchange using PD-10 column (GE Healthcare) against buffer D (25 mM HEPES pH 7.5, 0.1M NaCl, 10% glycerol and 2 mM β-mercaptoethanol). Final C-Met IC protein preparations concentrations are about 0.5 mg/ml with purity approximating 80%. Purified c-Met IC protein stocks are supplemented with BSA at 1 mg/ml, aliquoted and frozen at −80° C. prior to use in enzymatic assay.

In the case of VEGF receptor KDR a 1.6-kb cDNA corresponding to the catalytic domain of VEGFR2 or KDR (Genbank accession number AF035121 amino acid 806 to 1356) is cloned into the Pst I site of the pDEST20 Gateway vector (Invitrogen) for the production of a GST-tagged version of that enzyme. This construct is used to generate recombinant baculovirus using the Bac-to-Bac system according to the manufacturer's instructions (Invitrogen).

The GST-VEGFR2₈₀₆₋₁₃₅₆ protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×10⁶ cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Sf9 cell pellets infected with the GST-VEGFR2₈₀₆₋₁₃₅₆ recombinant baculovirus re thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4° C. The supernatant (cell extract) is used as starting material for purification of GST-VEGFR2₈₀₆₋₁₃₅₆.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50 mM Tris pH 8.0, 20% glycerol and 100 mM NaCl), bound proteins are step eluted with 5 CV of buffer B supplemented with 5 mM DTT and 15 mM glutathion. GST-VEGFR2₈₀₆₋₁₃₅₆ enriched fractions from this chromatography step are pooled based on U.V. trace i.e. fractions with high O.D⁻²⁸⁰⁻ Final GST-VEGFR2₈₀₆₋₁₃₅₆ protein preparations concentrations are about 0.7 mg/ml with purity approximating 70%. Purified GST-VEGFR2₈₀₆₋₁₃₅₆ protein stocks are aliquoted and frozen at −80° C. prior to use in enzymatic assay.

Inhibition of c-Met/HGF receptor and VEGFR/KDR is measured in a DELFIA™ assay (Perkin Elmer). The substrate poly(Glu₄,Tyr) is immobilized onto black high-binding polystyrene 96-well plates. The coated plates are washed and stored at 4° C. During the assay, enzymes are pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 10 uM for C-Met (5× the Kₘ) and 0.6 uM for VEGFR/KDR (2× the Kₘ). Enzyme concentration is 25 nM (C-Met) or 5 nM (VEGFR/KDR). After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations and IC₅₀'s (concentration of compounds giving 50% inhibition of enzymatic activity) are determined.

### C-Met Phosphorylation Cell-Based Assay

This test measures the ability of compounds to inhibit HGF Stimulated Auto-phosphorylation of the c-Met/HGF receptor itself in a whole cell system.

MNNGHOS cell line expressing TPR-MET fusion protein are purchased from ATCC. The TPR-MET is the product of a chromosomal translocation placing the TPR locus on chromosome 1 upstream of the MET gene on chromosome 7 encoding for its cytoplasmic region catalytic domain. Dimerization of the Mᵣ 65,000 TPR-Met oncoprotein through a leucine zipper motif encoded by the TPR portion leads to constitutive activation of the met kinase. Constitutive autophosphorylation occurs on residues Tyr361/365/366 of TPR-Met. These residues are homologous to Tyr1230/1234/1235 of MET which become phosphorylated upon dimerization of the receptor upon HGF binding.

Inhibitor of c-Met formulated as 30 mM stocks in DMSO. For MNNGHOS treatments, cells, compounds are added to tissue culture media at indicated doses for 3 hours prior to cell lysis. Cells are lysed in ice-cold lysis buffer containing 50 mM HEPES (pH 7.5), 150 mM NaCl, 1.5 mM MgCl2, 10% glycerol, 1% Triton X-100, 1 mM 4-(2-Aminoethyl)benzenesulfonyl fluoride hydrochloride, 200 μM sodium orthovanadate, 1 mM sodium fluoride, 10 μg/ml of leupeptin, 10 μg/ml of aprotinin/ml, 1 ug/ml of pepstatin and 50 ug/ml Na-p-Tosyl-L-lysine chloromethyl ketone hydrochloride.

Lysate are separated on 5-20% PAGE-SDS and immunoblots are performed using Immobilon P polyvinylidene difluoride membranes (Amersham) according to the manufacturer's instructions for handling. The blots are washed in Tris-buffered saline with 0.1% Tween 20 detergent (TBST). Tyr361/365/366 of TPR-Met are detected with polyclonal rabbit antibodies against tyrosine phosphorylated Met (Biosource International) and secondary antibodies anti-rabbit-horseradish peroxidase (Sigma) by chemiluminescence assays (Amersham, ECL) performed according to the manufacturer's instructions and followed by film exposure. Signal is quantitated by densitometry on Alpha-Imager. IC₅₀ values, as shown in Table 2, are defined as the dose required to obtain 50% inhibition of the maximal HGF stimulated phosphorylated c-Met level.

### In Vivo Solid Tumor Disease Model

This test measures the capacity of compounds to inhibit solid tumor growth.

Tumor xenografts are established in the flank of female athymic CD1 mice (Charles River Inc.), by subcutaneous injection of 1×10⁶ U87, A431 or SKLMS cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of ˜30 mg from donor tumors. When the tumors are approximately 100 mm³ in size (˜7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)³ 4/3π. The experiment is terminated when the control tumors reach a size of approximately 1500 mm³. In this model, the change in mean tumor volume for a compound treated group/the change in mean tumor volume of the control group (non-treated or vehicle treated)×100 (ΔT/ΔC) is subtracted from 100 to give the percent tumor growth inhibition (% TGI) for each test compound. In addition to tumor volumes, body weight of animals is monitored twice weekly for up to 3 weeks. The activities of a number of compounds according to the invention measured by various assays are displayed in the following table.

In the table, for potency in enzyme assays, “a” indicates inhibitory activity at a concentration of less than 250 nanomolar; “b” indicates inhibitory activity at a concentration ≧250 but <500 nanomolar, “c” indicates inhibitory activity at ≧500 but <1000 nanomolar; “d” indicates inhibitory activity ≧1000 nanomolar, and “e” indicates no activity as measured by that assay.

For potency in cell-based assays, “A” indicates inhibitory activity at a concentration of ≧1 μM; “B” indicates inhibitor activity at a concentration >1 μM but ≦5 μM; “C” indicates inhibitor activity at a concentration of >5 μM but ≦10 μM; and “D” indicates inhibitory activity >10 μM.

TABLE 2
Profile of selected compounds

| | | | | | Potency in cell-based | |
|---|---|---|---|---|---|---|
| | | | | | assays | |
| | | | | | A549 | |
| | | | Potency in | | wound | DU145 |
| | Ex- | | enzyme assays | | healing | scattering |
| Cpd | ample | | Cmet | VEGF | inhibition | inhibition |
| No. | No | Structure | (μM) | (μM) | (μM) | (μM) |
| 9a | 1 | 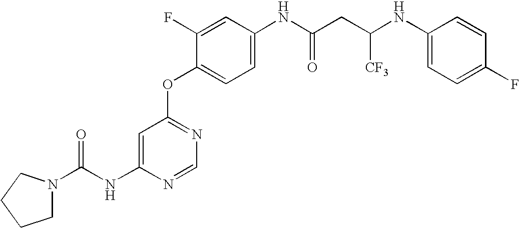 | d | d | B | C |
| 9b | 2 | 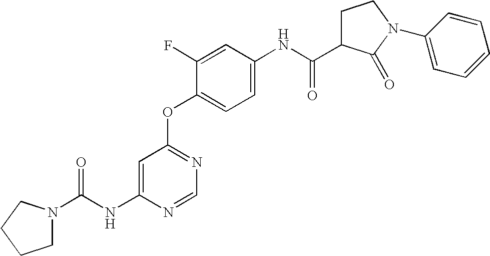 | d | d | C | C |
| 9c | 3 | 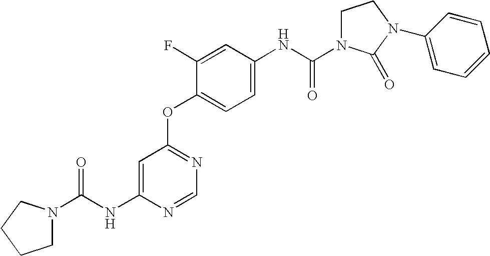 | a | a | B | C |
| 13 | 4 | 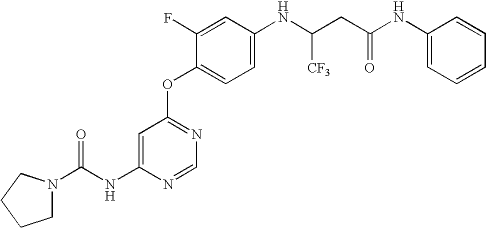 | d | a | C | C |
| 18a | 5 | 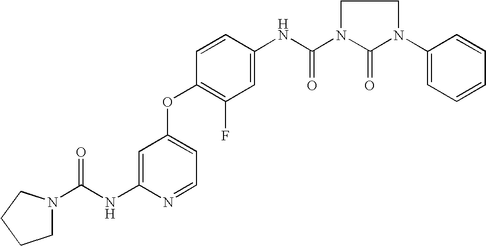 | a | b | A | A |
| 18b | 6 | 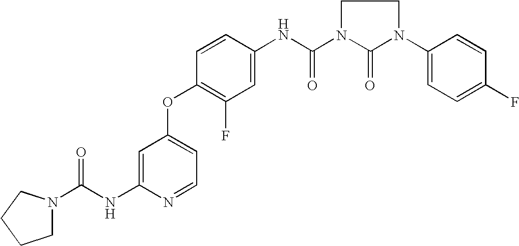 | a | a | A | A |
| 87a | 70 | 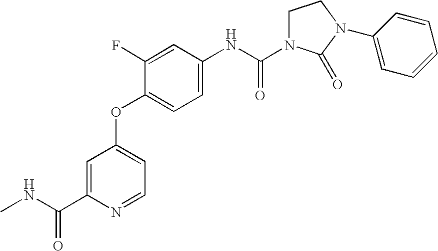 | b | b | B | C |
| 61a | 58 | 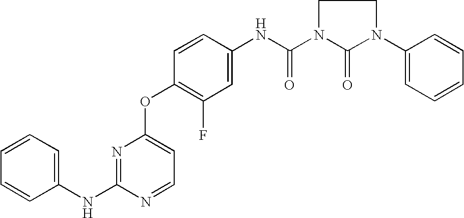 | c | d | C | D |
| 21c | 16 | 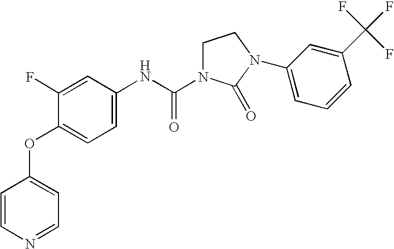 | d | d | C | C |
| 61b | 59 | 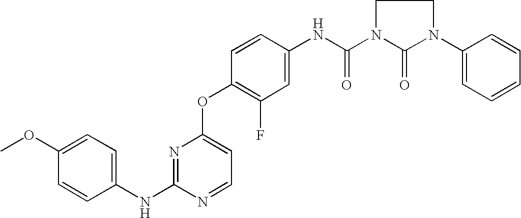 | c | d | C | C |
| 21f | 19 | 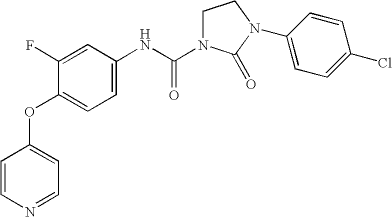 | b | d | B | C |
| 21g | 20 | 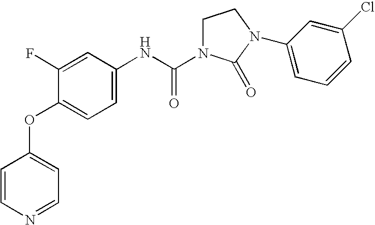 | c | d | C | C |
| 87c | 72 | 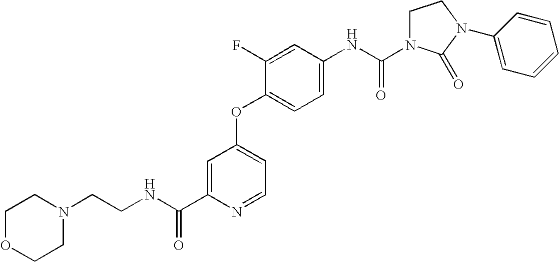 | d | d | D | D |
| 21h | 21 | 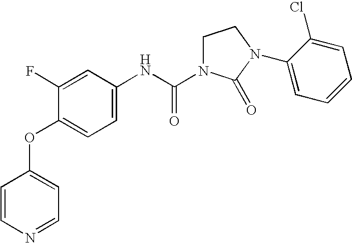 | c | d | B | A |
| 87b | 71 | 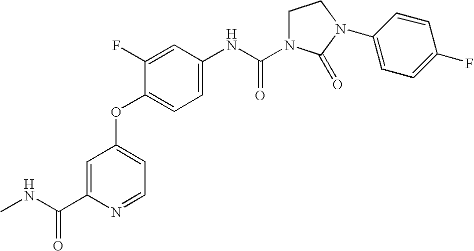 | c | d | B | B |
| 87d | 73 | 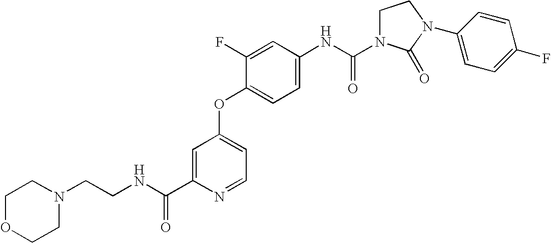 | d | D | n/d | n/d |
| 18c | 7 | 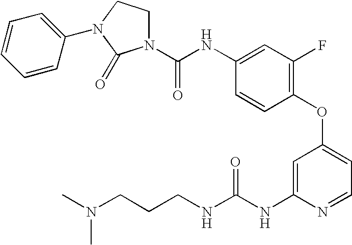 | c | d | B | A |
| 18d | 8 | 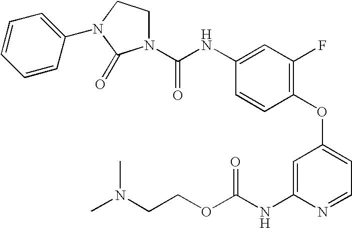 | b | d | D | D |
| 70 | 61 | 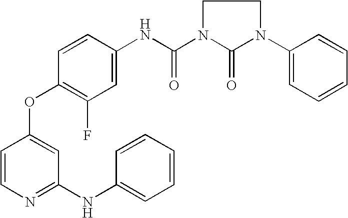 | a | d | B | C |
| 31a | 38 | 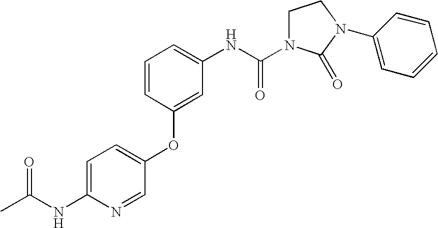 | b | d | B | C |
| 30a | 51 | 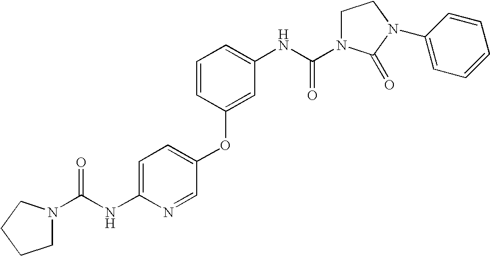 | d | d | n/d | n/d |
| 18e | 9 | 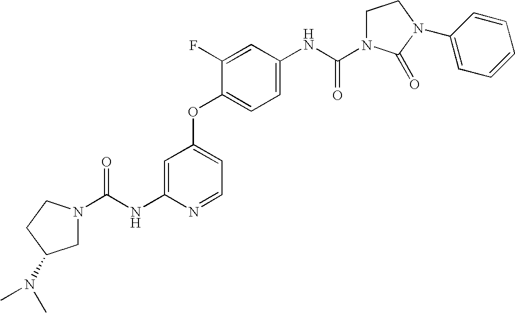 | a | b | A | A |
| 18f | 10 | 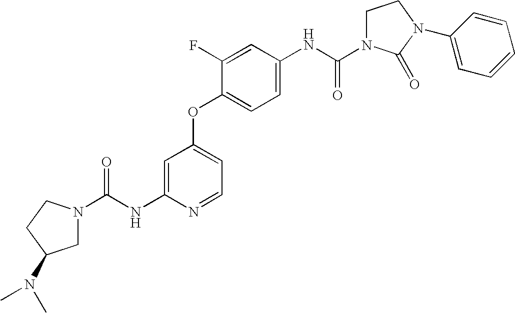 | a | a | B | A |
| 18g | 11 | 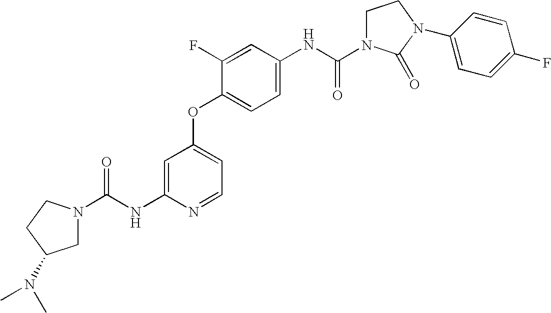 | a | a | A | A |
| 18h | 12 | 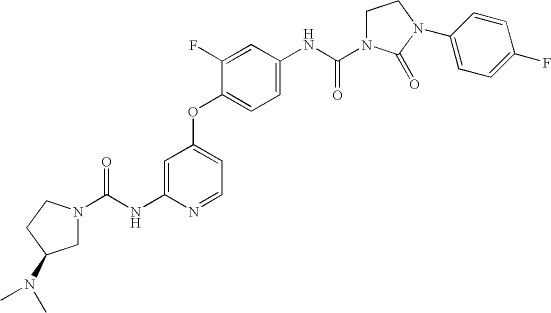 | a | a | A | A |
| 18i | 13 | 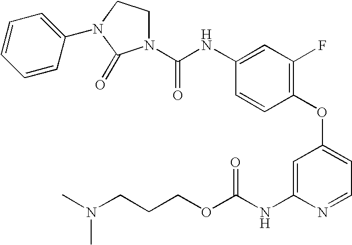 | a | d | B | B |

1. A compound of formula (A): 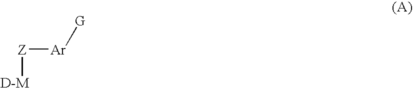
and N-oxides, hydrates, solvates, pharmaceutically acceptable salts, prodrugs and complexes thereof, and racemic and scalemic mixtures, diastereomers and enantiomers thereof, wherein,
M is an optionally substituted monocyclic moiety;
D is selected from the group consisting of R⁷, R¹ and R²¹, wherein
R⁷ is selected from the group consisting of —H, halogen, nitro, azido, C₁-C₆ alkyl, C₃-C₁₀ cycloalkyl, —C(O)NR⁴²R⁴³, —Y—NR⁴²R⁴³, —NR⁴²C(═O)R⁴³, —NR⁴²C(═O)NR⁴³, —NR⁴²C(═O)NR⁴³—R¹⁰¹, —SO₂R⁴², —SO₂NR⁴²R⁴³, —NR³⁷SO₂R⁴², —NR³SO₂NR⁴²R⁴³, —C(═N—OR⁴²)R⁴³, C(═NR⁴²)R⁴³, —NR³⁷C(═NR⁴²)R⁴³, —C(═NR⁴²)NR⁴²R⁴³, —NR³⁷C(═NR⁴²)NR³⁷R⁴³, —C(O)R⁴², —CO₂R¹², —C(O)(heterocyclyl), —C(O)(C₆-C₁₀ aryl), —C(O)(heteroaryl), —Y—(C₆-C₁₀ aryl), —Y-(heteroaryl), —Y-(5-10 membered heterocyclyl), —NR^{6a}R^{6b}, —NR^{6a}SO₂R^{6b}, —NR^{6a}C(O)R^{6b}, —OC(O)R^{6b}, —NR^{6a}C(O)OR^{6b}, —OC(O)NR^{6a}R^{6b}, —OR^{6a}, —SR^{6a}, —S(O)R^{6a}, —SO₂R^{6a}, —SO₃R^{6a}, —SO₂NR^{6a}R^{6b}, —SO₂NR⁴²R⁴³, —COR^{6a}, —CO₂R^{6a}, —CONR^{6a}R^{6b}, —(C₁-C₄)fluoroalkyl, —(C₁-C₄)fluoroalkoxy, —(CZ³Z⁴)ₙCN, wherein n is an integer ranging from 0 to 6, and the aforementioned R⁷ groups other than —H and halogen are optionally substituted by 1 to 5 R³⁸, or R⁷ is a moiety selected from the group consisting of —(CZ³Z⁴)ₐ-aryl, —(CZ³Z⁴)ₐ-heterocycle, (C₂-C₆)alkynyl, —(CZ³Z⁴)ₐ-(C₃-C₆)cycloalkyl, —(CZ³Z⁴)ₐ-(C₅-C₆)cycloalkenyl, (C₂-C₆) alkenyl and (C₁-C₆)alkyl, wherein said moiety is optionally substituted with 1 to 3 independently selected Y² groups, where a is 0, 1, 2, or 3, and wherein when a is 2 or 3, the CZ³Z⁴ units may be the same or different; wherein
each R^{6a} and R^{6b} is independently selected from the group consisting of hydrogen and a moiety selected from the group consisting of —(CZ⁵Z⁶)ᵤ-(C₃-C₆)cycloalkyl, —(CZ⁵Z⁶)ᵤ-(C₅-C₆)cycloalkenyl, —(CZ⁵Z⁶)ᵤ-aryl, —(CZ⁵Z⁶)ᵤ-heterocycle, (C₂-C₆)alkenyl, and (C₁-C₆)alkyl, wherein said moiety is optionally substituted with 1 to 3 independently selected Y³ groups, where u is 0, 1, 2, or 3, and wherein when u is 2 or 3, the CZ 5Z⁶ units may be the same or different, or
R^{6a} and R^{6b} taken together with adjacent atoms form a heterocycle;
each Z³, Z⁴, Z⁵ and Z⁶ is independently selected from the group consisting of H, F and (C₁-C₆)alkyl, or
each Z³ and Z⁴, or Z⁵ and Z⁶ are selected together to form a carbocycle, or
two Z³ groups on adjacent carbon atoms are selected together to optionally form a carbocycle;
each Y² and Y³ is independently selected from the group consisting of halogen, cyano, nitro, tetrazolyl, guanidino, amidino, methylguanidino, azido, —C(O)Z⁷, —OC(O)NH₂, —OC(O)NHZ⁷, —OC(O)NZ⁷Z⁸, —NHC(O)Z⁷, —NHC(O)NH₂, —NHC(O)NHZ⁷, —NHC(O)NZ⁷Z⁸, —C(O)OH, —C(O)OZ⁷, —C(O)NH₂, —C(O)NHZ⁷, —C(O)NZ⁷Z⁸, —P(OH)₃, —OP(OH)₃, —P(O)(OH)₂, OP(OZ⁷)₃, —S(O)₃H, —S(O)Z⁷, —S(O)₂Z⁷, —S(O)₃Z⁷, -Z⁷, —OZ⁷, —OH, —NH₂, —NHZ⁷, —NZ⁷Z⁸, —C(═NH)NH₂, —C(═NOH)NH₂, —N-morpholino, (C₂-C₆)alkenyl, (C₂-C₆)alkynyl, (C₁-C₆)haloalkyl, (C₂-C₆)haloalkenyl, (C₂-C₆)haloalkynyl, (C₁-C₆)haloalkoxy, —(CZ⁹Z¹⁰)ᵣNH₂, —(CZ⁹Z¹⁰)ᵣNHZ³, —(CZ⁹Z¹⁰)ᵣNZ⁷Z⁸, —X⁶(CZ⁹Z¹⁰)ᵣ-(C₃-C₈)cycloalkyl, —X⁶(CZ⁹Z¹⁰)ᵣ-(C₅-C₈)cycloalkenyl, —X⁶(CZ⁹Z¹⁰)ᵣ-aryl and —X⁶(CZ⁹Z¹⁰)ᵣheterocycle, wherein
r is 1, 2, 3 or 4; or
any two Y² or Y³ groups attached to adjacent carbon atoms may be taken together to be —O[C(Z⁹)(Z¹⁰)]ᵣO or —O[C(Z⁹)(Z¹⁰)]ᵣ₊₁, or
any two Y or Y groups attached to the same or adjacent carbon atoms may be selected together to form a carbocycle or heterocycle;
X⁶ is selected from the group consisting of O, S, NH, —C(O)—, —C(O)NH—, —C(O)O—, —S(O)—, S(O)₂— and —S(O)₃—;
Z⁷ and Z⁸ are independently selected from the group consisting of an alkyl of 1 to 12 carbon atoms, an alkenyl of 2 to 12 carbon atoms, an alkynyl of 2 to 12 carbon atoms, a cycloalkyl of 3 to 8 carbon atoms, a cycloalkenyl of 5 to 8 carbon atoms, an aryl of 6 to 14 carbon atoms, a heterocycle of 5 to 14 ring atoms, an aralkyl of 7 to 15 carbon atoms, and a heteroaralkyl of 5 to 14 ring atoms, or
Z⁷ and Z⁸ together may optionally form a heterocycle;
Z⁹ and Z¹⁰ are independently selected from the group consisting of H, halogen (preferably F), a (C₁-C₁₂)alkyl, a (C₆-C₁₄)aryl, a (C₅-C₁₄)heteroaryl, a (C₇-C₁₅)aralkyl and a (C₅-C₁₄)heteroaralkyl, or
Z⁹ and Z¹⁰ are taken together form a carbocycle, or
two Z⁹ groups on adjacent carbon atoms are taken together to form a carbocycle; and wherein
any of the above-mentioned substituents comprising a CH₃ (methyl), CH₂ (methylene), or CH (methine) group which is not attached to a halogen, SO or SO₂ group or to a N, O or S atom optionally bears on said group a substituent selected from hydroxy, halogen, (C₁-C₄)alkyl, (C₁-C₄)alkoxy and an —N[(C₁-C₄)alkyl][(C₁-C₄)alkyl];
R¹ is —C≡CH or —C≡C—(CR⁴⁵R⁴⁵)ₙ—R⁴⁶;
each R⁴⁵ is independently selected from the group consisting of H, a (C₁-C₆)alkyl and a (C₃-C₈)cycloalkyl;
R⁴⁶ is selected from the group consisting of heterocyclyl, —N(R⁴⁷)—C(O)—N(R⁴⁷)(R⁴⁸), —N(R⁴⁷)—C(S)—N(R⁴⁷)(R⁴⁸), —N(R⁴⁷)—C(O)—OR⁴⁸, —N(R⁴⁷)—C(O)—(CH₂)ₙ—R⁴⁸, —N(R⁴⁷)—SO₂R⁴⁷, —(CH₂)ₙNR⁴⁷R⁴⁸, -(CH₂)ₙOR⁴⁸, —(CH₂)ₙSR⁴⁹, —(CH₂)ₙS(O)R⁴⁹, —(CH₂)ₙS(O)₂R⁴⁹, —OC(O)R⁴⁹, —OC(O)OR⁴⁹, —C(O)NR⁴⁷R⁴⁸, heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁴⁷ and R⁴⁸ are independently selected from the group consisting of H, (C₁-C₆)alkyl, (C₃-C₈)cycloalkyl, heterocyclyl, —(CH₂)ₙNR⁵⁰R⁵¹, (CH₂)ₙOR⁵⁰, (CH₂)ₙC(O)R⁴⁹, —C(O)₂R⁴⁹, —(CH₂)ₙSR⁴⁹, —(CH₂)ₙS(O)R⁴⁹, —(CH₂)ₙS(O)₂R⁴⁹, —(CH₂)ₙR⁴⁹, —(CH₂)ₙCN, aryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —(CH₂)ₙOR⁴⁹, —(CH₂)ₙheterocyclyl, —(CH₂)ₙheteroaryl, —SO₂R⁵⁰ and —(CH₂)ₙNR⁵⁰R⁵¹, or
R⁴⁷ and R⁴⁸, together with the atom to which they are attached, form a 3-8 membered carbo- or hetero-cyclic ring;
R⁴⁹ is selected from the group consisting of (C₁-C₆)alkyl, (C₃-C₈)cycloalkyl, heterocyclyl(C₁-C₆)alkylene, aryl(C₁-C₆)alkylene wherein the aryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, heteroaryl(C₁-C₆)alkylene wherein the heteroaryl is optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, aryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹, and heteroaryl optionally substituted with one or more substituents selected from the group consisting of halo, —CF₃, (C₁-C₆)alkoxy, —NO₂, (C₁-C₆)alkyl, —CN, —SO₂R⁵⁰ and —(CH₂)ₙNR⁵⁰R⁵¹;
R⁵⁰ and R⁵¹ are independently selected from the group consisting of H, (C₁-C₆)alkyl, (C₃-C₈)cycloalkyl and —C(O)R⁴⁵, or
R⁵⁰ and R⁵¹ together with the atom to which they are attached, form a 3-8 membered carbo- or hetero-cyclic ring;
n is an integer ranging from 0 to 6; and
R²¹ is the group defined by -(Z¹¹)-(Z¹²)ₘ-(Z¹³)ₘ₁, wherein
Z¹¹ is heterocyclyl, when m and m1 are 0, or heterocyclylene, when either m or m1 are 1,
Z¹² is selected from the group consisting of OC(O), OC(S) and C(O);
Z¹³ is selected from the group consisting of heterocyclyl, aralkyl, N(H)R⁵², (C₁-C₃)alkyl, —OR⁵², halo, S(O)₂R⁵⁶, (C₁-C₃)hydroxyalkyl and (C₁-C₃)haloalkyl;
m is 0 or 1;
m1 is 0 or 1;
R⁵² is selected from the group consisting of H, —(CH₂)_{q}S(O)₂R⁵⁴, —(C₁-C₆) alkyl-NR⁵³R⁵³ (C₁-C₃)alkyl, —(CH₂)_{q}OR⁵³, —C(O)R⁵⁴ and —C(O)OR⁵³;
q is 0, 1, 2, 3 or 4;
each R⁵³ is independently (C₁-C₃)alkyl;
R⁵⁴ is (C₁-C₃)alkyl or N(H)R⁵³;
R⁵⁶ is selected from the group consisting of NH₂, (C₁-C₃)alkyl and OR⁵²;
Ar is a 5 to 7 membered cycloalkyl, aryl, heterocylic or heteroaryl ring system, any of which is optionally substituted with 0 to 4 R² groups;
R² at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T 2, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
G is a group B-L-T, wherein
B is selected from the group consisting of absent, —CH₂—NH—, —NH—CH₂—, —N(R¹³)—, —N(SO₂R¹³)—, —O—, —S(O)₀₋₂ and —C(═X)—;
L is selected from the group consisting of absent, —C(═X)N(R¹³)—, —SO₂N(R¹³)—, —SO₂—, —N(R¹³)—, —C(═X)C₁₋₂alkyl-N(R¹³)—, —N(R¹³)C₁₋₂alkyl-C(═X)—, —C(═X)C₀₋₁alkyl-C(═X)N(R¹³)—, —C₀₋₄alkylene, —C(═X)C₀₋₁alkyl-C(═X)O—, —C(═X)—C₀₋₁alkyl-C(═X)—, —C(═X)—, —C(═X)C₀₋₁alkyl-C(═X)—, —C(═X)—O—C(═X)— and an optionally substituted four to nine-membered heterocyclyl preferably containing between one and three annular heteroatoms and preferably including at least one nitrogen, and wherein an alkyl group of the aforementioned L group is optionally substituted; and
T is selected from the group consisting of —H, —R¹³, —C₀₋₅alkyl, —C₀₋₅alkyl-Q, —O—C₀₋₅alkyl-Q, —C₀₋₅alkyl-O-Q, —N(R¹³)—C₀₋₅alkyl-Q, —C₀₋₅alkyl-SO₂—C₀₋₅alkyl-Q, —C(═X)—C₀₋₅alkyl-Q, —C(═X)—C₀₋₅-alkyl-Q, —C(X)—C₀₋₅-alkyl-Q, —C₀₋₅alkyl-N(R¹³)-Q, —C(X)—N(R¹³)—C₀₋₅alkyl-Q, —C(X)—N(R¹³)—C₀₋₅alkyl-Q, —C(X)—N(R³)—C₀₋₅alkyl-Q, —(C₀₋₅alkyl-C(O))₀₋₁—C₀₋₅alkyl-Q wherein each C₀₋₅alkyl is optionally substituted;
wherein X is selected from the group consisting of O, S, NH, N-alkyl, N—OH, N—O-alkyl, and NCN;
or G is selected from the group consisting of 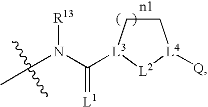
wherein
L¹ is selected from the group consisting of O, S and N(R¹⁴);
L² is selected from the group consisting of —C(O)—, —C(S)—, —C(NH)—, >C═N(C₁-C₆ alkyl) and —CH₂—;
L³ is selected from the group consisting of —CH—, —C(C₁-C₆ alkyl)- and N;
L⁴ is selected from the group consisting of —CH— and N; and
n1 is an integer from 0 to 5; 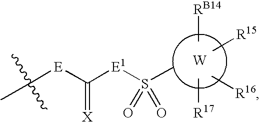
wherein
E is selected from the group consisting of —N(H)—, —N(C₁-C₆alkyl)-, —CH₂N(H)— and —N(H)CH₂—,
X is selected from the group consisting of O, S, NH, N-alkyl, N—OH, N—O-alkyl, and NCN,
E¹ is selected from the group consisting of —N(H)—, —N(C₁-C₆alkyl)-, —CH₂N(H)— and —N(H)CH₂—,
W is a five- to ten-membered cycloalkyl, aryl, heterocylic or heteroaryl ring system, which, is optionally substituted, and
R^{B14}, R¹⁵, R¹⁶ and R¹⁷ are independently selected from the group consisting of R²⁰; 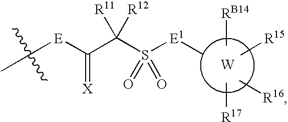
wherein
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl, or
R¹¹ and R¹² taken together with the atom to which they are attached form a C₃-C₇ ring system, wherein said ring system is optionally substituted; 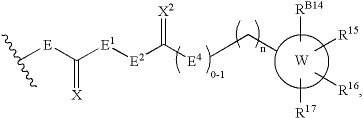
wherein
n is 0, 1, 2, 3 or 4;
X is selected from the group consisting of O, S, NH, NOH, NOMe, NOEt and NCN,
E is selected from the group consisting of —N(H)—, —N(C₁-C₆alkyl)-, —CH₂N(H)— and —N(H)CH₂—, and
E⁴ is —N(H)— or —N(C₁-C₆alkyl)-; and 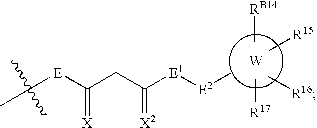
R¹³ is selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, —C(O)SR³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), —(CH₂)₀₋₅(cycloalkyl), C₁-C₆ alkyl, C₂-C₆ alkenyl, C₂-C₆ alkynyl, —CH₂(CH₂)₀₋₄-T 2, an optionally substituted C₁₋₄ alkylcarbonyl, and a saturated or unsaturated three- to seven-membered carboxyclic or heterocyclic group, wherein the aryl, heteroaryl, C₁-C₆ alkyl, C₂-C₆ alkenyl, and C₂-C₆ alkynyl are optionally substituted; wherein
two R³, together with the atom or atoms to which they are attached, can combine to form a heteroalicyclic optionally substituted with between one and four of R⁶⁰, wherein the heteroalicyclic can have up to four annular heteroatoms, and the heteroalicyclic can have an aryl or heteroaryl fused thereto, in which case the aryl or heteroaryl is optionally substituted with an additional one to four of R⁶⁰;
R¹⁴ is selected from the group —H, —NO₂, —NH₂, —N(R³)R⁴, —CN, —OR³, an optionally substituted (C₁-C₆)alkyl, an optionally substituted heteroalicyclylalkyl, an optionally substituted aryl, an optionally substituted arylalkyl and an optionally substituted heteroalicyclic,
each R³ is independently selected from the group consisting of —H and R⁴;
R⁴ is selected from the group consisting of a (C₁-C₆)alkyl, an aryl, a lower arylalkyl, a heterocyclyl and a lower heterocyclylalkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
R⁶⁰ is selected from the group consisting of —H, halogen, trihalomethyl, —CN, —NO₂, —NH₂, —OR³, —NR³R⁴, —S(O)₀₋₂R³, —SO₂NR³R³, —CO₂R³, —C(O)NR³R³, —N(R³)SO₂R³, —N(R³)C(O)R³, —N(R³)CO₂R³, —C(O)R³, an optionally substituted (C₁-C₆)alkyl, an optionally substituted aryl, an optionally substituted heteroarylalkyl and an optionally substituted arylalkyl; or
two R⁶⁰, when attached to a non-aromatic carbon, can be oxo;
Q is a five- to ten-membered ring system, optionally substituted with between zero and four of R²⁰;
R²⁰ is selected from the group consisting of —H, halogen, trihalomethyl, —O-trihalomethyl, oxo, —CN, —NO₂, —NH₂, —P(═O)(C₁-C₆alkyl)₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy and a saturated or unsaturated three- to seven-membered carboxyclic or heterocyclic group and wherein the aryl, heteroaryl, C₁-C₆ alkyl, C₂-C₆ alkenyl, and C₂-C₆ alkynyl are optionally substituted;
each R³⁸ is independently selected from halo, cyano, nitro, trifluoromethoxy, trifluoromethyl, azido, optionally substituted C₁-C₆ alkyl, —C(O)O—(CH₂)ₙNR³⁶R³⁹, —C(O)(CH₂)ⱼNR³⁹(CH₂)ₙR³⁶, —(CH₂)ₙP(═O)(C₁-C₆alkyl)₂, —(CH₂)ⱼNR³⁹CH₂(CH₂)ₙP(═O)(C₁-C₆alkyl)₂, —NR¹³C(X¹)NR¹³—C₁-C₆alkyl-P(═O)(C₁-C₆alkyl)₂, —NR¹³C(X³)NR¹³-arylP(═O)(C₁-C₆alkyl)₂ and —NR¹³C(X³)NR¹³-heteroarylP(═O)(C₁-C₆alkyl)₂, —(CH₂)ⱼNR³⁹(CH₂)ᵢ[O(CH₂)ᵢ]ₓ(CH₂)ⱼR⁹⁹, —(CH₂)ⱼNR³⁹(CH₂)ᵢSO₍₀₋₂₎(CH₂)ᵢ[O(CH₂)ᵢ]ⱼ(CH₂)ⱼR⁹⁹, —(CH₂)ⱼNR³⁹(CH₂)ⱼR¹⁰⁰, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹—OR³⁷, —SO₂NR³⁶R³⁹, C₁-C₆ alkyl, —(CH₂)ⱼO(CH₂)ᵢNR³⁶R³⁹, —(CH₂)ₙO(CH₂)ᵢOR³⁷, —(CH₂)ₙOR³⁷, —S(O)ⱼ(C₁-C₆ alkyl), —(CH₂)ₙ(C₆-C₁₀ aryl), —(CH₂)ₙ(5-10 membered heterocyclyl); —C(O)(CH₂)ₙ(C₆-C₁₀ aryl), —(CH₂)ₙO(CH₂)ⱼ(C₆-C₁₀ aryl), —(CH₂)ₙO(CH₂)ᵢ(5-10 membered heterocyclyl), —C(O)(CH₂)ₙ(5-10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR³⁹(CH₂)ₙR³⁶, —SO₂(CH₂)ₙ(C₆-C₁₁ aryl), —SO₂(CH₂)ₙ(5-10 membered heterocyclyl), —(CH₂)ₙNR³⁶R³⁹, —NR³⁷SO₂NR³⁶R³⁹, SO₂R³⁶, C₂-C₆ alkenyl, C₃-C₁₀ cycloalkyl and C₁-C₆ alkylamino, wherein j is an integer ranging from 0 to 4 and preferably 0-2, n is an integer ranging from 0 to 6, x is an integer ranging from 1-6 and preferably 2-3, and i is an integer ranging from 2 to 6, preferably 2-3, the —(CH₂)ᵢ— and —(CH₂)ₙ— moieties of the foregoing R³⁸ groups optionally include a carbon-carbon double or triple bond where n is an integer between 2 and 6, and the alkyl, aryl and heterocyclyl moieties of the foregoing R³⁸ groups are optionally substituted by one or more substituents independently selected from halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —(CH₂)ₙNR³⁶R³⁹, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙO(CH₂)ᵢOR³⁷, and —(CH₂)ₙOR³⁷;
X³ is selected from the group consisting of O, S, CH₂, N—CN, N—O-alkyl, NH and N(C₁-C₆alkyl);
each R³⁶ and R³⁹ is independently selected from the group consisting of H, —OH, C₁-C₆ alkyl, C₃-C₁₀ cycloalkyl, —(CH₂)ₙ(C₆-C₁₀ aryl), —(CH₂)ₙ(5-10 membered heterocyclyl), —(CH₂)ₙ—O—(CH₂)ᵢOR³⁷, —(CH₂)ₙCN(CH₂)ₙOR³⁷, —(CH₂)ₙCN(CH₂)ₙR³⁷, and —(CH₂)ₙA⁴R³⁷, wherein n is an integer ranging from 0 to 6 and i is an integer ranging from 2 to 6, A⁴ is selected from the group consisting of O, S, SO, SO₂, and the alkyl, aryl and heterocyclyl moieties of the foregoing R³⁶ and R³⁹ groups are optionally substituted by one or more substituents independently selected from —OH, halo, cyano, nitro, trifluoromethyl, azido, —C(O)R⁴⁰, —C(O)OR⁴⁰, —CO(O)R⁴⁰, —OC(O)OR⁴⁰, —NR³⁷C(O)R⁴¹, —C(O)NR³⁷R⁴¹, —NR³⁷R⁴¹, —C₁-C₆ alkyl, —(CH₂)ₙ(C₆-C₁₀ aryl), —(CH₂)ₙ(5 to 10 membered heterocyclyl), —(CH₂)ₙ—O—(CH₂)ᵢOR³⁷, and —(CH₂)ₙOR³⁷, with the proviso that when R³⁶ and R³⁹ are both attached to the same nitrogen, then R³⁶ and R³⁹ are not both bonded to the nitrogen directly through an oxygen;
each R⁴⁰ is independently selected from H, C₁-C₁₀ alkyl, —(CH₂)ₙ(C₆-C₁₀ aryl), C₃-C₁₀ cycloalkyl, and —(CH₂)ₙ(5-10 membered heterocyclyl), wherein n is an integer ranging from 0 to 6;
each R³⁷ and R⁴¹ is independently selected from H, OR³⁶, C₁-C₆ alkyl and C₃-C₁₀ cycloalkyl;
each R⁴² and R⁴³ is independently selected from the group consisting of H, C₁-C₆ alkyl, —Y—(C₃-C₁₀ cycloalkyl), —Y—(C₆-C₁₀ aryl), —Y—(C₆-C₁₀ heteroaryl), —Y-(5-10 membered heterocyclyl), —Y—O—Y¹—OR³⁷, —Y¹—CO₂—R³⁷, and —Y—OR³⁷;
Y is a bond or is —(C(R³⁷)(H))ₙ, wherein n is an integer ranging from 1 to 6;
Y¹ is —(C(R³⁷)(H))ₙ;
and the alkyl, cycloalkyl, aryl, heteroaryl and heterocyclyl moieties of the foregoing R⁴² and R⁴³ groups are optionally substituted by 1 or more substituents independently selected from R⁴⁴; or
R⁴² and R⁴³ taken together with the nitrogen to which they are attached form a C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring, wherein said C₅-C₉ azabicyclic, aziridinyl, azetidinyl, pyrrolidinyl, piperidinyl, piperazinyl, morpholinyl, thiomorpholinyl, isoquinolinyl, or dihydroisoquinolinyl ring are optionally substituted by 1 to 5 R⁴⁴ substituents, with the proviso that R⁴² and R⁴³ are not both bonded to the nitrogen directly through an oxygen;
each R⁴⁴ is independently selected from the group consisting of halo, cyano, nitro, trifluoromethoxy, trifluoromethyl, azido, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹, —NR³⁶R³⁹, —OR³⁷, —SO₂NR³⁶R³⁹, —SO₂R³⁶, —NR³⁶SO₂R³⁹, —NR³⁶SO₂NR³⁷R⁴¹, C₁-C₆ alkyl, C₂-C₆ alkenyl, C₂-C₆ alkynyl, C₃-C₁₀ cycloalkyl, —C₁-C₆ alkylamino, —(CH₂)ⱼO(CH₂)ᵢNR³⁶R³⁹, —(CH₂)ₙ—O—(CH₂)ᵢOR³⁷, —(CH₂)ₙOR³⁷, —S(O)(C₁-C₆ alkyl), —(CH₂)ₙ(C₆-C₁₀ aryl), —(CH₂)ₙ(5-10 membered heterocyclyl), —C(O)(CH₂)ₙ(C₆-C₁₀ aryl), —(CH₂)ₙO(CH₂)j(C₆-C₁₀ aryl), —(CH₂)ₙO(CH₂)ᵢ(5 to 10 membered heterocyclyl), —C(O)(CH₂)ₙ(5 to 10 membered heterocyclyl), —(CH₂)ⱼNR³⁹(CH₂)ᵢNR³⁶R³⁹, —(CH₂)ⱼNR³⁹CH₂C(O)NR³⁶R³⁹, —(CH₂)ⱼNR³⁹(CH₂)ᵢNR³⁷C(O)R⁴⁰, —(CH₂)ⱼNR³⁹(CH₂)ₙO(CH₂)ᵢOR³⁷, —(CH₂)ⱼNR³⁹(CH₂)ᵢS(O)ⱼ(C₁-C₆ alkyl), —(CH₂)ⱼNR⁹(CH₂)ₙR³⁶, —SO₂(CH₂)ₙ(C₆-C₁₀ aryl), and —SO₂(CH₂)ₙ(5 to 10 membered heterocyclyl) wherein, j is an integer from 0 to 2, n is an integer from 0 to 6 and i is an integer ranging from 2 to 6, the —(CH₂)ᵢ— and —(CH₂)ₙ— moieties of the foregoing R⁴⁴ groups optionally include a carbon-carbon double or triple bond wherein n is then an integer from 2 to 6, and the alkyl, aryl and heterocyclyl moieties of the foregoing R⁴⁴ groups are optionally substituted by 1 or more substituents independently selected from the group consisting of halo, cyano, nitro, trifluoromethyl, azido, —OH, —C(O)R⁴⁰, —C(O)OR⁴⁰, —OC(O)R⁴⁰, —OC(O)OR⁴⁰, —NR³⁶C(O)R³⁹, —C(O)NR³⁶R³⁹—(CH₂)ₙNR³⁶R³⁹, —SO₂R³⁶, —SO₂NR³⁶R³⁹, C₁-C₆ alkyl, C₃-C₁₀ cycloalkyl, —(CH₂)ₙ(C₆-C₁₀ aryl), —(CH₂)ₙ(5 to 10 membered heterocyclyl), —(CH₂)ₙO(CH₂)ᵢOR³⁷ and —(CH₂)ₙOR³⁷;
Z is selected from the group consisting of covalent bond, —O—, —O—CH₂—, —CH₂—O—, —S—, —CH₂—, —N(R⁵)—, —N(R⁵)—CH₂—, —CH₂—N(R⁵)—, —N(R⁵)—C(O)—N(R⁵)—, C₂alkynylene, —N(R⁵)—C(O)—, —C(O)—N(R⁵)—, —N(R⁵)—SO₂— and —SO₂—N(R⁵)—, wherein R⁵ is selected from the group consisting of H, an optionally substituted (C₁-C₅)acyl and C₁-C₆ alkyl-O—C(O), wherein C₁-C₆ alkyl is optionally substituted;
R⁹⁹ at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, P(═O)(OH)₂, —P(═O)(C₁-C₆alkyl)₂, —SO₃H—C(O)R³, C₁-C₄ alkoxy, C₁-C₄ alkylthio, —O(CH₂)₀₋₆aryl, —O(CH₂)₀₋₆heteroaryl, —(CH₂)₀₋₅(aryl), —(CH₂)₀₋₅(heteroaryl), -, C₁-C₆ alkyl, C₂-C₆ alkenyl, C₂-C₆ alkynyl, —CH₂(CH₂)₀₋₄-T², wherein the aryl, heteroaryl, C₁-C₆ alkyl, C₂-C₆ alkenyl, and C₂-C₆ alkynyl are optionally substituted;
R¹⁰⁰ is a 12 to 24-membered optionally substituted heteroalicyclic macrocycle containing 4 to 8 oxygen atoms, preferentially 15-crown-5, 18-crown-6, or 21-crown-7; and
R¹⁰¹ is selected from the group consisting of H, C₁-C₆alkyl, C₂-C₆alkenyl, —C₁-C₆alkyl-heterocycle and C₁-C₆alkyl-P(O)(C₁-C₆alkyl)₂,
with the proviso that when B is —N(R¹³)—; L is —C(═O)N(R¹³)— or —C(S)—N(R¹³); T is C(═O)-Q; R¹³ is H or C₁₋₆alkyl; R²⁰ is other than trihalomethyl, —O-trihalomethyl, —N(R³)C(O)OR³, C(O)SR³, —O—(CH₂)₀₋₆aryl and —O—(CH₂)₀₋₆heteroaryl; and D-NHC(O)R^{p1}, then R^{p1} is not C₁₋₆alkyl, C₂₋₆alkenyl, C₂₋₆alkynyl, C₃₋₁₀cycloalkyl, C₆₋₁₀aryl, C₁₋₆alkoxy, 5- to 10-membered heteoraryl, 3- to 10-membered non-aromatic heterocyclic group or a group represented by the formula —NR^{p2}R^{p3}, wherein R^{p2} and R^{p3} may be the same or different and each represents H, C₁₋₆alkyl, C₃₋₆alkenyl, C₃₋₆alkynyl, C₃₋₁₀cycloalkyl, C₆₋₁₀aryl, C₁₋₆alkoxy, 5- to 10-membered heteroaryl or a 4- to 1-membered non-aromatic heterocyclic group, and wherein R^{p1}, R^{p2} and R^{p3} are optionally substituted;
and with the proviso that Formula (A) excludes those compounds wherein Z is O or —CH₂—O—; and Ar is 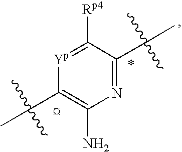
wherein [symbol] represents the point of attachment to Z, and * represents the point of attachment to G; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; when Y^{p} is —N(R³)CO₂R³; or when L² is —C(O)—, —C(S)—, —C(NH)— or >C═N(₁₋₆alkyl); and
with the proviso that Formula (A) excludes those compounds having the following structures 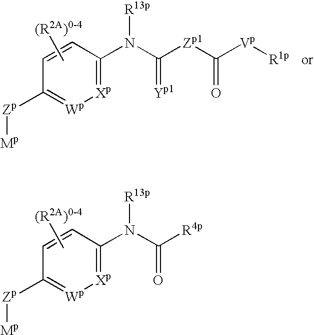
wherein Mp is selected from the group consisting of 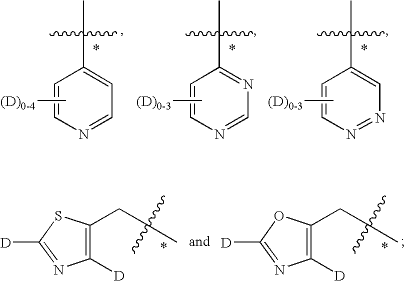
D is selected from the group consisting of H, halogen, NR^{p5}R^{p6}, OR^{p7}, CO2R^{p8}, CONR^{p9}R^{p10}, SO2R^{p11}, alkyl, cycloalkyl, alkenyl, alkynyl, CN, aryl, heteroaryl and heterocycloalkyl, wherein the alkyl, cycloalkyl, alkenyl, alkynyl, aryl, heteroaryl and heterocycloalkyl are optionally substituted; wherein R^{p5} to R^{P11} are independently selected from the group consisting of H, alkyl, alkenyl, alkynyl, cycloalkyl, alkoxycarbonyl, aryl, heteroaryl, heterocyclo and heterocycloalkyl, wherein the alkyl, alkenyl, alkynyl, cycloalkyl, aryl, heteroaryl, heterocyclo and heterocycloalkyl are optionally substituted; Z^{p} is selected from the group consisting of O, S and NH; W^{p} and X^{p} are each independently C or N; each R^{2A} is independently H, halogen, cyano, NO₂, OR^{p5}, NR^{p6}R^{p7}, alkyl, cycloalkyl, aryl, heteroaryl, heterocyclo, aryalkyl and heterocycloalkyl, wherein each of the alkyl, cycloalkyl, aryl, heteroaryl, heterocyclo, aryalkyl and heterocycloalkyl are optionally substituted; Y^{p1} is O, S and NP¹⁴ when Z comprises an N; or Y^{P1} is O when Z is alkyl or substituted alkyl; V^{p} is NR^{11p} or —(CR^{37p}R^{38p})₁₋₄, wherein if V^{p} is NR^{11p} then R^{1p} is alkyl or cycloalkyl; R^{11p} and R^{13p} are independently selected from the group consisting of H, alkyl, alkenyl, alkynyl, cycloalkyl, aryl, heteroaryl and heterocyclo, each of which is optionally substituted; R^{1p} is H, alkyl, cycloalkyl, arylalkyl, aryl, alkenyl, alkynyl, heteroaryl, heterocyclo, heteroarylalkyl, heterocycloalkyl, each of which is optionally substituted; R^{37p} and R^{38p} are independently selected from H, halogen and alkyl; and R^{4p} is selected from the group consisting of aryl, heteroaryl, heterocycloalkyl, each of which is optionally substituted; and
with the proviso that Formula (A) excludes those compounds wherein M is an optionally substituted pyrrole or an optionally substituted imidazole, Z is a covalent bond, and Ar is an optionally substituted pyrazole;
with the proviso that Formula (A) excludes 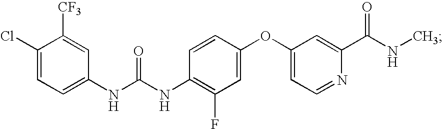
and
with the proviso that Formula (A) excludes those compounds wherein M is six-membered aryl or heteroaryl, wherein the heteroatom is N, and wherein M is optionally substituted with alkyl, alkenyl, alhythio, mercapto, free, etherified or esterified hydroxyl, unsubstituted, mono or disubstituted amino, or halogen; Z is —O—, —S— or —NH—; Ar is an optionally substituted pyridine; and G is —N(R³³¹)—(CH₂)₀₋₂—Y³³¹ or —N(R³³¹)—(C(alkyl)(alkyl))₀₋₂—Y³³¹; wherein R³³¹ is H or alkyl and Y³³¹ is H, aryl, heterocyclic or optionally substituted cycloalkyl; and
with the proviso that Formula (A) excludes those compounds wherein (1) M is pyridine substituted with morpholinyl, NHC(O)C₁₋₆alkyl or O-phenyl, wherein said phenyl is optionally substituted with C₁₋₆alkyl, C₁₋₆alkoxy, halo or CF₃; Z is NH; Ar is pyrimidine substituted with halo; and G is —N(H)—(CH₂)₀₋₂-phenyl, wherein said phenyl is substituted with 1 or 2 substituents independently selected from SO₂NH₂ and halo; and (2) M is phenyl substituted with a substituent selected from —C(O)OH, —NHC(O)-phenyl, a five membered heterocycle and imadazol[1,2-a]pyridinyl; Z is —NH—; Ar is pyrimidine substituted with halo; and G is —N(H)-pyridine-O-phenyl, wherein said phenyl is substituted with one of H, C₁₋₆alkoxy, CF₃ or halo; and
with the proviso that Formula (A) excludes those compounds wherein D is —C(O)—NR⁴²R⁴³ or —C(O)NR^{6a}R^{6b}; M is phenyl optionally substituted with halogen or alkyl; Z is —NH—; and G is pyrimidine-pyridine; and
with the proviso that Formula (A) excludes those compounds wherein Z is selected from the group consisting of —O—, —O—CH₂—, —CH₂—O—, —S—, —CH₂—, —N(H)—, —N(H)—CH₂— and —CH₂—N(H)—; and G is selected from the group consisting of —N(R¹³)—C(O)—C(O)—N(R¹³)-Q, —N(R¹³)—C(═NR¹⁴)—C(O)—N(R¹³)-Q, —N(R¹³)—C(O)—C(S)—N(R¹³)-Q and —N(R¹³)—C(O)—C(═NR¹⁴)—N(R¹³)-Q.

2. The compound according to claim 1, of Formula (B): 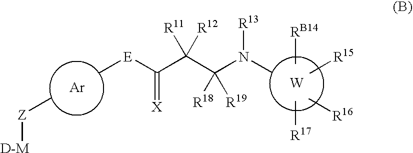
and N-oxides, hydrates, solvates, pharmaceutically acceptable salts, prodrugs and complexes thereof, and racemic and scalemic mixtures, diastereomers and enantiomers thereof, wherein,
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; or
R¹² and R¹³ taken together with the atoms to which they are attached optionally form a 4 to 8 membered cycloalkyl or heterocyclic ring system, which ring system is optionally substituted; or
R¹³ and R^{B14} taken together with the atoms to which they are attached optionally form a 4 to 8 membered cycloalkyl or heterocyclic ring system, which ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F.

3. The compound of claim 1, of Formula (C): 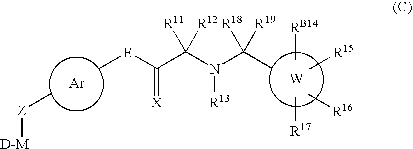
and N-oxides, hydrates, solvates, pharmaceutically acceptable salts, prodrugs and complexes thereof, and racemic and scalemic mixtures, diastereomers and enantiomers thereof, wherein
R¹¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F.

4. The compound according to claim 1, of Formula (D): 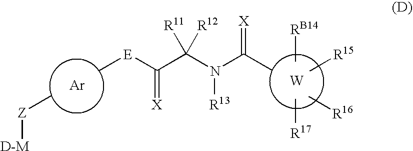
and N-oxides, hydrates, solvates, pharmaceutically acceptable salts, prodrugs and complexes thereof, and racemic and scalemic mixtures, diastereomers and enantiomers thereof.

5. The compound according to claim 1, of Formula (E): 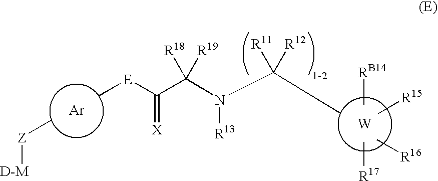
and N-oxides, hydrates, solvates, pharmaceutically acceptable salts, prodrugs and complexes thereof, and racemic and scalemic mixtures, diastereomers and enantiomers thereof, wherein
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F.

6. The compound according to claim 1, of Formula (F): 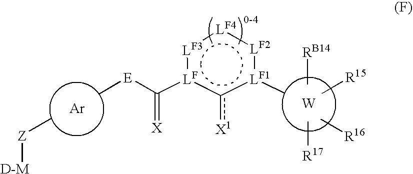
and N-oxides, hydrates, solvates, pharmaceutically acceptable salts, prodrugs and complexes thereof, and racemic and scalemic mixtures, diastereomers and enantiomers thereof, wherein [symbol] is a single or double bond;
X¹ is selected from the group consisting of O, S, CH₂, N—CN, N—O-alkyl, NH and N(C₁-C₆alkyl) when [symbol] is a double bond, or
X¹ is selected from the group consisting of H, halogen, alkyl, alkenyl, alkynyl, CN, alkoxy, NH(alkyl) and alkyl-thio, when [symbol] is a single bond;
L^{F} and L^{F1} are independently selected from the group consisting of —CH—, —N—, —C(halogen)- and —C(C₁-C₆alkyl)-;
L^{F2} and L^{F3} are independently selected from the group consisting of CH, CH₂, N, O and S;
L^{F4} is selected from the group consisting of absent, CH, CH₂, N, O and S; and
the group 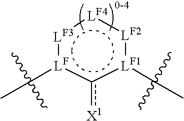
is aromatic or non-aromatic, provided that two 0 are not adjacent to each other;
and with the proviso that Formula (F) excludes those compounds wherein Z is O or —CH₂—O—; Ar is 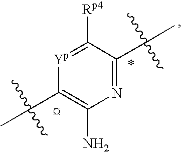
wherein [symbol] represents the point of attachment to Z, and * represents the point of attachment to E; E is —N(H)— or —N(alkyl)-; X is O; [symbol] is a single bond; and X¹ is H, halogen, alkyl, alkenyl, alkynyl, CN, alkoxy; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³;
with the proviso that Formula (F) excludes those compounds having the following structure 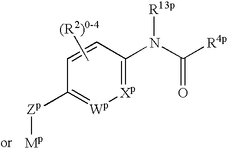
wherein Mp is selected from the group consisting of 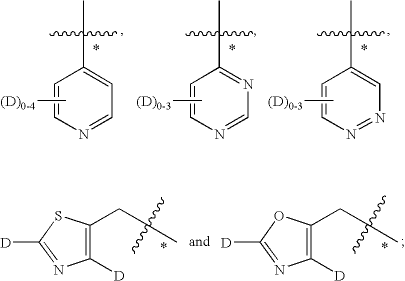
D is selected from the group consisting of H, halogen, NR^{p5}R^{p6}, OR^{p7}, CO2R^{p8}, CONR^{p9}R^{p10}, SO2R^{p11}, alkyl, cycloalkyl, alkenyl, alkynyl, CN, aryl, heteroaryl and heterocycloalkyl, wherein the alkyl, cycloalkyl, alkenyl, alkynyl, aryl, heteroaryl and heterocycloalkyl are optionally substituted; wherein R^{p5} to R^{p11} are independently selected from the group consisting of H, alkyl, alkenyl, alkynyl, cycloalkyl, alkoxycarbonyl, aryl, heteroaryl, heterocyclo and heterocycloalkyl, wherein the alkyl, alkenyl, alkynyl, cycloalkyl, aryl, heteroaryl, heterocyclo and heterocycloalkyl are optionally substituted; Z^{p} is selected from the group consisting of O, S and NH; W^{p} and X^{p} are each independently C or N; each R² is independently H, halogen, cyano, NO₂, OR^{p5}, NR^{p6}R^{p7}, alkyl, cycloalkyl, aryl, heteroaryl, heterocyclo, aryalkyl and heterocycloalkyl, wherein each of the alkyl, cycloalkyl, aryl, heteroaryl, heterocyclo, aryalkyl and heterocycloalkyl are optionally substituted; R^{13p} is selected from the group consisting of H, alkyl, alkenyl, alkynyl, cycloalkyl, aryl, heteroaryl and heterocyclo, each of which is optionally substituted; and R^{4p} is selected from the group consisting of aryl, heteroaryl, heterocycloalkyl, wherein the aryl is optionally substituted with halogen, alkyl, alkoxy, amino, cycloalkyl, aryl, heteroaryl, cyano, alkyl S(O)₀₋₂ or thiol, the heteroaryl is optionally substituted with halogen, alkyl, alkenyl, alkynyl, aryl, cyano, alkoxy, thioalkyl, ═O, phenyl, benzyl, phenylethyl, phenyloxy, phenylthio, cycloalkyl, heterocyclo, heteroaryl and NH(alkyl), and the heterocycloalkyl is optionally substituted with alkyl, alkoxy, nitro, monoalkylamino, dialkylamino, cyano, halo, haloalkyl, alkanoyl, aminocarbonyl, monoalkylaminocarbonyl, dialkylaminocarbonyl, alkyl amido, alkoxyalkyl, alkoxycarbonyl, alkylcarbonyloxy and aryl, said aryl further optionally substituted with halo, C₁₋₆alkyl or C₁₋₆alkoxy; and
with the proviso that Formula (F) excludes those compounds wherein M is an optionally substituted pyrrole or an optionally substituted imidazole, Z is a covalent bond, and Ar is an optionally substituted pyrazole.

7. The compound according to claim 1, of Formula (G): 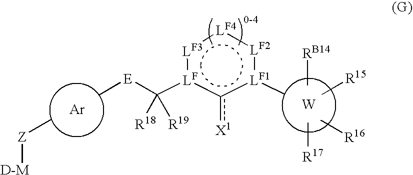
and N-oxides, hydrates, solvates, pharmaceutically acceptable salts, prodrugs and complexes thereof, and racemic and scalemic mixtures, diastereomers and enantiomers thereof, wherein
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F. [symbol] is a single or double bond;
X¹ is selected from the group consisting of O, S, CH₂, N—CN, N—O-alkyl, NH and N(C₁-C₆alkyl) when [symbol] is a double bond or
X¹ is selected from the group consisting of H, halogen, alkyl, alkenyl, alkynyl, CN, alkoxy, NH(alkyl) and alkyl-thio, when [symbol] is a single bond;
L^{F} and L^{F1} are independently selected from the group consisting of —CH—, —N—, —C(halogen)- and —C(C₁-C₆alkyl)-;
L^{F2} and L^{F3} are independently selected from the group consisting of CH, CH₂, N, O and S;
L^{F4} is selected from the group consisting of absent, CH, CH₂, N, O and S; and
the group 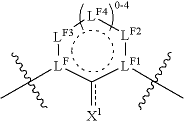
is aromatic or non-aromatic, provided that two 0 are not adjacent to each other;
and with the proviso that Formula (G) excludes those compounds wherein Z is O or —CH₂—O—; Ar is 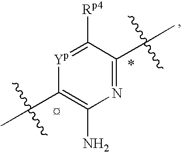
wherein [symbol] represents the point of attachment to Z, and * represents the point of attachment to E; E is —N(H)— or —N(alkyl)-; [symbol] is a single bond; and X¹ is H, halogen, alkyl, alkenyl, alkynyl, CN, alkoxy; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

8. The compound according to claim 1, of Formula (H): 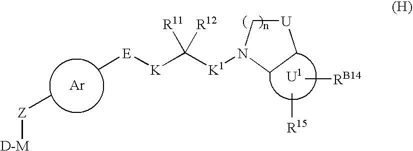
and N-oxides, hydrates, solvates, pharmaceutically acceptable salts, prodrugs and complexes thereof, and racemic and scalemic mixtures, diastereomers and enantiomers thereof, wherein
K and K¹ are independently selected from the group consisting of —C(O)—, —C(S)—, —C(NH)—, —C(NCN)— and —C(R¹⁸R¹⁹)—;
wherein
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F;
U is selected from the group consisting of O, S, SO₂, NH, and N(C₁-C₆alkyl), wherein the C₁-C₆alkyl is optionally substituted with a substituent selected from the group consisting of —OH, -alkoxy, amino, NH(C₁-C₆alkyl), N(C₁-C₆alkyl)₂, 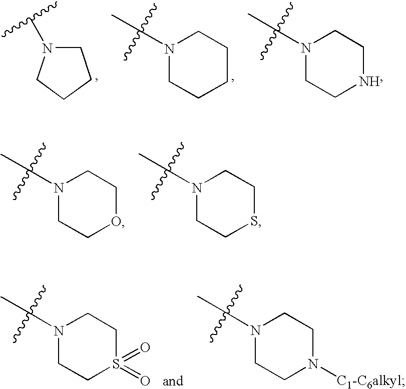
and
U¹ is a ring system selected from the group consisting of cycloalkyl, substituted cycloalkyl, heterocyclyl, substituted heterocyclyl, aryl, substituted aryl, heteroaryl and substituted heteroaryl;
and with the proviso that Formula (H) excludes those compounds wherein Z is 0 or —CH₂—O—; Ar is 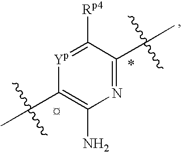
wherein [symbol] represents the point of attachment to Z, and * represents the point of attachment to E; E is —N(H)— or —N(alkyl)-; K is C(O) and K¹ is —C(R¹⁸R¹⁹)—, or K and K¹ are both —C(R¹⁸R¹⁹)—; and R¹⁸ and R¹⁹ are independently selected from the group consisting of H, halogen, —O-alkyl, alkyl, fluorinated alkyl and CN; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

9. The compound according to claim 1, of Formula (I): 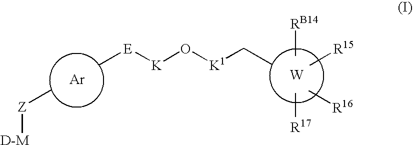
and N-oxides, hydrates, solvates, pharmaceutically acceptable salts, prodrugs and complexes thereof, and racemic and scalemic mixtures, diastereomers and enantiomers thereof, wherein
K and K¹ are independently selected from the group consisting of —C(O)—, —C(S)—, —C(NH)—, —C(NCN)— and —C(R¹⁸R¹⁹)—;
and with the proviso that Formula (I) excludes those compounds wherein Z is O or —CH₂—O—; Ar is 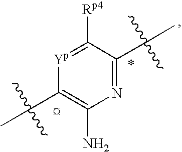
wherein [symbol] represents the point of attachment to Z, and * represents the point of attachment to E; E is —N(H)— or —N(alkyl)-; K and K¹ are both —C(R¹⁸R¹⁹)—; and R¹⁸ and R¹⁹ are independently selected from the group consisting of H, halogen, —O-alkyl, alkyl, fluorinated alkyl and CN; with the further proviso that compounds are not excluded when R^{p4} is H, halogen, —NH₂, —NR³R⁴, —N(R³)SO₂R⁵, —N(R³)CO₂R³, C₁₋₄alkoxy and C₁₋₄alkylthio; or when Y^{p} is —N(R³)CO₂R³.

10. The compound according to claim 1, of Formula (J): 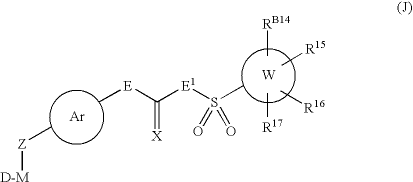
and N-oxides, hydrates, solvates, pharmaceutically acceptable salts, prodrugs and complexes thereof, and racemic and scalemic mixtures, diastereomers and enantiomers thereof.

11. The compound according to claim 1, of Formula (K): 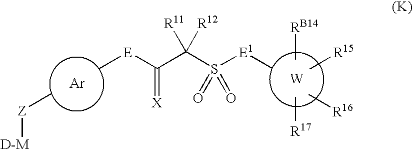
and N-oxides, hydrates, solvates, pharmaceutically acceptable salts, prodrugs and complexes thereof, and racemic and scalemic mixtures, diastereomers and enantiomers thereof.

12. The compound according to claim 1, of Formula (L): 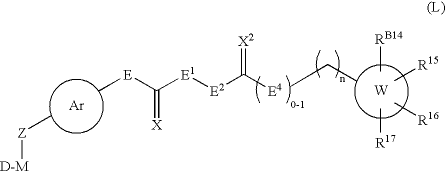
and N-oxides, hydrates, solvates, pharmaceutically acceptable salts, prodrugs and complexes thereof, and racemic and scalemic mixtures, diastereomers and enantiomers thereof,
n is 0, 1, 2, 3 or 4;
X² is selected from the group consisting of O, S, NH, NOH, NOMe, NOEt and NCN;
E¹ and E² are independently selected from the group consisting of —N(H)—, —N(C₁-C₆alkyl)-, —CH₂N(H)— and —N(H)CH₂—; and
E⁴ is —N(H)— or —N(C₁-C₆alkyl)-.

13. The compound according to claim 1, of Formula (M): 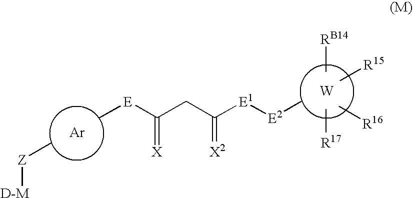
and N-oxides, hydrates, solvates, pharmaceutically acceptable salts, prodrugs and complexes thereof, and racemic and scalemic mixtures, diastereomers and enantiomers thereof, wherein
X² is selected from the group consisting of O, S, NH, NOH, NOMe, NOEt and NCN; and
E¹ and E² are independently selected from the group consisting of —N(H)—, —N(C₁-C₆alkyl)-, —CH₂N(H)— and —N(H)CH₂—.

14. The compound according to claim 1, of Formula (N): 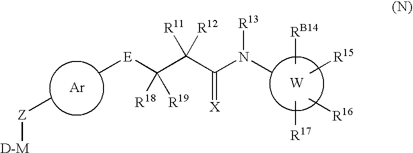
and N-oxides, hydrates, solvates, pharmaceutically acceptable salts, prodrugs and complexes thereof, and racemic and scalemic mixtures, diastereomers and enantiomers thereof, wherein
R¹ and R¹² are independently selected from the group consisting of H, halogen, —OH, unsubstituted —O—(C₁-C₆alkyl), substituted —O—(C₁-C₆alkyl), unsubstituted —O-(cycloalkyl), substituted —O-(cycloalkyl), unsubstituted —NH(C₁-C₆alkyl), substituted —NH(C₁-C₆alkyl), —NH₂, —SH, unsubstituted —S—(C₁-C₆alkyl), substituted —S—(C₁-C₆alkyl), unsubstituted C₁-C₆alkyl and substituted C₁-C₆alkyl; or
R¹¹ and R¹² taken together with the atom to which they are attached form a C₃-C₇ ring system, wherein said ring system is optionally substituted; or
R¹² and R¹³ taken together with the atoms to which they are attached optionally form a 4 to 8 membered cycloalkyl or heterocyclic ring system, which ring system is optionally substituted; or
R¹³ and R^{B14} taken together with the atoms to which they are attached optionally form a 4 to 8 membered cycloalkyl or heterocyclic ring system, which ring system is optionally substituted; and
R¹⁸ and R¹⁹ are independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F.

15. The compound according to claim 1, of Formula (O): 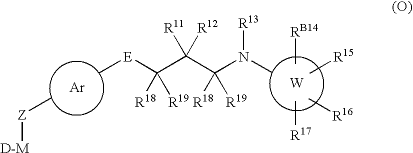
and N-oxides, hydrates, solvates, pharmaceutically acceptable salts, prodrugs and complexes thereof, and racemic and scalemic mixtures, diastereomers and enantiomers thereof, wherein
R¹⁸ and R¹⁹ are each independently selected from the group consisting of H, OH, halogen, NO₂, unsubstituted —O—(C₁-C₆alkyl), substituted —O—(C₁-C₆alkyl), CH₃, CH₂F, CHF₂, CF₃, CN, C₁-C₆alkyl, substituted C₁-C₆alkyl, partially fluorinated C₁-C₆alkyl, per-fluorinated C₁-C₆alkyl, heteroalkyl, substituted heteroalkyl and —SO₂R;
R is a lower alkyl); or
R¹⁸ and R¹⁹ together with the atom to which they are attached form a 3 to 6 membered cycloalkyl or heterocycle, each of which is optionally substituted with 1 to 4 halo, preferably F.

16. A composition comprising a compound according to claim 1 and a pharmaceutically acceptable carrier.

17. A method of inhibiting kinase activity comprising contacting the kinase with a compound according to claim 1.

18. A method of inhibiting cell proliferation, comprising contact the cell with a compound according to claim 1.

19. A method of treating a cell proliferative disease, comprising administering to a patient having a cell proliferative disease a compound according to claim 1.

20. A method of inhibiting kinase activity comprising contacting the kinase with a composition according to claim 16.

21. A method of inhibiting cell proliferation, comprising contact the cell with a composition according to claim 16.

22. A method of treating a cell proliferative disease, comprising administering to a patient having a cell proliferative disease a composition according to claim 16.
